(19) United States
(12) Patent Application Publication
(10) Pub. No.: US 2023/0056135 A1
(43) Pub. Date: Feb. 23, 2023

(54) MONOMER AND MULTIMERIC ANTI-HBV AGENTS

(71) Applicant: Emory University, Atlanta, GA (US)

(72) Inventors: Raymond F. Schinazi, Miami, FL (US); Franck Amblard, Tucker, GA (US); Bryan Cox, Suwanee, GA (US); Che Chen, Decatur, GA (US); Leda Bassit, Smyrna, GA (US)

(73) Assignee: Emory University, Atlanta, GA (US)

(21) Appl. No.: 17/281,940

(22) PCT Filed: Oct. 4, 2019
(86) PCT No.: PCT/US2019/054790

§ 371 Date: Mar. 31, 2021

Related U.S. Application Data
(60) Provisional application No. 62/741,822, filed on Oct. 5, 2018.

(51) Int. Cl. C07D 207/22; C07D 403/12; C07D 487/04; C07D 417/14; C07D 401/14; C07D 249/04; C07D 417/04; C07D 401/04; C07D 471/04; C07D 471/18; C07D 498/18; A61K 45/06

(52) U.S. Cl. CPC C07D 207/22; C07D 403/12; C07D 487/04; C07D 417/14; C07D 401/14; A61K 45/06; C07D 417/04; C07D 401/04; C07D 471/04; C07D 471/18; C07D 498/18; C07D 249/04

(57) ABSTRACT

The present invention is directed to compounds, compositions and methods for preventing, treating or curing hepatitis B (HBV) infection in human subjects or other animal hosts. The compounds are as also pharmaceutically acceptable, salts, prodrugs, and other derivatives thereof as pharmaceutical compositions and methods for treatment, prevention or eradication of HBV infection.

### CROSS REFERENCE TO RELATED APPLICATIONS

This application is a 35 U.S.C. § 371 U.S. national phase entry of International Application No. PCT/US2019/054790, filed Oct. 4, 2019, which is related to and claims priority to U.S. Provisional Application No. 62/741,822, filed on Oct. 5, 2018; the entire disclosures of which are incorporated herein by reference.

### STATEMENT REGARDING FEDERALLY FUNDED RESEARCH

This invention was made with government support under AI132833 awarded by the National Institutes of Health. The government has certain rights in the invention.

### FIELD OF THE INVENTION

The present invention is directed to compounds, methods and compositions for preventing, treating and/or curing hepatitis B virus (HBV) infections. More specifically, the invention describes specifically substituted aromatic/heteroaromatic compounds, pharmaceutically acceptable salts, or other derivatives thereof, and the use thereof in the treatment or curing of HBV infections as monomer or as linked multimeric agents.

### BACKGROUND OF THE INVENTION

Hepatitis B virus (HBV) causes a serious human health problem and is second only to tobacco as a cause of human cancer. The mechanism by which HBV induces cancer is unknown. It is postulated that it may directly trigger tumor development, or indirectly trigger tumor development through chronic inflammation, cirrhosis, and cell regeneration associated with the infection.

After a 2- to 6-month incubation period, during which the host is typically unaware of the infection, HBV infection can lead to acute hepatitis and liver damage, resulting in abdominal pain, jaundice and elevated blood levels of certain enzymes. HBV can cause fulminant hepatitis, a rapidly progressive, often fatal form of the disease in which large sections of the liver are destroyed.

Subjects typically recover from the acute phase of HBV infection. In some persons, however, the virus continues replication for an extended or indefinite period, causing a chronic infection. Chronic infections can lead to chronic persistent hepatitis. Subjects infected with chronic persistent HBV are most common in developing countries. By mid-1991, there were approximately 225 million chronic carriers of HBV in Asia alone and worldwide almost 300 million carriers. Chronic persistent hepatitis can cause fatigue, cirrhosis of the liver, and hepatocellular carcinoma, a primary liver cancer.

In industrialized countries, the high-risk group for HBV infection includes those in contact with HBV carriers or their blood samples. The epidemiology of HBV is very similar to that of HIV/AIDS, which is a reason why HBV infection is common among persons infected with HIV or suffering from AIDS. However, HBV is much more contagious than HIV. 3TC (lamivudine), interferon alpha-2b, peginterferon alpha-2a, hepsera (adefovir dipivoxil), baraclude (entecavir), Tyzeka (Telbivudine), and Viread (Tenofovir disoproxil fumarate) are currently FDA-approved drugs for treating HBV infection. Another nucleoside, tenofovir alafenamide fumarate (TAF) (formerly GS-7340) was also recently approved. All these drugs are highly effective in reducing viral load, but none of them can provide a high remission rate. In addition, their impact can be limited by drug resistance, low efficacy and tolerability issues. The low cure rates of HBV are attributed primarily to the presence and persistence of covalently closed circular DNA (cccDNA) in the nucleus of infected hepatocytes.

Accordingly, there is an urgent need for new HBV drugs that are potent, safe, that work by a different mechanism than nucleoside analogs, and can reduce the latent form of HBV known as cccDNA.

It would be advantageous to provide new antiviral agents, compositions including these agents, and methods of treatment using these agents to treat HBV and prevent the emergence of drug-resistant HBV. The present invention provides such agents, compositions and methods.

### SUMMARY OF THE INVENTION

The present invention provides compounds, methods and compositions for preventing, treating and/or curing HBV infection in a host, or reducing the activity of HBV in the host. The methods involve administering a therapeutically or prophylactically-effective amount of at least one compound as described herein to treat, cure or prevent an infection by, or an amount sufficient to reduce the biological activity of, an HBV infection.

The pharmaceutical compositions include one or more of the compounds described herein, in combination with a pharmaceutically acceptable carrier or excipient, for treating a host infected with HBV. These compounds can be used in combination with nucleoside and non-nucleoside inhibitors of HBV. The formulations can further include at least one other therapeutic agent. In addition, the present invention includes processes for preparing such compounds.

Also disclosed are pharmaceutical compositions that include one or more of the compounds of Formulas 1-10, and a pharmaceutically-acceptable carrier. The carrier can be, for example, an oral composition, an injectable composition, a transdermal composition, or a nanoparticulate composition. The compositions can further include a second antiviral agent, particularly where the agent is active against HBV infection, and more particularly where the second antiviral agent is active against HBV infection via a different mechanism than the instantly-described compounds.

Representative types of second antiviral agents include polymerase inhibitors, viral entry inhibitors, viral maturation inhibitors, capsid assembly modulators, IMPDH inhibitors, protease inhibitors, immune-based therapeutic agents, reverse transcriptase inhibitors, TLR-agonists, and agents of distinct or unknown mechanism. Combinations of these agents can be used.

The compounds described herein can be used to prepare medicaments for treating HBV infection, preventing an HBV infection, or reducing the biological activity of an infection with HBV. The medicaments can further include another anti-HBV agent.

The compounds and compositions can be used in methods for treating a host infected with HBV, preventing an infection from a HBV, and reducing the biological activity of an infection with HBV in a host. The methods can also involve the co-administration of another anti-HBV agent, which co-administration can be simultaneous or sequential.

These and other aspects of the invention are further explained in the following detailed description.

### DETAILED DESCRIPTION

Compounds and compositions useful in treating, preventing, or curing HBV infection are disclosed. Methods for treating, preventing, or curing HBV infection are also disclosed.

The compounds described herein show inhibitory activity against HBV in cell-based assays. Therefore, the compounds can be used to treat or prevent HBV in a host, or reduce the biological activity of the virus. The host can be a mammal, and in particular, a human, infected with HBV. The methods involve administering an effective amount of one or more of the compounds described herein.

Pharmaceutical formulations including one or more compounds described herein, in combination with a pharmaceutically acceptable carrier or excipient, are also disclosed. In one embodiment, the formulations include at least one compound described herein and at least one further therapeutic agent.

The present invention will be better understood with reference to the following definitions:

### I. Definitions

Hepatitis B Virus (HBV) capsid assembly is a critical step in virus production. As used herein, a “HBV capsid assembly effector” is a compound which inhibits assembly of the HBV capsid. In some embodiments, these compounds target and misdirect capsid assembly, and/or disrupt pre-formed capsids, resulting in the suppression of HBV replication and virion production.

As used herein, the phrase “inhibition of assembly” of an HBV capsid assembly effector includes, but is not limited to, modulation of the rate of capsid assembly, stabilizing or destabilizing assembly, and disrupting pre-formed capsids.

Small-molecule compounds targeting HBV core, or capsid assembly effectors, can be grouped into two main classes according to their effect on assembly: the phenylpropenamide and sulfamoylbenzamide chemical series accelerate formation of capsid-like particles (Campagna et al., “Sulfamoylbenzamide derivatives inhibit the assembly of hepatitis B virus nucleocapsids,” J Virol 87:6931-6942 (2013); Katen et al., “Trapping of hepatitis B virus capsid assembly intermediates by phenylpropenamide assembly accelerators. ACS Chem Biol 5:1125-1136 (2010), while members of the heteroaryldihydropyrimidine (HAP) family of compounds induce formation of aggregated and aberrant capsid structures (Stray and Zlotnick, “BAY 41-4109 has multiple effects on hepatitis B virus capsid assembly,” J Mol Recognit 19:542-548 (2006), Wang et al., “In vitro inhibition of HBV replication by a novel compound, GLS4, and its efficacy against adefovir-dipivoxil-resistant HBV mutations. Antivir Ther 17:793-803 (2012).

Representative examples include AB-506 (Arbutus Biopharma), JNJ-379 (also known as JNJ-56136379, a compound which binds to the HBV core protein, disrupts early and late-stage processes in the virus life cycle, prevents the encapsidation of RNA, and blocks hepatitis B virus replication), AT-61, AT-130, Bay 41-4109, NVR 3-778, GLS4, HAP-12, bis-ANS, and RO7049389 (a core allosteric modulator (Roche)).

Additional HBV capsid assembly inhibitors are described, for example, in U.S. Publication No. 20180000824 identifies the following HBV capsid assembly inhibitors: 3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid; 3-[(8aS)-7-[[(4S)-5-ethoxycarbonyl-4-(3-fluoro-2-methyl-phenyl)-2-thiazol-2-yl-1,4-dihyd-ropyrimidin-6-yl]methyl]-3-oxo-5,6,8,8a-tetrahydro-1H-imidazo[1,5-a]pyrazin-2-yl]-2,2-dimethyl-propanoic acid; 2-[(1R,3S,5S)-8-[[(4R)-4-(2-chloro-3-fluoro-phenyl)-5-methoxycarbonyl-2-thiazol-2-yl-1,4-dihydropyrimidin-6-yl]methyl]-6,6-difluoro-8-azabicyclo[3.2.1]octan-3-yl]-acetic acid, 2-[(1S,3R,5R)-8-[[(4R)-4-(2-chloro-3-fluoro-phenyl)-5-methoxycarbonyl-2-thiazol-2-yl-1,4-dihydropyrimidin-6-yl]methyl]-6,6-difluoro-8-azabicyclo[3.2.1]-octan-3-yl]acetic acid; or (S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid; or pharmaceutically acceptable salt, enantiomer or diastereomer thereof. It also identifies compounds using the following formula:

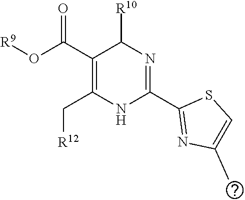

wherein R⁹ is C₁₋₆ alkyl; R¹⁰ is phenyl, which is once or twice or three times substituted by halogen or C₁₋₆ alkyl; R¹¹ is hydrogen or C₁₋₆ alkyl; R¹² is monocyclic, bicyclic fused or bicyclic bridged heterocyclyl; or pharmaceutically acceptable salt, enantiomer or diastereomer thereof.

Another representive HBV capsid inhibitor, disclosed in Wang et al., Antimicrob. Agents Chemother. November 2015 vol. 59, no. 11, 7061-7072 has the following structure:

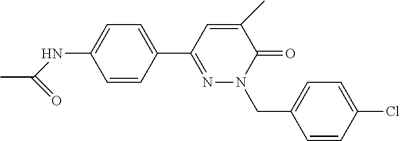 [SMILES: CC(=O)Nc1ccc(-c2cc(C)c(=O)n(Cc3ccc(Cl)cc3)n2)cc1]

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a compound composition that comprises at least approximately 95%, and, preferably, approximately 97%, 98%, 99% or 100% of a single enantiomer of that compound.

As used herein, the term “substantially free of” or “substantially in the absence of” refers to a compound composition that includes at least 85 to 90% by weight, preferably 95% to 98% by weight, and, even more preferably, 99% to 100% by weight, of the designated enantiomer of that compound. In a preferred embodiment, the compounds described herein are substantially free of enantiomers.

Similarly, the term “isolated” refers to a compound composition that includes at least 85 to 90% by weight, preferably 95% to 98% by weight, and, even more preferably, 99% to 100% by weight, of the compound, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphonate, either unprotected, or protected as necessary, as known to those skilled in the art, for example, as taught in Greene, et al., Protective Groups in Organic Synthesis. John Wiley and Sons, Second Edition, 1991, hereby incorporated by reference. Specifically included are CF₃ and CH₂CF₃.

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

As used herein, a “bridged alkyl” refers to a bicyclo- or tricyclo alkane, for example, a 2:1:1 bicyclohexane.

As used herein, a “spiro alkyl” refers to two rings that are attached at a single (quaternary) carbon atom.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moieties. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from the group consisting of straight, branched, or cyclic alkyl or lower alkyl, alkoxyalkyl, including, but not limited to methoxymethyl, aralkyl, including, but not limited to, benzyl, aryloxyalkyl, such as phenoxymethyl, aryl, including, but not limited to, phenyl, optionally substituted with halogen (F, Cl, Br, or I), alkyl (including but not limited to C₁; C₂, C₃, and C₄) or alkoxy (including but not limited to C₁; C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The term “aliphatic” refers to hydrocarbons which are not aromatic, including those having an open chain structure, such as alkanes, alkenes, and alkynes, ideally those with from 1-10 carbons, and cyclic hydrocarbons, ideally those with from 3-10 carbons.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituents selected from the group consisting of halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds of the present invention. The term host specifically refers to infected cells, cells transfected with all or part of the viral genome and animals, in particular, primates (including but not limited to chimpanzees) and humans. In most animal applications of the present invention, the host is a human being. Veterinary applications, in certain indications, however, are clearly contemplated by the present invention (such as for use in treating chimpanzees).

The term “peptide” refers to a natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester) compound which, upon administration to a patient, provides the compound. Pharmaceutically-acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

The term “pharmaceutically acceptable salt or prodrug” is used throughout the specification to describe any pharmaceutically acceptable form (such as an ester) compound which, upon administration to a patient, provides the compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, sulfated, desulfated, phosphorylated, or dephosphorylated to produce the active compound. The prodrug forms of the compounds of this invention can possess antiviral activity, can be metabolized to form a compound that exhibits such activity, or both.

### II. Active Compounds

HBV is an enveloped, partially double-stranded DNA (dsDNA) virus of the Hepadnavirus family (Hepadnaviridae). Its genome contains 4 overlapping reading frames: the precore/core gene; the polymerase gene; the L, M, and S genes, which encode for the 3 envelope proteins; and the X gene.

Upon infection, the partially double-stranded DNA genome (the relaxed circular DNA; rcDNA) is converted to a covalently closed circular DNA (cccDNA) in the nucleus of the host cell, and the viral mRNAs are transcribed. Once encapsidated, the pregenomic RNA (pgRNA), which also codes for core protein and Pol, serves as the template for reverse transcription, which regenerates the partially dsDNA genome (rcDNA) in the nucleocapsid.

Following hepatitis B infections, cccDNA can remain following clinical treatment in liver cells, and can reactivate. The relative quantity of cccDNA present is an indicator for HBV treatment (Bourne, et al., (January 2007). “Quantitative analysis of HBV cccDNA from clinical specimens: correlation with clinical and virological response during antiviral therapy”. Journal of Viral Hepatitis 14 (1): 56-63).

A capsid is the protein shell of a virus, and includes oligomeric structural subunits made of proteins called protomers. The observable 3-dimensional morphological subunits, which may or may not correspond to individual proteins, are called capsomeres. The capsid encloses the genetic material of the virus.

In vivo, HBV capsids assemble around an RNA-reverse transcriptase complex. Assembly of the capsid is required for reverse transcription of the RNA pregenome to the mature DNA form. In HBV, the dominant form of capsid is composed of 120 copies of the capsid protein dimer. Even modest mutations of the capsid protein can have dramatic effects on the viability of progeny virus.

Most of the compounds described herein are active as capsid inhibitors. Inhibiting capsid assembly, for example, by disrupting or adversely affecting capsid assembly, can reduce cccDNA, the main reservoir for HBV, and can also decrease the levels of HBV DNA, HBeAg and HBsAg. Compounds which do not necessarily inhibit formation of the capsid, but rather, modulate or affect the formation of capsids, are also known as capsid assembly modulators or capsid assembly effectors, and their use in forming the dimers and trimers described herein is intended to be within the scope of the invention.

In one embodiment, the compounds are dimers or trimers of the formulas:

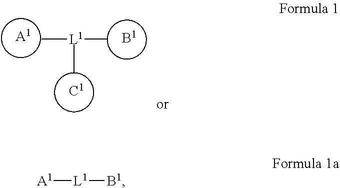

or an enantiomer, a diasteroisomer, a tautomer, a pharmaceutically acceptable salt or prodrug thereof, wherein:

A¹, B¹ and C¹ are, independently, an HBV capsid assembly effector,

each A¹, and B¹, and C¹ may be the same or different;

L¹ is a chemical linker selected from the group consisting of —W¹—(C₃₋₆ alkyl-O)ₛ—C₁₋₆ alkyl-W¹—; —W¹—(CH₂—CH₂—O)ₜ—C₁₋₆ alkyl-W¹—; —W¹—((CH₂—CH₂—O)ₛ)ₘ—; W¹—C₁₋₃₀ alkyl-W¹—; W¹—(C₃₋₆ alkyl-O)ₛ—C₁₋₆ alkyl-X—(C₃₋₆ alkyl-O)ₛ—C₁₋₆ alkyl-W¹; —W¹—(CH₂—CH₂—O)ₜ—C₁₋₆ alkyl-X—(CH₂—CH₂—O)ₜ—C₁₋₆ alkyl-W¹—, W¹—C₁₋₃₀ alkyl-W¹—;

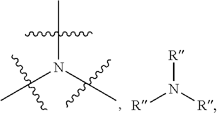

NR′—; —N(R′)C(O)—; —C(O)N(R′)—; —N(R′)SO₂—; —SO₂N(R′); —O—; —C(O)—; —OC(O)—; —C(O)O—; —S—; —S(O)—; —SO₂—; —C(═S)—; —C(═NR′); —Si(R⁷)(R⁸)—, —Si(R⁷)(R⁸)—O—, —O—Si(R⁷)(R⁸)—, —NR′—; —N(R′)C(O)—; —C(O)N(R′)—; —N(R′)SO₂—; —SO₂N(R′); —O—; —C(O)—; —OC(O)—; —C(O)O—; —S—; —S(O)—; —SO₂—; —C(═S)—; —C(═NR′)—; an optionally substituted unsaturated or partially saturated aliphatic moiety, an optionally substituted unsaturated or partially saturated cycloalkyl moiety; an optionally substituted unsaturated or partially saturated heteroaliphatic moiety; an optionally substituted unsaturated or partially saturated heterocycloalkyl moiety; optionally substituted aryl, optionally substituted heteroaryl, and a covalently bonded combination thereof;

wherein X is Si(R⁷)(R⁸)—, —Si(R⁷)(R⁸)—O—, —O—Si(R⁷)(R⁸)—, —NR′—; —N(R′)C(O)—; —C(O)N(R′)—; —N(R′)SO₂—; —SO₂N(R′); —O—; —C(O)—; —OC(O)—; —C(O)O—; —S—; —S(O)—; —SO₂—; —C(═S)—; or —C(═NR′)—;

wherein R″ is, independently, H, alkylene, arylene, —C(O)—, —(C₃₋₆ alkyl-O)ₛ—C₁₋₆ alkyl-, —(CH₂—CH₂—O)ₛ—; C(O)N(R′)—; —SO₂N(R′)—; —C(O)—; —OC(O)—; —C(O)O—; —S(O)—; —SO₂—; —C(═S)—; —C(═NR′)—; or an optionally substituted unsaturated or partially saturated aliphatic moiety or an optionally substituted unsaturated or partially saturated heteroaliphatic moiety; wherein only one R″ can be H,

wherein the optional substituents for the heteroaliphatic, aliphatic, aryl, and heteroaryl moieties are selected from the group consisting of halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, phosphonyl, phosphinyl, phosphoryl, phosphine, thioester, thioether, oxime, hydrazine, carbamate, silyl ether, boronic ester, boronic acid, phosphonic acid, phosphonamidate, and phosphonate,

W¹ is, independently for each occurrence, aryl, heteroaryl, CHR′, NR′, N, O or S;

s and m are integers from 1 to 15;

t is an integer from 3 to 15;

R′ is H, C₁₋₆ alkyl, optionally unsaturated C₃₋₈ cycloalkyl, C₁₋₆ haloalkyl, C₂₋₈ alkoxyalkyl, C₂₋₆ alkenyl, C₂₋₆ alkynyl, aryl, heteroaryl, alkylaryl, or arylalkyl,

R⁷ and R⁸ are, independently, —OH, optionally unsaturated —O—C₁₋₆ alkyl, C₁₋₆ alkyl, C₂₋₆ alkenyl, C₂₋₆ alkynyl, optionally unsaturated C₃₋₈ cycloalkyl, aryl, heteroaryl, alkylaryl, or arylalkyl, and

alternatively, R⁷ and R⁸, together with the silicon to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, cyano, oxo and OH;

or an enantiomer, diasteroisomer, tautomer, pharmaceutically acceptable salt or prodrug thereof.

In another embodiment, the compound has the following formula:

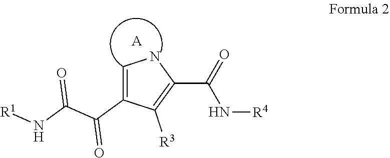

or an enantiomer, a diasteroisomer, a tautomer, a pharmaceutically acceptable salt or prodrug thereof,

wherein:

R¹ is C₂₋₆ alkenyl optionally substituted with one or more R^{a} or C₂₋₆ alkynyl optionally substituted with one or more R^{a},

R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —OR′, —C(O)OR′, oxo, —SF₅,

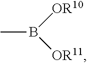

N(R′)S(O)₂R′, —S—R′; S(O)₂R′, S(O)₂N(R′)₂,

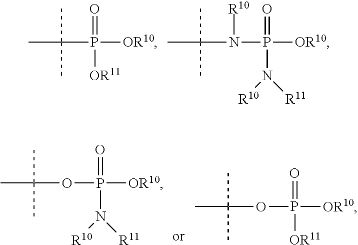

R^{b} is independently C₁₋₆ alkyl optionally substituted with cyano N₃, —O—R′, —C(O)O—R′, oxo, —SF₅,

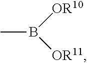

N(R′)S(O)₂R′, —S—R′, S(O)₂R′, S(O)₂N(R′)₂,

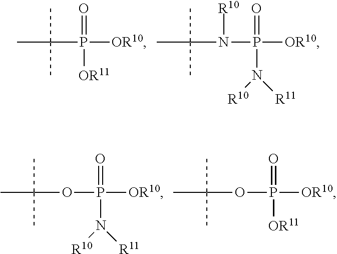

or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;

R^{d} is, independently, cyclopropanyl, alkylamino carbonyl, amino carbonyl, alkyl carbonyl, cyclopropanyloxy, halocyclopropanyl, or halocyclopropanyloxy;

A is a pyrrolidine or a 5-7 membered bicyclic heterocycle having one nitrogen, optionally substituted with one or more R²;

R² is, independently, hydrogen, halogen, OH, C₁₋₆ alkyl, C₂₋₆ alkene, C₂₋₆ alkyne, haloalkyl, cycloalkyl, CN, amino, alkoxy, alkylsulfonylamino, azido or cycloalkyloxyl;

R³ is hydrogen, halogen, C₁₋₆ alkyl, haloalkyl, alkyloxyl, haloalkyloxyl or cycloalkyl,

R⁴ is an C₆₋₁₀ aryl optionally substituted with 1 to 5 R^{4a} or a 5-12 membered heteroaryl having one or more heteroatoms selected from the group consisting of N, O, and S, optionally substituted with one or more R^{4b};

R^{4a} and R^{4b} are, independently, H, CN, halogen, —O—C₁₋₆ alkyl, C₁₋₆ alkyl, C₂₋₆ alkenyl, C₂₋₆ alkynyl, C₃₋₆ cycloalkyl, alkylamino carbonyl, amino carbonyl, alkyl carbonyl, cyclopropanyloxy, halocyclopropanyl, or halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms; and

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

In another embodiment, the compound has the same general formula as Formula 2 shown above, or is an enantiomer, a diasteroisomer, a tautomer, a pharmaceutically acceptable salt or prodrug thereof, but the variables are defined as follows:

R¹ is C₁₋₆ alkyl substituted with one or more R^{a}, C₃₋₆ cycloalkyl substituted with one or more R^{b}, or monocyclic or bicyclic heterocyclyl containing one or more heteroatoms selected from the group consisting of N, O, and S, optionally substituted with one or more R^{c};

R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —OR′, —C(O)O—R′, oxo, —SF₅,

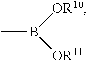

N(R′)S(O)₂R′, —S—R′; S(O)₂R′, S(O)₂N(R′)₂,

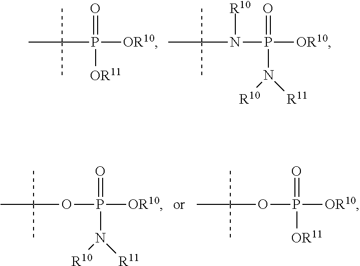

R^{b} is independently C₁₋₆ alkyl optionally substituted with cyano, N₃, —O—R′, —C(O)O—R′, oxo, —SF₅,

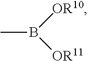

N(R′)S(O)₂R′, —S—R′, S(O)₂R′, S(O)₂N(R′)₂,

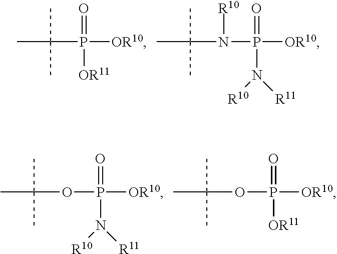

or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;

R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅,

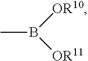

N(R′)S(O)₂R′, —S—R′, S(O)₂R′, S(O)₂N(R′)₂,

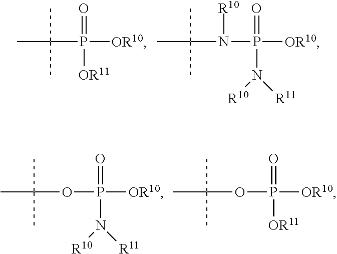

### or —C(O)N(R′)₂,

R^{d} is, independently, cyclopropanyl, alkylamino carbonyl, amino carbonyl, alkyl carbonyl, cyclopropanyloxy, halocyclopropanyl, or halocyclopropanyloxy;

A is a pyrrolidine or a 5-7 membered bicyclic heterocycle having one nitrogen, optionally substituted with one or more R²;

R² is, independently, hydrogen, halogen, OH, C₁₋₆ alkyl, C₂₋₆ alkene, C₂₋₆ alkyne, haloalkyl, cycloalkyl, CN, amino, alkoxy, alkylsulfonylamino, azido or cycloalkyloxyl;

R³ is hydrogen, halogen, C₁₋₆ alkyl, haloalkyl, alkyloxyl, haloalkyloxyl or cycloalkyl,

R⁴ is an C₆₋₁₀ aryl optionally substituted with 1 to 5 R^{4a} or a 5-12 membered heteroaryl having one or more heteroatoms selected from the group consisting of N, O, and S, optionally substituted with one or more R^{4b};

R^{4a} and R^{4b} are, independently, H, CN, halogen, —O—C₁₋₆ alkyl, C₁₋₆ alkyl, C₂₋₆ alkenyl, C₂₋₆ alkynyl, C₃₋₆ cycloalkyl, alkylamino carbonyl, amino carbonyl, alkyl carbonyl, cyclopropanyloxy, halocyclopropanyl, or halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms; and

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

wherein at least one of R², R′, R^{a}, or R^{c} is C₂₋₆ alkenyl or C₂₋₆ alkynyl, or

wherein when R¹ is C₁₋₆ alkyl, R^{a} is not OR¹, or

wherein when R¹ is cycloalkyl, R^{b} is not unsubstituted C₁₋₆ alkyl or unsubstituted heteroaryl.

In one aspect of this embodiment, R³ is haloalkyl, alkyloxyl, haloalkyloxyl, or cycloalkyl.

In another aspect of this embodiment, R¹ is C₁₋₆ alkyl substituted with one or more R^{a}, C₃₋₆ cycloalkyl substituted with one or more R^{b}, or monocyclic or bicyclic heterocyclyl containing one or more heteroatoms selected from the group consisting of N, O, and S, optionally substituted with one or more R^{c};

R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —OR′, —C(O)O—R′, oxo, —SF₅,

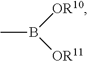

N(R′)S(O)₂R′, —S—R′; S(O)₂R′, S(O)₂N(R′)₂,

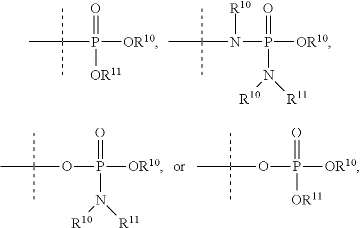

R^{b} is independently C₁₋₆ alkyl optionally substituted with cyano, N₃, —O—R′, —C(O)O—R′, oxo, —SF₅,

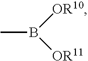

N(R′)S(O)₂R′, —S—R′, S(O)₂R′, S(O)₂N(R′)₂,

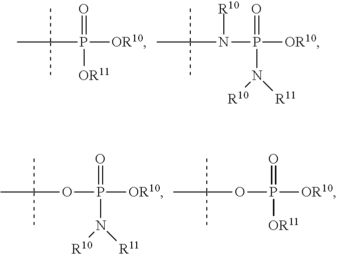

or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;

R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅,

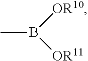

N(R′)S(O)₂R′, —S—R′, S(O)₂R′, S(O)₂N(R′)₂,

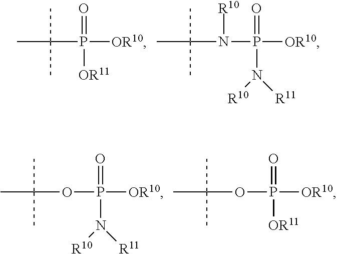

### or —C(O)N(R′)₂,

R^{d} is, independently, cyclopropanyl, alkylamino carbonyl, amino carbonyl, alkyl carbonyl, cyclopropanyloxy, halocyclopropanyl, or halocyclopropanyloxy;

A is a pyrrolidine or a 5-7 membered bicyclic heterocycle having one nitrogen, optionally substituted with one or more R²;

R² is, independently, hydrogen, halogen, OH, C₁₋₆ alkyl, C₂₋₆ alkene, C₂₋₆ alkyne, haloalkyl, cycloalkyl, CN, amino, alkoxy, alkylsulfonylamino, azido or cycloalkyloxyl;

R³ is hydrogen, halogen, C₁₋₆ alkyl, haloalkyl, alkyloxyl, haloalkyloxyl or cycloalkyl,

R⁴ is an C₆₋₁₀ aryl optionally substituted with 1 to 5 R^{4a} or a 5-12 membered heteroaryl having one or more heteroatoms selected from the group consisting of N, O, and S, optionally substituted with one or more R^{4b};

R^{4a} and R^{4b} are, independently, H, CN, halogen, —O—C₁₋₆ alkyl, C₁₋₆ alkyl, C₂₋₆ alkenyl, C₂₋₆ alkynyl, C₃₋₆ cycloalkyl, alkylamino carbonyl, amino carbonyl, alkyl carbonyl, cyclopropanyloxy, halocyclopropanyl, or halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms; and

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

wherein at least one of R², R′, R^{a}, or R^{c} is C₂₋₆ alkenyl or C₂₋₆ alkynyl, or

wherein when R¹ is C₁₋₆ alkyl, R^{a} is not OR¹, or

wherein when R¹ is cycloalkyl, R^{b} is not unsubstituted C₁₋₆ alkyl or unsubstituted heteroaryl.

In one aspect of this embodiment, R³ is haloalkyl, alkyloxyl, haloalkyloxyl, or cycloalkyl.

In another aspect of this embodiment, R¹ is C₁₋₆ alkyl, and R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —C(O)O—R′, oxo, —SF₅,

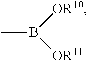

N(R′)S(O)₂R′, —S—R′; S(O)₂R′, S(O)₂N(R′)₂,

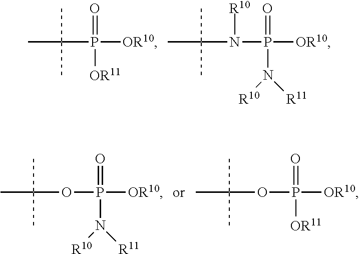

R^{b} is independently C₁₋₆ alkyl optionally substituted with cyano, N₃, —O—R′, —C(O)O—R′, oxo, —SF₅,

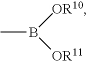

N(R′)S(O)₂R′, —S—R′, S(O)₂R′, S(O)₂N(R′)₂,

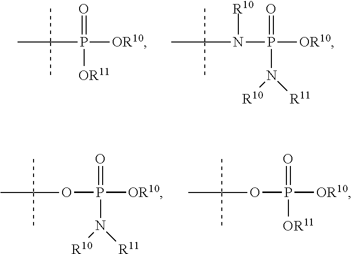

or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl.

In another aspect of this embodiment, R¹ is cycloalkyl, and

R^{b} is C₁₋₆ alkyl substituted with cyano, N₃, —O—R′, —C(O)O—R′, oxo, —SF₅,

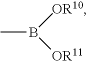

N(R′)S(O)₂R′, —S—R′, S(O)₂R′, S(O)₂N(R′)₂,

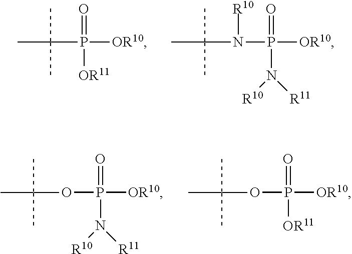

or C₅₋₈ heteroaryl containing one or more heteroatoms selected from the group consisting of N, O, and S, substituted with one or more R^{d}, cycloalkylalkynyl, cycloalkyloxyl, or halocycloalkylalkyl.

In still a further aspect of any of these embodiments, at least one of R², R′, R^{a}, or R^{c} is C₂₋₆ alkenyl or C₂₋₆ alkynyl.

In another embodiment, the compound has the formula:

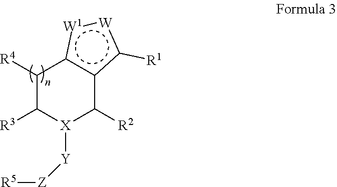

or an enantiomer, a diasteroisomer, a tautomer, a pharmaceutically acceptable salt or prodrug thereof,

wherein:

W¹ and W are each independently selected from the group consisting of N, NR^{m}, CR^{m}, O and S, with the proviso that when one of W and W¹ is O or S, the other is CR^{m}; and wherein when one of W¹ or W is N, the other is NR^{m},

X is N or CRⁿ

Y is a bond, —C(O)— or —SO₂—;

Z is NH or —(CR⁶R⁷)ₘ—NH—;

n is 0, 1, 2 or 3,

m is 0, 1, 2, 3 or 4,

R^{m} and Rⁿ are, independently, H, C₁₋₆ alkyl, C₂₋₆ alkynyl, or C₂₋₆ alkenyl;

R¹ is C₂₋₆ alkynyl, optionally substituted with one or more R^{z},

R^{z} is C₁₋₆ alkyl, C₁₋₆ haloalkyl, C₁₋₆ alkyl-OH, aryl, heteroaryl, C₃₋₆ cycloalkyl, C₃₋₆ halocycloalkyl, C₃₋₆ heterocyclyl, CN, —C(O)R′, —C(O)O—R′,

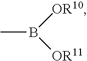

N(R′)S(O)₂R′, —S—R′; S(O)₂R′, S(O)₂N(R′)₂,

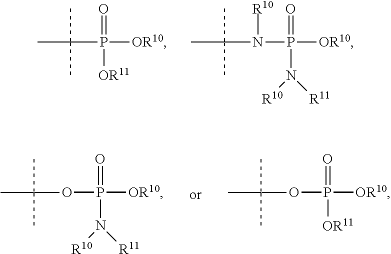

R², R³ and R⁴ are independently selected from the group consisting of H, OH, halo, C₁₋₆ alkyl, C₁₋₆haloalkyl, O—C₁₋₆ alkyl, —O—C₁₋₆haloalkyl, C₂₋₆alkynyl, C₂₋₆alkenyl, C₃₋₆cycloalkyl, C₃₋₆ halocycloalkyl, C₃₋₆ heterocyclyl, —CN, O—C₃₋₆ cycloalkyl, and O—C₃₋₆ halocycloalkyl; alternatively, R² and R³ can come together to form a 5-7 heterocyclic ring containing 1 to 3 heteroatoms selected from the group consisting of N, O and S, alternatively, R² and R⁴ can come together to form a C₃₋₆ cycloalkyl ring or a 3-7 heterocyclic ring containing 1 to 3 heteroatoms selected from the group consisting of N, O and S,

R⁵ is C₁₋₆ alkyl, optionally substituted with one or more R^{a}, C₂₋₆ alkenyl, optionally substituted with one or more R^{a}, C₂₋₆ alkynyl, optionally substituted with one or more R^{a}, C₃₋₆ cycloalkyl optionally substituted with one or more R^{b}, or monocyclic or bicyclic heterocyclyl containing one or more heteroatoms selected from the group consisting of N, O, and S, optionally substituted with one or more R^{c};

R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;

R⁷ is selected from the group consisting of H, C₁-C₆-alkyl, and C₁-C₆-alkyl-OH;

R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —O—R′, —C(O)O—R′, oxo, —SF₅,

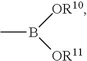

N(R′)S(O)₂R′, —S—R′; S(O)₂R′, S(O)₂N(R′)₂,

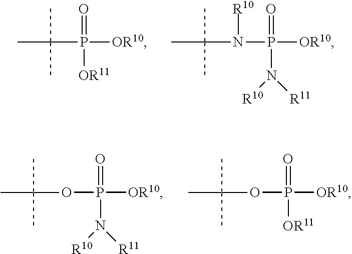

R^{b} is independently C₁₋₆ alkyl optionally substituted with cyano N₃, —O—R′, —C(O)O—R′, oxo, —SF₅,

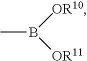

N(R′)S(O)₂R′, —S—R′, S(O)₂R′, S(O)₂N(R′)₂;

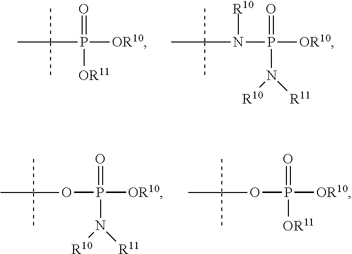

C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;

R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅,

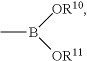

N(R′)S(O)₂R′, —S—R′, S(O)₂R′, S(O)₂N(R′)₂,

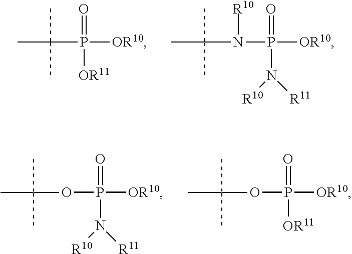

### —C(O)N(R′)₂,

R^{d} is independently cyclopropanyl, alkylamino carbonyl, amino carbonyl, alkyl carbonyl, cyclopropanyloxy, halocyclopropanyl, halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S; alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

In another embodiment, the compound has the same Formula 3 as shown above, or is an enantiomer, a diasteroisomer, a tautomer, a pharmaceutically acceptable salt or prodrug thereof, but the variables are defined as follows:

W¹ and W are each independently selected from the group consisting of N, NR^{m}, CR^{m}, O and S, with the proviso that when one of W and W¹ is O or S, the other is CR^{m}; and wherein when one of W¹ or W is N, the other is NR^{m},

X is N or CRⁿ

Y is a bond, —C(O)— or —SO₂—;

Z is NH or —(CR⁶R⁷)ₘ—NH—;

n is 0, 1, 2 or 3,

m is 0, 1, 2, 3 or 4,

R^{m} and Rⁿ are, independently, H, C₁₋₆ alkyl, C₂₋₆ alkynyl, or C₂₋₆ alkenyl;

R¹ is C₂₋₆ alkenyl substituted with one or more R^{y}, C₃₋₈-cycloalkyl substituted with one or more R^{y}, C₃₋₆ heterocyclyl substituted with one or more R^{y}, or C₁₋₆ alkyl substituted with one or more R^{y},

R^{y} is C₂₋₆ alkynyl, C₂₋₆ alkenyl, aryl, heteroaryl, C₃₋₆ cycloalkyl, C₃₋₆halocycloalkyl, C₃₋₆

heterocyclyl, —CN, —C(O)R′, —C(O)O—R′,

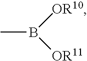

—N(R′)S(O)₂R′, —SR′; —S(O)₂R′, —S(O)₂N(R′)₂,

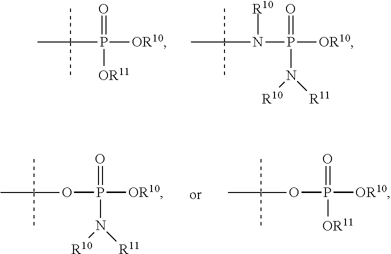

R², R³ and R⁴ are independently selected from the group consisting of H, OH, halo, C₁₋₆ alkyl, C₁₋₆haloalkyl, O—C₁₋₆ alkyl, —O—C₁₋₆haloalkyl, C₂₋₆alkynyl, C₂₋₆alkenyl, C₃₋₆cycloalkyl, C₃₋₆ halocycloalkyl, C₃₋₆ heterocyclyl, —CN, O—C₃₋₆ cycloalkyl, and O—C₃₋₆halocycloalkyl;

alternatively, R² and R³ can come together to form a 5-7 heterocyclic ring containing 1 to 3 heteroatoms selected from the group consisting of N, O and S,

alternatively, R² and R⁴ can come together to form a C₃₋₆ cycloalkyl ring or a 3-7 heterocyclic ring containing 1 to 3 heteroatoms selected from the group consisting of N, O and S,

R⁵ is C₁₋₆ alkyl, optionally substituted with one or more R^{a}, C₂₋₆ alkenyl, optionally substituted with one or more R^{a}, C₂₋₆ alkynyl, optionally substituted with one or more R^{a}, C₃₋₆ cycloalkyl optionally substituted with one or more R^{b}, or monocyclic or bicyclic heterocyclyl containing one or more heteroatoms selected from the group consisting of N, O, and S, optionally substituted with one or more R^{c};

R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;

R⁷ is selected from the group consisting of H, C₁-C₆-alkyl, and C₁-C₆-alkyl-OH;

R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —O—R′, —C(O)O—R′, oxo, —SF₅,

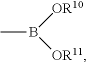

N(R′)S(O)₂R′, —S—R′; S(O)₂R′, S(O)₂N(R′)₂,

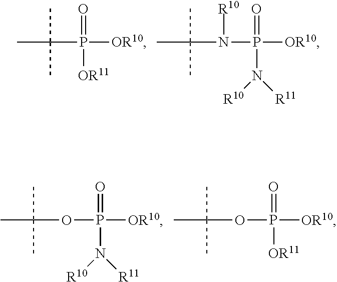

R^{b} is independently C₁₋₆ alkyl optionally substituted with cyano N₃, —O—R′, —C(O)O—R′, oxo, —SF₅,

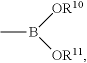

N(R′)S(O)₂R′, —S—R′, S(O)₂R′, S(O)₂N(R′)₂;

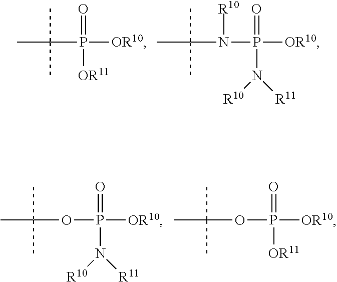

C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;

R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅,

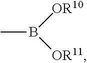

N(R′)S(O)₂R′, —S—R′, S(O)₂R′, S(O)₂N(R′)₂,

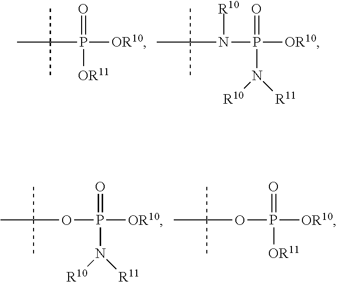

### —C(O)N(R′)₂,

R^{d} is independently cyclopropanyl, alkylamino carbonyl, amino carbonyl, alkyl carbonyl, cyclopropanyloxy, halocyclopropanyl, halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S; alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

In still another embodiment, the compound has the formula:

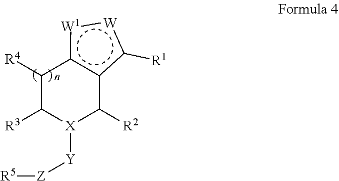

or is an enantiomer, a diasteroisomer, a tautomer, a pharmaceutically acceptable salt or prodrug thereof, wherein:

W¹ and W are each, independently, N, NR^{m}, CR^{m}, O or S; with the proviso that when one of W and W¹ is O or S, the other is CR^{m}, and wherein when one of W¹ or W is N, the other is NR^{m},

X is NR^{m} or CRⁿ,

Y is a bond, —C(O)— or —SO₂—;

Z is NH or —(CR⁶R⁷)ₘ—NH—;

n is 0, 1, 2 or 3,

m is 0, 1, 2, 3 or 4,

R^{m} and Rⁿ are, independently, H, C₁₋₆ alkyl, C₂₋₆ alkynyl, or C₂₋₆ alkenyl;

R², R³ and R⁴ are, independently, —O—C₁₋₆ haloalkyl, C₂₋₆ alkynyl, C₂₋₆ alkenyl, C₃₋₆ cycloalkyl, C₃₋₆ halocycloalkyl, C₃₋₆ heterocyclyl, —CN, —O—C₃₋₆ cycloalkyl, or —O—C₃₋₆ halocycloalkyl;

alternatively, R² and R³ can come together to form a 5-7 heterocyclic ring containing 1 to 3 heteroatoms selected from the group consisting of N, O and S;

alternatively, R² and R⁴ can come together to form a C₃₋₆ cycloalkyl ring or a 3-7 heterocyclic ring containing 1 to 3 heteroatoms selected from the group consisting of N, O and S;

R¹ is C₃₋₆ cycloalkyl, C₃₋₆ heterocyclyl, C₁₋₆ alkyl, halo, C₂₋₆ alkynyl optionally substituted with one or more R^{z}, C₂₋₆ alkenyl optionally substituted with one or more R^{y}, C₃₋₈-cycloalkyl optionally substituted with one or more R^{y}, or C₃₋₆ heterocyclyl, optionally substituted with one or more R^{y}, or C₁₋₆ alkyl optionally substituted with one or more R^{y},

R^{z} is C₁₋₆ alkyl, C₁₋₆ haloalkyl, C₁₋₆ alkyl-OH, C₂₋₆ alkynyl, C₂₋₆ alkenyl, aryl, heteroaryl, C₃₋₆ cycloalkyl, C₃₋₆ halocycloalkyl, C₃₋₆ heterocyclyl, —CN, —C(O)R′, —C(O)O—R′,

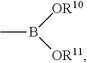

—N(R′)S(O)₂R′, —S—R′; —S(O)₂R′, —S(O)₂N(R′)₂,

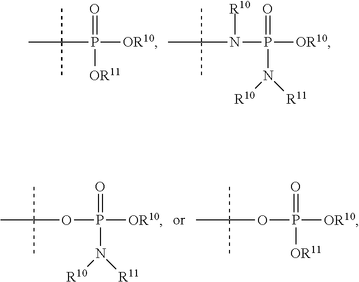

R^{y} is C₁₋₆ alkyl, C₁₋₆haloalkyl, C₁₋₆ alkyl-OH, C₂₋₆ alkynyl, C₂₋₆ alkenyl, aryl, heteroaryl, C₃₋₆ cycloalkyl, C₃₋₆halocycloalkyl, C₃₋₆ heterocyclyl, —CN, —C(O)R′, —C(O)O—R′,

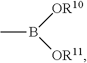

—N(R′)S(O)₂R′, —SR′; —S(O)₂R′, —S(O)₂N(R′)₂,

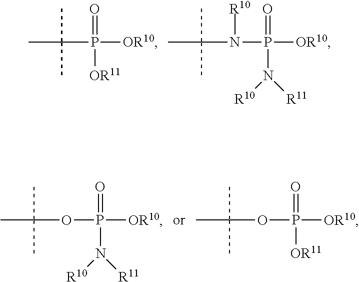

R⁵ is C₁₋₆ alkyl, optionally substituted with one or more R^{a}, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{b}, or a monocyclic or bicyclic heterocyclyl containing one or more heteroatoms selected from the group consisting of N, O, and S, optionally substituted with one or more R^{c};

R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;

R⁷ is selected from the group consisting of H, C₁-C₆-alkyl, and C₁-C₆-alkyl-OH;

R^{a} is C₁₋₆ alkyl, optionally substituted with one or more R′, C₂₋₆ alkenyl, optionally substituted with one or more R′, C₂₋₆ alkynyl, optionally substituted with one or more R′, or C₃₋₆ cycloalkyl, optionally substituted with one or more R^{b}, —N₃, —OR′, —C(O)O—R′, oxo, —SF₅,

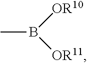

N(R′)S(O)₂R′, —S—R′; S(O)₂R′, S(O)₂N(R′)₂,

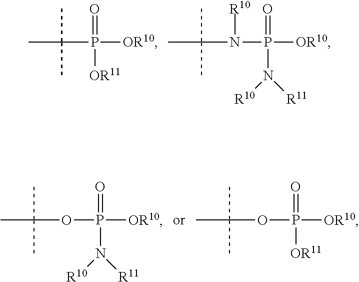

R^{b} is independently C₁₋₆ alkyl, optionally substituted with cyano, —N₃, —OR′, —C(O)O—R′, oxo, —SF₅,

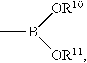

—N(R′)S(O)₂R′, —SR′, —S(O)₂R′, —S(O)₂N(R′)₂,

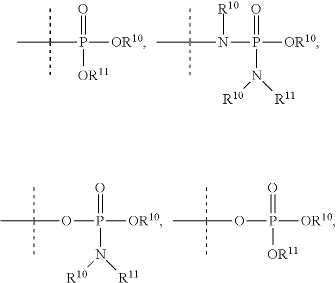

or C₅₋₈ heteroaryl containing one or more heteroatoms selected from the group consisting of N, O, and S, optionally substituted with one or more R^{d}, cycloalkylalkynyl, cycloalkyloxyl, halocycloalkylalkyl;

R^{c} is, independently, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{b}, —N₃, —OR′, —SF₅,

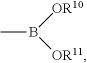

—N(R′)S(O)₂R′, —SR′, S(O)₂R′, —S(O)₂N(R′)₂,

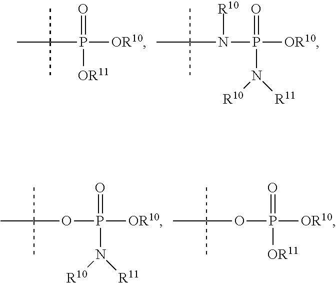

### or —C(O)N(R′)₂,

R^{d} is, independently, cyclopropanyl, alkylamino carbonyl, amino carbonyl, alkyl carbonyl, cyclopropanyloxy, halocyclopropanyl, or halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

In one aspect of this embodiment, at least one of R′, R^{a}, R^{c}, R^{m}, Rⁿ, R^{y}, R^{z}, R¹, R², R³, R⁴, or R⁵ is C₂₋₆ alkynyl.

In yet another embodiment, the compound has the formula:

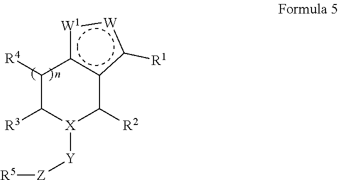

or is an enantiomer, a diasteroisomer, a tautomer, a pharmaceutically acceptable salt or prodrug thereof, wherein:

W¹ and W are each independently N, NR^{m}, CR^{m}, O or S, with the proviso that when one of W and W¹ is O or S, the other is CR^{m}, and with the proviso that when one of W and W¹ is O or S, the other is CR^{m}, and wherein when one of W¹ or W is N, the other is NR^{m},

X is N or CRⁿ

Y is a bond, —C(O)—, or —SO₂—;

Z is NH or —(CR⁶R⁷)ₘ—NH—;

n is 0, 1, 2 or 3,

m is 0, 1, 2, 3 or 4

R^{m} and Rⁿ are, independently, H, C₁₋₆ alkyl, C₂₋₆ alkynyl, or C₂₋₆ alkenyl;

R⁵ is C₁₋₄ alkyl, optionally substituted with one or more R^{b}, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{a}, or monocyclic or bicyclic heterocyclyl containing one or more heteroatoms selected from the group consisting of N, O, and S, optionally substituted with one or more R^{c};

R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;

R⁷ is selected from the group consisting of H, C₁-C₆-alkyl, and C₁-C₆-alkyl-OH;

R^{a} is C₁₋₆ alkyl, optionally substituted with one or more R′, C₂₋₆ alkenyl, optionally substituted with one or more R′, C₂₋₆ alkynyl, optionally substituted with one or more R′, C₃₋₆ cycloalkyl, optionally substituted with one or more R′, N₃, —O—R′, —C(O)O—R′, oxo, —SF₅,

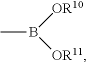

N(R′)S(O)₂R′, —S—R′; S(O)₂R′, S(O)₂N(R′)₂,

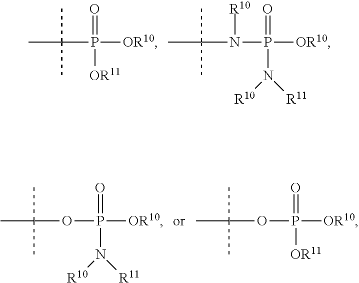

R^{b} is, independently, C₁₋₆ alkyl, optionally substituted with one or more R^{a}, —N₃, —OR′, —C(O)O—R′, oxo, —SF₅,

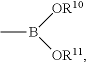

—N(R′)S(O)₂R′, —SR′, —S(O)₂R′, —S(O)₂N(R′)₂;

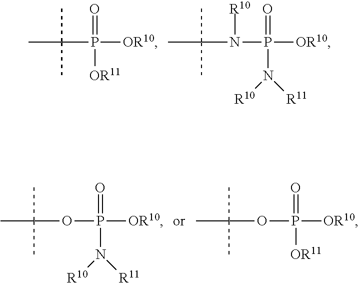

R^{c} is, independently, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{b}, —N₃, —OR′, —SF₅,

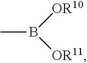

—N(R′)S(O)₂R′, —SR′, S(O)₂R′, —S(O)₂N(R′)₂,

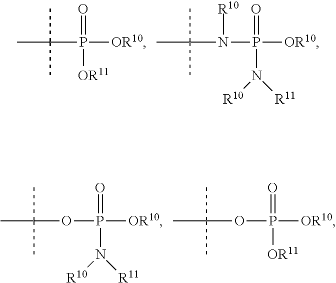

### or —C(O)N(R′)₂,

R^{d} is, independently, cyclopropanyl, alkylamino carbonyl, amino carbonyl, alkyl carbonyl, cyclopropanyloxy, halocyclopropanyl, or halocyclopropanyloxy;

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatoms which are, independently N, O, or S;

alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate;

R¹ is C₁₋₆ alkyl, halo, C₂₋₆ alkynyl optionally substituted with one or more R^{z}, C₂₋₆ alkenyl, optionally substituted with one or more R^{y}, C₃₋₈ cycloalkyl, optionally substituted with one or more R^{y}, C₃₋₆ heterocyclyl, optionally substituted with one or more R^{y}, C₁₋₆ alkyl optionally substituted with one or more R^{y},

R^{z} is C₁₋₆ alkyl, C₁₋₆ haloalkyl, C₁₋₆ hydroxyalkyl, C₂₋₆ alkynyl, C₂₋₆ alkenyl, aryl, heteroaryl, C₃₋₆ cycloalkyl, C₃₋₆halocycloalkyl, C₃₋₆ heterocyclyl, —CN, —C(O)R′, —C(O)O—R′,

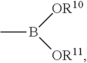

N(R′)S(O)₂R′, —S—R′; S(O)₂R′, S(O)₂N(R′)₂,

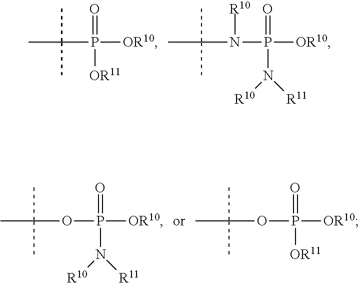

R^{y} is C₁₋₆ alkyl, C₁₋₆ haloalkyl, C₁₋₆ hydroxyalkyl, C₂₋₆ alkynyl, C₂₋₆ alkenyl, aryl, heteroaryl, C₃₋₆ cycloalkyl, C₃₋₆ halocycloalkyl, C₃₋₆ heterocyclyl, CN, —C(O)R′, —C(O)O—R′,

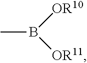

—N(R′)S(O)₂R′, —SR′; S(O)₂R′, —S(O)₂N(R′)₂,

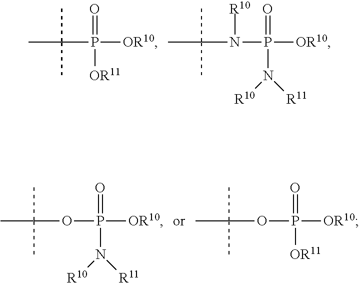

R², R³ and R⁴ are, independently, H, OH, halo, C₁₋₆ alkyl, C₁₋₆haloalkyl, —O—C₁₋₆ alkyl, C₁₋₆ alkyl-OH, —O—C₁₋₆haloalkyl, C₂₋₆alkynyl, C₂₋₆alkenyl, C₃₋₆cycloalkyl, C₃₋₆halocycloalkyl, C₃₋₆ heterocyclyl, —CN, —O—C₃₋₆ cycloalkyl, or —O—C₃₋₆halocycloalkyl;

alternatively, R² and R³ can come together to form a 5-7 heterocyclic ring containing 1 to 3 heteroatoms selected from the group consisting of N, O and S; and

alternatively, R² and R⁴ can come together to form a C₃₋₆ cycloalkyl ring or a 3-7 heterocyclic ring containing 1 to 3 heteroatoms selected from the group consisting of N, O and S,

wherein at least one of R′, R^{a}, R^{c}, R^{m}, Rⁿ, R^{y}, R^{z}, R¹, R², R³, R⁴, or R⁵ is C₂₋₆ alkynyl.

In still another embodiment, the compound has the formula:

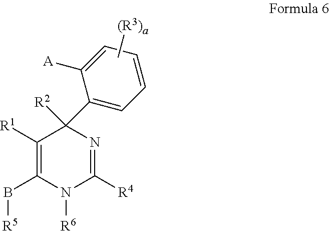

or is an enantiomer, diasteroisomer, tautomer, pharmaceutically acceptable salt or prodrug thereof,

wherein:

A is H, CN, OH, C₁₋₃ alkyl, C₁₋₃ haloalkyl, C₁₋₃ hydroxyalkyl, C₁₋₃ thioalkyl or halogen;

R¹ is

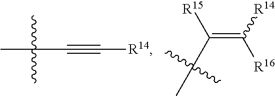

or an optionally substituted C₁₋₆ alkyl;

R² is hydrogen, deuterium or C₁₋₃ alkyl optionally substituted with one or more fluoro atoms;

or R² and A, together with the carbons to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, oxo and OH;

each R³ is, independently, H, —CN, —OH, C₁₋₃ alkyl, C₁₋₃ haloalkyl, C₁₋₃ hydroxy alkyl, C₁₋₃ thioalkyl or halogen;

R⁶ is H, C₁₋₃ alkyl, or C₁₋₃ haloalkyl,

B is —(CR⁷R^{7a})ₙ— or —C(O)—, where n is 0, 1, 2 or 3,

each a is independently 0, 1, 2, 3 or 4;

each R⁷ and R^{7a} is independently H, C₁₋₆ alkyl or C₁₋₆ haloalkyl;

alternatively, R⁷ and R^{7a} can come together with the carbon to which they are attached to form a 6-10 membered bicyclic or bridged ring, a 3 to 8 saturated ring, or a 5 membered unsaturated ring; such bicyclic, bridged, saturated and unsaturated rings optionally containing one or more additional heteroatoms, where each is, independently, O, S or N, and optionally being substituted with one or more substituents, wherein each, independently, is halogen (including F, Cl, Br, I), —CF₃, hydroxy, —N(R′)S(O)₂R′, —S(O)₂R′, —S(O)₂N(R′)₂, C₁₋₆ alkoxy, cyano, azido, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, or C₁₋₆ hydroxyalkyl,

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

R⁴ is an optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

wherein the substituents on the optionally substituted C₁₋₆ alkyl are, independently, halo, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate;

R⁵ is H, —O—R¹⁰,

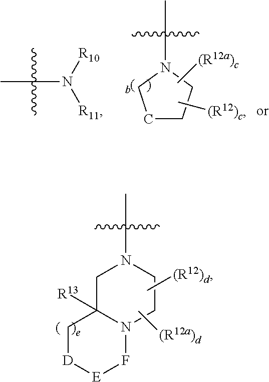

R¹⁰ and R¹¹ are, independently, H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatoms which are, independently, N, O, or S; —COOR′, —(C═O)—R′, —S(O)₂R′, —S(O)₂N(R′)₂, C(═O)NR′₂; —S(═O)₂—NR′₂; or P(═O)(OR′)₂;

R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

R¹² and R^{12a} are, independently, H, F, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(CO)—R¹⁰, —N(R′)S(O)₂R′, —S(O)₂R′, —S(O)₂N(R′)₂, —C(═O)NR¹⁰R¹¹; —NR′—S(O)₂—R¹⁰; —NR′—S(O)₂—NR¹⁰R¹¹; or —NR¹⁰—P(O)(OR¹⁰)₂,

alternatively, R¹² and R^{12a} can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

if R¹² and R^{12a} are on the same carbon, they can come together to form, with the carbon to which they are attached, —C═O;

R¹² and R^{12a} groups can optionally be substituted with one or more substituents, which substituents are, independently, halogen, C₁₋₆ haloalkyl, C₁₋₆ hydroxy alkyl, hydroxyl, carboxyl, acyl, aryl, acyloxy, amino, amido, azido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

b is 0, 1, 2 or 3,

c is independently 0, 1, 2, 3, 4 or 5,

C is O, S, NR¹⁰, —C(O), or —C(R¹²R^{12a})—,

d is 0, 1, 2 or 3,

F is sulfonyl or —C(R¹²R^{12a})—,

D is —C(R¹²R^{12a})— or —O—,

E is N—R¹⁰, —C═O, or —C(R¹²R^{12a})—,

e is 0 or 1,

R¹⁴, R¹⁵ and R¹⁶ are, independently, H, optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(C═O)—R¹⁰, —N(R′)S(O)₂R′, —S(O)₂R′, —S(O)₂N(R′)₂, —C(═O)NR¹⁰R¹¹; —NR′—S(═O)₂—R¹⁰; —NR′—S(═O)₂—NR¹⁰R¹¹; —P(═O)(OR¹⁰)₂, or —NR¹⁰—P(═O)(OR¹⁰)₂,

wherein the optional substituents for the C₁₋₆ alkyl are selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,

R¹⁴, R¹⁵ and R¹⁶ can optionally, and independently, be substituted with one or more substituents selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,

alternatively, R¹⁴ and R¹⁶ can come together to form, with the carbon to which they are attached, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring,

wherein R³ is C₁₋₃ alkyl, C₁₋₃ haloalkyl, C₁₋₃ hydroxy alkyl, or C₁₋₃ thioalkyl, or

wherein R⁴ is C₁₋₆ alkyl, C₁₋₆ haloalkyl, C₂₋₈ alkoxyalkyl, C₂₋₆ alkenyl, or C₂₋₆ alkynyl.

In one aspect of this embodiment, R⁴ is C₁₋₆ alkyl, C₁₋₆ haloalkyl, C₂₋₈ alkoxyalkyl, C₂₋₆ alkenyl, or C₂₋₆ alkynyl, and in another aspect, R⁴ is C₂₋₆ alkynyl.

In another aspect of this embodiment, R³ is C₁₋₃ alkyl, C₁₋₃ haloalkyl, C₁₋₃ hydroxy alkyl, or C₁₋₃ thioalkyl.

In another embodiment, the compound has the formula:

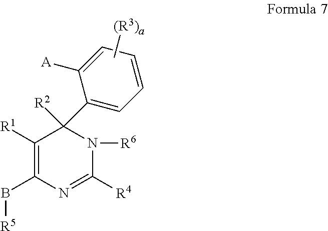

or is an enantiomer, diasteroisomer, tautomer, pharmaceutically acceptable salt or prodrug thereof,

wherein:

A is H, CN, OH, C₁₋₃ alkyl, C₁₋₃ haloalkyl, C₁₋₃ hydroxyalkyl, C₁₋₃ thioalkyl or halogen;

R¹ is

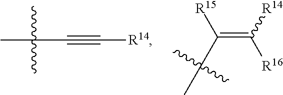

or an optionally substituted C₁₋₆ alkyl;

R² is hydrogen, deuterium or C₁₋₃ alkyl optionally substituted with one or more fluoro atoms;

or R² and A, together with the carbons to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, oxo and OH;

each R³ is, independently, H, —CN, —OH, C₁₋₃ alkyl, C₁₋₃ haloalkyl, C₁₋₃ hydroxy alkyl, C₁₋₃ thioalkyl or halogen;

R⁶ is H, C₁₋₃ alkyl, or C₁₋₃ haloalkyl,

B is —(CR⁷R^{7a})ₙ— or —C(O)—, where n is 0, 1, 2 or 3,

each a is independently 0, 1, 2, 3 or 4;

each R⁷ and R^{7a} is independently H, C₁₋₆ alkyl or C₁₋₆ haloalkyl;

alternatively, R⁷ and R^{7a} can come together with the carbon to which they are attached to form a 6-10 membered bicyclic or bridged ring, a 3 to 8 saturated ring, or a 5 membered unsaturated ring; such bicyclic, bridged, saturated and unsaturated rings optionally containing one or more additional heteroatoms, where each is, independently, O, S or N, and optionally being substituted with one or more substituents, wherein each, independently, is halogen (including F, Cl, Br, I), —CF₃, hydroxy, —N(R′)S(O)₂R′, —S(O)₂R′, —S(O)₂N(R′)₂, C₁₋₆ alkoxy, cyano, azido, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, or C₁₋₆ hydroxyalkyl,

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

R⁴ is an optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

wherein the substituents on the optionally substituted C₁₋₆ alkyl are, independently, halo, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate;

R⁵ is H, —O—R¹⁰,

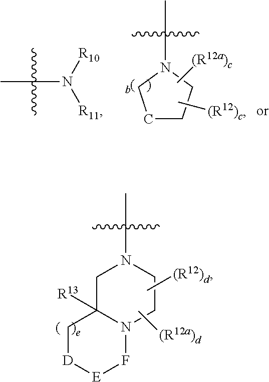

R¹⁰ and R¹¹ are, independently, H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatoms which are, independently, N, O, or S; —COOR′, —(C═O)—R′, —S(O)₂R′, —S(O)₂N(R′)₂, C(═O)NR′₂; —S(═O)₂—NR′₂; or P(═O)(OR′)₂;

R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

R¹² and R^{12a} are, independently, H, F, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(CO)—R¹⁰, —N(R′)S(O)₂R′, —S(O)₂R′, —S(O)₂N(R′)₂, —C(═O)NR¹⁰R¹¹; —NR′—S(O)₂—R¹⁰; —NR′—S(O)₂—NR¹⁰R¹¹; or —NR¹⁰—P(O)(OR¹⁰)₂,

alternatively, R¹² and R^{12a} can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

if R¹² and R^{12a} are on the same carbon, they can come together to form, with the carbon to which they are attached, —C═O;

R¹² and R^{12a} groups can optionally be substituted with one or more substituents, which substituents are, independently, halogen, C₁₋₆ haloalkyl, C₁₋₆ hydroxy alkyl, hydroxyl, carboxyl, acyl, aryl, acyloxy, amino, amido, azido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

b is 0, 1, 2 or 3,

c is independently 0, 1, 2, 3, 4 or 5,

C is O, S, NR¹⁰, —C(O), or —C(R¹²R^{12a})—,

d is 0, 1, 2 or 3,

F is sulfonyl or —C(R¹²R^{12a})—,

D is —C(R¹²R^{12a})— or —O—,

E is N—R¹⁰, —C═O, or —C(R¹²R^{12a})—,

e is 0 or 1,

R¹⁴, R¹⁵ and R¹⁶ are, independently, H, optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(C═O)—R¹⁰, —N(R′)S(O)₂R′, —S(O)₂R′, —S(O)₂N(R′)₂, —C(═O)NR¹⁰R¹¹; —NR′—S(═O)₂—R¹⁰; —NR′—S(═O)₂—NR¹⁰R¹¹; —P(═O)(OR¹⁰)₂, or —NR¹⁰—P(═O)(OR¹⁰)₂,

wherein the optional substituents for the C₁₋₆ alkyl are selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,

R¹⁴, R¹⁵ and R¹⁶ can optionally, and independently, be substituted with one or more substituents selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,

alternatively, R¹⁴ and R¹⁶ can come together to form, with the carbon to which they are attached, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring.

In still another embodiment, the compound has the formula:

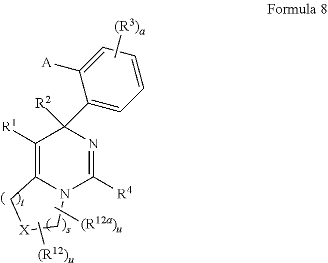

or is an enantiomer, a diasteroisomer, a tautomer, a pharmaceutically acceptable salt or prodrug thereof,

wherein:

A is H, CN, OH, C₁₋₃ alkyl, C₁₋₃ haloalkyl, C₁₋₃ hydroxyalkyl, C₁₋₃ thioalkyl or halogen;

R¹ is

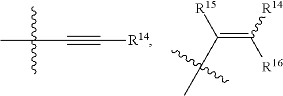

or an optionally substituted C₁₋₆ alkyl;

wherein the substituents on the optionally substituted C₁₋₆ alkyl are selected from the group consisting of halogen, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,

R² is hydrogen, deuterium or C₁₋₃ alkyl optionally substituted with one or more fluoro atoms;

or R² and A, together with the carbons to which they are attached, form a 4-7 membered ring, optionally containing one or more heteroatoms, where each is, independently, O, S or N, wherein the 4-7 membered ring is optionally substituted with one or more substituent selected from the group consisting of C₁-C₃ alkyl, halogen, oxo and OH;

each R³ is, independently, H, CN, OH, C₁₋₃ alkyl, C₁₋₃ haloalkyl, C₁₋₃ hydroxyalkyl, C₁₋₃ thioalkyl or halogen;

each a is independently 0, 1, 2, 3 or 4;

R⁴ is an optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

wherein the substituents on the optionally substituted C₁₋₆ alkyl are selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,

R¹² and R^{12a} are, independently, H, F, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(C═O)—R¹⁰, —N(R′)S(O)₂R′, —S(O)₂R′, —S(O)₂N(R′)₂, —C(═O)NR¹⁰R¹¹; —NR′—S(═O)₂—R¹⁰; —NR′—S(═O)₂—NR¹⁰R¹¹; or —NR¹⁰—P(═O)(OR¹⁰)₂,

R¹⁰ and R¹¹ are, independently, H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatoms which are, independently, N, O, or S; —COOR′, —(CO)—R′, —S(O)₂R′, —S(O)₂N(R′)₂, C(═O)NR′₂; —S(O)₂—NR′₂; or P(O)(OR′)₂

R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

alternatively, R¹² and R^{12a} can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;

if R¹² and R^{12a} are on the same carbon, they can come together to form, with the carbon to which they are attached, —C═O;

R¹² and R^{12a} groups can optionally be substituted with one or more substituents, which substituents are, independently, halogen, C₁₋₆ haloalkyl, C₁₋₆ hydroxy alkyl, hydroxyl, carboxyl, acyl, aryl, acyloxy, amino, amido, azido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,

R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatoms which are, independently, N, O, or S,

s is 0, 1, 2, or 3,

t is 1, 2, 3, or 4,

X is —C(R¹²R^{12a})—, —C(═O)N(R¹⁰)—, —N(R¹⁰)—, —C(═NR¹⁰)—, —S(═O)N(R¹⁰)—, —O—, —S—, —C(═O)O—, —C(═O)—; —C(═S)—; —S(═O)—; or —S(═O)₂—;

u is independently 0, 1, 2, 3, 4, 5, 6, or 7.

In one aspect of this embodiment, R¹ is

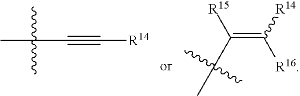

In yet another embodiment, the compounds have the formula:

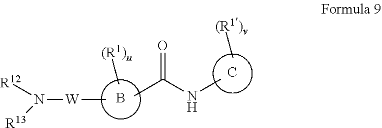

or a pharmaceutically acceptable salt or prodrug thereof, wherein

when R¹ and R^{1′} are attached to a carbon they are, independently, hydrogen, halogen, CF₃, hydroxy, SF₅, N(R′)S(O)₂R′, S(O)₂R′, S(O)₂N(R′)₂, C₁₋₆ alkoxy, cyano, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, or C₁₋₆ hydroxyalkyl;

when R¹ and R^{1′} are attached to a nitrogen they are, independently, hydrogen, C₁₋₆ alkoxy, C₃₋₆ alkoxyalkyl, alkoxycarbonyl, carbonylalkyl, carbonyl aryl, C₁₋₆ alkyl, C₂₋₆ alkynyl, C₂₋₆ alkenyl, heterocyclylalkyl, C₁₋₆ hydroxyalkyl, or S(O)₂R′;

each R′ is independently H, C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, or if two R′ reside on the same nitrogen atom they can come together to form a C₃₋₆ alkyl ring optionally containing a N, O, or S; wherein the R′ groups can be substituted with one or more substituents as defined above, for example, C₁₋₆ hydroxyalkyl, aminoalkyl, and alkoxyalkyl;

u and v are independently 0, 1, 2, 3, 4 or 5;

C is phenyl, a six-membered heteroaromatic ring containing one, two, or three nitrogen atoms, a five-membered heteroaromatic ring containing one, two, or three heteroatoms, which are, independently, N, O, or S, a C₄₋₁₄ bicyclic ring; alkylheteroaryl, or alkylaryl;

B is selected from the group consisting of five-membered heteroaromatic rings containing one, two, or three heteroatoms, which are, independently, N, O, or S, six or seven-membered rings or six or seven-membered bridged or spiro-fused rings containing zero, one, or two heteroatoms which are, independently, N, O, or S, five-membered rings containing zero, one, or two heteroatoms which are, independently, N, O, or S; four-membered rings containing zero, one, or two heteroatoms which are, independently, N, O, or S,

W is

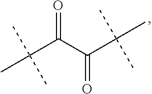

R¹² is H, C₁₋₆ alkyl, C₃₋₇ cycloalkyl, C₁₋₆ haloalkyl, C₂₋₆ alkenyl, or C₂₋₆ alkynyl,

R¹³ is C₇₋₂₀ alkyl, C₇₋₂₀ alkenyl, C₇₋₂₀ alkynyl,

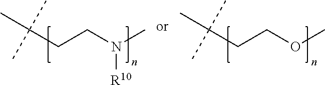

n is 1, 2, 3, 4, 5, 6, 7 or 8,

R¹³ is optionally substituted with one or more substituents each independently selected from the group consisting of hydrogen, halogen (F, Cl, Br, I), CF₃, SF₅, hydroxy,

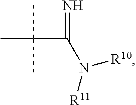

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

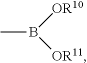

### —S—R¹⁰;

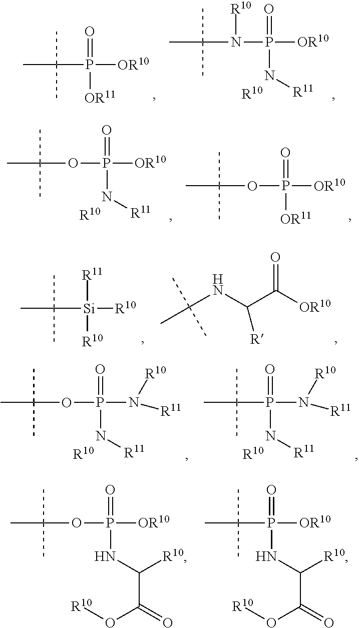

N(R¹⁰)S(O)₂R¹⁰, S(O)₂R¹⁰, S(O)₂N(R¹⁰)₂, C(O)R¹⁰, a saturated or unsaturated five or six or seven-membered ring or a six or seven-membered bridged or spiro-fused ring containing one, or two heteroatoms which are, independently, N, O, S, Se or Si, C₁₋₆ alkoxy, C₁₋₆ haloalkoxy, cyano, azido, C₂₋₆ alkynyl, C₃₋₆ alkoxyalkyl, alkoxycarbonyl, alkoxycarbonylalkyl, C₁₋₆ alkyl, cycloalkyl, arylalkoxycarbonyl, carboxyl, haloalkyl, heterocyclylalkyl, C₁₋₆ hydroxyalkyl, aryl, substituted aryl, heteroaryl, or substituted heteroaryl,

where substituents on the substituted aryl and substituted heteroaryl are selected from the group consisting of halogen, SF₅, CF₃, hydroxy,

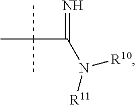

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

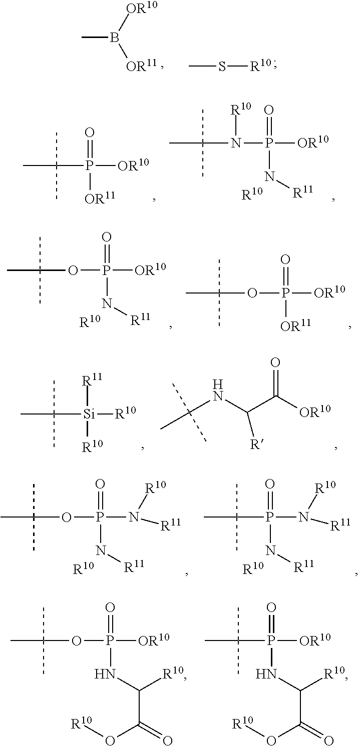

N(R¹⁰)S(O)₂R¹⁰, S(O)₂R¹⁰, S(O)₂N(R¹⁰)₂, C(O)R¹⁰, a saturated or unsaturated five or six or seven-membered ring or a six or seven-membered bridged or spiro-fused ring containing one, or two heteroatoms which are, independently, N, O, S, Se or Si, C₃-C₇ cycloalkyl, C₁₋₆ alkoxy, cyano, azido, C₂₋₆ alkynyl, C₃₋₆ alkoxyalkyl, alkoxycarbonyl, alkoxycarbonylalkyl, and C₁₋₆ alkyl;

or R¹² and R¹³, together with the nitrogen to which they are attached, form a saturated or unsaturated 4 to 7 membered ring optionally substituted with one or more substituents each independently selected from the group consisting of hydrogen, halogen (F, Cl, Br, I), CF₃, hydroxy,

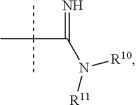

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

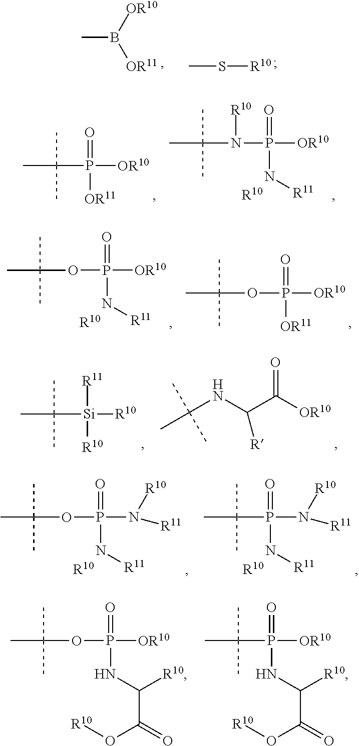

N(R¹⁰)S(O)₂R¹⁰, S(O)₂R¹⁰, S(O)₂N(R¹⁰)₂, C(O)R¹⁰, C₁₋₆ alkoxy, cyano, azido, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, and C₁₋₆ hydroxyalkyl;

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a saturated or partially unsaturated 5 to 8 membered ring or a six-membered bridged or spiro-fused ring containing zero, one, or two heteroatoms which are, independently, N, O, or S, a seven-membered bridged or spiro-fused a saturated or partially unsaturated ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms, a saturated or partially unsaturated 6 to 12 membered hetero-bicyclic ring; a saturated or partially unsaturated 6 to 12 membered hetero-tricyclic ring; and

R¹⁰ and R¹¹ groups can optionally be substituted with one or more substituents, which substituents are, independently, halo, C₁₋₆ alkyl, C₁₋₆ haloalkyl, C₂₋₆ alkenyl, C₂₋₆ alkynyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphoramidate or phosphonate.

In still a further embodiment, the compounds have the following following formula:

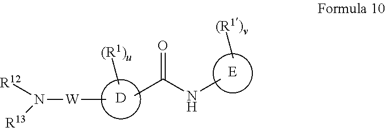

or a pharmaceutically acceptable salt or prodrug thereof, wherein

when R¹ and R^{1′} are attached to a carbon they are, independently, hydrogen, halogen, CF₃, hydroxy, SF₅, N(R′)S(O)₂R′, S(O)₂R′, S(O)₂N(R′)₂, C₁₋₆ alkoxy, cyano, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, or C₁₋₆ hydroxyalkyl;

when R¹ and R^{1′} are attached to a nitrogen they are, independently, hydrogen, C₁₋₆ alkoxy, C₃₋₆ alkoxyalkyl, alkoxycarbonyl, carbonylalkyl, carbonyl aryl, C₁₋₆ alkyl, C₂₋₆ alkynyl, C₂₋₆ alkenyl, heterocyclylalkyl, C₁₋₆ hydroxyalkyl, or S(O)₂R′;

each R′ is independently H, C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, or if two R′ reside on the same nitrogen atom they can come together to form a C₃₋₆ alkyl ring optionally containing a N, O, or S; wherein the R′ groups can be substituted with one or more substituents as defined above, for example, C₁₋₆ hydroxyalkyl, aminoalkyl, and alkoxyalkyl;

u and v are, independently, 0, 1, 2, 3, 4 or 5;

E is phenyl, a six-membered heteroaromatic ring containing one, two, or three nitrogen atoms, a five-membered heteroaromatic ring containing one, two, or three heteroatoms, which are, independently, N, O, or S, a C₄₋₁₄ bicyclic ring; alkylheteroaryl, or alkylaryl;

D is selected from the group consisting of a five-membered heteroaromatic ring containing one, two, or three heteroatoms, which are, independently, N, O, or S, a six or seven-membered ring or a six or seven-membered bridged or spiro-fused ring containing zero, one, or two heteroatoms which are, independently, N, O, or S, a five-membered ring containing zero, one, or two heteroatoms which are, independently, N, O, or S; and a four-membered ring containing zero, one, or two heteroatoms which are, independently, N, O, or S,

W is

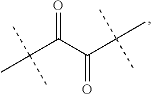

R¹² is H, C₁₋₆ alkyl, C₁₋₆ haloalkyl, C₂₋₆ alkenyl, or C₂₋₆ alkynyl,

R¹³ is C₁₋₆ alkyl, C₂₋₆ alkenyl, C₂₋₆ alkynyl, aryl, heteroaryl, alkylaryl, arylalkyl, a C₄₋₁₄ bicyclic ring; a six-membered bridged or spiro-fused ring containing zero, one, or two heteroatoms which are, independently, N, O, S, or a 3-7 membered saturated or partially unsaturated ring containing one or more heteroatoms each independently selected from the group of N, O or S,

R¹³ is optionally substituted with one or more substituents each independently selected from the group consisting of

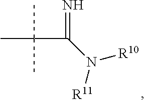

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

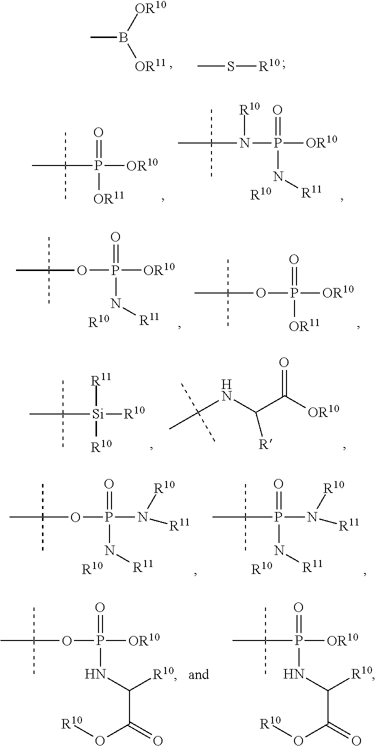

where substituents on the substituted aryl and substituted heteroaryl are selected from the group consisting of

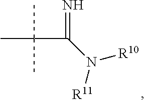

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

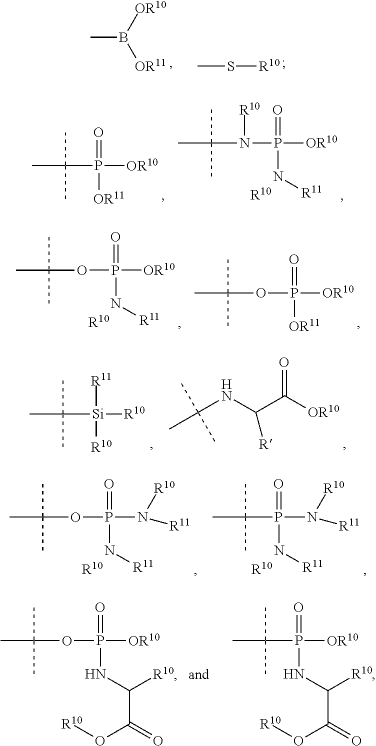

or R¹² and R¹³ together with the nitrogen to which they are attached form a saturated or unsaturated 4 to 7 membered ring optionally substituted with one or more substituents each independently selected from the group consisting of

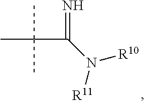

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

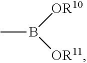

### —S—R¹⁰;

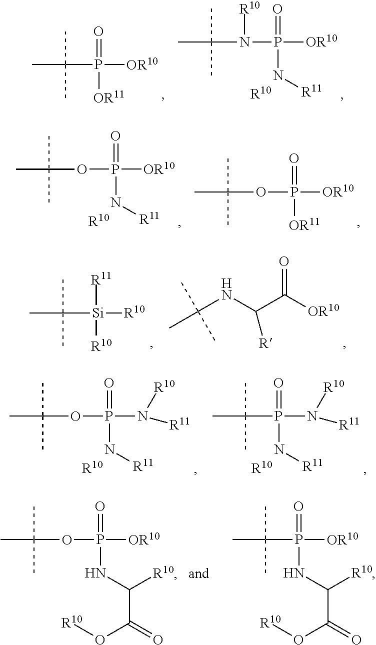

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a saturated or partially unsaturated 5 to 8 membered ring or a six-membered bridged or spiro-fused ring containing zero, one, or two heteroatoms which are, independently, N, O, or S, a seven-membered bridged or spiro-fused a saturated or partially unsaturated ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms, a saturated or partially unsaturated 6 to 12 membered hetero-bicyclic ring; or a saturated or partially unsaturated 6 to 12 membered hetero-tricyclic ring; and

R¹⁰ and R¹¹ groups can optionally be substituted with one or more substituents, which substituents are, independently, halo, C₁₋₆ alkyl, C₃₋₆ alkyl, C₁₋₆ haloalkyl, C₂₋₆ alkenyl, C₂₋₆ alkynyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphoramidate or phosphonate.

In still a further embodiment, the compounds have the following formula:

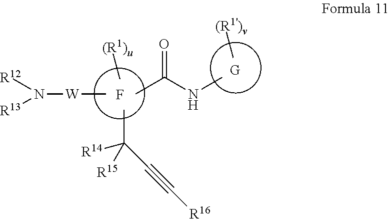

or a pharmaceutically acceptable salt or prodrug thereof, wherein

when R¹ and R^{1′} are attached to a carbon they are, independently, hydrogen, halogen, CF₃, hydroxy, SF₅, N(R′)S(O)₂R′, S(O)₂R′, S(O)₂N(R′)₂, C₁₋₆ alkoxy, cyano, C₂₋₁₀ alkynyl, C₃₋₆ alkoxyalkyl, alkoxycarbonyl, alkoxycarbonylalkyl, C₁₋₆ alkyl, C₁₋₆ haloalkyl, arylalkoxycarbonyl, carboxy, C₁₋₆ haloalkyl, heterocyclylalkyl, or C₁₋₆ hydroxyalkyl;

when R¹ and R^{1′} are attached to a nitrogen they are, independently, hydrogen, C₁₋₆ alkoxy, C₃₋₆ alkoxyalkyl, alkoxycarbonyl, carbonylalkyl, carbonyl aryl, C₁₋₆ alkyl, C₂₋₁₀ alkynyl, C₂₋₆ alkenyl, heterocyclylalkyl, C₁₋₆ hydroxy alkyl, or S(O)₂R′;

each R′ is independently H, C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, or if two R′ reside on the same nitrogen atom they can come together to form a C₃₋₆ alkyl ring optionally containing a N, O, or S; wherein the R′ groups can be substituted with one or more substituents as defined above, for example, C₁₋₆ hydroxyalkyl, aminoalkyl, and alkoxyalkyl;

u and v are, independently, 0, 1, 2, 3, 4 or 5;

G is phenyl, a six-membered heteroaromatic ring containing one, two, or three nitrogen atoms, a five-membered heteroaromatic ring containing one, two, or three heteroatoms, which are, independently, N, O, or S, a C₄₋₁₄ bicyclic ring; cubane, alkylheteroaryl, or alkylaryl;

F is selected from the group consisting of a five-membered heteroaromatic ring containing one, two, or three heteroatoms, which are, independently, N, O, or S, a six or seven-membered ring or a six or seven-membered bridged or spiro-fused ring containing zero, one, or two heteroatoms which are, independently, N, O, or S, a five-membered ring containing zero, one, or two heteroatoms which are, independently, N, O, or S; and a four-membered ring containing zero, one, or two heteroatoms which are, independently, N, O, or S,

W is

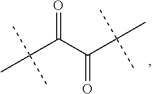

R¹² is H, C₁₋₆ alkyl, C₁₋₆ haloalkyl, C₂₋₆ alkenyl, or C₂₋₆ alkynyl,

R¹³ is C₁₋₆ alkyl, C₂₋₆ alkenyl, C₂₋₆ alkynyl, aryl, heteroaryl, alkylaryl, arylalkyl, a C₄₋₁₄ bicyclic ring; cubane, a six-membered bridged or spiro-fused ring containing zero, one, or two heteroatoms which are, independently, N, O, S, or a 3-7 membered saturated or partially unsaturated ring containing one or more heteroatoms each independently selected from the group of N, O or S,

R¹³ is optionally substituted with one or more substituents each independently selected from the group consisting of

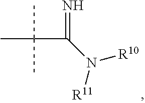

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

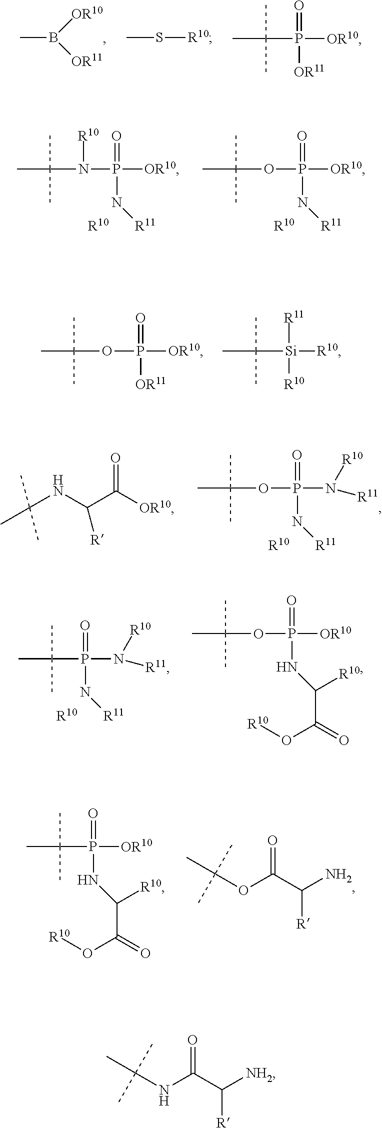

### and —O—S(O)₂—OH,

wherein, when G is substituted aryl, or F is substituted five membered ring heteroaryl, the substituents are selected from the group consisting of

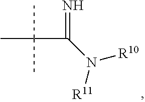

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

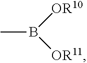

### —S—R¹⁰;

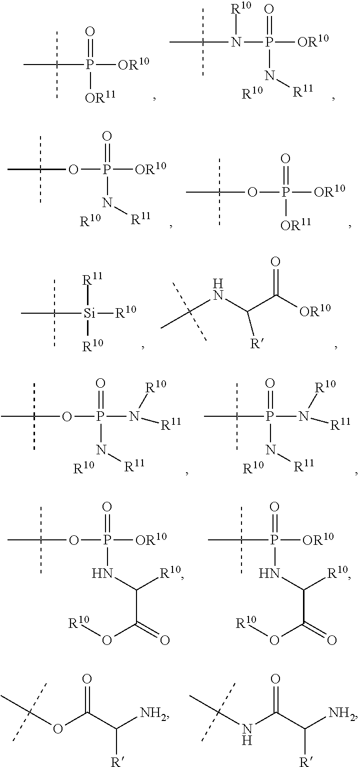

### and —O—S(O)₂—OH,

or R¹² and R¹³ together with the nitrogen to which they are attached form a saturated or unsaturated 4 to 7 membered ring optionally substituted with one or more substituents each independently selected from the group consisting of

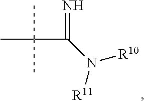

—NH—C(O)—N(R¹⁰)₂, —N(R¹⁰)₂, —OR¹¹, —C(O)OR¹⁰, —C(O)—N(R¹⁰)₂, —C(O)—NHOH, oxo,

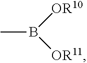

### —S—R¹⁰;

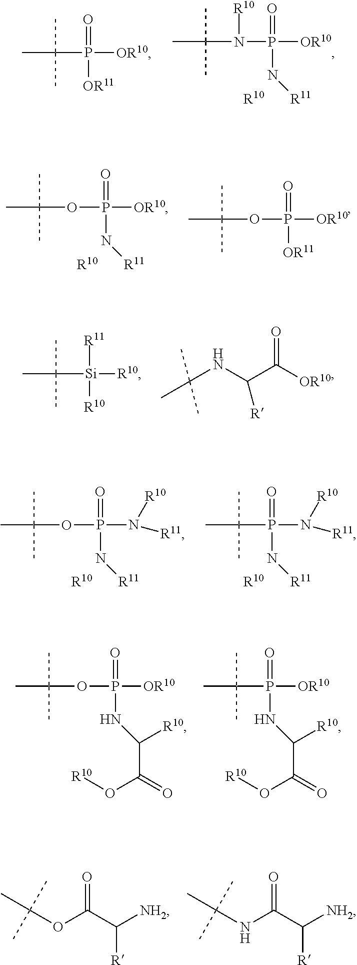

### and —O—S(O)₂—OH,

R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,

alternatively, R¹⁰ and R¹¹ can come together to form a saturated or partially unsaturated 5 to 8 membered ring or a six-membered bridged or spiro-fused ring containing zero, one, or two heteroatoms which are, independently, N, O, or S, a seven-membered bridged or spiro-fused a saturated or partially unsaturated ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms, a saturated or partially unsaturated 6 to 12 membered hetero-bicyclic ring; or a saturated or partially unsaturated 6 to 12 membered hetero-tricyclic ring; and

R¹⁰ and R¹¹ groups can optionally be substituted with one or more substituents, which substituents are, independently, halo, C₁₋₆ alkyl, C₃₋₆ alkyl, C₁₋₆ haloalkyl, C₂₋₆ alkenyl, C₂₋₆ alkynyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphoramidate or phosphonate.

R¹⁴ and R¹⁵ are independently, H, C₁₋₆ alkyl, C₁₋₆ haloalkyl, C₂₋₆ alkenyl, C₂₋₆ alkynyl,

R¹⁴ and R¹⁵ can come together to form an optionally substituted 3 to 5 membered ring, and

R¹⁶ is H or C₁₋₆ alkyl.

In one aspect of this embodiment, G is phenyl.

In another aspect of this embodiment, F is pyrrolyl. The

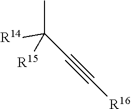

substituent can be directly attached to the ring nitrogen.

In one aspect of this embodiment, R¹ is selected from the group consisting of F, CN and OH.

In one aspect of this embodiment, R¹ is methyl.

In one aspect of this embodiment, R¹⁶ is H.

In addition to the monomers, dimers and trimers discussed above, in another embodiment, the compound is a tetramer of the following formula:

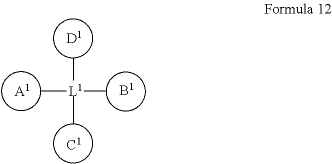

wherein:

A¹, B¹, C¹ and D¹ are, independently, an HBV capsid assembly effector,

each A¹, B¹, C¹ and D¹ may be the same or different;

L¹ is a chemical linker selected from the group consisting of —C—((C₂₋₆ alkyl-O)ₚ)₄—, —C—(C₁₋₁₆ alkyl-)₄, —C(NR′)₃—)₄, C(C₁₋₆ alkyl-W¹—)₄; —C((CH₂—CH₂—O)ₜ—C₁₋₆ alkyl-W¹—)₄; —C((CH₂—CH₂—O)ₛ)₄—; C(C₁₋₃₀ alkyl-W¹—)₄; C—((C₃₋₆ alkyl-O)ₛ—C₁₋₆ alkyl-X—(C₃₋₆ alkyl-O)ₛ—C₁₋₆ alkyl-)₄; C((CH₂—CH₂—O)ₜ—C₁₋₆ alkyl-X—(CH₂—CH₂—O)ₜ—C₁₋₆ alkyl-W¹—)₄, or C(C₁₋₃₀ alkyl-X—C₁₋₃₀ alkyl-W¹—)₄;

p, s and m are integers from 1 to 15;

t is an integer from 3 to 15;

X is Si(R⁷)(R⁸)—, —Si(R⁷)(R⁸)—O—, —O—Si(R⁷)(R⁸)—, —NR′—; —N(R′)C(O)—; —C(O)N(R′)—; —N(R′)SO₂—; —SO₂N(R′)—; —O—; —C(O)—; —OC(O)—; —C(O)O—; —S—; —S(O)—; —SO₂—; —C(═S)—; or —C(═NR′);

wherein the optional substituents for the heteroaliphatic, aliphatic, aryl, and heteroaryl moieties are selected from the group consisting of halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, phosphonyl, phosphinyl, phosphoryl, phosphine, thioester, thioether, oxime, hydrazine, carbamate, silyl ether, boronic ester, boronic acid, phosphonic acid, phosphonamidate, and phosphonate,

W¹ is, independently for each occurrence, aryl, heteroaryl, CHR′, NR′, N, O or S;

R′ is H, C₁₋₆ alkyl, optionally unsaturated C₃₋₈ cycloalkyl, C₁₋₆ haloalkyl, C₂₋₈ alkoxyalkyl, C₂₋₆ alkenyl, C₂₋₆ alkynyl, aryl, heteroaryl, alkylaryl, or arylalkyl,

R⁷ and R⁸ are, independently, —OH, optionally unsaturated —O—C₁₋₆ alkyl, C₁₋₆ alkyl, C₂₋₆ alkenyl, C₂₋₆ alkynyl, optionally unsaturated C₃₋₈ cycloalkyl, aryl, heteroaryl, alkylaryl, or arylalkyl, and

alternatively, R⁷ and R⁸, together with the silicon to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, cyano, oxo and OH;

or an enantiomer, diasteroisomer, tautomer, pharmaceutically acceptable salt or prodrug thereof.

Specific compounds include the following, as well as tautomers, stereoisomers, diastereomers, pharmaceutically-acceptable salts, and prodrugs of these compounds:

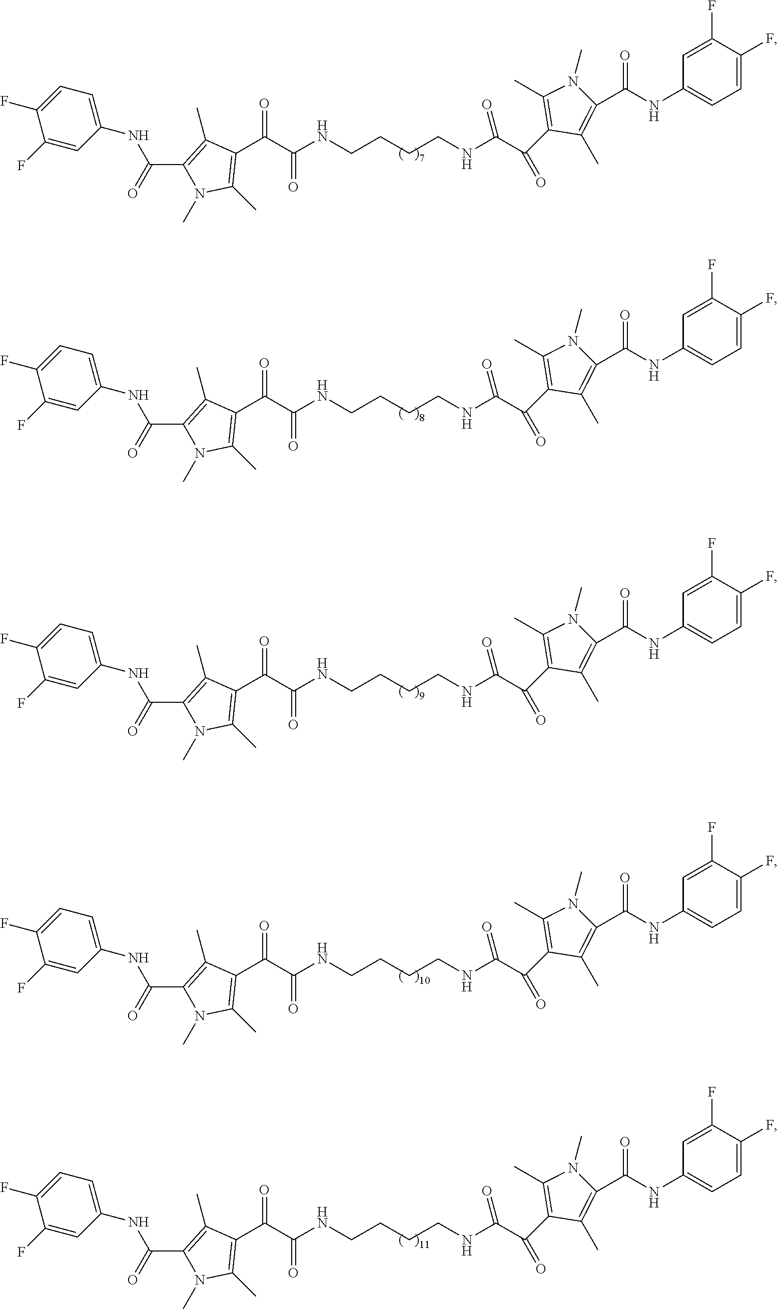

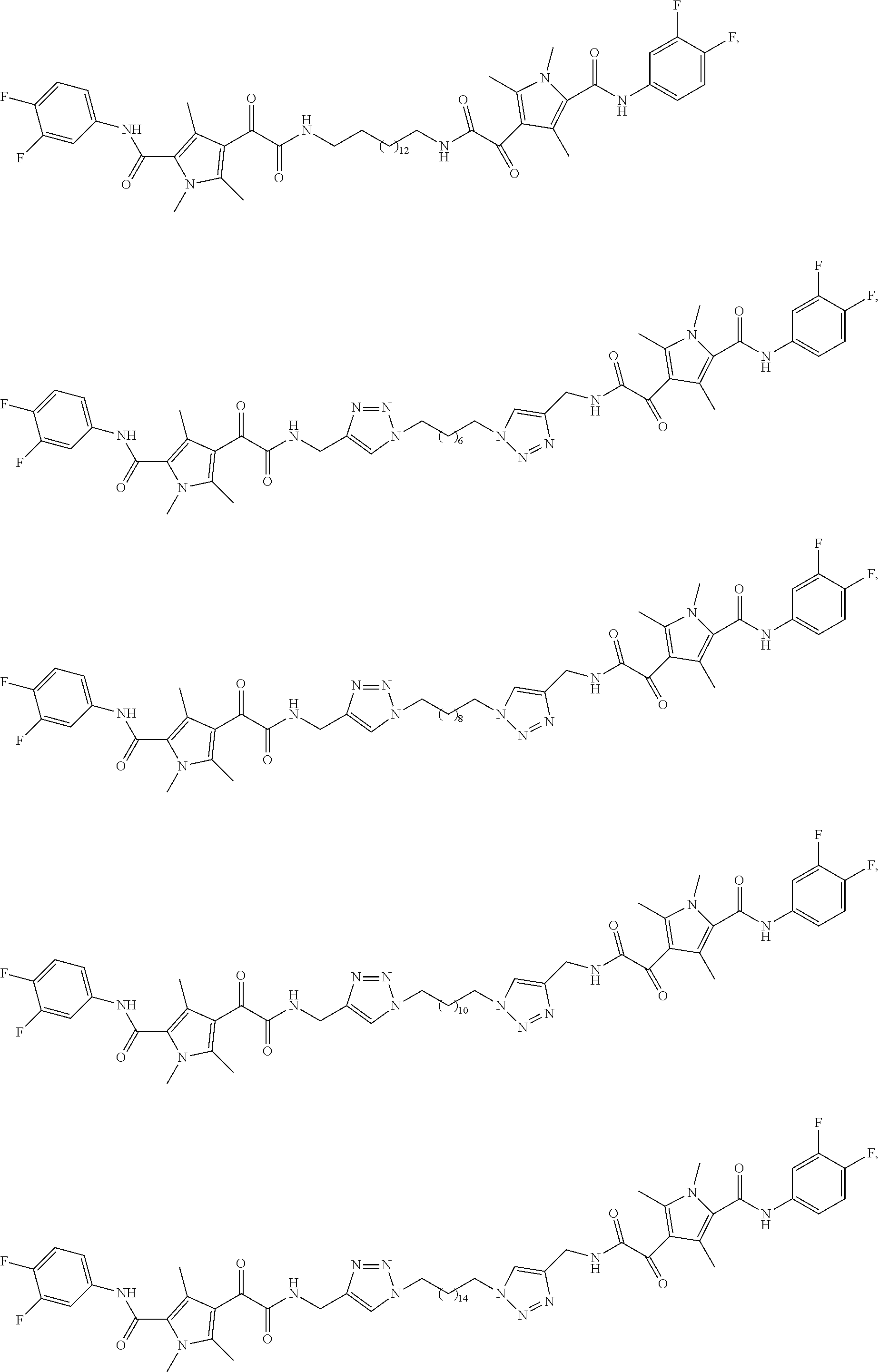

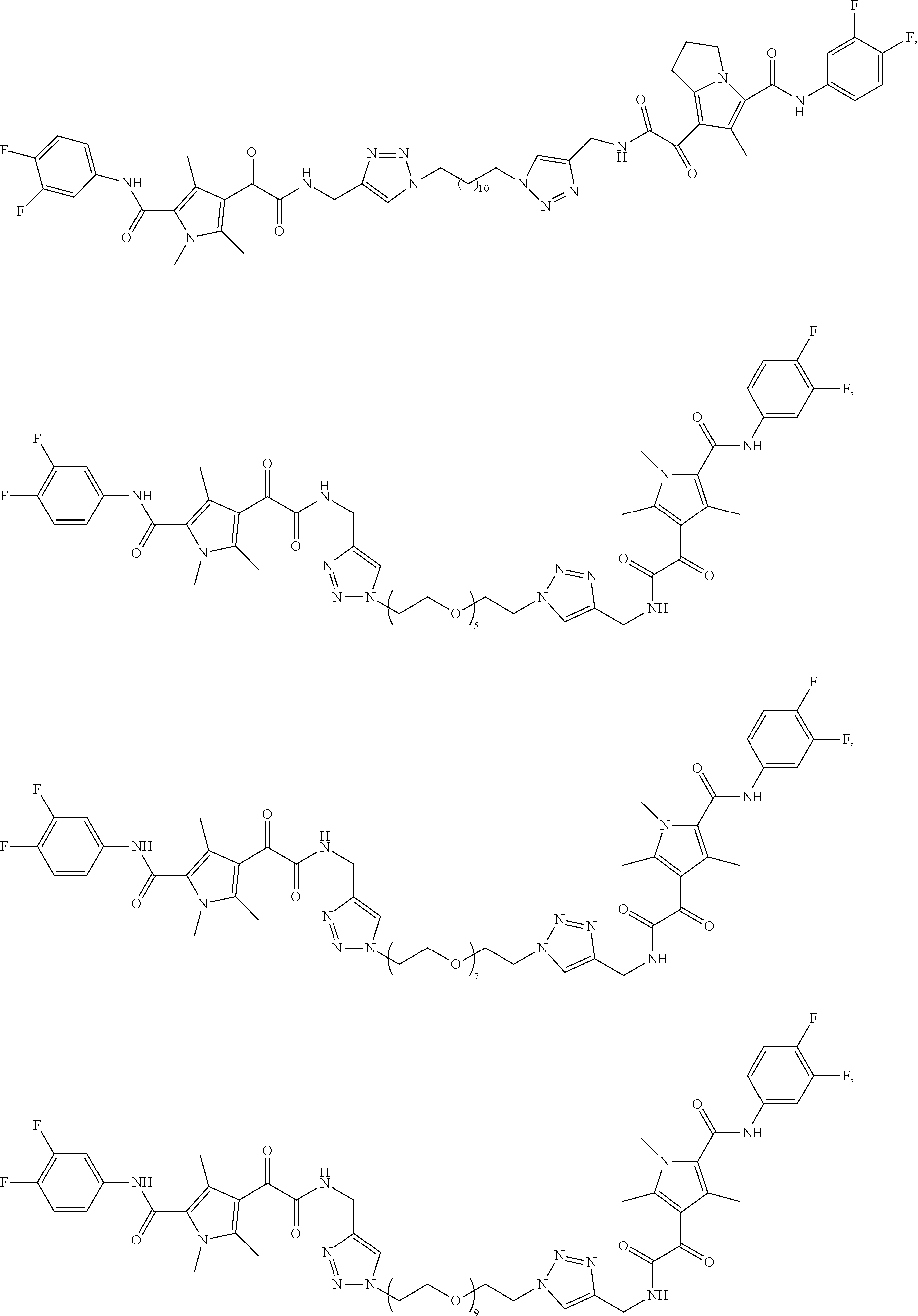

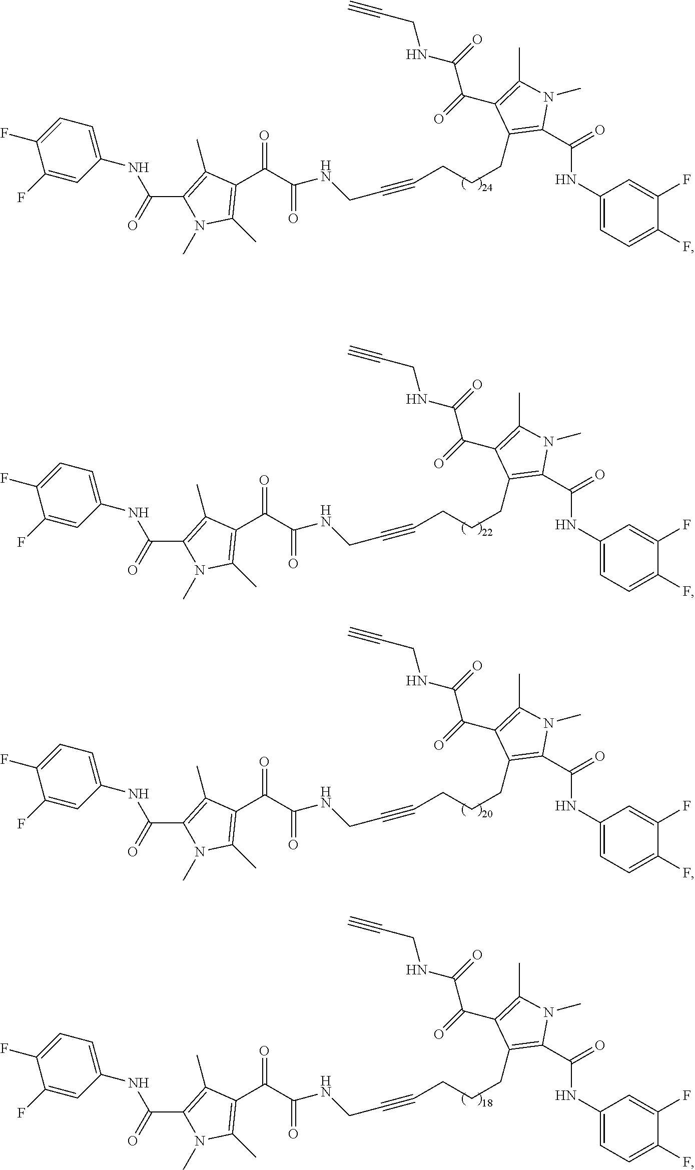

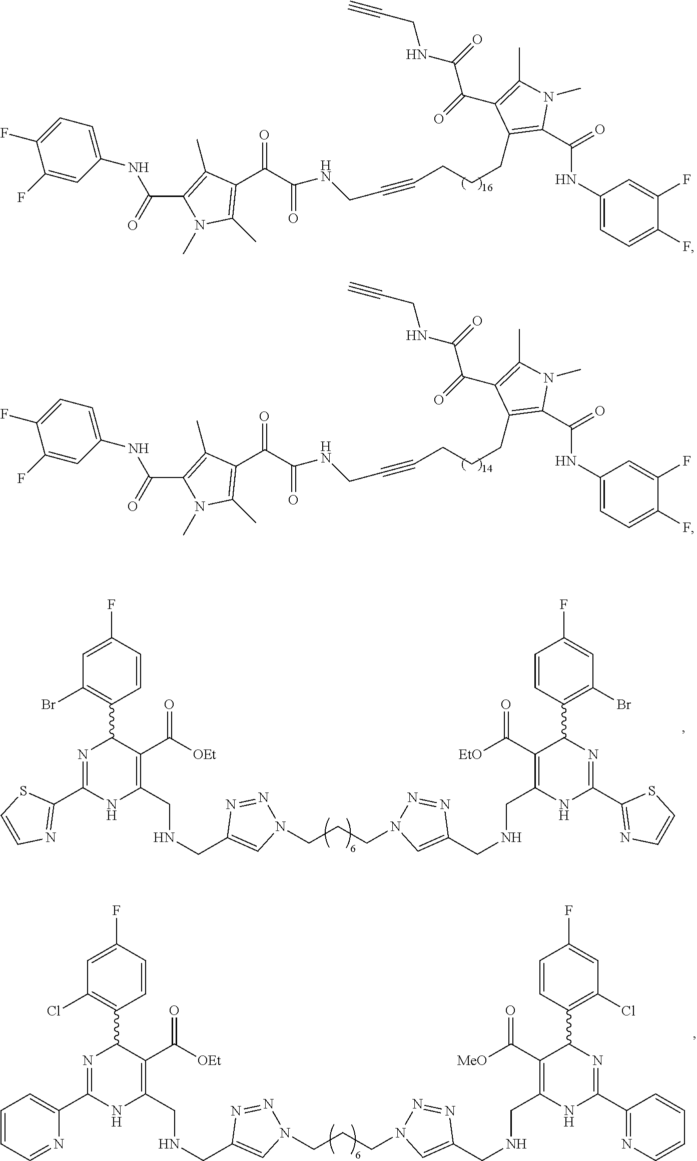

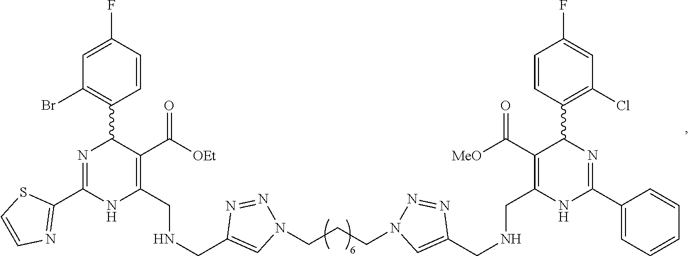

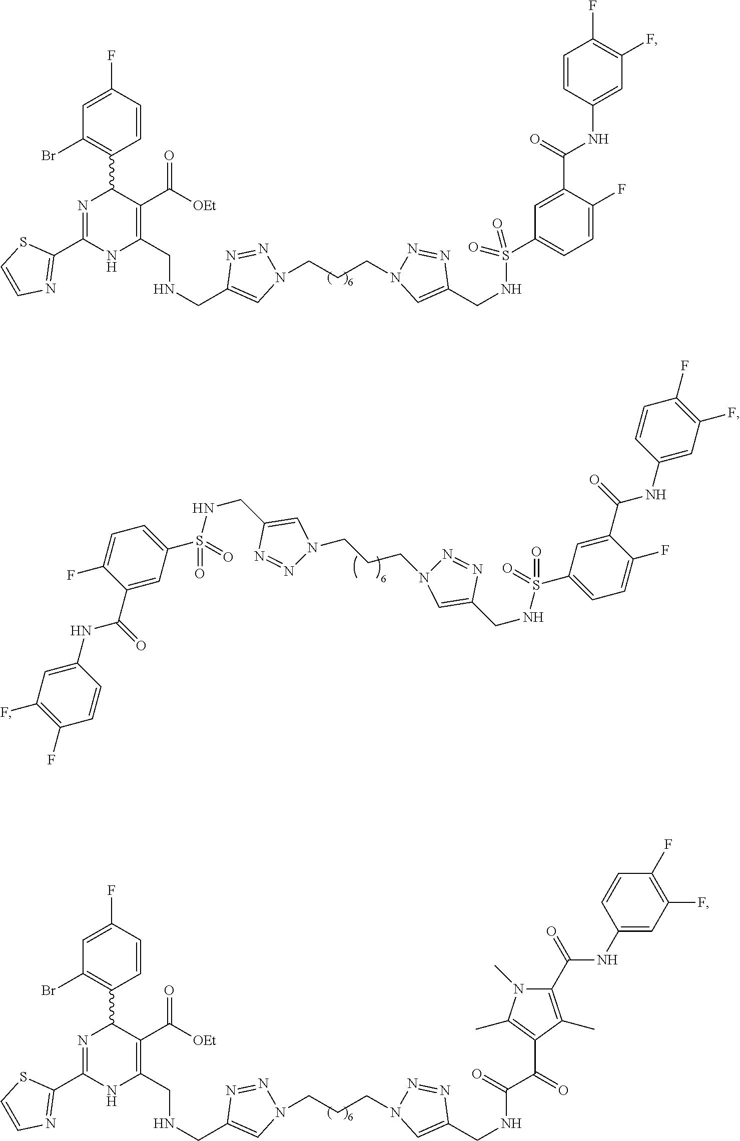

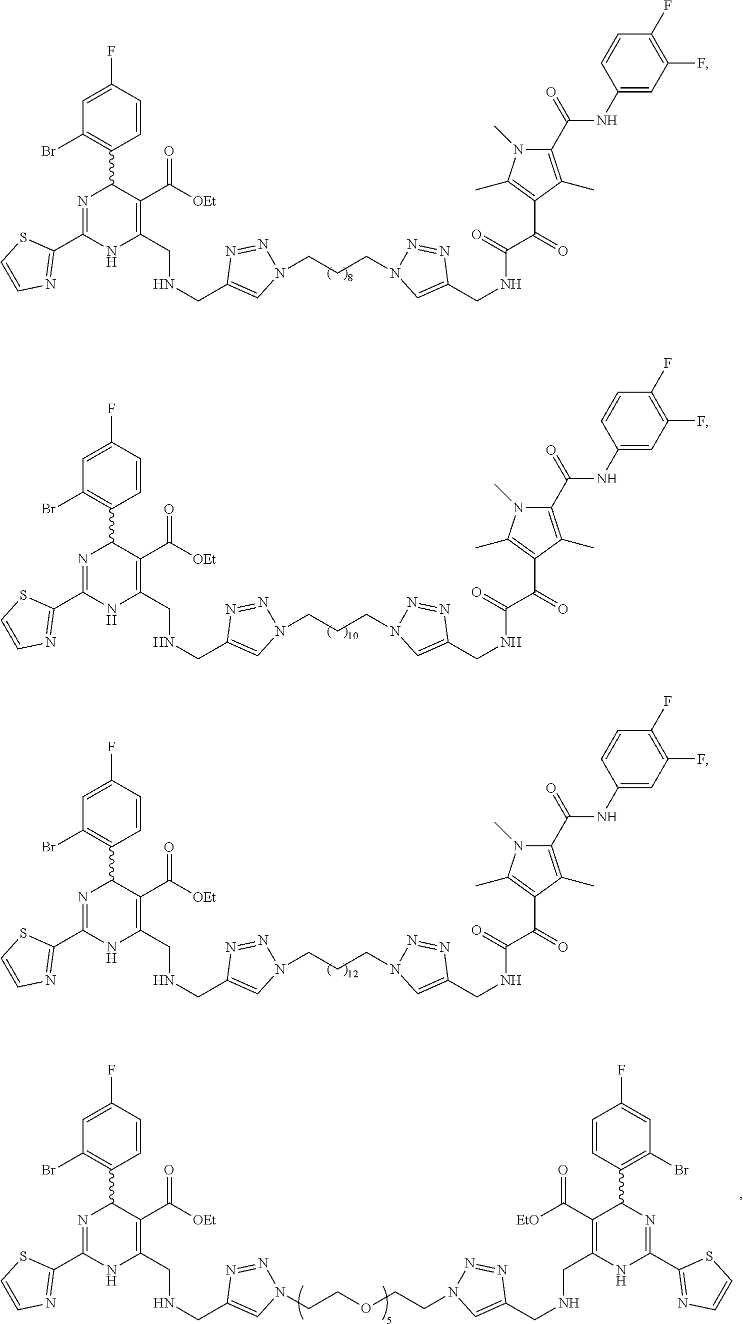

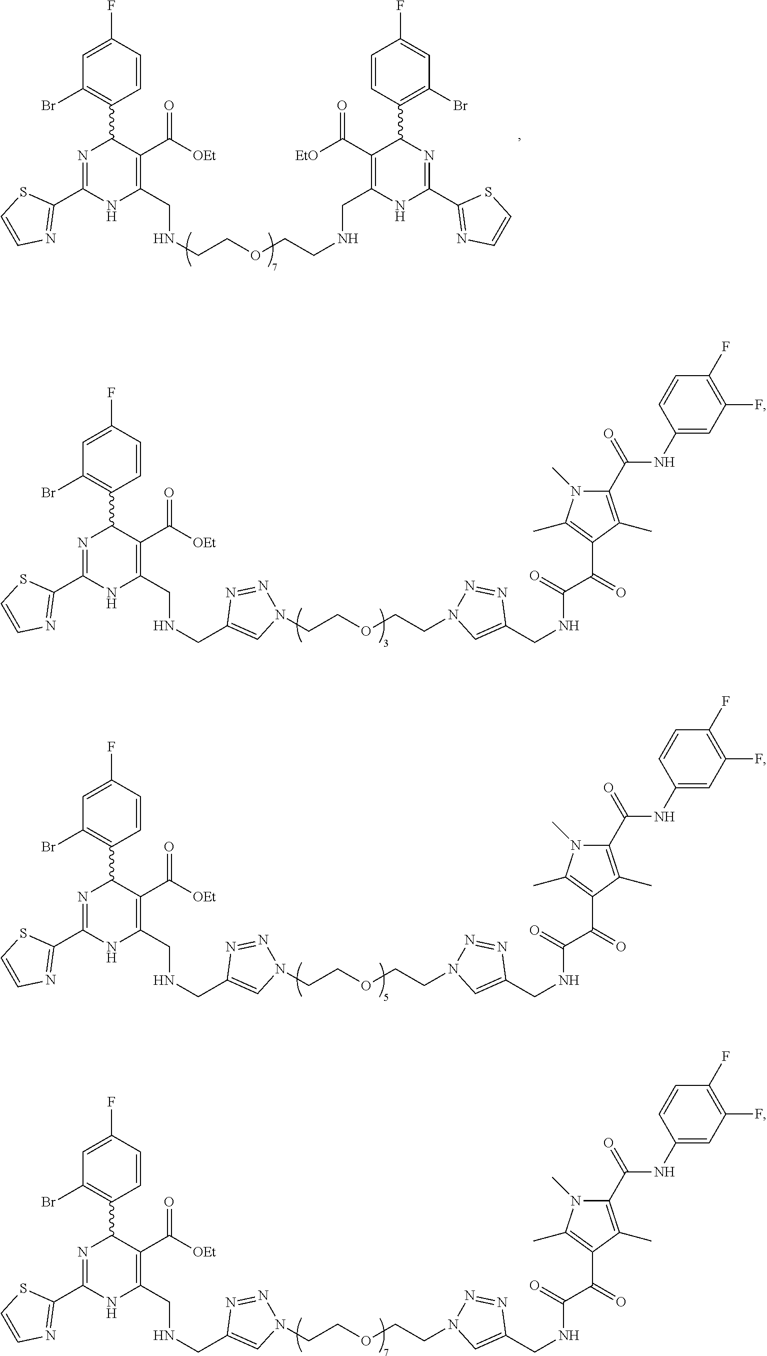

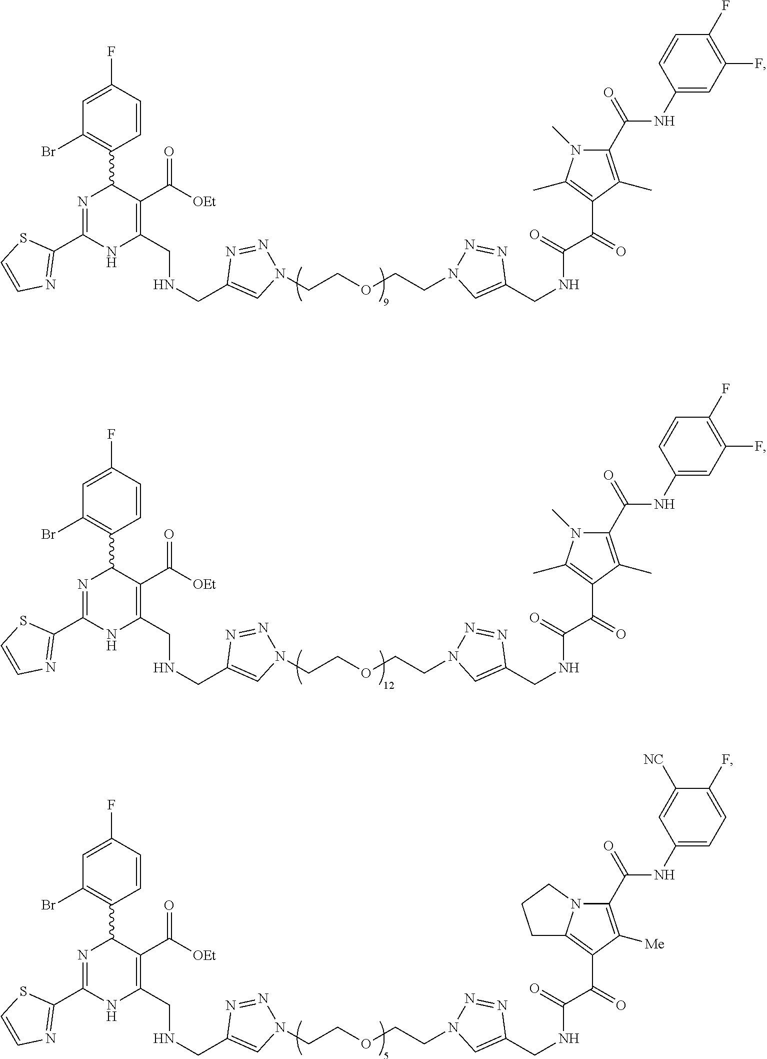

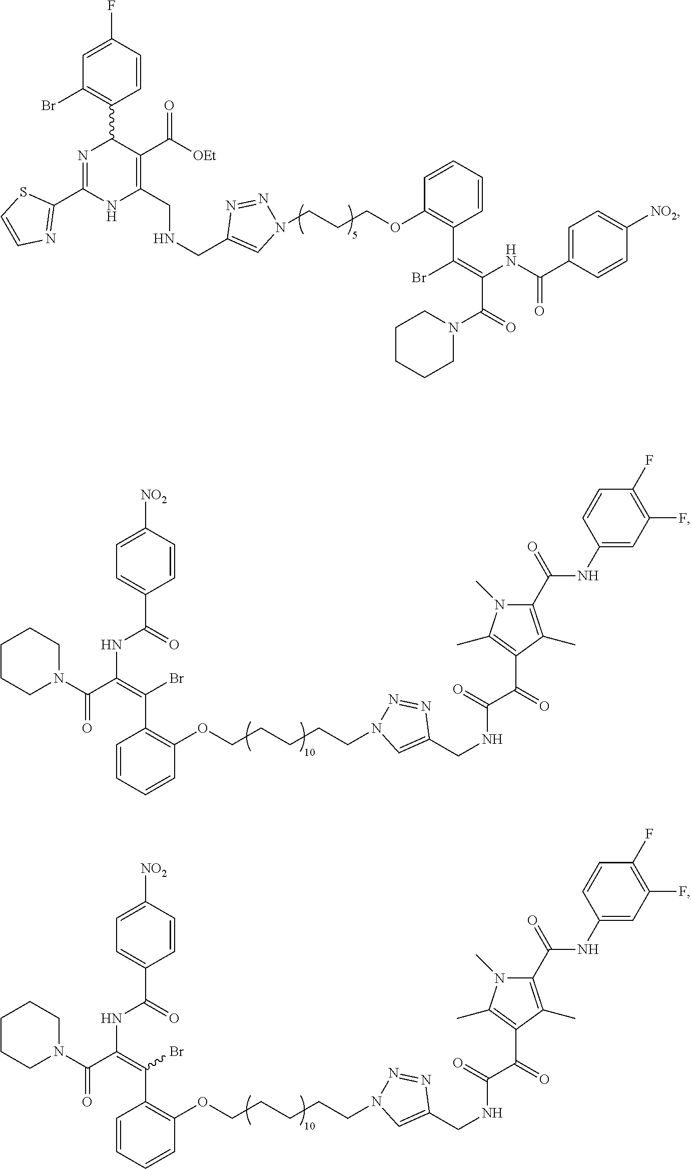

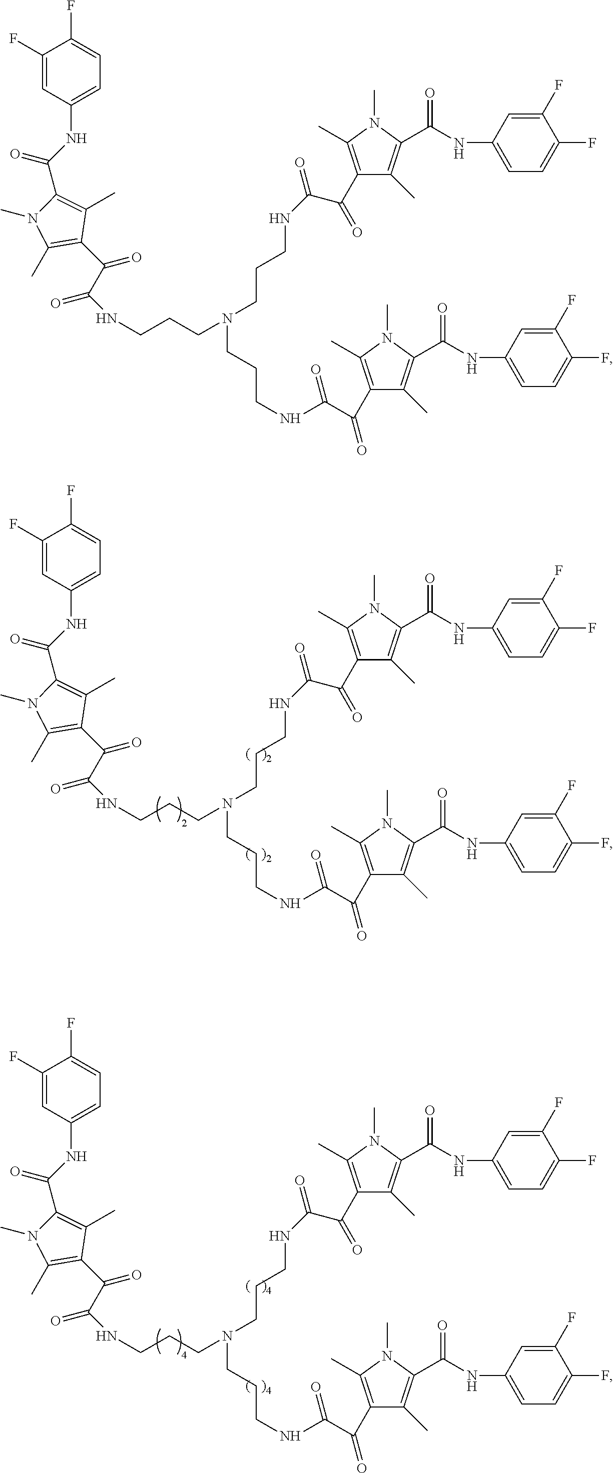

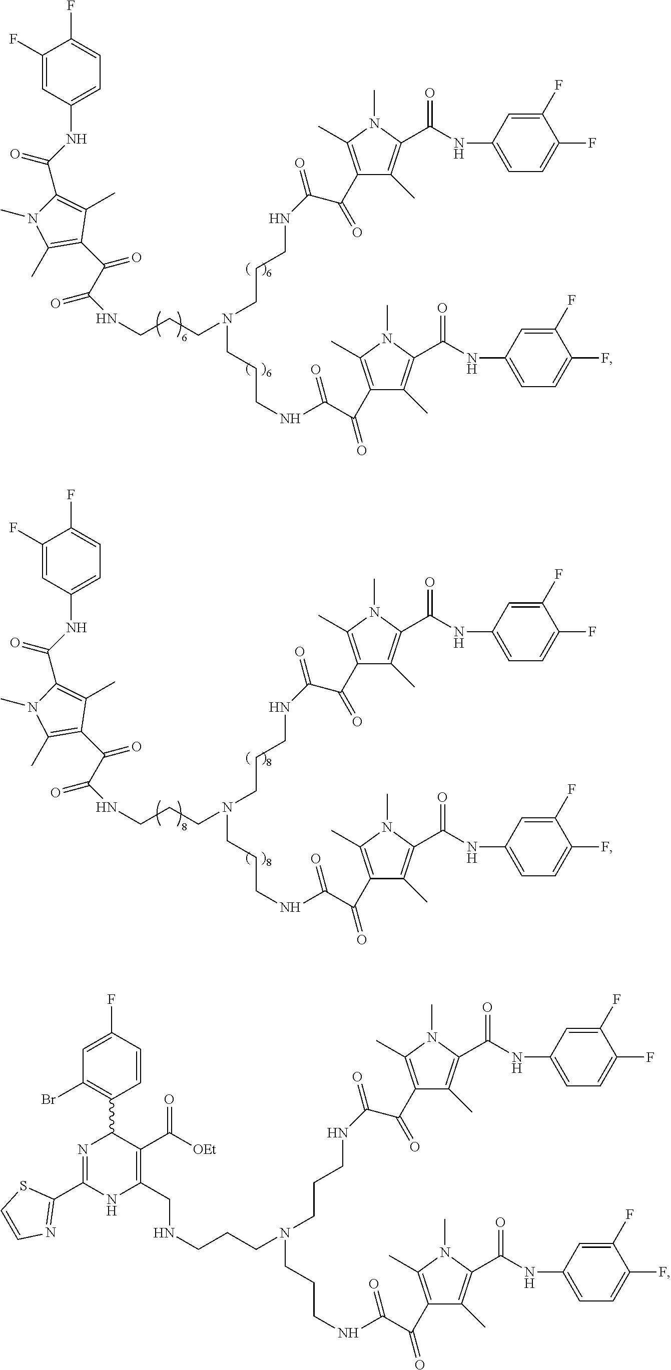

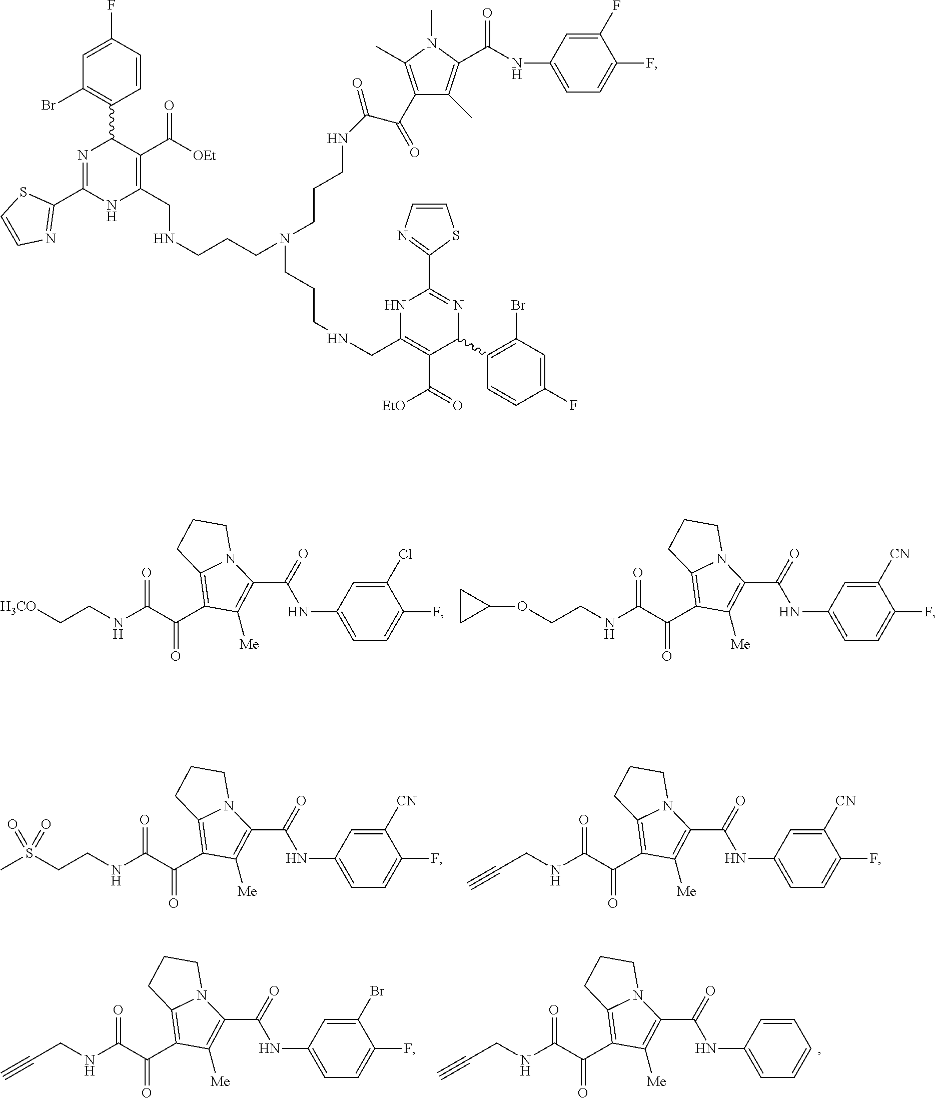

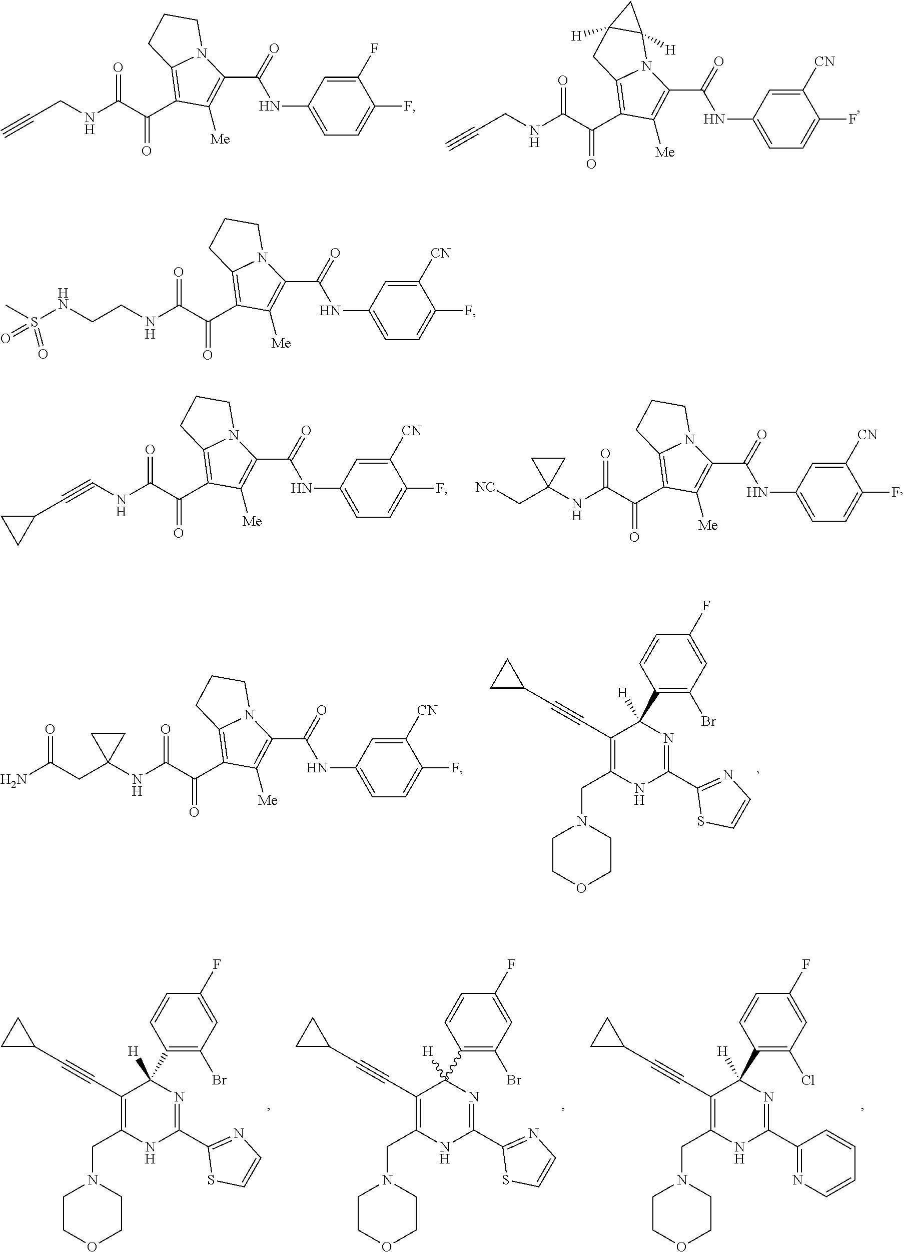

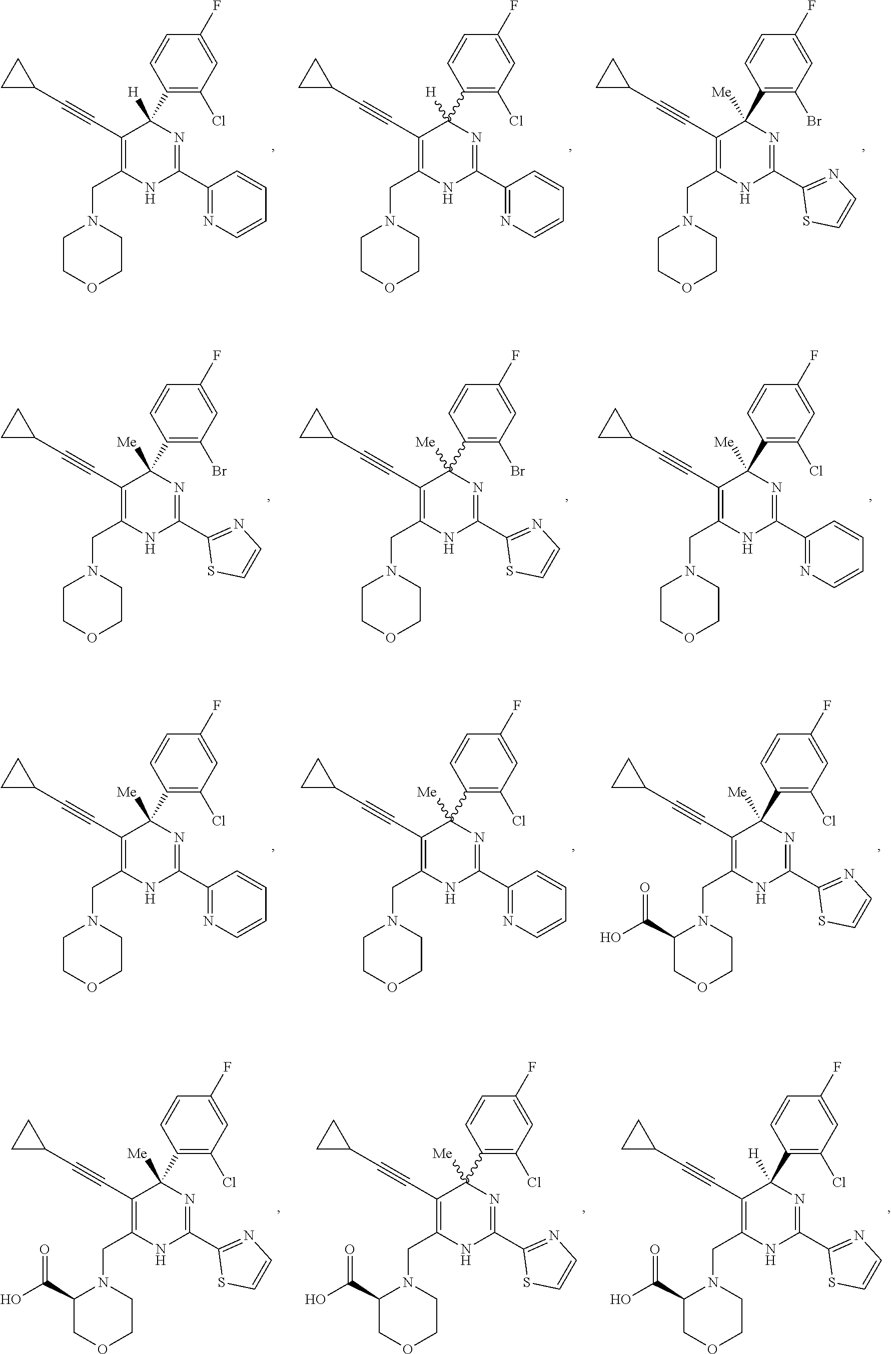

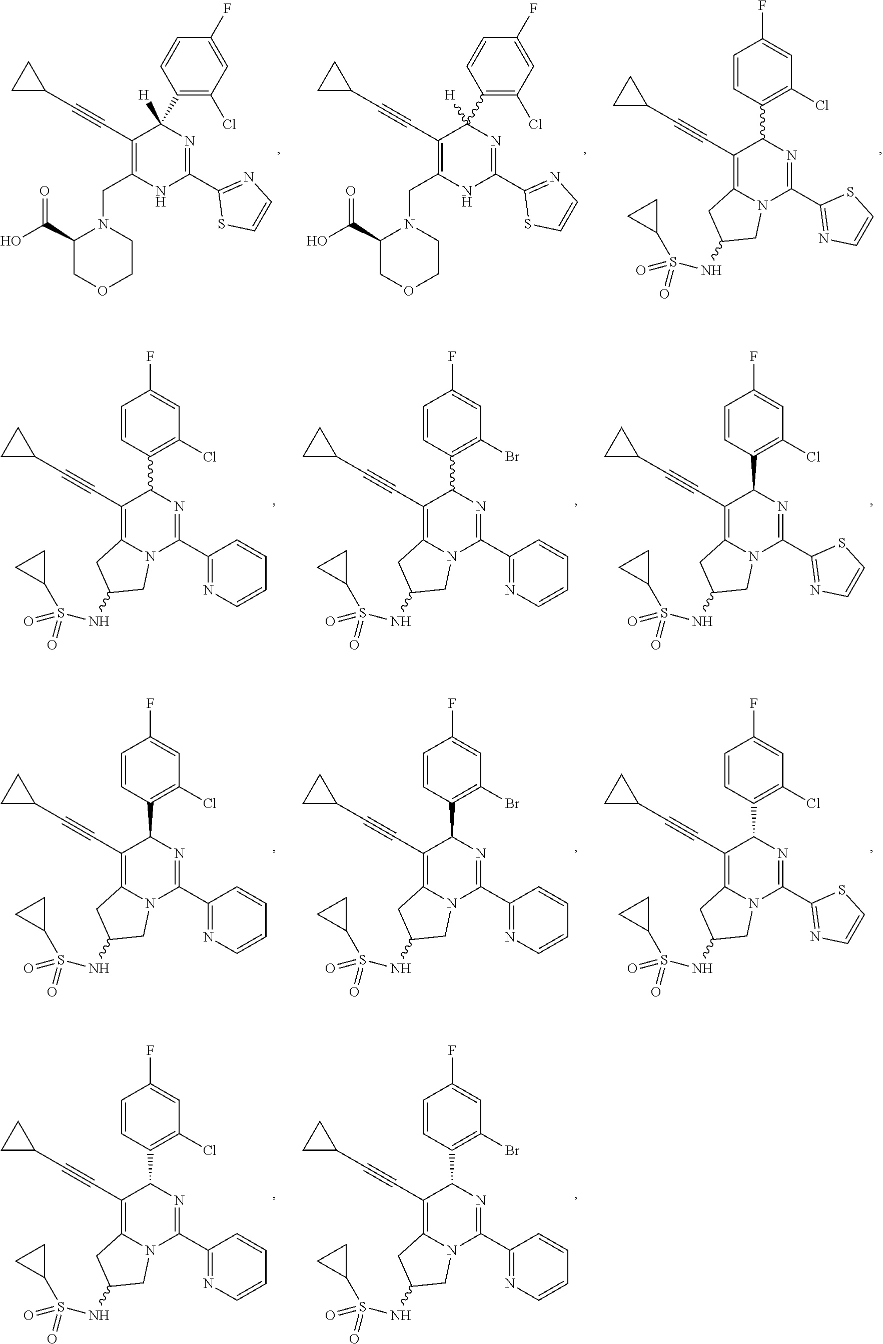

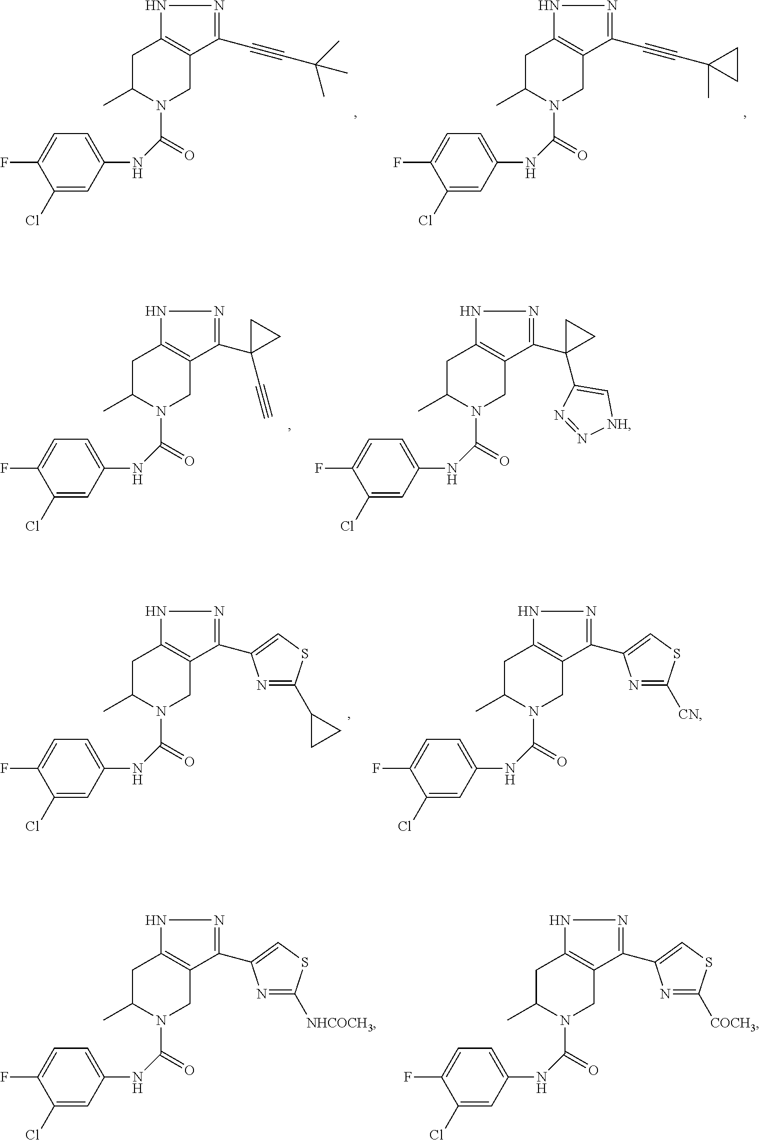

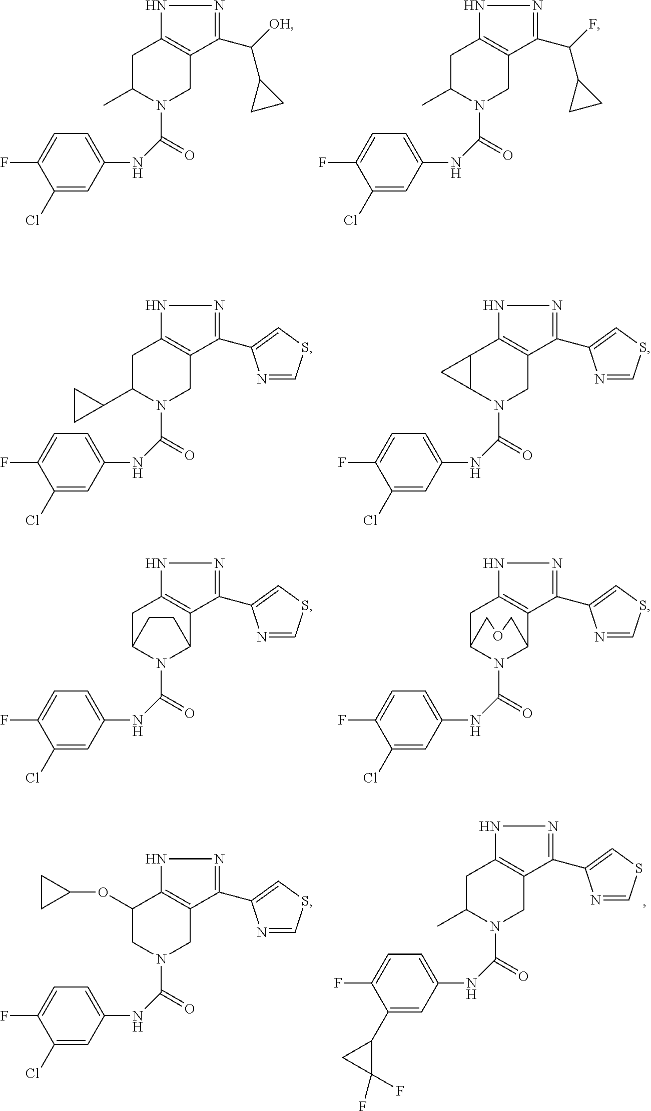

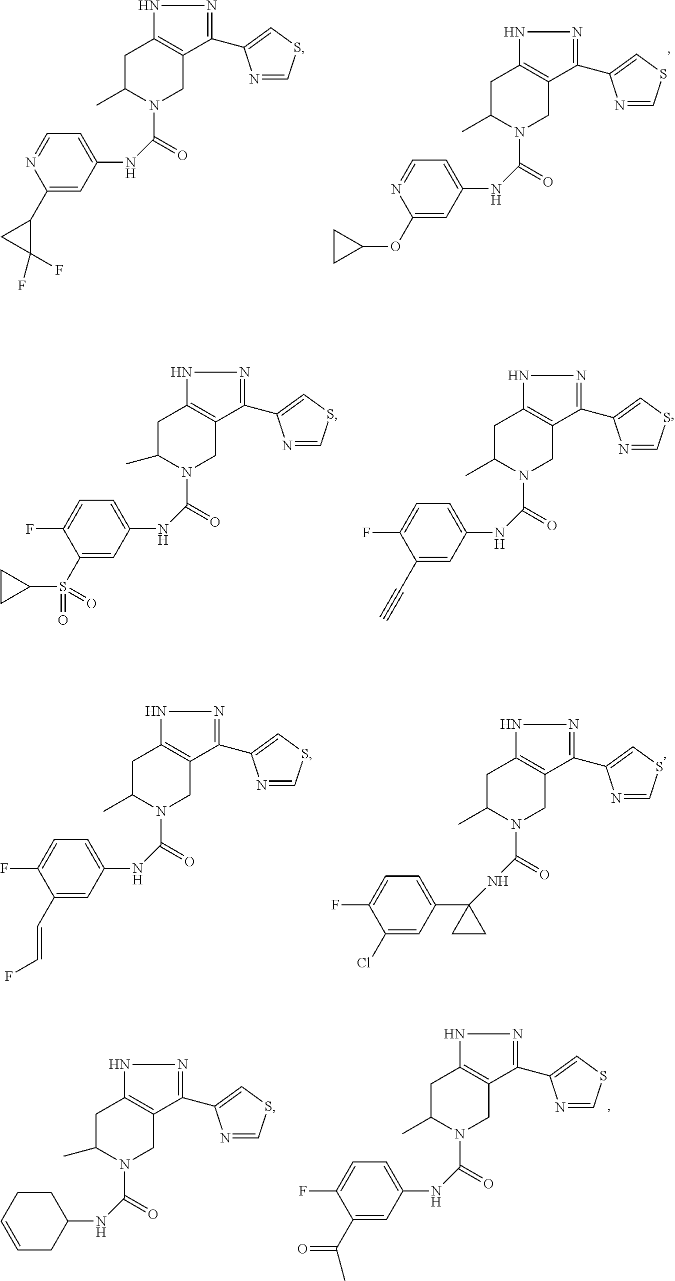

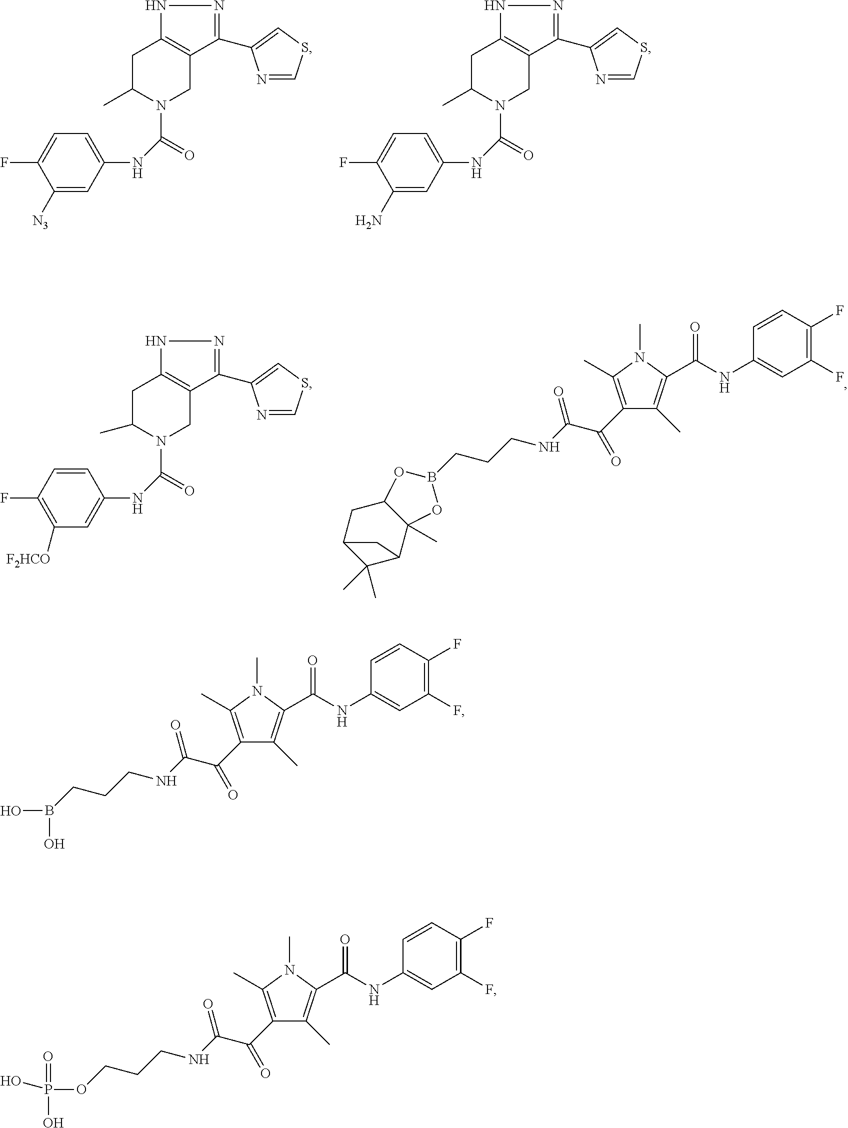 [SMILES: CC1Cc2[nH]nc(-c3cscn3)c2CN1C(=O)Nc1ccc(F)c(N=[N+]=[N-])c1 ; CC1Cc2[nH]nc(-c3cscn3)c2CN1C(=O)Nc1ccc(F)c(N)c1 ; CC1Cc2[nH]nc(-c3cscn3)c2CN1C(=O)Nc1ccc(F)c(OC(F)F)c1 ; Cc1c(C(=O)C(=O)NCCCB2OC3CC4CC(C4(C)C)C3(C)O2)c(C)n(C)c1C(=O)Nc1ccc(F)c(F)c1 ; Cc1c(C(=O)C(=O)NCCCB(O)O)c(C)n(C)c1C(=O)Nc1ccc(F)c(F)c1 ; Cc1c(C(=O)C(=O)NCCCOP(=O)(O)O)c(C)n(C)c1C(=O)Nc1ccc(F)c(F)c1]

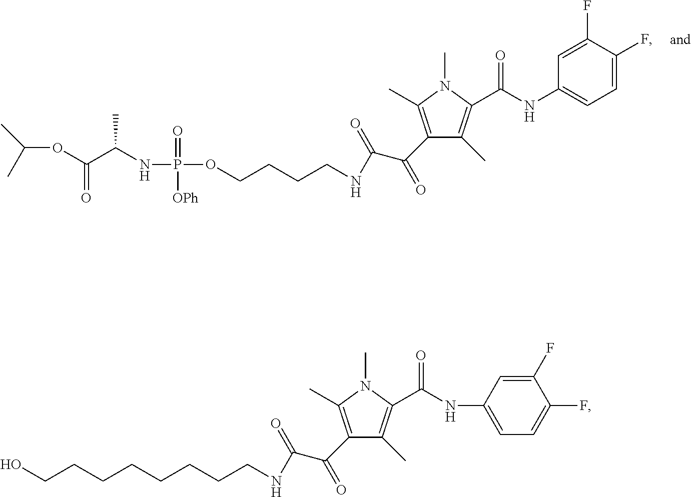 [SMILES: Cc1c(C(=O)C(=O)NCCCCOP(=O)(N[C@@H](C)C(=O)OC(C)C)Oc2ccccc2)c(C)n(C)c1C(=O)Nc1ccc(F)c(F)c1 ; Cc1c(C(=O)C(=O)NCCCCCCCCO)c(C)n(C)c1C(=O)Nc1ccc(F)c(F)c1]

and pharmaceutically acceptable salts or prodrugs thereof.

In another aspect, the compounds are selected from the group consisting of:

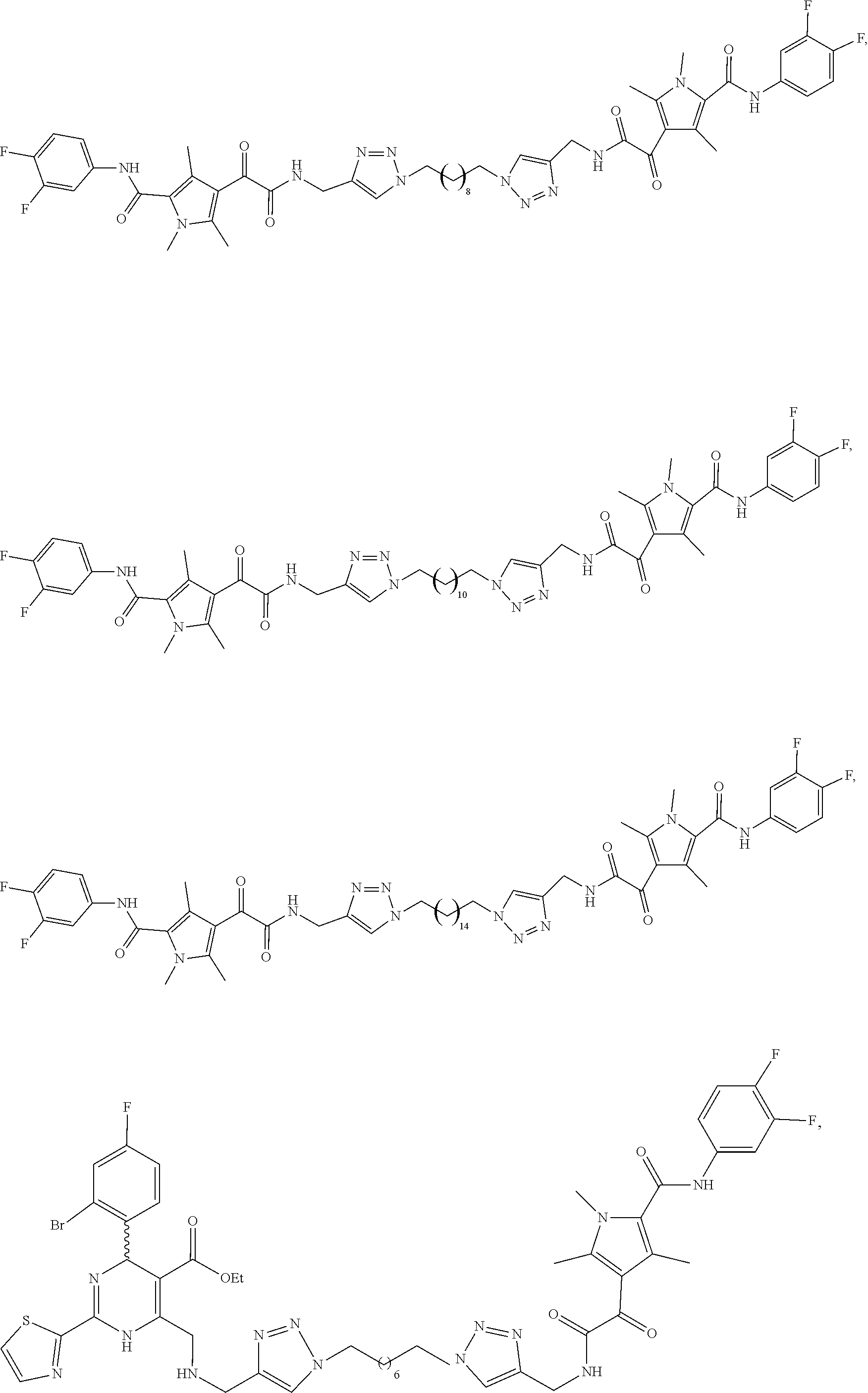

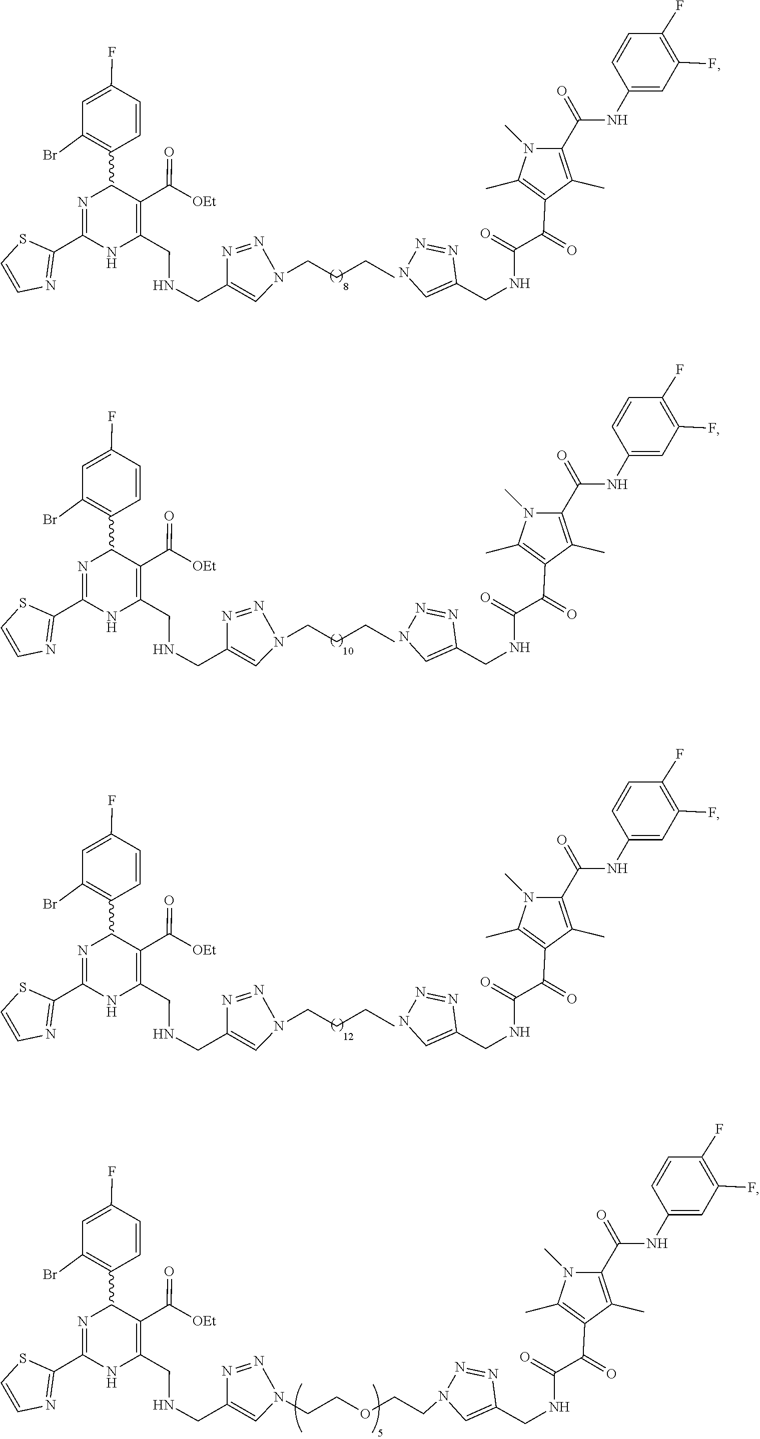

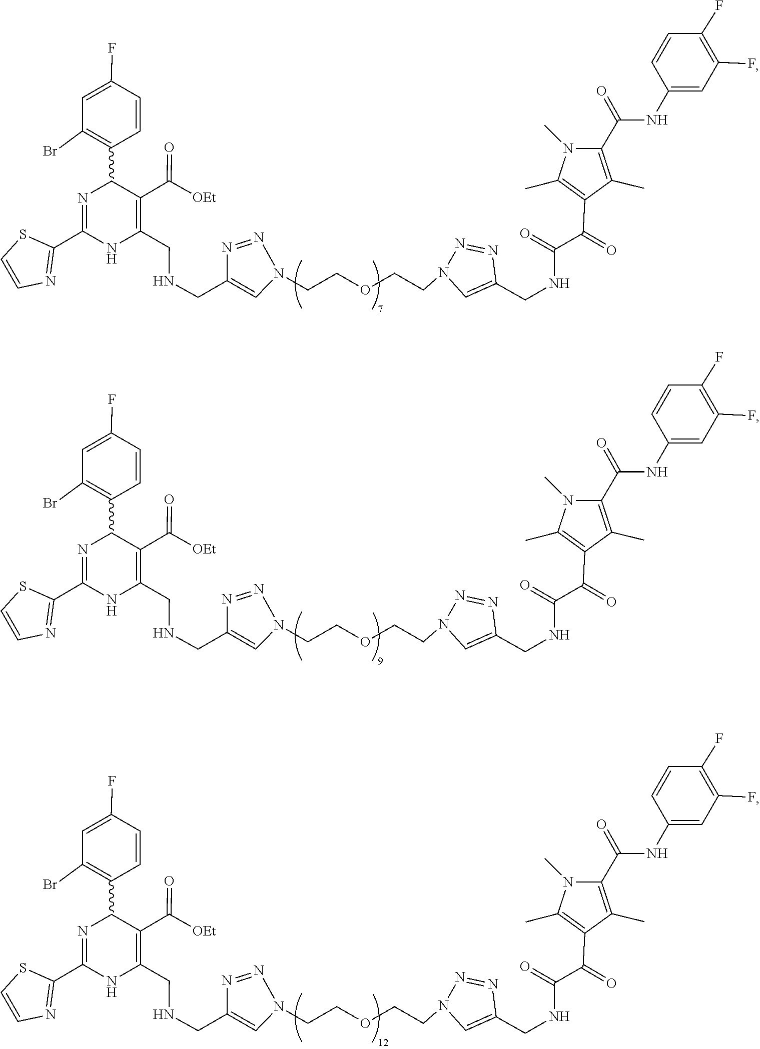

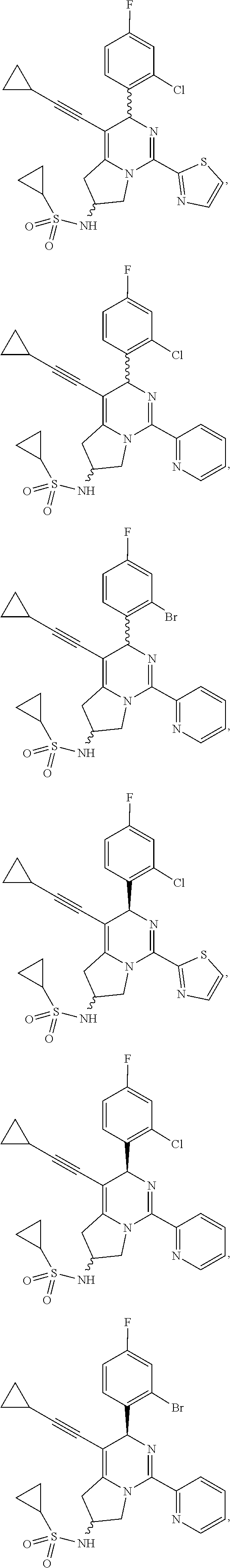 [SMILES: O=S(=O)(NC1CC2=C(C#CC3CC3)C(c3ccc(F)cc3Cl)N=C(c3nccs3)N2C1)C1CC1 ; O=S(=O)(NC1CC2=C(C#CC3CC3)C(c3ccc(F)cc3Cl)N=C(c3ccccn3)N2C1)C1CC1 ; O=S(=O)(NC1CC2=C(C#CC3CC3)C(c3ccc(F)cc3Br)N=C(c3ccccn3)N2C1)C1CC1 ; O=S(=O)(NC1CC2=C(C#CC3CC3)[C@@H](c3ccc(F)cc3Cl)N=C(c3nccs3)N2C1)C1CC1 ; O=S(=O)(NC1CC2=C(C#CC3CC3)[C@@H](c3ccc(F)cc3Cl)N=C(c3ccccn3)N2C1)C1CC1 ; O=S(=O)(NC1CC2=C(C#CC3CC3)[C@@H](c3ccc(F)cc3Br)N=C(c3ccccn3)N2C1)C1CC1]

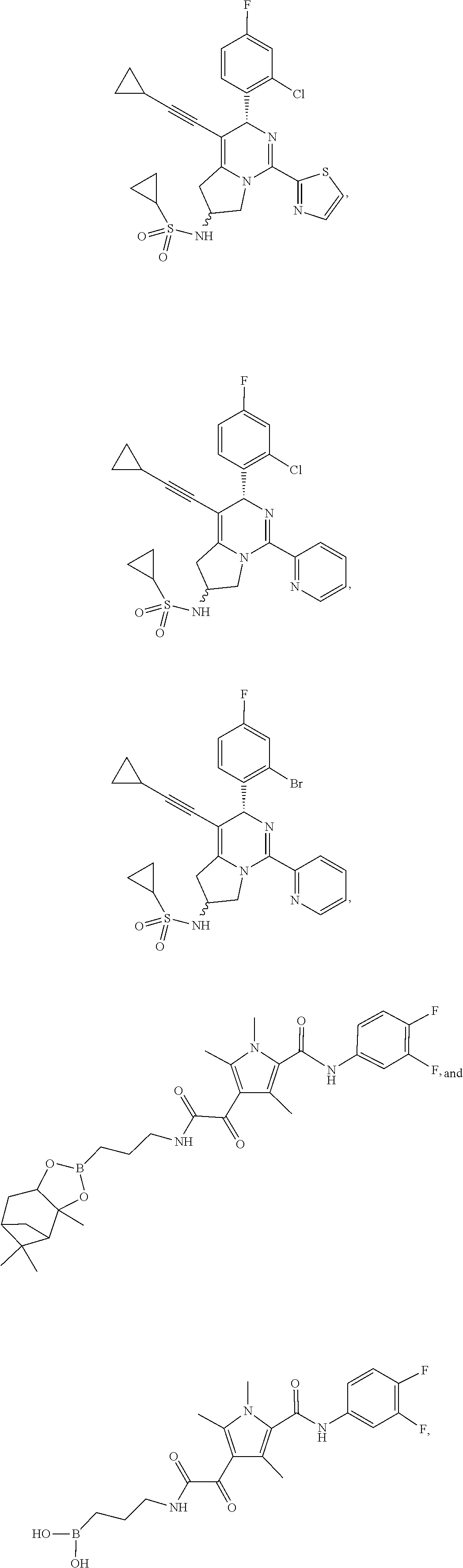 [SMILES: O=S(=O)(NC1CC2=C(C#CC3CC3)[C@H](c3ccc(F)cc3Cl)N=C(c3nccs3)N2C1)C1CC1 ; O=S(=O)(NC1CC2=C(C#CC3CC3)[C@H](c3ccc(F)cc3Cl)N=C(c3ccccn3)N2C1)C1CC1 ; O=S(=O)(NC1CC2=C(C#CC3CC3)[C@H](c3ccc(F)cc3Br)N=C(c3ccccn3)N2C1)C1CC1 ; Cc1c(C(=O)C(=O)NCCCB2OC3CC4CC(C4(C)C)C3(C)O2)c(C)n(C)c1C(=O)Nc1ccc(F)c(F)c1 ; Cc1c(C(=O)C(=O)NCCCB(O)O)c(C)n(C)c1C(=O)Nc1ccc(F)c(F)c1]

and pharmaceutically-acceptable salts and prodrugs thereof.

Examples of compounds of Formula 11 include:

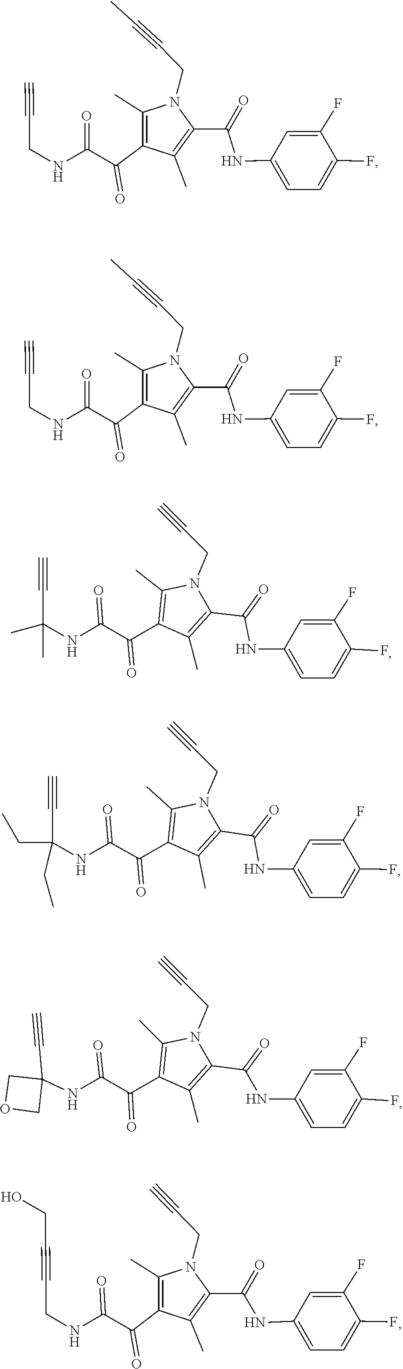 [SMILES: C#CCNC(=O)C(=O)c1c(C)c(C(=O)Nc2ccc(F)c(F)c2)n(CC#CC)c1C ; C#CCNC(=O)C(=O)c1c(C)c(C(=O)Nc2ccc(F)c(F)c2)n(CC#CC)c1C ; C#CCn1c(C)c(C(=O)C(=O)NC(C)(C)C#C)c(C)c1C(=O)Nc1ccc(F)c(F)c1 ; C#CCn1c(C)c(C(=O)C(=O)NC(C#C)(CC)CC)c(C)c1C(=O)Nc1ccc(F)c(F)c1 ; C#CCn1c(C)c(C(=O)C(=O)NC2(C#C)COC2)c(C)c1C(=O)Nc1ccc(F)c(F)c1 ; C#CCn1c(C)c(C(=O)C(=O)NCC#CCO)c(C)c1C(=O)Nc1ccc(F)c(F)c1]

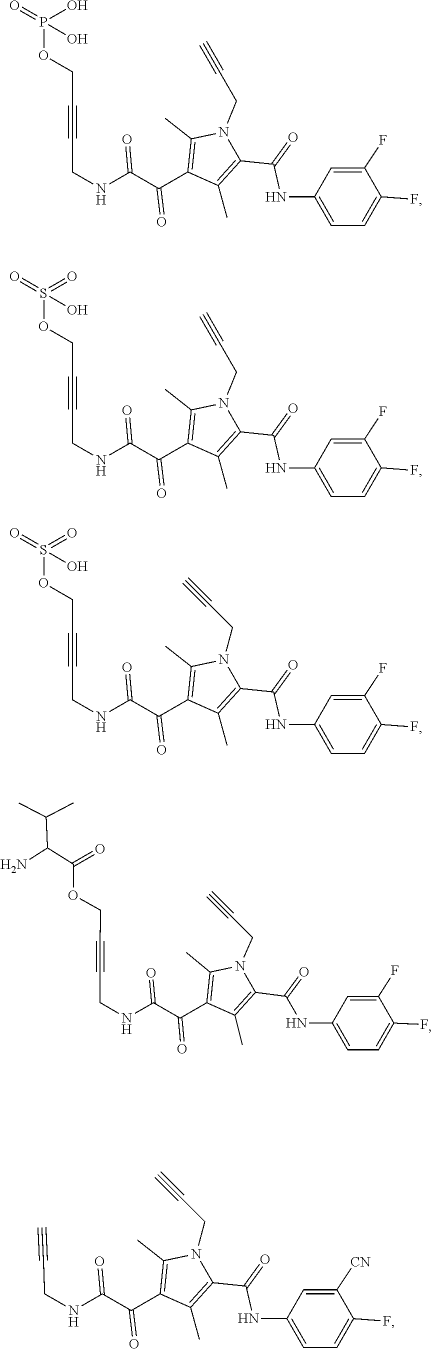 [SMILES: C#CCn1c(C)c(C(=O)C(=O)NCC#CCOP(=O)(O)O)c(C)c1C(=O)Nc1ccc(F)c(F)c1 ; C#CCn1c(C)c(C(=O)C(=O)NCC#CCOS(=O)(=O)O)c(C)c1C(=O)Nc1ccc(F)c(F)c1 ; C#CCn1c(C)c(C(=O)C(=O)NCC#CCOS(=O)(=O)O)c(C)c1C(=O)Nc1ccc(F)c(F)c1 ; C#CCn1c(C)c(C(=O)C(=O)NCC#CCOC(=O)C(N)C(C)C)c(C)c1C(=O)Nc1ccc(F)c(F)c1 ; C#CCNC(=O)C(=O)c1c(C)c(C(=O)Nc2ccc(F)c(C#N)c2)n(CC#C)c1C]

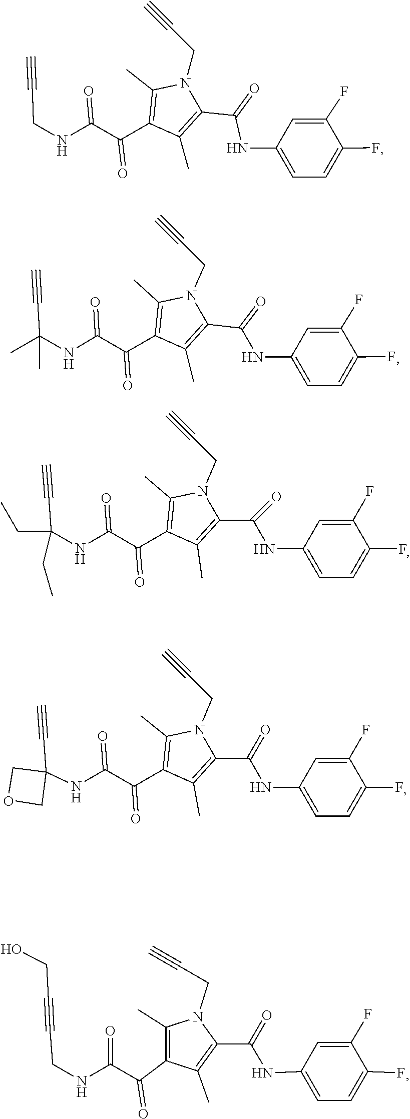

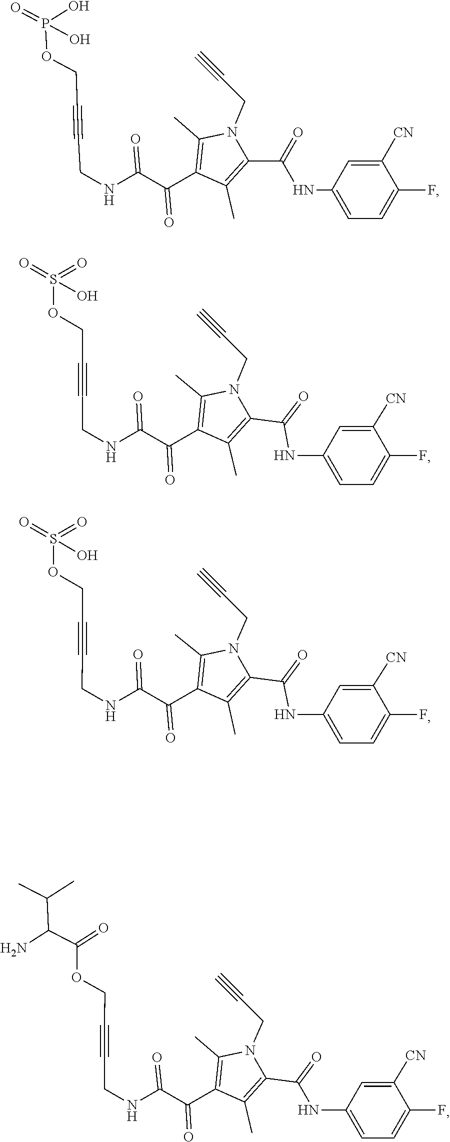 [SMILES: C#CCn1c(C)c(C(=O)C(=O)NCC#CCOP(=O)(O)O)c(C)c1C(=O)Nc1ccc(F)c(C#N)c1 ; C#CCn1c(C)c(C(=O)C(=O)NCC#CCOS(=O)(=O)O)c(C)c1C(=O)Nc1ccc(F)c(C#N)c1 ; C#CCn1c(C)c(C(=O)C(=O)NCC#CCOS(=O)(=O)O)c(C)c1C(=O)Nc1ccc(F)c(C#N)c1 ; C#CCn1c(C)c(C(=O)C(=O)NCC#CCOC(=O)C(N)C(C)C)c(C)c1C(=O)Nc1ccc(F)c(C#N)c1]

and pharmaceutically-acceptable salts, tautomers, and prodrugs thereof.

Additional compounds include the following:

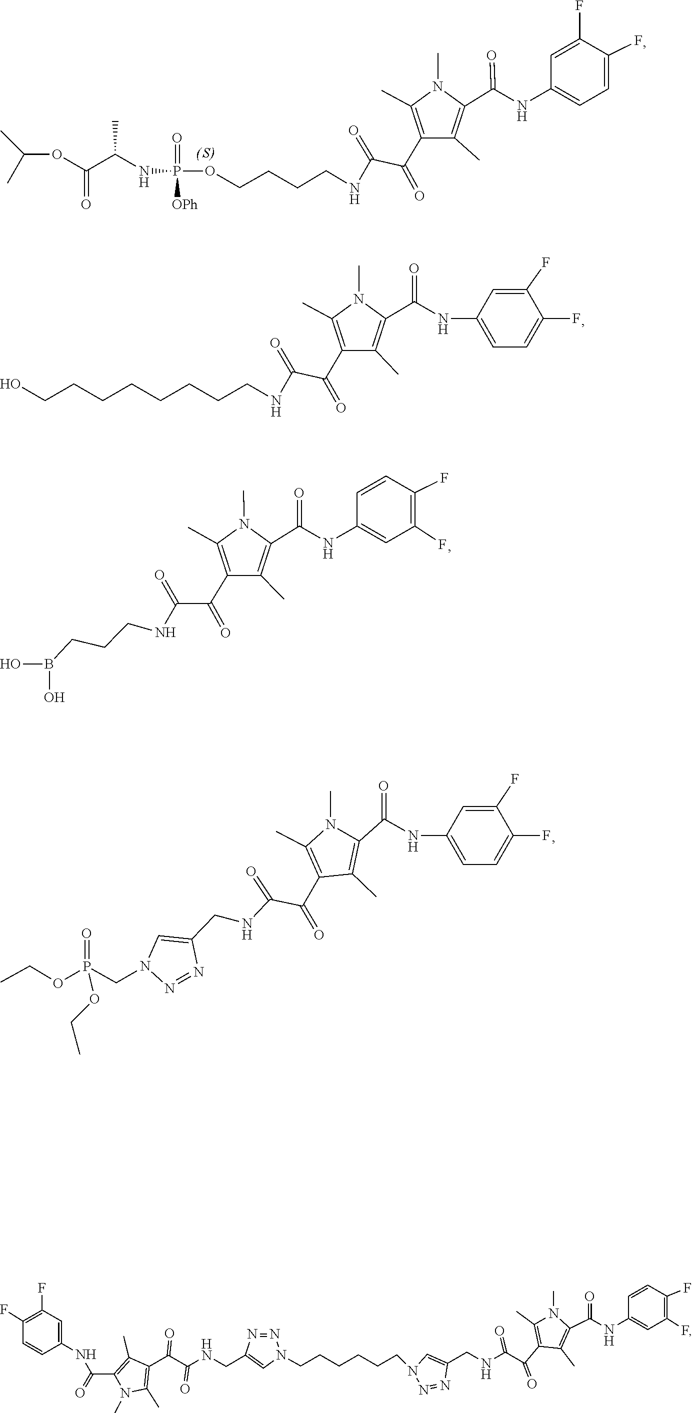

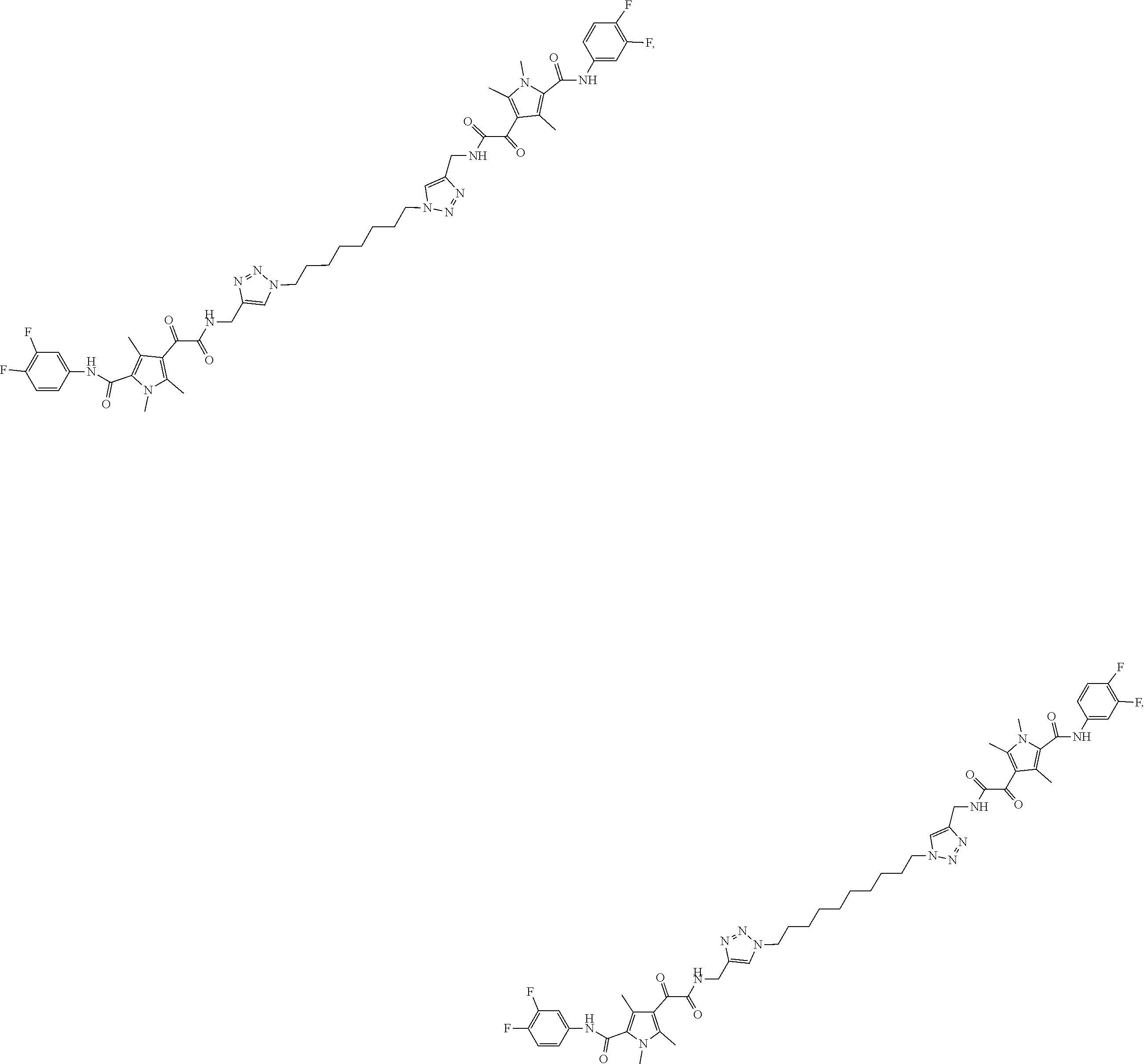

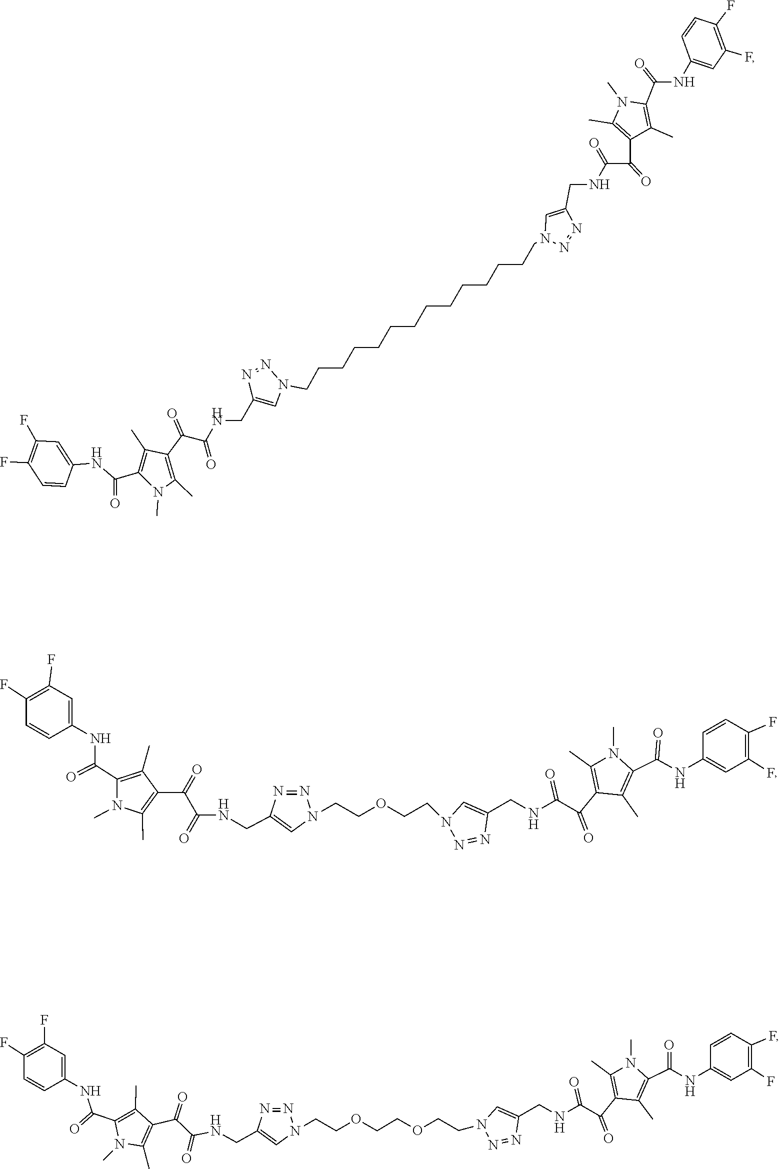

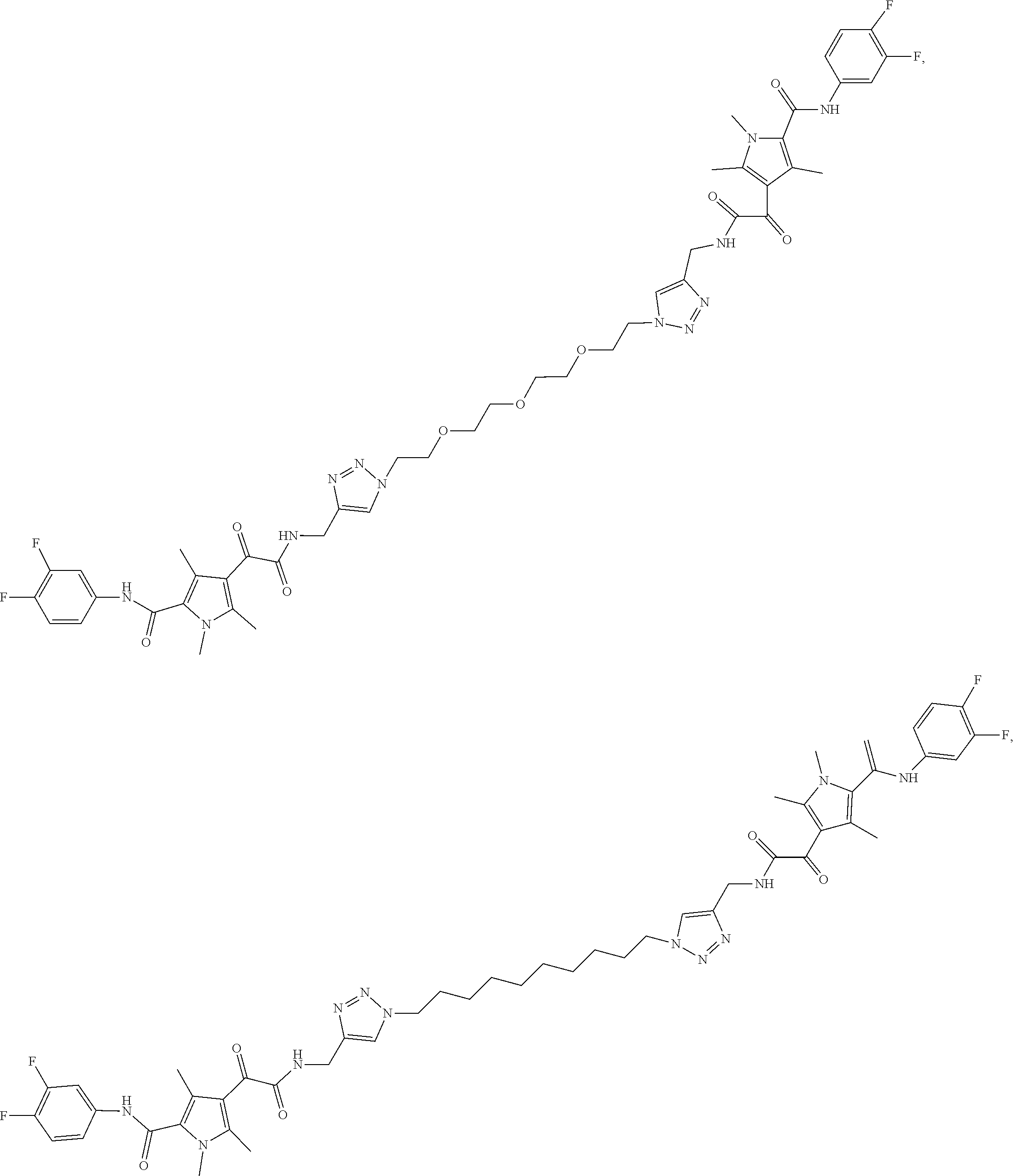 [SMILES: Cc1c(C(=O)C(=O)NCc2cn(CCOCCOCCOCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1 ; C=C(Nc1ccc(F)c(F)c1)c1c(C)c(C(=O)C(=O)NCc2cn(CCCCCCCCCCn3cc(CNC(=O)C(=O)c4c(C)c(C(=O)Nc5ccc(F)c(F)c5)n(C)c4C)nn3)nn2)c(C)n1C]

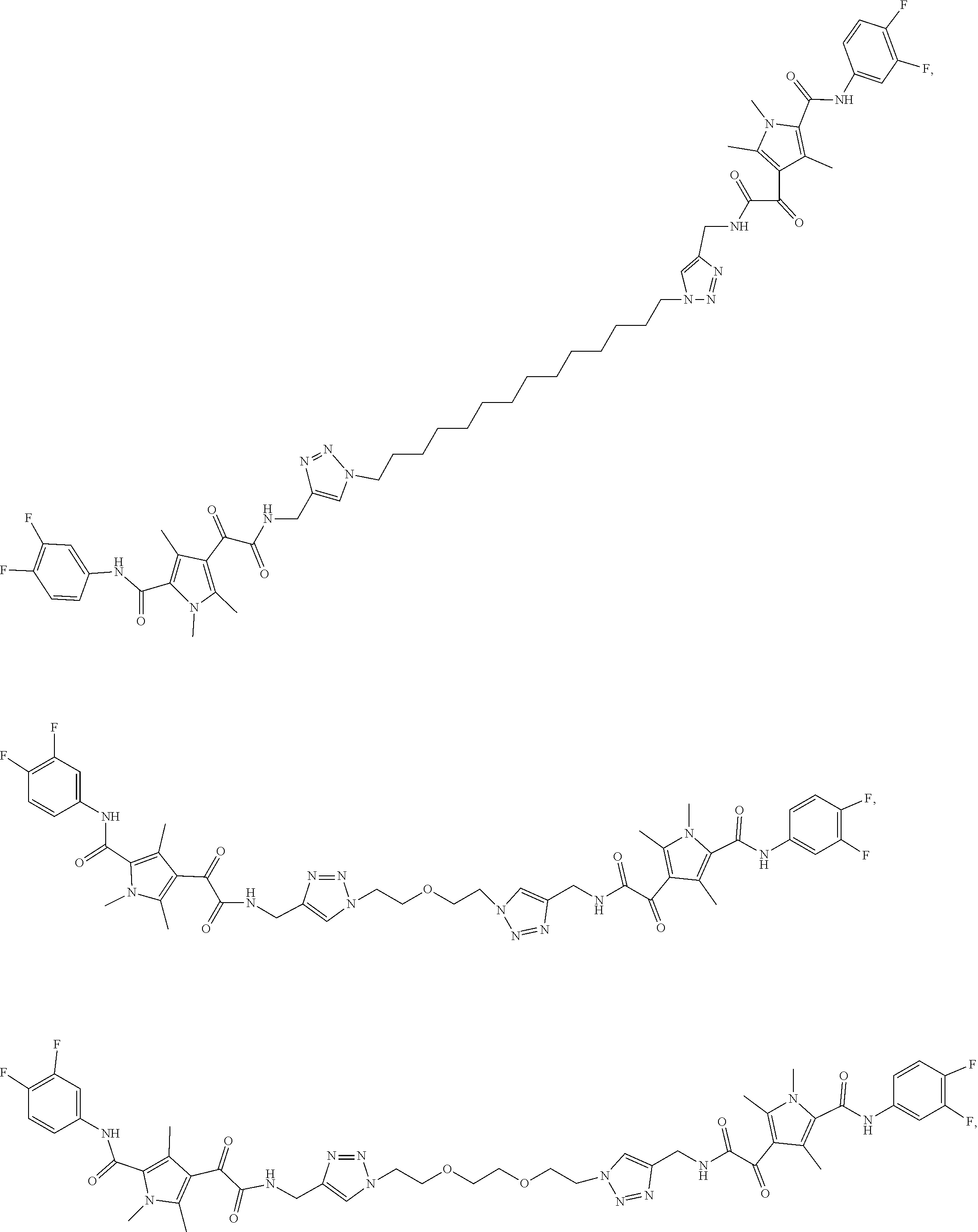

and pharmaceutically-acceptable salts or prodrugs thereof.

### III Stereoisomerism and Polymorphism

The compounds described herein can have asymmetric centers and occur as racemates, racemic mixtures, individual diastereomers or enantiomers, with all isomeric forms being included in the present invention. Compounds of the present invention having a chiral center can exist in and be isolated in optically active and racemic forms. Some compounds can exhibit polymorphism. The present invention encompasses racemic, optically-active, polymorphic, or stereoisomeric forms, or mixtures thereof, of a compound described herein, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals: a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization: a technique whereby the individual enantiomers are separately crystallized from a solution of the racemate, possible only if the latter is a conglomerate in the solid state;

iii) enzymatic resolutions: a technique whereby partial or complete separation of a racemate by virtue of differing rates of reaction for the enantiomers with an enzyme;

iv) enzymatic asymmetric synthesis: a synthetic technique whereby at least one step of the synthesis uses an enzymatic reaction to obtain an enantiomerically pure or enriched synthetic precursor of the desired enantiomer;

v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;

vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors: a synthetic technique whereby the desired enantiomer is obtained from non-chiral starting materials and where the stereochemical integrity is not or is only minimally compromised over the course of the synthesis;

x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;

xi) chiral gas chromatography: a technique whereby the racemate is volatilized and enantiomers are separated by virtue of their differing interactions in the gaseous mobile phase with a column containing a fixed non-racemic chiral adsorbent phase;

xii) extraction with chiral solvents: a technique whereby the enantiomers are separated by virtue of preferential dissolution of one enantiomer into a particular chiral solvent;

xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### IV. Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts can also be formed, including but not limited to, sulfate, nitrate, bicarbonate and carbonate salts. For certain transdermal applications, it can be preferred to use fatty acid salts of the compounds described herein. The fatty acid salts can help penetrate the stratum comeum. Examples of suitable salts include salts of the compounds with stearic acid, oleic acid, lineoleic acid, palmitic acid, caprylic acid, and capric acid.

Pharmaceutically acceptable salts can be obtained using standard procedures well known in the art, for example by reacting a sufficiently basic compound such as an amine with a suitable acid, affording a physiologically acceptable anion. In those cases where a compound includes multiple amine groups, the salts can be formed with any number of the amine groups. Alkali metal (e.g., sodium, potassium or lithium) or alkaline earth metal (e.g., calcium) salts of carboxylic acids can also be made.

A prodrug is a pharmacological substance that is administered in an inactive (or significantly less active) form and subsequently metabolized in vivo to an active metabolite. Getting more drug to the desired target at a lower dose is often the rationale behind the use of a prodrug and is generally attributed to better absorption, distribution, metabolism, and/or excretion (ADME) properties. Prodrugs are usually designed to improve oral bioavailability, with poor absorption from the gastrointestinal tract usually being the limiting factor. Additionally, the use of a prodrug strategy can increase the selectivity of the drug for its intended target thus reducing the potential for off target effects.

### V. Isotopes

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. In other embodiments are examples of isotopes that are incorporated into the present compounds including isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ²H are incorporated, are useful in drug and/or substrate tissue distribution assays. Further, in some embodiments, substitution with isotopes such as deuterium, i.e., ²H, can affords certain therapeutic advantages resulting from greater metabolic stability, such as, for example, increased in vivo half-life or reduced dosage requirements.

### VI. Methods of Treatment

The compounds described herein can be used to prevent, treat or cure HBV infections.

Hosts, including but not limited to humans, suffering from one of these cancers, or infected with one of these viruses, such as HBV, or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, transdermally, subcutaneously, or topically, in liquid or solid form.

### VII. Combination of Alternation Therapy

In one embodiment, the compounds described herein can be employed together with at least one other antiviral agent. Representative antiviral agents include, but are not limited to, polymerase inhibitors, anti-HBV nucleosides and their prodrugs, viral entry inhibitor, viral maturation inhibitor, literature described capsid assembly modulator, IMPDH inhibitors, protease inhibitors, immune-based therapeutic agents, reverse transcriptase inhibitor, a TLR-agonist, and agents of distinct or unknown mechanism. They can also be used in conjunction with CRISPR/CAS9 approaches, for example, using AAV as the human delivery vector.

For example, when used to treat or prevent HBV infection, the active compound or its prodrug or pharmaceutically acceptable salt can be administered in combination or alternation with another anti-HBV agent including, but not limited to, those of the formula above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Nonlimiting examples of antiviral agents that can be used in combination with the compounds disclosed herein include those in the tables below.

| FAMILY/DRUG NAME | MECHANISM | COMPANY/STATUS |
|---|---|---|
| Intron A | Immunomodulator | Merck, |
| (Interferon | | Whitehouse Station, NJ |
| alfa-2b) | | Approved |
| Pegasys | Immunomodulator | Genentech, |
| (Peginterferon | | South San Francisco, CA |
| alfa-2a) | | Approved |
| Epivir-HBV | Inhibits viral DNA | GlaxoSmithKline, |
| (Lamivudine) | polymerase | Philadelphia, PA |
| | | Approved |
| Hepsera | Inhibits viral DNA | Gilead Sciences, |
| (Adefovir | polymerase | Foster City, CA |
| Dipivoxil) | | Approved |
| Baraclude | Inhibits viral DNA | Bristol-Myers Squibb, |
| (Entecavir) | polymerase | Princeton, NJ |
| | | Approved |
| Tyzeka | Inhibits viral DNA | Novartis, Switzerland |
| (Telbivudine) | polymerase | Approved |
| Viread | Inhibits viral DNA | Gilead Sciences, |
| (Tenofovir) | polymerase | Foster City, CA |
| | | Approved |
| Clevudine | Inhibits viral DNA | Bukwang, South Korea |
| (L-FMAU) | polymerase | Eisai, Japan |
| | | Approved S. Korea |
| | | 2006 (Levovir) |
| Tenofovir | Prodrug of | Gilead Sciences, |
| alafenamide | Tenofovir | Foster City, CA |
| (TAF) | | Phase III |
| CMX157 | Prodrug of | ContraVir Pharmaceuticals, |
| | Tenofovir | Edison, NJ |
| | | Phase II |
| AGX-1009 | Prodrug of | Agenix, Australia |
| | Tenofovir | Phase I, China |
| Myrcludex B | Entry Inhibitor | Hepatera, Russia |
| | | with Myr-GmbH, |
| | | Germany Phase II |
| | | for HBV and HDV |
| ARC520 | RNAi gene silencer | Arrowhead Research, |
| | | Pasadena, CA |
| | | Phase II/III |
| NVR 3-778 | Capsid inhibitor | Novira Therapeutics, |
| | | Doylestown, PA |
| | | Phase IIa |
| Morphothiadine | Capsid inhibitor | Sunshine Lake Pharma |
| mesilate (GLS4) | | of HEC, China |
| | | Phase II |
| ISIS-HBVRx | Antisense drug | ISIS Pharma (w/GSK), |
| | | Carlsbad, CA |
| | | Phase II |
| SB 9200 HBV | Small molecule nucleic | Spring Bank Pharma, |
| | acid hybrids or | Milford, MA |
| | “SMNH” | Phase II |
| Rep 2139-Ca | HBsAg release | REPLICor Inc., Canada |
| | inhibitor | Phase II |
| Bay 41-4109 | Capsid Inhibitor | AiCuris, Germany |
| | | Phase I |
| TKM-HBV | RNAi gene silencer | Tekmira, Canada |
| | | Phase I |
| Alinia | Small molecule | Romark Labs, |
| (Nitazoxanide) | | Tampa, FL |
| | | Preclinical |
| CpAMS | HBV Core Protein | Assembly Biosciences, NY, |
| | | NY Preclinical |
| ALN-HBV | RNAi gene silencer | Alnylam, |
| | | Cambridge, MA |
| | | PreclinicaL |
| CPI-431-32 | Cyclophilin | Ciclofilin Pharma, |
| | inhibitor | San Diego, CA |
| | | Preclinical |
| Hepbarna | RNAi gene silencing | Benitec, Australia |
| | | Preclinical |
| OCB-030 | Cyclophilin | Arbutus Biopharma |
| | inhibitor | (formerly Tekmira), Canada |
| | | Preclinical |
| GS-9620 | TLR7 agonist | Gilead Sciences, |
| | | Foster City, CA |
| | | Phase II |
| RO6864018 | TLR7 agonist | Roche, Switzerland |
| (formerly RG7795 | | Phase II |
| and ANA773) | | |
| CYT107 | Immunomodulator | Cytheris, France |
| (Interleukin-7) | | Phase I/IIa |
| NCT01641536 | Therapeutic vaccine | Ichor Medical Systems |
| | | (w/Janssen), San Diego, CA |
| | | Phase I |
| TG 1050 | Immunotherapeutic | Transgene, Shanghai |
| | | Phase I |
| CYT-003 | TLR9 agonist | Arbutus Biopharma |
| | | (formerly Tekmira), Canada |
| | | Preclinical |
| ARB 1467 | TKM-HBV | Arbutus Biopharma |
| | | (formerly Tekmira), Canada |
| | | Preclinical |
| ARB-1468 | TKM-HBV | Arbutus Biopharma |
| | | (formerly Tekmira), Canada |
| | | Preclinical |
| Vemlidy (TAF or | Prodrug of tenofovir | Gilead Sciences |
| tenofovir alafenamide) | | Approved |
| Levovir (Clevudine) | Inhibits viral DNA | Bukwang, S. Korea |
| | polymerase | Approved in Korea |
| Besivo (formerly | Inhibits viral DNA | Ildong Pharma, S. Korea |
| ANA380/LB80380) | polymerase | Approved in Korea |
| Zadaxin | Immunomodulator | SciClone, USA |
| | | Approved outside USA |
| ARB-1740 | RNAi gene silencer (2.0) | Arbutus Biopharma |
| | | Phase II |
| RG6004 (HBV LNA) | RNA targeted via Locked | Roche |
| | Nucleic Acid | Phase I/II |
| ARO-HBV | RNAi gene silencer | Arrowhead Pharma, USA |
| | | Phase I/II |
| Lunar-HBV | RNAi gene silencer | Arcturus, USA with Janssen |
| | | Preclinical |
| AIC 649 | Capsid inhibitor | AiCuris, Germany |
| | | Phase I |
| JNJ56136379 | Capsid Inhibitor | Janssen, USA |
| | | Phase I |
| ABI-H0731 | Capsid inhibitor | Assembly Biosciences, USA |
| | | Phase I |
| AB-423 | Capsid inhibitor | Arbutus Biopharma, Canada |
| | | Phase I |
| EP-027367 | Capsid inhibitor | Enanta Pharmaceuticals, USA |
| | | Preclinical |
| REP 2165 | sAg inhibitor | Replicor, Canada |
| | | Phase II |
| IONIS-HBVRx | Viral protein inhibitor | Ionis Pharma, USA with GSK |
| (GSK3228836) | | Phase I |
| IONIS-HBVLRx | Viral protein inhibitor | Ionis Pharma with GSK |
| (GSK33389404) | | Phase I |
| RNaseH Inhibitor | Viral RNase Inhibitor | Arbutus Biopharma, Canada |
| | | Preclincal |
| GS 4774 | Therapeutic vaccine | GlobeImmune, USA |
| | | Phase II |
| INO-1800 | Therapeutic vaccine | Inovio, USA |
| | | Phase I |
| HB-110 | Therapeutic vaccine | Ichor Medical Systems |
| | | with Janssen, US Phase I |
| HepTcell | Therapeutic vaccine | Altimmune, US |
| | | Phase I |
| TomegaVax HBV | Therapeutic vaccine | TomegaVax, USA |
| | | Preclinical |
| MVA-VLP | Therapeutic vaccine | GeoVax Labs, USA |
| | | Preclinical |
| GS 9620 | TLR-7 agonist | Gilead Sciences |
| | | Phase II |
| Inarigivir | RIG -1 and | Spring Bank Pharmaceuticals, USA |
| (SB9200) | NOD2 agonist | Phase II |
| GS9688 | TLR-8 agonist | Gilead Sciences |
| | | Phase I |
| APG-1387 | Apoptosis Inducer | Ascentage Pharma, China |
| | | Phase I |
| CRV 431 | Ciclofillin inhibitor | ContraVir, USA |
| (CPI 431-32) | | Preclinical |
| EBT106 | CRISPR/Cas | Excision Biotherapeutics, USA |
| | | Preclinical |
| HBV | CRISPR/Cas | Intellia Therapeutics, USA |
| | | Preclinical |
| GC1102 | sAg monoclonal antibody | Green Cross, South Korea |
| | | Phase II |
| EYP001 | FXR agonist | Enyo Pharma, France |
| | | Phase I |
| LTCR-H2-1 | T Cell immunotherapy | Lion TCR Singapore |
| | | Preclinical |
| Lambda (Pegylated | Immunomodulator | Eiger Biopharma, USA |
| Interferon) | | Orphan drug designation |
| Lonafarnib | Prenylation inhibitor | Eiger Biopharma, USA |
| | | Phase II |
| REP 2139 | HBsAg inhibitor | Replicor, Canada |
| | | Phase II |
| Ezetimibe | NTCP inhibitor | Ziauddin University Hospital, |
| | | Pakistan |
| | | Phase II |

Additional Anti-HBV Treatments which can be Used in Combination or Alternation

In addition to the compounds described herein, which can function by inhibiting cccDNA, and the combination therapies described above, which combine the compounds described herein with approved anti-HBV drugs such as TAF, approaches like siRNA, shRNA, Talens, Crisper/Cas9, ARCUS and mir (microRNA) compounds can also be used.

siRNA and shRNA Therapy

siRNA therapy for treating HBV is described, for example, in Chen and Mahato, “siRNA Pool Targeting Different Sites of Human Hepatitis B Surface Antigen Efficiently Inhibits HBV Infection;” J Drug Target. 2008 February; 16(2): 140-148 and Morrissey et al., “Potent and persistent in vivo anti-HBV activity of chemically modified siRNAs,” Nature Biotechnology 23, 1002-1007 (2005).

RNAi is a sequence-specific, post-transcriptional gene silencing mechanism, which is triggered by double-stranded synthetic siRNA or short hairpin RNA (shRNA) expressed intracellularly from a vector. HBV replication and expression can be inhibited by administration of synthetic siRNAs or endogenously expressed shRNAs. See, for example, Giladi et al., “Small interfering RNA inhibits hepatitis B virus replication in mice,” Mol Ther. 2003; 8(5):769-76; McCaffrey et al., “Inhibition of hepatitis B virus in mice by RNA interference,” Nat Biotechnol. 2003; 21(6):639-44; and Shlomai and Shaul, “Inhibition of hepatitis B virus expression and replication by RNA interference,” Hepatology. 2003; 37(4):764-70). HBV gene silencing may depend, for example, on siRNA dosing and sequences, and targets for gene silencing include, for example, the inhibition of virus replication, and suppression of HBsAg expression.

In one embodiment, a combination of several siRNAs and/or shRNAs are used, targeting two or more of the HBV S, C, P and X genes. In this manner, multiple targets for inhibition of HBV replication and gene expression can be accessed.

Once an appropriate target has been identified, for example, the human hepatitis B virus surface antigen (HBsAg) (Gene Bank Accession #NM_U95551), siRNAs can be designed according to the guide provided by Ambion (http://www.ambion.com/techlib/misc/siRNA_finder.html) and Invitrogen (https://maidesigner.invitrogen.com/maiexpress/design.do). The sequence specificity of siRNAs can be checked by performing a BLAST search (www.ncbi.nlm.nih.gov).

Once siRNA sequences are identified, they can be converted into shRNAs. To express shRNA, control vectors can be constructed, for example, using psiSTRIKE™, which is a linearized plasmid and contains a U6 RNA polymerase promoter. These shRNAs contain two complementary oligonucleotides that can be annealed to form double-stranded DNA for ligation into psiSTRIKE™ vector corresponding sites, under a suitable promoter, such as the U6 promoter, using an appropriate ligase, such as T4 DNA ligase. Plasmids can be purified, for example, using the QIAGEN® Plasmid Mini Kit (QIAGEN, Valencia, Calif.).

Talens/CRISPR

As discussed above, chronic HBV viral infections often persist due to the presence of long-lived forms of viral DNA in infected cells. Current therapies can suppress viral replication, but have little or no effect on long-lived DNA forms, so viral replication resumes as soon as therapy is stopped.

In addition to targeting long-lived DNA forms using the capsid inhibitors described herein, targeted endonucleases, such as homing endonucleases, zinc-finger nucleases (ZFNs), transcription activator-like effector nucleases (TALENs), and the CRISPR (clustered regularly interspaced short palindromic repeats) system can be used. The use of TALENS to target HBV is described, for example, in Weber et al., “TALENs Targeting HBV: Designer Endonuclease Therapies for Viral Infections,” Molecular Therapy (2013); 21 10, 1819-1820.

These nucleases function by specifically recognizing and cleaving selected DNA sequences, which results in gene disruption upon imprecise DNA repair. TALENs targeting of the hepatitis B virus (HBV) genome can result in TALEN-induced mutations in the long-lived HBV covalently closed circular DNA (cccDNA). Mutation and/or disruption of cccDNA prevents viral replication by blocking expression of functional viral proteins.

CRISPR

CRISPR, or clustered regularly interspaced short palindromic repeats, is another way to mutate HBV DNA, by providing targeted genome editing. In addition to the programmable editing tools, such as zinc finger nucleases and transcription activator-like effector nucleases (TALENs) described above, CRISPR (clustered regularly interspaced short palindromic repeats)/Cas9 technology also allows for genome editing, and allows for site-specific genomic targeting in HBV.

The type II CRISPR/Cas system is a prokaryotic adaptive immune response system that uses non-coding RNAs to guide the Cas9 nuclease to induce site-specific DNA cleavage. This DNA damage is repaired by cellular DNA repair mechanisms, either via the non-homologous end joining DNA repair pathway (NHEJ) or the homology directed repair (HDR) pathway.

The CRISPR/Cas9 system provides a simple, RNA-programmable method to generate gene knockouts (via insertion/deletion) or knockins (via HDR), and allows for site-specific genomic targeting in HBV. The type II CRISPR/Cas system is a prokaryotic adaptive immune response system that uses non-coding RNAs to guide the Cas9 nuclease to induce site-specific DNA cleavage.

To create gene disruptions, a single guide RNA (sgRNA) is generated to direct the Cas9 nuclease to a specific genomic location. Cas9-induced double strand breaks are repaired via the NHEJ DNA repair pathway. The repair is error prone, and thus insertions and deletions (INDELs) may be introduced that can disrupt gene function.

Thus, targeting hepatitis B virus cccDNA using a CRISPR/Cas9 nuclease can efficiently inhibits viral replication.

ARCUS

ARCUS is a next-generation genome editing platform derived from a natural genome editing enzyme called a homing endonuclease. They are site-specific DNA-cutting enzymes encoded in the genomes of many eukaryotic species. Homing endonucleases have the unusual ability to precisely recognize long DNA sequences (12-40 base pairs) that are typically rare enough to occur only once in a complex genome. These non-destructive enzymes trigger gene conversion events that modify the genome in a very precise way, most frequently by the insertion of a new DNA sequence. The backbone of the ARCUS technology is the ARC nuclease—a fully synthetic enzyme similar to a homing endonuclease but significantly improved to be the starting point for the first therapeutic-grade genome editing platform. The ARC nuclease is small, has unparalleled specificity, and can be customized to recognize a DNA sequence within any target gene. ARC nucleases are created using a set of proprietary in silico and lab-based techniques to ensure maximum gene editing efficiency with minimum off-target activity. Importantly, ARC nucleases can be optimized to control potency and specificity based on the analysis of cutting activity in a relevant model organism such as HBV.

Mir/MicroRNA

MicroRNAs (miRNAs) are tiny noncoding RNAs that regulate gene expression primarily at the post-transcriptional level by binding to mRNAs. miRNAs contribute to a variety of physiological and pathological processes. A number of miRNAs have been found to play a pivotal role in the host-HBV interaction. HBV infection can change the cellular miRNA expression patterns, and different stages of HBV associated disease have displayed distinctive miRNA profiles. The differential expressed miRNAs are involved in the progression of HBV-related diseases. For instance, some miRNAs are involved in liver tumorigenesis and tumor metastasis. Circulating miRNA in serum or plasma can be a very useful biomarker for the diagnosis and prognosis of HBV-related diseases. In addition, miRNA-based therapy can be used to treat, prevent, or cure HBV-related diseases. See, for example, Ying-Feng Wei, “MicroRNAs may solve the mystery of chronic hepatitis B virus infection,” World J Gastroenterol. 2013 Aug. 14; 19(30): 4867-4876. http://www.ncbi.nlm.nih.gov/pmc/articles/PMC3740416/

In the interaction between virus and host, miRNAs can be divided into cellular miRNAs and viral miRNAs. Cellular miRNAs' expression profiles change at the infected state, and abnormal miRNAs often closely relate to the viral life cycle as well as the host disorder. Viral miRNAs can evolve to regulate both viral and cellular gene expression.

Sometimes, viruses exploit cellular miRNAs to facilitate certain steps of their life cycle. For example, miR-122 serves an antiviral role in the HBV life cycle. MiR-122 over-expression inhibits HBV expression, whereas depletion of endogenous miR-122 results in increased HBV production in transfected cells. MiR-122 inhibitors cause an increase in cellular heme oxygenase-1, which can decrease HBV covalently closed circular DNA (cccDNA) levels by reducing the stability of the HBV core protein. MiR-122 expression in the liver can be significantly down-regulated in patients with HBV infection compared with healthy controls. MiR-122 is significantly up-regulated in HBV-infected patients, and can inhibit HBV replication in Huh7 and HepG2 cells. Cyclin G1 is a miR-122 target that specifically interacts with p53, resulting in the specific binding of p53 to the HBV enhancer elements and simultaneous abrogation of the p53-mediated inhibition of HBV transcription.

HBV is a noncytopathic virus that replicates preferentially in the hepatocytes. cccDNA serves as a template for transcription of all viral RNA that is synthetized after HBV DNA enters the hepatocyte nucleus. The HBV genome is 3.2 kb in length and contains four overlapping open reading frames. It can transcribe viral pregenomic RNA that reverses transcription to synthesize the viral DNA genome and encode the hepatitis B virus surface antigen (HBsAg), hepatitis B virus core protein, viral reverse DNA polymerase (Pol) and X protein.

Hsa-miR-125a-5p interferes with HBV translation and down-regulates the expression of the HBV surface antigen. Accordingly, cellular miRNAs can alter HBV gene expression by targeting to HBV transcripts.

Cellular miRNAs can affect viral translation and change viral replication. In addition to the instance of the miR-122 inhibition of HBV replication, there are other examples where host miRNAs alter HBV replication. MiR-141 suppresses HBV replication by reducing HBV promoter activities, by down-regulating peroxisome proliferator-activated receptor alpha. DNA hypermethylation may be closely related to the suppression of HBV cccDNA transcription, and miR-152 may be a factor involved in the regulation of the methylation of HBV cccDNA.

Accordingly, miRNAs can directly or indirectly alter HBV replication. The close relationship between miRNAs and HBV-related diseases offers an opportunity to use miRNAs or antagomir in combination therapies to treat, cure, or prevent HBV.

### VIII. Pharmaceutical Compositions

Hosts, including but not limited to humans, infected with HBV can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound will be in the range of between about 0.01 and about 10 mg/kg, more generally, between about 0.1 and 5 mg/kg, and, preferably, between about 0.5 and about 2 mg/kg, of body weight of the recipient per day. The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 600 mg, preferably 60 to 600 mg of active ingredient per unit dosage form. An oral dosage of 1-400 mg is usually convenient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral, although for certain patients a sterile injectable form can be given sc, ip or iv. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antiviral compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

Transdermal Formulations

In some embodiments, the compositions are present in the form of transdermal formulations, such as that used in the FDA-approved agonist rotigitine transdermal (Neupro patch). Another suitable formulation is described in U.S. Publication No. 20080050424, entitled “Transdermal Therapeutic System for Treating Parkinsonism.” This formulation includes a silicone or acrylate-based adhesive, and can include an additive having increased solubility for the active substance, in an amount effective to increase dissolving capacity of the matrix for the active substance.

The transdermal formulations can be single-phase matrices that include a backing layer, an active substance-containing self-adhesive matrix, and a protective film to be removed prior to use. More complicated embodiments contain multiple-layer matrices that may also contain non-adhesive layers and control membranes. If a polyacrylate adhesive is used, it can be crosslinked with multivalent metal ions such as zinc, calcium, aluminum, or titanium ions, such as aluminum acetylacetonate and titanium acetylacetonate.

When silicone adhesives are used, they are typically polydimethylsiloxanes. However, other organic residues such as, for example, ethyl groups or phenyl groups may in principle be present instead of the methyl groups. Because the active compounds are amines, it may be advantageous to use amine-resistant adhesives. Representative amine-resistant adhesives are described, for example, in EP 0 180 377.

Representative acrylate-based polymer adhesives include acrylic acid, acrylamide, hexylacrylate, 2-ethylhexylacrylate, hydroxyethylacrylate, octylacrylate, butyl acrylate, methylacrylate, glycidylacrylate, methacrylic acid, methacrylamide, hexylmethacrylate, 2-ethylhexylmethacrylate, octylmethacrylate, methylmethacrylate, glycidylmethacrylate, vinylacetate, vinylpyrrolidone, and combinations thereof.

The adhesive must have a suitable dissolving capacity for the active substance, and the active substance most be able to move within the matrix, and be able to cross through the contact surface to the skin. Those of skill in the art can readily formulate a transdermal formulation with appropriate transdermal transport of the active substance.

Certain pharmaceutically acceptable salts tend to be more preferred for use in transdermal formulations, because they can help the active substance pass the barrier of the stratum comeum. Examples include fatty acid salts, such as stearic acid and oleic acid salts. Oleate and stearate salts are relatively lipophilic, and can even act as a permeation enhancer in the skin.

Permeation enhancers can also be used. Representative permeation enhancers include fatty alcohols, fatty acids, fatty acid esters, fatty acid amides, glycerol or its fatty acid esters, N-methylpyrrolidone, terpenes such as limonene, alpha-pinene, alpha-terpineol, carvone, carveol, limonene oxide, pinene oxide, and 1,8-eucalyptol.

The patches can generally be prepared by dissolving or suspending the active agent in ethanol or in another suitable organic solvent, then adding the adhesive solution with stirring. Additional auxiliary substances can be added either to the adhesive solution, the active substance solution or to the active substance-containing adhesive solution. The solution can then be coated onto a suitable sheet, the solvents removed, a backing layer laminated onto the matrix layer, and patches punched out of the total laminate.

Nanoparticulate Compositions

The compounds described herein can also be administered in the form of nanoparticulate compositions.

In one embodiment, the controlled release nanoparticulate formulations comprise a nanoparticulate active agent to be administered and a rate-controlling polymer which functions to prolong the release of the agent following administration. In this embodiment, the compositions can release the active agent, following administration, for a time period ranging from about 2 to about 24 hours or up to 30 days or longer. Representative controlled release formulations including a nanoparticulate form of the active agent are described, for example, in U.S. Pat. No. 8,293,277.

Nanoparticulate compositions comprise particles of the active agents described herein, having a non-crosslinked surface stabilizer adsorbed onto, or associated with, their surface.

The average particle size of the nanoparticulates is typically less than about 800 nm, more typically less than about 600 nm, still more typically less than about 400 nm, less than about 300 nm, less than about 250 nm, less than about 100 nm, or less than about 50 nm. In one aspect of this embodiment, at least 50% of the particles of active agent have an average particle size of less than about 800, 600, 400, 300, 250, 100, or 50 nm, respectively, when measured by light scattering techniques.

A variety of surface stabilizers are typically used with nanoparticulate compositions to prevent the particles from clumping or aggregating. Representative surface stabilizers include gelatin, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, poloxamines, poloxamine 908, dialkylesters of sodium sulfosuccinic acid, sodium lauryl sulfate, an alkyl aryl polyether sulfonate, a mixture of sucrose stearate and sucrose distearate, p-isononylphenoxypoly-(glycidol),

SA9OHCO, decanoyl-N-methylglucamide, n-decyl-D-glucopyranoside, n-decyl-D-maltopyranoside, n-dodecyl-D-glucopyranoside, n-dodecyl-D-maltoside, heptanoyl-N-methylglucamide, n-heptyl-D-glucopyranoside, n-heptyl-D-thioglucoside, n-hexyl-D-glucopyranoside, nonanoyl-N-methylglucamide, n-nonyl-D-glucopyranoside, octanoyl-N-methylglucamide, n-octyl-D-glucopyranoside, and octyl-D-thioglucopyranoside. Lysozymes can also be used as surface stabilizers for nanoparticulate compositions. Certain nanoparticles such as poly(lactic-co-glycolic acid) (PLGA)-nanoparticles are known to target the liver when given by intravenous (IV) or subcutaneously (SQ).

Because HBV causes damage to, and are present in the liver, in one embodiment, the nanoparticles or other drug delivery vehicles are targeted to the liver. One such type of liver-targeted drug delivery vehicle is described in Park, et al., Mol Imaging. February 2011; 10(1): 69-77, and uses Glypican-3 (GPC3) as a molecular target. Park taught using this target for hepatocellular carcinoma (HCC), a primary liver cancer frequently caused by chronic persistent hepatitis.

In one aspect of this embodiment, this drug delivery vehicle is also used to target therapeutics to the liver to treat viral infections. Further, since the compounds described herein have indirect anti-cancer uses, this type of system can target the compounds to the liver and treat liver cancers or reverse the cancer. GPC3 is a heparan sulfate proteoglycan that is not expressed in normal adult tissues, but significantly over-expressed in up to 80% of human HCC's. GPC3 can be targeted, for example, using antibody-mediated targeting and binding (See Hsu, et al., Cancer Res. 1997; 57:5179-84).

Another type of drug delivery system for targeting the liver is described in U.S. Pat. No. 7,304,045. The '045 patent discloses a dual-particle tumor or cancer targeting system that includes a first ligand-mediated targeting nanoparticle conjugated with galactosamine, with the ligand being on a target cell. The first nanoparticle includes poly(γ-glutamic acid)/poly(lactide) block copolymers and n antiviral compound, which in this case is a compound described herein, and in the '045 patent, was ganciclovir. A second nanoparticle includes poly(γ-glutamic acid)/poly(lactide) block copolymers, an endothelial cell-specific promoter, and a (herpes-simplex-virus)-(thymidine kinase) gene constructed plasmid, and provides enhanced permeability and retention-mediated targeting. The first and said second nanoparticles are mixed in a solution configured for delivering to the liver. When the disorder to be treated is a liver tumor or cancer, the delivery can be directly to, or adjacent to, the liver tumor or cancer.

Representative rate controlling polymers into which the nanoparticles can be formulated include chitosan, polyethylene oxide (PEO), polyvinyl acetate phthalate, gum arabic, agar, guar gum, cereal gums, dextran, casein, gelatin, pectin, carrageenan, waxes, shellac, hydrogenated vegetable oils, polyvinylpyrrolidone, hydroxypropyl cellulose (HPC), hydroxyethyl cellulose (HEC), hydroxypropyl methylcelluose (HPMC), sodium carboxymethylcellulose (CMC), poly(ethylene) oxide, alkyl cellulose, ethyl cellulose, methyl cellulose, carboxymethyl cellulose, hydrophilic cellulose derivatives, polyethylene glycol, polyvinylpyrrolidone, cellulose acetate, cellulose acetate butyrate, cellulose acetate phthalate, cellulose acetate trimellitate, polyvinyl acetate phthalate, hydroxypropylmethyl cellulose phthalate, hydroxypropylmethyl cellulose acetate succinate, polyvinyl acetaldiethylamino acetate, poly(alkylmethacrylate), poly(vinyl acetate), polymers derived from acrylic or methacrylic acid and their respective esters, and copolymers derived from acrylic or methacrylic acid and their respective esters.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(-)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” and U.S. Pat. No. 6,432,381 for “Methods for targeting drug delivery to the upper and/or lower gastrointestinal tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

The nanoparticle formulations including the compounds described herein, and also in the form of a prodrug or a salt, can be used to treat or prevent infections by hepatitis B virus.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Controlled Release Formulations

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

The terms used in describing the invention are commonly used and known to those skilled in the art. As used herein, the following abbreviations have the indicated meanings:

Boc₂O Di-tert-butyl dicarbonate
CDI carbonyldiimidazole
DCC N,N′-dicyclohexylcarbodiimide
DIPEA diisopropyl ethyl amine (Hünig's base)

### DMAP 4-Dimethylaminopyridine

### DMF N,N-dimethylformamide

DMSO dimethylsulfoxide
EDC 1-ethyl-3-(3-dimethylaminopropyl)carbodiimide hydrochloride
EtOAc ethyl acetate
h hour
HATU 1-[Bis(dimethylamino)methylene]-1H-1,2,3-triazolo[4,5-b]pyridinium-3-oxide hexafluorophosphate
M molar
min minute
rt or RT room temperature
TFA trifluoroacetic acid
THF tetrahydrofuran

### DMA Dimethylacetamide

### IX. General Methods for Preparing Active Compounds

Methods for the facile preparation of active compounds are known in the art and result from the selective combination of known methods. The compounds disclosed herein can be prepared as described in detail below, or by other methods known to those skilled in the art. It will be understood by one of ordinary skill in the art that variations of detail can be made without departing from the spirit and in no way limiting the scope of the present invention.

The various reaction schemes are summarized below.

Scheme 1 is a non-limiting example of the synthesis of intermediate IX and X.

Scheme 2 is a non-limiting example of the synthesis of active compounds of the present invention, and in particular, a synthetic approach to compounds of Formulas A-C.

Scheme 3 is an alternate synthetic approach to intermediate IV.

Scheme 4 is an alternate synthetic approach to intermediate IV.

Scheme 5 is a synthetic approach to intermediate XXIII

Scheme 6 is a synthetic approach to intermediate XXIV

Scheme 7 is a synthetic approach to intermediate XXVII.

Scheme 8 is a synthetic approach to compound D, which can be used to make the compounds of Formula 8.

Scheme 9 is a non-exhaustive list of reagents that can be used to link HBV Capsid Assembly Effectors (“CAE”).

Schemes 10 and 11 are synthetic approaches to diazo linkers.

Scheme 12 is a synthetic approach to compounds of Formula HD (dimers where a central linker moiety is attached to two CAE through a heteroaryl moiety formed by coupling an azide with an alkyne).

Scheme 13 is a synthetic approach to form compounds of formula DAM (dimers where a central linker moiety is attached to two CAE moieties through amide linkages).

Scheme 14 is a synthetic approach to compounds of Formula DHAR (dimers where a central linker moiety is attached to two CAE through a heteroaryl-alkenyl or alkynyl moiety), formed by coupling a heteroaryl ring to an alkene or alkyne moiety attached to the CAE using transition metal catalyzed coupling chemistry, and Formula DHAR2 (dimers where a central linker moiety is attached to two CAE through a heteroaryl-alkyl moiety) formed by reducing the double or triple bonds in the compounds of Formula DHAR.

Scheme 15 is a synthetic approach to compounds of Formulas LO and LA (dimers where a central linker moiety which includes two double bonds (Formula LDB) is attached to two CAE via olefin (LO) or alkyl (LA) linkages).

Scheme 16 is a synthetic approach to compounds C₅-C₇.

Scheme 17 is a synthetic approach to compounds A2-A9.

Scheme 18 is a synthetic approach to compounds of formulas B1, B2, B3, B3B, B4, B5, B6, B6B, and B7.

Scheme 19 is a synthetic approach to compound 8.

Scheme 20 is a synthetic approach to compound 17.

Scheme 21 is a synthetic approach to compound 9.

Scheme 22 is a synthetic approach to compound 16a.

Scheme 23 is a synthetic approach to compound 17c.

Scheme 24 is a synthetic approach to compound 20.

Scheme 25 is a synthetic approach to compound 23.

Scheme 26 is a synthetic approach to compound 28.

Scheme 27 is a synthetic approach to compound 33.

Scheme 28 is a synthetic approach to compound 45.

Scheme 29 is a synthetic approach to compound 50.

Scheme 30 is a synthetic approach to compound 53.

Scheme 31 is a synthetic approach to compounds 58 and 60.

Scheme 32 is a synthetic approach to compounds 62 and 64.

Compounds of general formula A, B, C can be synthesized from common intermediates IX or X. As outlined in Scheme 1, intermediates IX and X can be prepared by first reaction of ketoester II with an aldehyde or ketone of general formula III and an amidine of general formula I to form a dihydropyrimidine of general formula IV. Halogenation of the 6-methyl by treatment, for example, with N-bromosuccinimide (NBS) or other N-halosuccinimides (NXS) and subsequent reaction with an amine of general formula V in presence of an organic base such as pyridine can afford compound of general formula VI. Deprotection of this intermediate using, for example, BCh, or through hydrogenation, subsequent halodecarboxylation using, for example, NaI, oxone and K₂CO₃ and final N-protection with, for example, carbobenzoxy chloride (CBzCl) in the presence of a base such as KHMDS (potassium bis(trimethylsilyl)amide) can give compounds of general formula IX or X.

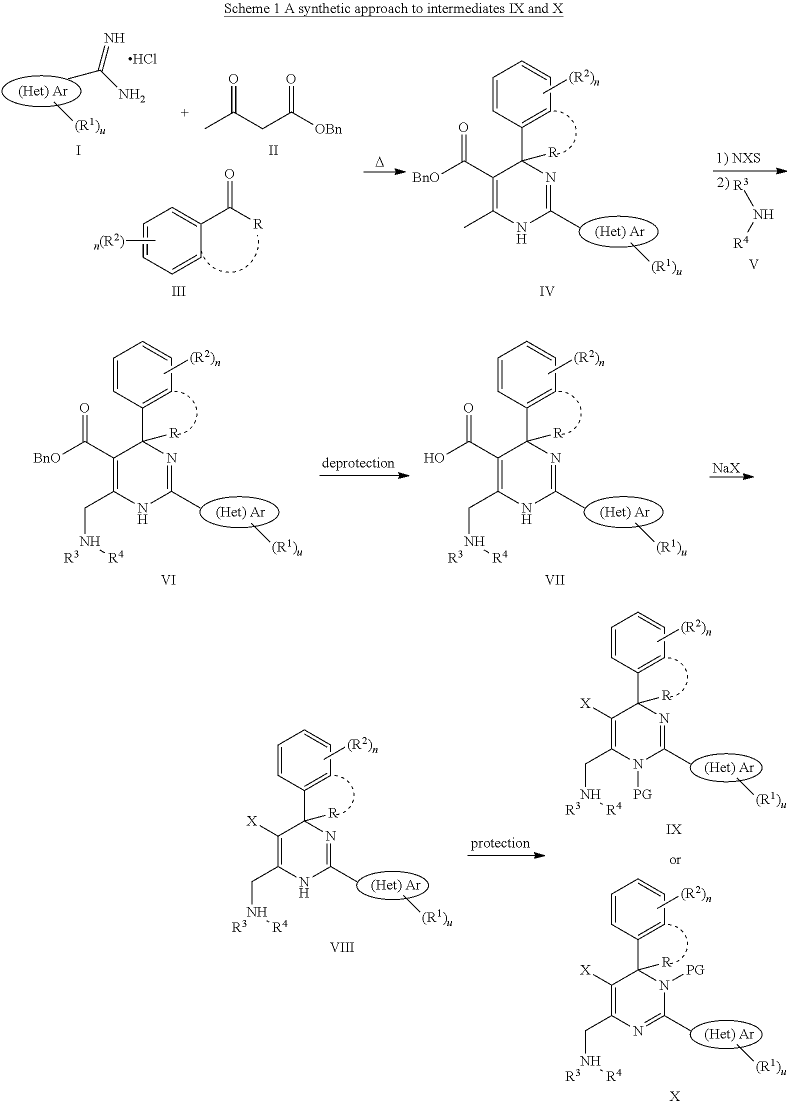

Compounds of general formulas A-C can be prepared from intermediates IX and X through a Sonogashira type reaction with an alkyne of general formula XI followed by a deprotection step. For example, BCh can be used when carbobenzoxy (Cbz) is used as a protecting group. Compounds of general formula B can be obtained by reduction of compounds A using, for instance, H₂ in the presence of Lindlar catalyst. Compounds of general formula C can be synthesized, for example, via a palladium catalyzed reaction with an alkene of general formula XIV under Heck, Suzuki-Myaura or Stille coupling conditions.

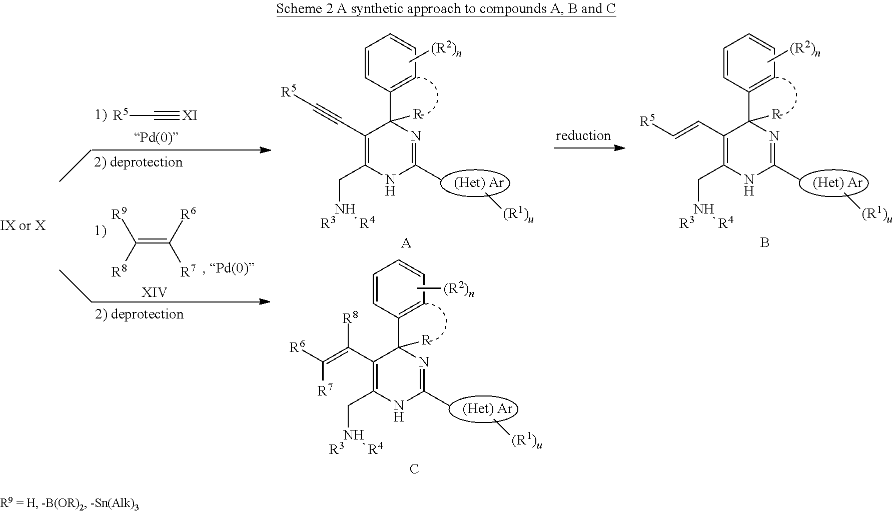

Alternatively, intermediate IV can be prepared by first reacting a ketone or aldehyde of general formula II with ketoacid I in presence of a Lewis acid, for example, TiCl₄, then reaction with amidine XIV to form a thioether intermediate of general formula XV, which can finally react in a transition metal-catalyzed C—C bond formation reaction such as, for example, a Heck type coupling.

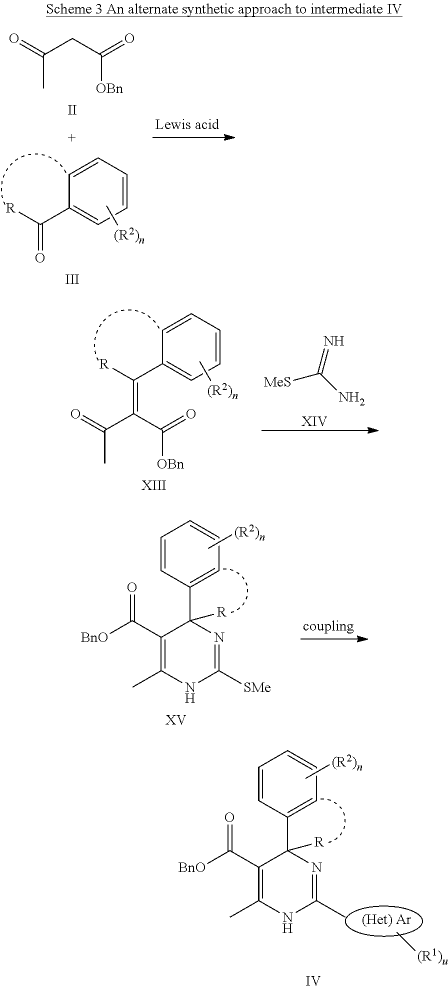

Key intermediate IV can also be prepared by reacting compound XIII with urea, halogenating intermediate XVII with, for instance, POCl₃ and, finally, transition metal-catalyzed coupling reactions such as those described above.

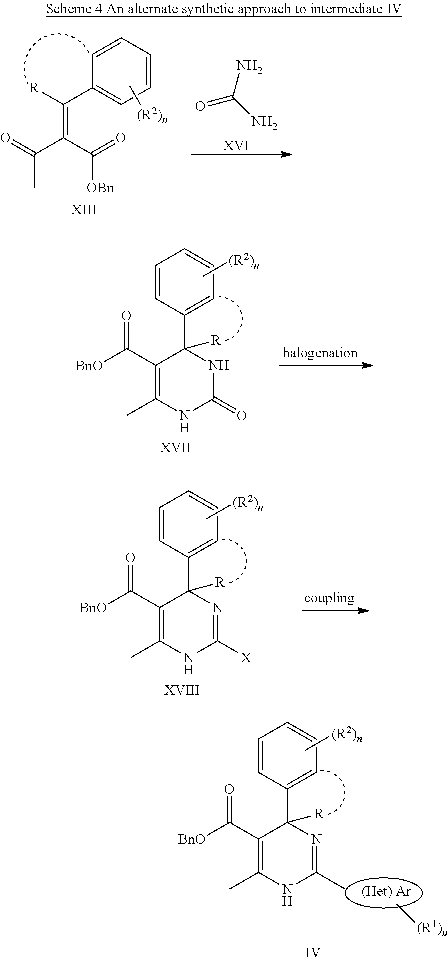

Intermediates of general formula XXIII can be prepared from compound IV by first halogenating it with, for example, N-bromosuccinimide (NBS) in 1,2-dichloroethane, and then reacting the halogenated compound with a protected amine of general formula XX in the presence of a base such as K₂CO₃. The hydroxyl group in compound XXI can be converted to a leaving group, for example, by reaction with mesyl chloride (MsCl) and an organic base such as triethylamine (Et₃N), followed by cyclization in the presence of an inorganic base such as K₂CO₃. Other leaving groups, including tosylate, triflate, brosylate, nosylate, and the like, can also be used.

Deprotection of a bicyclic compound of general formula XXII with, for example, 1-chloroethyl chloroformate when PMB (p-methoxybenzyl) is used as a protecting group, and, finally, reaction with an electrophile, for example, ethyl chloroformate, in the presence of an organic base such as Et₃N, can afford intermediate of general formula XXIII.

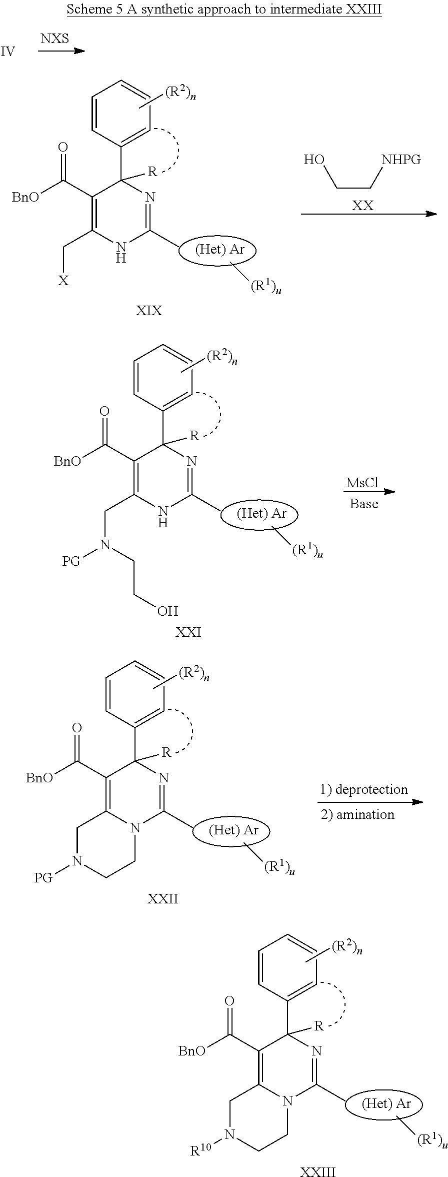

Intermediates of general formula XXIV can be prepared from compound XIX by first reacting Compound XIX with an amine, such as A-methylamine, in the presence of a base, such as t-BuOK, followed by reaction with 2-chloroacetyl chloride in presence of an organic base such as Et₃N and final cyclization under basic conditions.

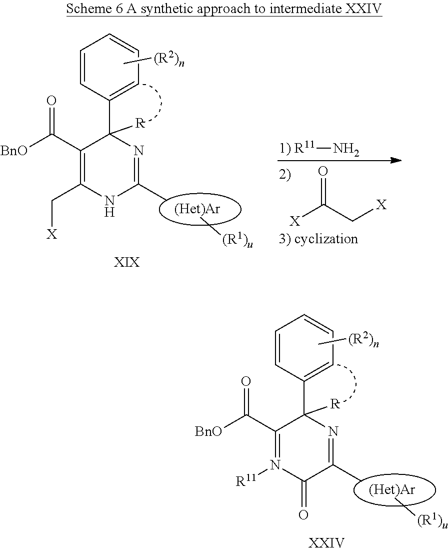

Activated compound of general formula XIX can be reacted with a malonate in presence of base such as NaH to from intermediate XXV which can then be reduced with for instance NaBH₄. Reaction of the resulting diol with MsCl in the presence of an organic base can give access to a cyclized derivative of general formula XXVI which can finally be reacted with a nucleophile, such as NaSMe, to produce a compound of general formula XXVII.

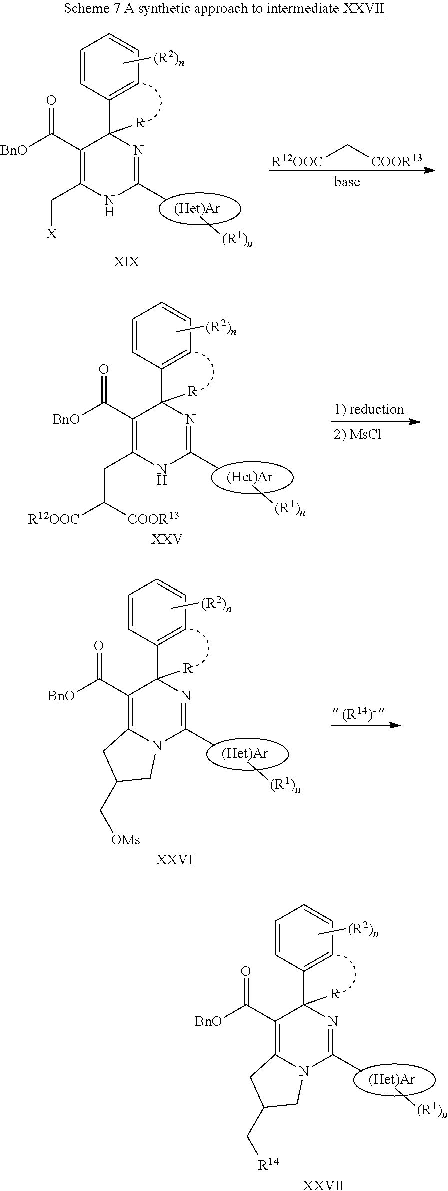

Bicyclic compounds of general structure D can be prepared, for example, from intermediates XXIII, XXIV or XXVII by first deprotecting the ester group with, for instance, BCh in dichloromethane, followed by subsequent halodecarboxylation, using, for example, NaI, oxone and K₂CO₃, and final transition metal-catalyzed coupling reactions such as Sonogashira, Heck, Suzuki-Myaura or Stille reactions.

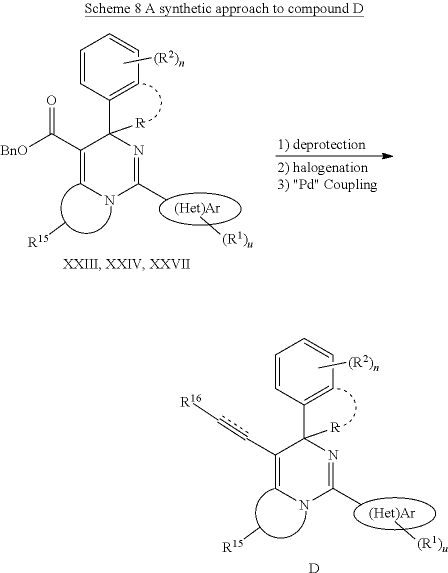

Linked HBV Capsid Assembly Effectors (CAE) of general formula 1 can be prepared using bi or tri functional, homo or heterofunctional linkers and cross linking reagents. The preparation of such compounds of general Formula 1 can be accomplished by one of ordinary skill in the art, using, for instance, methods and linkers outlined in TermoFisher Scientific Crosslinking Reagents Technical Handbook (https://tools.thermofisher.com/content/sfs/brochures/1602163-Crosslinking-Reagents-Handbook.pdf); Bioconjugate Techniques, 3rd Edition (Academic Press, 2013, ISBN: 978-0-12-382239-0) by Greg T. Hermanson.

As shown below in Scheme 9, there are many types of reagents which can be used to link HBV CAE to a central linker. For example, amines (i.e., X═NH₂) on a linker moiety can be reacted with leaving groups (LG) on an HBV CAE, or on a moiety attached to the HBV CAE, and vice versa. Azides on a linker moiety can be reacted with alkyne moieties on an HBV CAE, or on a moiety attached to the HBV CAE, and vice versa. Carboxylic acid groups on a linker moiety can be reacted with hydroxyl, thiol, or amine groups on an HBV CAE, or on a moiety attached to the HBV CAE, and vice versa. Alkene or alkyne moieties on a linker moiety can be reacted with halides on an HBV CAE, or on a moiety attached to the HBV CAE, for example, using transition metal catalyzed coupling reactions, and vice versa. Double or triple bonds remaining after the coupling step can be reduced, for example, using hydrogen and an appropriate catalyst, should such be desired.

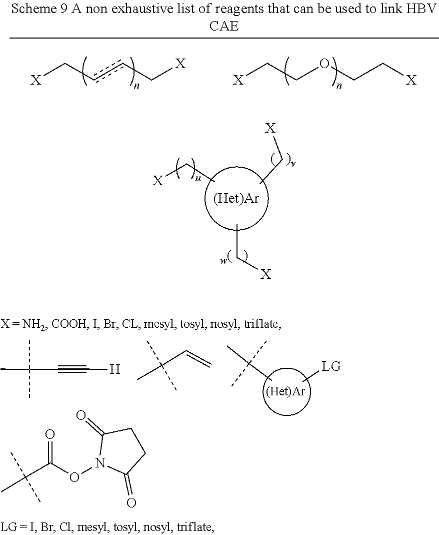

Diazido linkers of Formula DAL can be prepared, for example, through the activation of diols of general formula DOL using for instance, mesyl chloride (MesCl) in presence of an organic base such as pyridine, followed by substitution with NaN₃, for example, in a polar solvent, such as DMF.

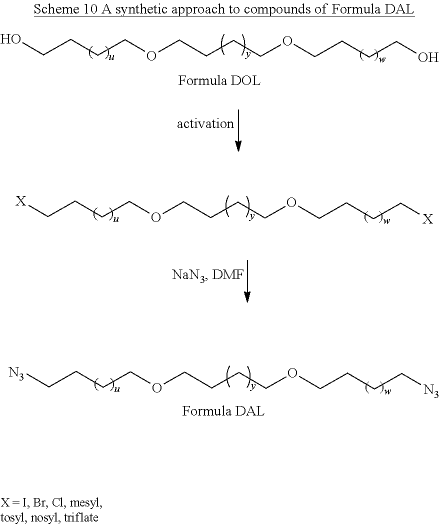

Diazido linkers of general Formula DAL2 can be prepared, for example, through the activation of diols of general Formula DOL2, using for instance, triflic anhydride (Tf₂O) in presence of an organic base, such as pyridine, followed by substitution with an azide salt, such as NaN₃, in a polar solvent, such as DMF.

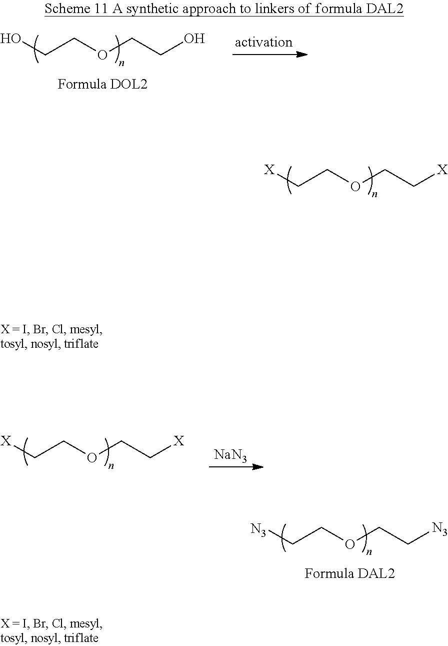

Dimers or heterodimers of general Formula HD can be prepared, for example, through the copper(I)-catalyzed alkyne-azide cycloaddition of a diazido linker of general formula LAZ (azido linker) with an alkyne derivative of a HBV CAE, in the presence, for example, of CuSO₄ and sodium ascorbate in a mixture of water and tBuOH.

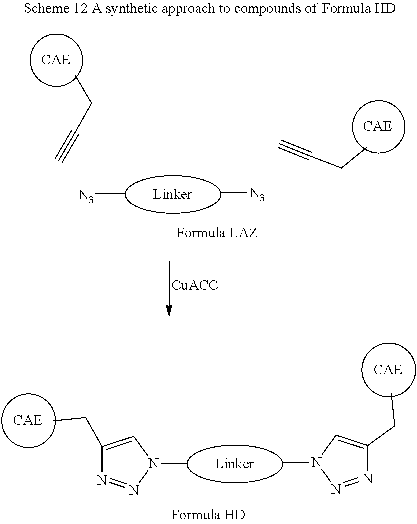

Dimers or heterodimers of general Formula DAM (dimers with amide linkages) can be prepared, for example, through the coupling of a diamino linker of general Formula LAM (linkers with amine groups) with a carboxylic acid derivative of a HBV CAE, in the presence of a peptide coupling reagent like, for example, HATU (1-[Bis(dimethylamino)methylene]-1H-1,2,3-triazolo[4,5-b]pyridinium 3-oxid hexafluorophosphate, Hexafluorophosphate Azabenzotriazole Tetramethyl Uronium) in the presence of an organic amine base such as DIPEA (N,N-Diisopropylethylamine, also known as Hunig's base). Dimers or heterodimers of general DAM can also be prepared, for example, through the coupling of a diamino linker of general formula LAM with a N-hydroxysuccinimide ester derivative of a HBV CAE in presence of an organic amine base such as DIPEA.

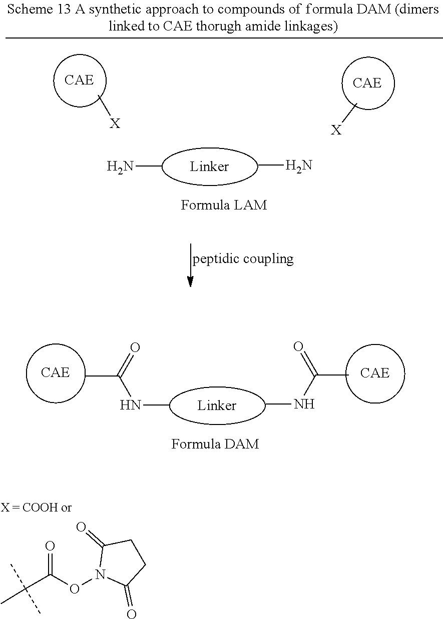

Dimers or heterodimers of general Formula DHAR (dimer linked through heteroaryl-alkenyl or alkynyl linkages to HBV CAEs) can be prepared, for example, through a transition metal catalyzed coupling reaction, such as a Heck or a Sonogashira reaction, between a correctly functionalized HBV CAE of general Formula AC (alkene/alkyne-functionalized CAE), and a linker with two aromatic or heteroamantic rings substituted with a leaving group such as I, Br, Cl, mesylate, tosylate or triflate. Reduction of the unsaturation formed, using for instance, H₂ and Pd/C in MeOH, can produce compounds of general formula DHAR2 (dimer linked to CAE through a di-heteroaryl-alkyl linker).

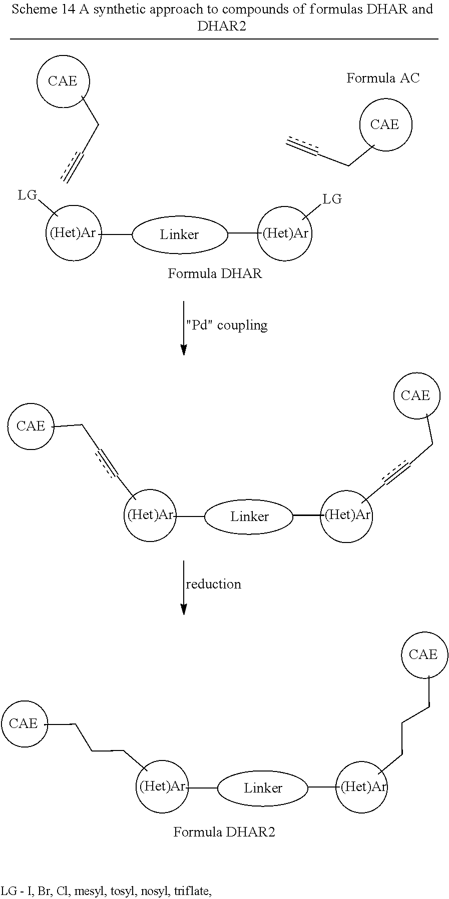

Dimers or heterodimers of general Formula DO (dimers linked to HBV CAE via olefin linkages) can be prepared, for example, through a ruthenium or tungsten catalyzed cross-metathesis reaction between a correctly functionalized HBV CAE of general Formula CO (CAE attached to an olefin) and a linker with two vinyl or allyl substitutions (Formula LO, linkers attached to olefins). Reduction of the unsaturation formed, using for instance, H₂ and Pd/C in MeOH, can produce compound of general Formula DA (Dimers linked to CAE via alkyl linkages)

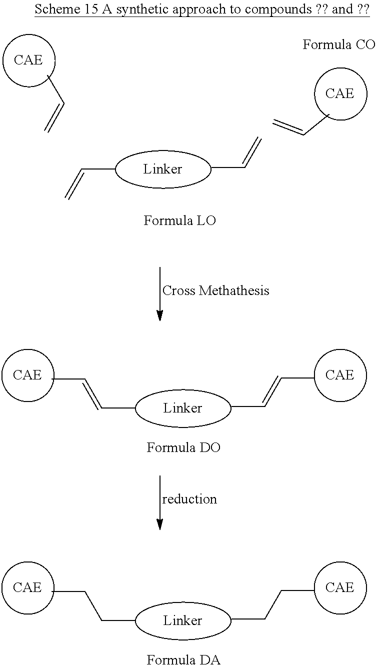

Compounds of general formulas C₅₋₇ can be prepared, for example, by first reaction of a correctly functionalized and protected piperidinone or a correctly functionalized and protected bicyclic piperidinone of general structure C1 with a functionalized acyl chloride derivative in presence of a base such as LiHMDS in a solvent such as THF. Reaction of the diketo intermediate C2 with hydrazine, to form intermediate C3, and subsequent deprotection of the amino group can afford key intermediate C4. Reaction of C4 with a correctly functionalized isocyanate, acyl chloride or sulfonyl chloride reagent in presence of an organic base such as Et₃N can give compounds of structure C5, C6, and C7. In the generalized reaction scheme shown below, the dashed line symbolizes an optional ring formed by linking R² and R³ via an alkyl bridge, and R¹-R⁵ are as defined in the compound section.

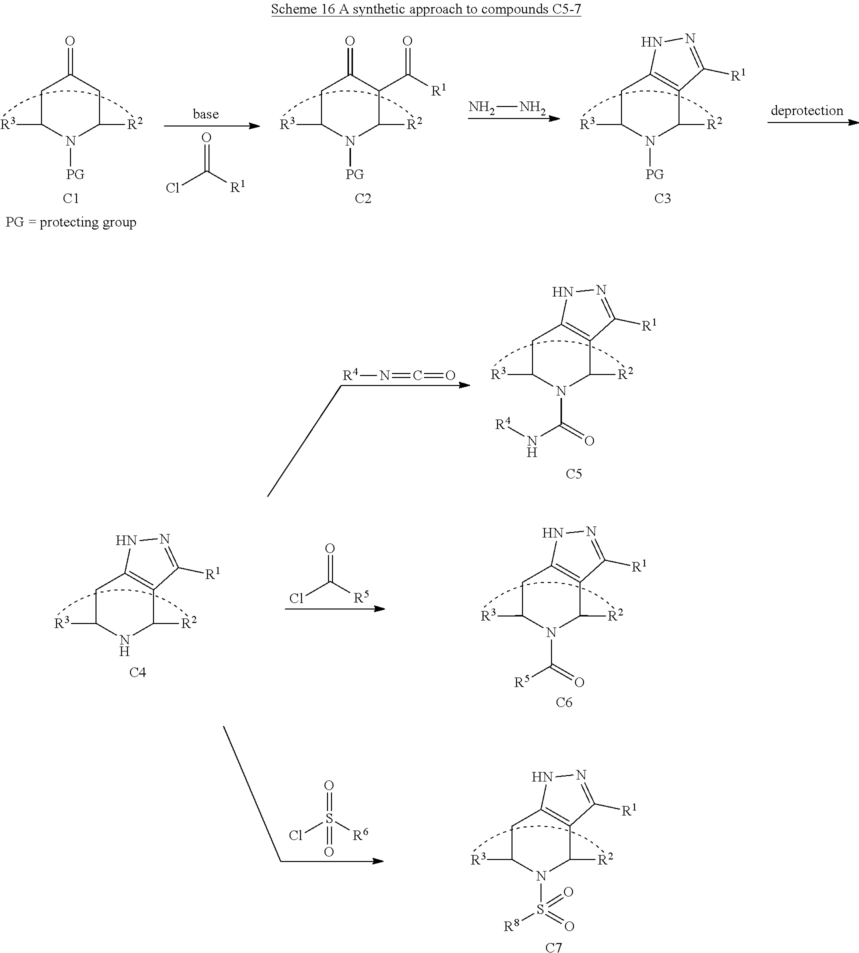

In Schemes 17 and 18 shown below, R, R¹, R³, R⁴, and A are defined as in the Compound section herein.

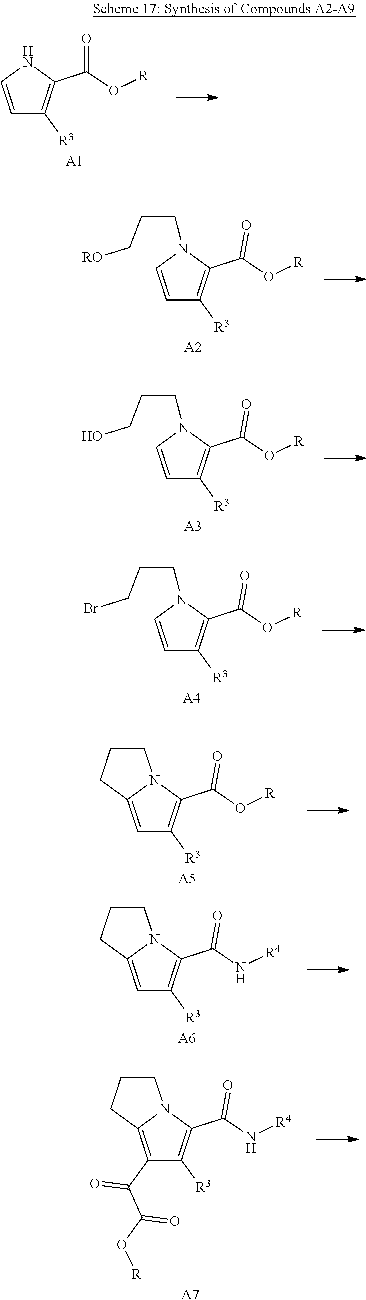

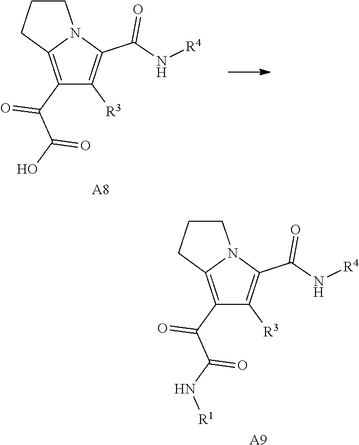

As shown in Scheme 17, A1 can be converted to A2 by treatment with an appropriate alkyl halide such as (3-bromopropoxy)(tert-butyl)diphenylsilane in the presence of a suitable catalyst such as palladium dichloride bis(acetonitrile. A2 can be converted to A3 through a deprotection with a suitable reagent such as tetrabutylammonium fluoride and further halogenated to A4 with a suitable reagents such as carbon tetrabromide and triphenylphosphine. Cyclization to A5 can be effected with an appropriate radical initiator such as 2,2′-azobis(2-methylpropionitrile) in the presence other reagents such as tributyl tin hydride. Ester hydrolysis with a suitable reagent such as lithium hydroxide followed by amide formation via treatment with an appropriate coupling reagent such as HATU and the appropriate aniline or by conversion to the acid chloride with a reagent such a thionyl chloride or oxalyl chloride followed by treatment with the appropriate aniline gives A6. The aniline may be varied based on the R4 groups disclosed herein. Formation of A7 can be effected by treatment with a suitable reagent such as oxalyl chloride or ethyl2-chloro-2-oxoacetate and may or may not require the addition of catalyst such as aluminum chloride. Hydrolysis to AS via a suitable reagent such as lithium hydroxide is followed by preparation of A9 by the coupling of the appropriate amine such as 1,1,1-trifluoropropan-2-amine in the presence of a coupling reagent such as HATU. The amine may be varied for particular R1 groups disclosed herein.

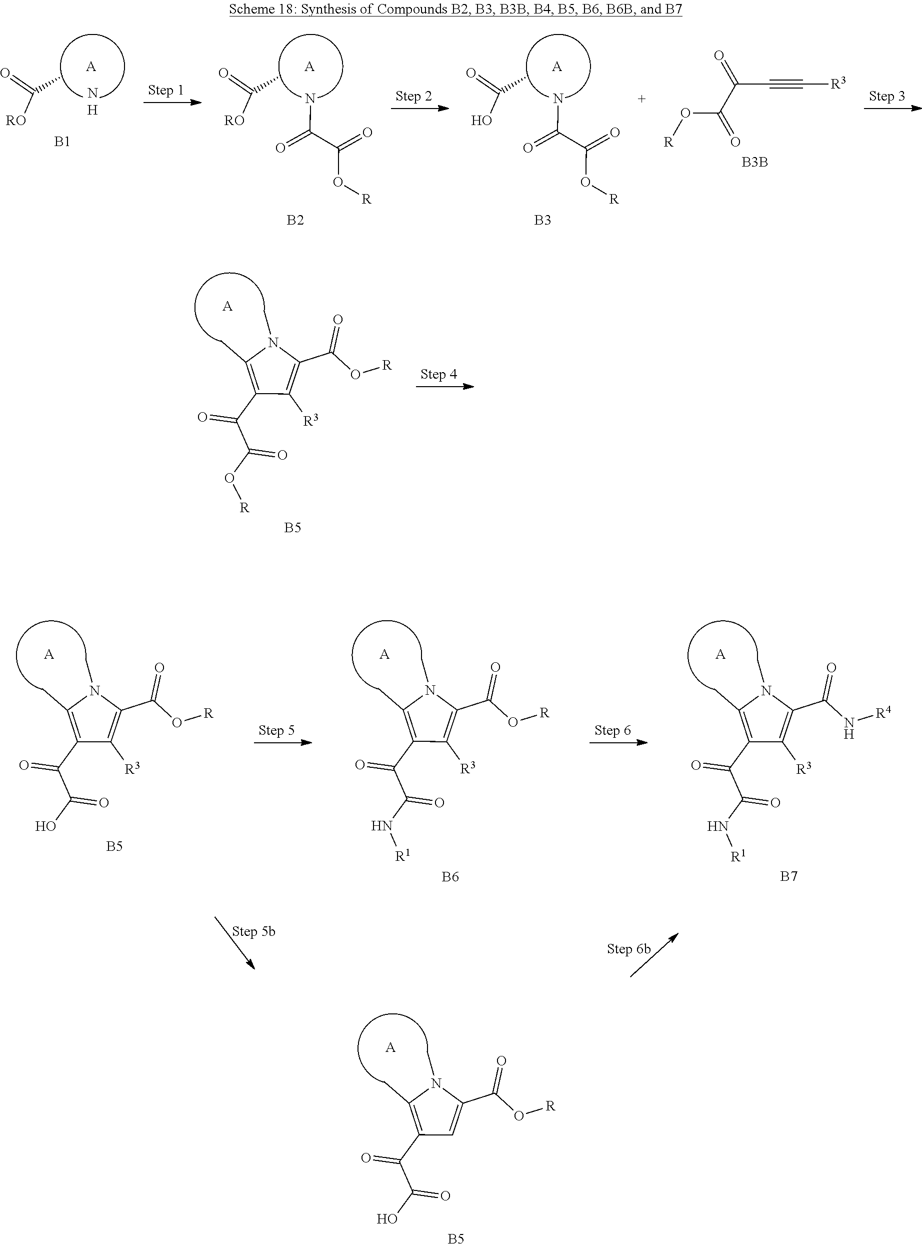

As shown in Scheme 18, B1 can be converted to B2, for example, by treatment with methyl 2-chloro-2-oxoacetate. Conversion to B3 can be carried out with an appropriate hydroxide reagent such as lithium hydroxide or in some cases with hydrogen gas and a suitable catalyst if the ester is a benzyl ester. B3 can be converted to B4 by treatment with a suitable reagent such as N,N′-diisopropylcarbodiimide and alkyne reagent B3B at elevated temperatures. Alternatively, B3 can be treated with oxalyl chloride, worked up and treated with B3B and a suitable base such as 2,6-di-tertbutylpyridine to give B4. Hydrolysis to B5 can be effected with a suitable reagent such as lithium hydroxide. Conversion to B6 occurs by addition of an appropriate amine (e.g. R1-NH₂) and an amide coupling reagent such as HATU. B6 can be converted to desired B7 by treatment with a suitable reagent such as lithium hydroxide at elevated temperatures followed a second amide coupling with an appropriate aniline or heteroaryl amine (e.g R4-NH₂) and a coupling reagent such as HATU. Alternatively, B5 can be treated with an aniline or heteroaryl amine in the presence of a reagent such as Lithium bis(trimethylsilyl)amide to give B6B followed by treatment with an appropriate amine and a coupling reagent such as HATU to give desired B7.

As shown in Scheme 18A, it is relatively straightforward to provide ester, such as amino acid ester, sulfate, phosphate and phosphoramidate terminated alkynes. Reaction of an acid with an acetylene functionalized with both a hydroxyl and an amine moiety results in formation of the amide linkage to the core molecule, with retention of the hydroxyl group at the terminus of the alkyne. This hydroxyl group can then be esterified, sulfated, or phosphorylated using known chemistry.

While shown below with a representative compound, the synthesis can be applied to other compounds described herein, as long as any groups which would react with the amine moiety on the alkyne are suitably protected, or such groups are added to the molecule after the coupling chemistry, in this case, amidation chemistry, is performed.

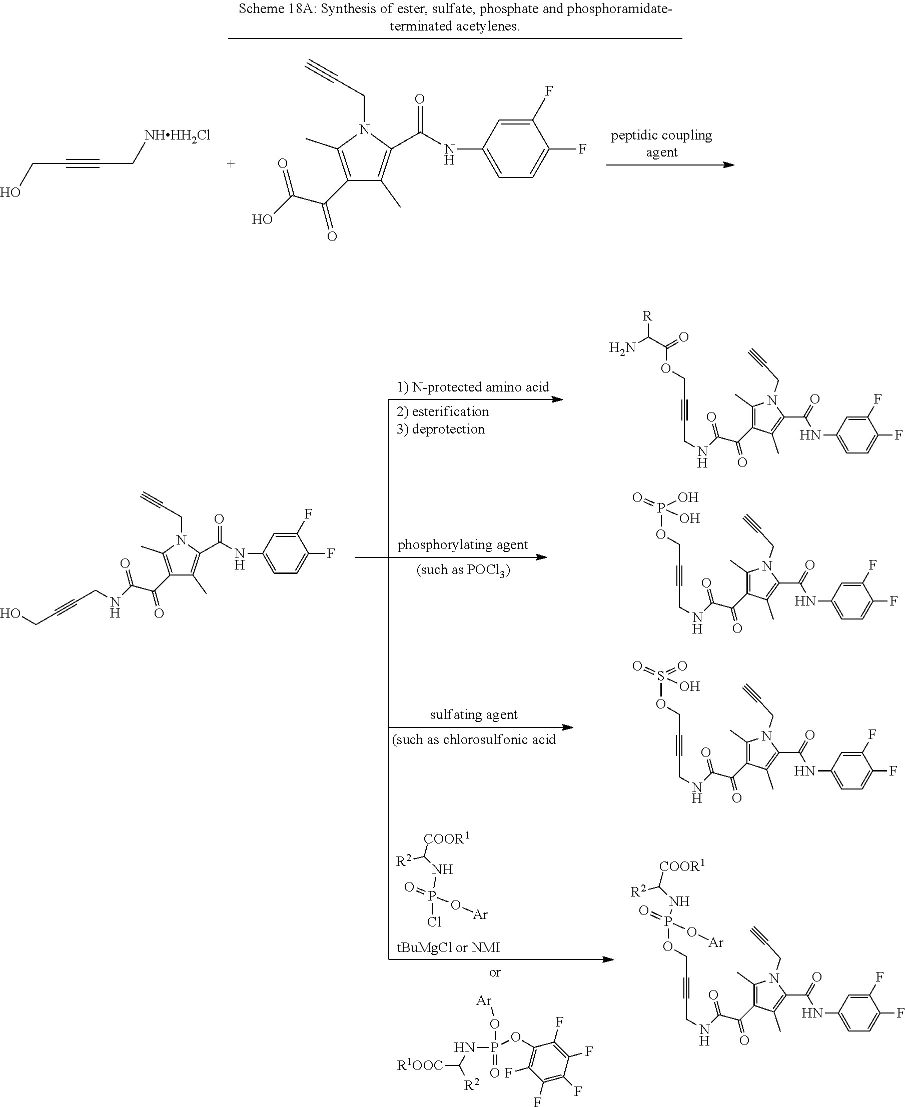

As shown in Scheme 18B, W1 can be converted to W2 by reaction with a base such as KOH in presence of the appropriate alkylating agent, such as Mel. W2 can then be saponified, in presence of NaOH for instance, and then reacted with a substituted aniline in presence of a coupling agent such as HATU and a base such as DIPEA. Conversion of W4 to W5 can be affected by treatment with a suitable reagent such as oxalyl chloride or ethyl2-chloro-2-oxoacetate and may or may not require the addition of catalyst such as aluminum chloride. Hydrolysis to W6 via a suitable reagent such as lithium hydroxide is followed by preparation of W7 by the coupling of the appropriate amine such as propargyl amine in the presence of a coupling reagent such as HATU.

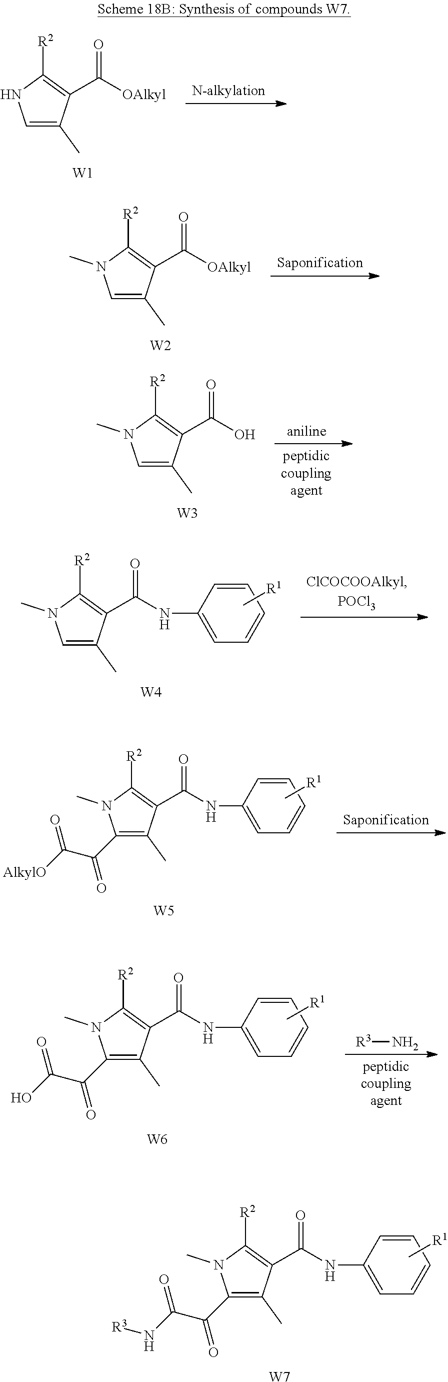

Incorporation of Deuterium:

It is expected that single or multiple replacement of hydrogen with deuterium (carbon-hydrogen bonds to carbon-deuterium bond) at site(s) of metabolism on a HBV CAE, a homo or hetero HBV CAE dimer, a homo or hetero HBV CAE trimer will slow down the rate of metabolism. This can provide a relatively longer half-life, and slower clearance from the body. The slow metabolism of a HBV CAE, a homo or hetero HBV CAE dimer or a homo or hetero HBV CAE trimer is expected to add extra advantage to a therapeutic candidate, while other physical or biochemical properties are not affected.

Methods for incorporating deuterium into organic derivatives are well known to those of skill in the art. Representative methods are disclosed in Angew. Chem. Int. Ed. Engl. 2007, 46, 7744-7765. Accordingly, using these techniques, one can provide one or more deuterium atoms in the HBV CAE, homo or hetero HBV CAE dimers or homo or hetero HBV CAE trimers described herein.

### SPECIFIC EXAMPLES

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The present compounds can also be used as intermediates in subsequent examples to produce additional compounds of the present invention. No attempt has necessarily been made to optimize the yields obtained in any of the reactions. One skilled in the art would know how to increase such yields through routine variations in reaction times, temperatures, solvents and/or reagents.

Anhydrous solvents were purchased from Aldrich Chemical Company, Inc. (Milwaukee, Wis.) and EMD Chemicals Inc. (Gibbstown, N.J.). Reagents were purchased from commercial sources. Unless noted otherwise, the materials used in the examples were obtained from readily available commercial suppliers or synthesized by standard methods known to one skilled in the art of chemical synthesis. ¹H and ¹³C NMR spectra were taken on a Bruker Ascend™ 400 MHz Fourier transform spectrometer at room temperature and reported in ppm downfield from internal tetramethylsilane. Deuterium exchange, decoupling experiments or 2D-COSY were performed to confirm proton assignments. Signal multiplicities are represented by s (singlet), d (doublet), dd (doublet of doublets), t (triplet), q (quadruplet), br (broad), bs (broad singlet), m (multiplet). All J-values are in Hz. Mass spectra were determined on a Micromass Platform LC spectrometer using electrospray techniques. Analytic TLC were performed on Sigma-Aldrich® aluminum supported silica gel (25 μm) plates. Column chromatography was carried out on Silica Gel or via reverse-phase high performance liquid chromatography.

### Example 1

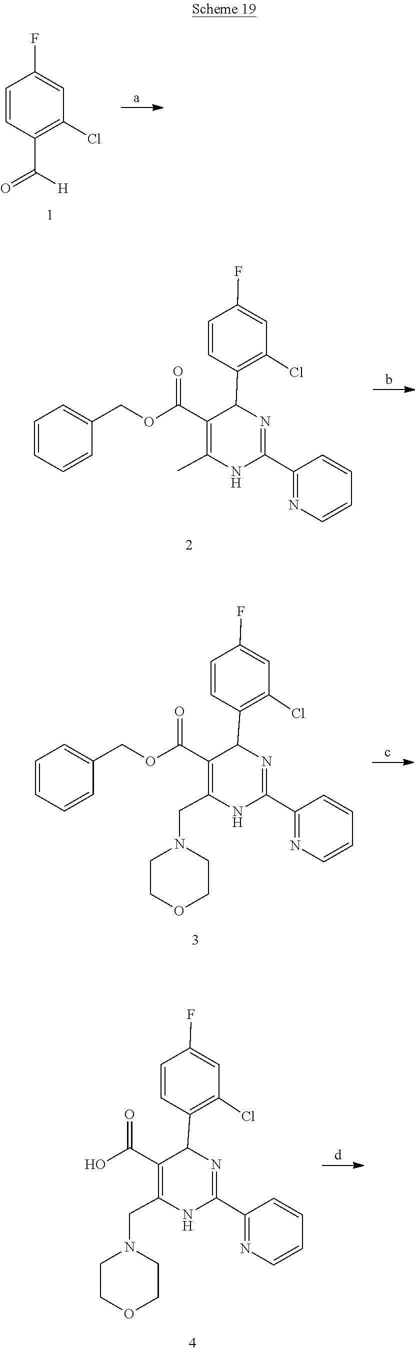 [SMILES: O=Cc1ccc(F)cc1Cl ; CC1=C(C(=O)OCc2ccccc2)C(c2ccc(F)cc2Cl)N=C(c2ccccn2)N1 ; O=C(OCc1ccccc1)C1=C(CN2CCOCC2)NC(c2ccccn2)=NC1c1ccc(F)cc1Cl ; O=C(O)C1=C(CN2CCOCC2)NC(c2ccccn2)=NC1c1ccc(F)cc1Cl]

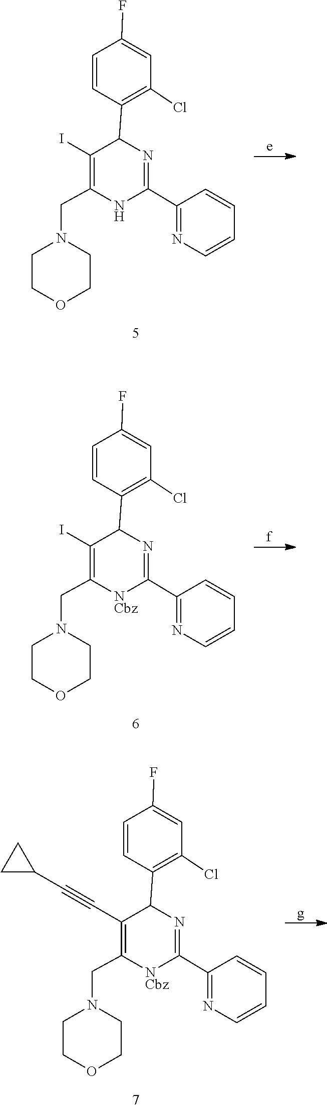 [SMILES: Fc1ccc(C2N=C(c3ccccn3)NC(CN3CCOCC3)=C2I)c(Cl)c1 ; O=C(OCc1ccccc1)N1C(c2ccccn2)=NC(c2ccc(F)cc2Cl)C(I)=C1CN1CCOCC1 ; O=C(OCc1ccccc1)N1C(c2ccccn2)=NC(c2ccc(F)cc2Cl)C(C#CC2CC2)=C1CN1CCOCC1]

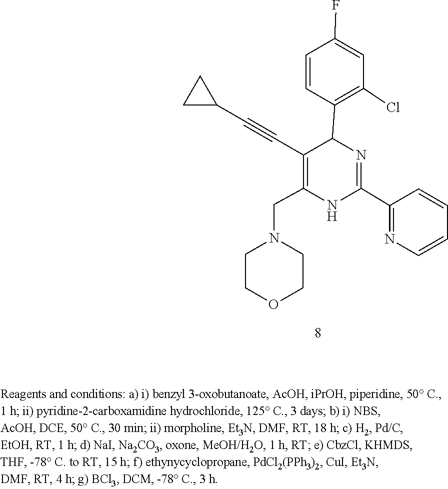 [SMILES: Fc1ccc(C2N=C(c3ccccn3)NC(CN3CCOCC3)=C2C#CC2CC2)c(Cl)c1]

### Benzyl 4-(2-chloro-4-fluorophenyl)-6-methyl-2-(pyridin-2-yl)-1,4-dihydropyrimidine-5-carboxylate (2)

To a suspension of 2-chloro-4-fluorobenzaldehyde (6 g, 37.8 mmol), piperidine (3 mL), acetic acid (1 mL) in anhydrous isopropanol (60 mL) was added benzyl 3-oxobutanoate (10.9 g, 56.8 mmol). The mixture was heated at 50° C. for 1 h before pyridine-2-carboxamidine hydrochloride (5.96 g, 37.8 mmol) was added. The resulting solution was at 125° C. for 3 days and then concentrated in vacuo. The residue was washed with MeOH (50 mL) to yield compound 2 as a yellow solid. (3.8 g, 8.7 mmol). ¹H NMR (400 MHz, Chloroform-d) δ 8.64 (s, 1H), 8.54 (dt, J=4.9, 1.2 Hz, 1H), 8.20 (d, J=8.0 Hz, 1H), 7.76 (t, J=7.8 Hz, 1H), 7.42-7.31 (m, 2H), 7.28 (dd, J=4.2, 2.3 Hz, 3H), 7.12 (dt, J=8.5, 3.1 Hz, 3H), 6.90 (td, J=8.3, 2.6 Hz, 1H), 6.33 (s, 1H), 5.13 (d, J=12.6 Hz, 1H), 5.04 (d, J=12.6 Hz, 1H), 2.58 (s, 3H). MS (ESI): m/z [M+H]⁺ calcd for C₂₄H₂₀ClFN₃O₂: 436.9, found: 436.3, 438.4.

### 4-(2-Chloro-4-fluorophenyl)-6-(morphohnomethyl)-2-(pyri din-2-yl)-1,4-dihydropyrimidine-5-carboxylic acid (4)

To a solution of 2 (1.65 g, 3.7 mmol) in dichloroethane (30 mL) and acetic acid (1 mL) at 50° C. was added N-bromosuccinimide (0.67 g, 3.8 mmol). The mixture was stirred at 50° C. for 30 min then poured into a saturated solution of sodium thiosulfate (50 mL). The aqueous phase was extracted with dichloromethane (3×50 mL) and the combined organic layers were washed with a saturated solution of sodium carbonate (2×50 mL), dried over sodium sulfate and concentrated in vacuo. The residue was finally purified by flash chromatography (Hexanes/AcOEt) to give the desired brominated intermediate. To a solution a this intermediate in N. A-dimethyl formamide (60 mL) were added morpholine (0.97 mL, 11.3 mmol) and triethylamine (1.54 mL, 11.3 mmol). The mixture was stirred at room temperature for 18 h then poured into a saturated solution of ammonium chloride (50 mL). The mixture was extracted with ethyl acetate (3×50 mL) and the combined organic phases were dried over sodium sulfate. After concentration in vacuo. The residue was finally purified by flash chromatography hexanes/EtOAc to yield compound 3 (0.8 g, 1.5 mmol) as a yellow foam. Through a solution of intermediate 3 (0.8 g, 1.5 mmol) in EtOH (100 mL) was bubbled argon for 5 minutes before addition of palladium on charcoal 10% (0.13 g). The reaction mixture was then stirred for 2 h under an atmosphere of hydrogen before being filtered through celite. Concentration in vacuo gave carboxylic acid derivative 4 (0.64 g, 1.5 mmol) in 98% yield. ¹H NMR (400 MHz, Chloroform-d) δ 8.60-8.54 (m, 1H), 8.36-8.26 (m, 1H), 7.81 (t, J=7.8 Hz, 1H), 7.45-7.38 (m, 1H), 7.35-7.29 (m, 1H), 7.16 (dd, J=8.4, 2.6 Hz, 1H), 6.93 (td, J=8.2, 2.6 Hz, 1H), 6.25 (s, 1H), 3.84 (brs, 4H), 3.73 (s, 2H), 3.18 (brs, 1H), 3.03 (brs, 1H), 2.72 (brs, 2H). MS (ESI): m/z [M+H]⁺ calcd for C₂₁H₂₁ClFN₄O₃: 431.9, found: 431.4, 433.4.

### 4-((6-(2-Chloro-4-fluorophenyl)-5-iodo-2-(pyridin-2-yl)-3,6-dihydropyrimidin-4-yl)methyl)morpholine (5)

A suspension of carboxylic acid 4 (0.5 mg, 1.2 mmol), sodium iodide (0.87 mg, 5.8 mmol) and sodium carbonate (184 mg, 1.7 mmol) in water (35 mL) and methanol (35 mL) was stirred and sonicated until a clear, colorless solution was obtained. To this solution, protected from light, was added OXONE (159 mg, 1.0 mmol). After stirring for 20 min at room temperature, a saturated solution of sodium thiosulfate (50 mL) was added. The reaction was then extracted with ethyl acetate (3×50 mL); the combined organic phases were dried over sodium sulfate and concentrated in vacuo. Purification of the residue by flash chromatography on silica gel (hexanes/ethyl acetate: 8/2) gave 5 (435 mg, 73% yield) as a yellow solid. ¹H NMR (400 MHz, Chloroform-d) δ 9.25 (s, 1H), 8.59 (d, J=4.8 Hz, 1H), 8.18 (d, J=8.0 Hz, 1H), 7.76 (td, J=7.7, 1.7 Hz, 1H), 7.47-7.34 (m, 2H), 7.17 (dd, J=8.6, 2.6 Hz, 1H), 6.99 (td, J=8.3, 2.6 Hz, 1H), 6.08 (s, 1H), 3.81 (t, J=4.6 Hz, 4H), 3.41 (s, 2H), 2.60 (d, J=5.4 Hz, 4H). MS (ESI): m/z [M+H]⁺ calcd for C₂₀H₂₀ClFIN₄O: 513.7, found: 513.2.

### Benzyl 4-(2-chloro-4-fluorophenyl)-5-iodo-6-(morpholinomethyl)-2-(pyridin-2-yl)pyrimidine-1(4H)-carboxylate (6)

To a solution of 5-iodo-dihydropyrimidine 5 (0.4 g, 0.8 mmol) in THF (20 mL) was added at 0° C., KHMDS (1M in THF, 1.16 mL, 1.16 mmol) and benzyl chloroformate (0.22 mL, 1.6 mmol). The mixture was stirred for 1 h at 0° C. and then for 15 h at room temperature. The reaction was quenched with a saturated solution of ammonium chloride (50 mL) and extracted with ethyl acetate (3×50 mL). The combined organic layers were dried over sodium sulfate, concentrated in vacuo and the residue purified by flash chromatography on silica gel (hexanes/ethyl acetate: 8/2) to yield 6 as a yellow solid (0.3 g, 0.5 mmol, 59%). ¹H NMR (400 MHz, Chloroform-d) δ 8.44 (d, J=4.8 Hz, 1H), 7.69 (dt, J=15.3, 7.8 Hz, 2H), 7.41-7.12 (m, 7H), 6.89 (dd, J=19.7, 7.9 Hz, 3H), 6.52 (s, 1H), 5.06-4.93 (m, 2H), 3.77 (t, J=4.6 Hz, 4H), 3.64-3.47 (m, 2H), 2.66 (t, J=4.6 Hz, 4H). ¹⁹F NMR (377 MHz, Chloroform-d) 5-111.32 (q, J=7.1 Hz). MS (ESI): m/z [M+H]⁺ calcd for C₂₈H₂₆ClFIN₄O₃: 647.9, found: 647.3, 649.3.

### 4-((6-(2-Chloro-4-fluorophenyl)-5-(cyclopropylethynyl)-2-(pyridin-2-yl)-3,6-dihydropyrimidin-4-yl)methyl)morpholine (8)

Through a solution of 6 (50 mg, 77 μmol), copper iodide (3 mg, 15.4 μmol) and triethylamine (30 μL, 232 μmol) in DMF (2 mL) was bubbled argon for 5 minutes. After addition of bis(triphenylphosphine) palladium dichloride (5.4 mg, 8 μmol), the reaction mixture was stirred for 4 h at room temperature. The reaction was then diluted with ethyl acetate (50 mL) and washed with a saturated solution of ammonium chloride (50 mL). The organic layer was dried over sodium sulfate and concentrated under reduced pressure. The residue was purified by flash chromatography on silica gel to give crude compound 7. To a solution of 7 in dichloromethane (20 mL) was added boron trichloride (1M in dichloromethane, 232 μL, 232 μmol) at −78° C. The mixture was stirred for 3 h, quenched with methanol (2 mL), and concentrated in vacuo. The residue was purified by flash chromatography on silica gel (DCM/MeOH: 95/5) to give compound 8 (10 mg, 22 μmol, 29%). ¹H NMR (400 MHz, Chloroform-d) δ 9.10 (s, 1H), 8.58 (d, J=4.8 Hz, 1H), 8.18 (d, J=7.9 Hz, 1H), 7.74 (td, J=7.8, 1.8 Hz, 1H), 7.37 (ddd, J=8.2, 5.4, 3.0 Hz, 2H), 7.11 (dd, J=8.7, 2.6 Hz, 1H), 6.95 (td, J=8.3, 2.6 Hz, 1H), 5.82 (s, 1H), 3.76 (t, J=4.6 Hz, 4H), 3.48-3.34 (m, 2H), 2.52 (q, J=4.1 Hz, 4H), 1.30-1.22 (m, 1H), 0.90-0.66 (m, 2H), 0.59 (dt, J=8.5, 3.6 Hz, 1H), 0.52 (dt, J=6.1, 3.6 Hz, 1H). MS (ESI): m/z [M+H]⁺ calcd for C₂₅H₂₅ClFN₄O: 451.9, found: 451.4, 453.3.

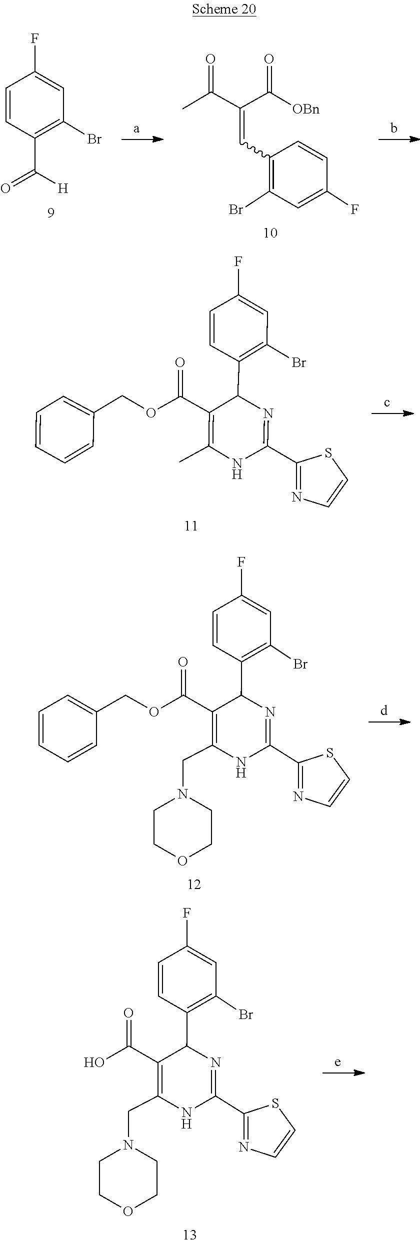 [SMILES: O=Cc1ccc(F)cc1Br ; CC(=O)C(=Cc1ccc(F)cc1Br)C(=O)OCc1ccccc1 ; CC1=C(C(=O)OCc2ccccc2)C(c2ccc(F)cc2Br)N=C(c2nccs2)N1 ; O=C(OCc1ccccc1)C1=C(CN2CCOCC2)NC(c2nccs2)=NC1c1ccc(F)cc1Br ; O=C(O)C1=C(CN2CCOCC2)NC(c2nccs2)=NC1c1ccc(F)cc1Br]

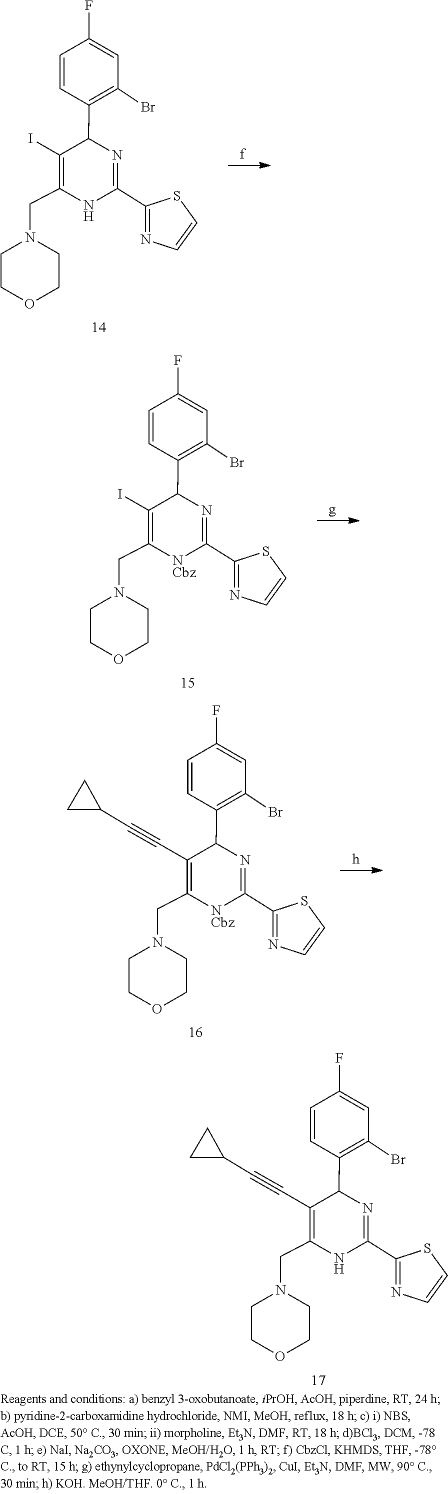 [SMILES: Fc1ccc(C2N=C(c3nccs3)NC(CN3CCOCC3)=C2I)c(Br)c1 ; O=C(OCc1ccccc1)N1C(c2nccs2)=NC(c2ccc(F)cc2Br)C(I)=C1CN1CCOCC1 ; O=C(OCc1ccccc1)N1C(c2nccs2)=NC(c2ccc(F)cc2Br)C(C#CC2CC2)=C1CN1CCOCC1 ; Fc1ccc(C2N=C(c3nccs3)NC(CN3CCOCC3)=C2C#CC2CC2)c(Br)c1]

### Benzyl 2-(2-bromo-4-fluorobenzylidene)-3-oxobutanoate (10)

To a solution of 2-bromo-4-fluorobenzaldehyde (5 g, 24.6 mmol) in iPrOH (60 mL) were added benzyl 3-oxobutanoate (5.2 g, 27.1 mmol), piperidine (1 mL) and acetic acid (1 mL). The reaction mixture was stirred at room temperature for 24 h and then concentrated in vacuo. The residue was solubilized in ethyl acetate (100 mL) and washed with a saturated solution of ammonium chloride (50 mL) and a saturated solution of sodium carbonate (50 mL). The organic layer was then dried over sodium sulfate and concentrated in vacuo. The resulting oil was purified by flash chromatography (hexanes/EtOAc: 9:1) to yield compound 10 as a 2.3/1 mixture of E and Z isomers (7.5 g, 19.9 mmol, 80%). ¹H NMR (400 MHz, Chloroform-d) δ 7.89 (s, 1H), 7.77 (s, 2H), 7.45-7.30 (m, 18H), 7.30-7.23 (m, 7H), 7.19 (dd, J=8.7, 5.8 Hz, 3H), 7.04 (td, J=8.3, 2.6 Hz, 1H), 6.69 (td, J=8.3, 2.6 Hz, 3H), 5.32 (s, 2H), 5.24 (s, 5H), 2.47 (s, 8H), 2.24 (s, 3H).

### Benzyl 4-(2-bromo-4-fluorophenyl)-6-methyl-2-(thiazol-2-yl)-1,4-dihydropyrimidine-5-carboxylate (11)

A solution of 10 (2.54, 6.7 mmol), NMI (977 μL, 12.2 mmol) and thiazole-2-carboxamidine hydrochloride (1 g, 6.1 mmol) in THF (30 mL) was refluxed for 18 h. After concentration in vacuo, the residue was diluted in EtOAc (50 mL) and the organic layer washed with a saturated solution of ammonium chloride (50 mL). The aqueous phase was finally extracted with ethyl acetate (2×50 mL) and the combined organic layers were dried over sodium sulfate. After concentration in vacuo, the residue was purified by flash chromatography (hexanes/EtOAc: 7/3) to yield compound 10 as a 2.6/1 mixture of tautomers (1.5 g, 3.1 mmol, 50%). ¹H NMR (400 MHz, Chloroform-d) δ 7.86 (d, J=3.1 Hz, 4H), 7.82 (d, J=3.1 Hz, 3H), 7.55 (s, 1H), 7.53 (d, J=3.1 Hz, 1H), 7.46 (d, J=3.1 Hz, 3H), 7.37-7.24 (m, 21H), 7.16-7.06 (m, 8H), 6.97 (dtd, J=11.1, 8.3, 2.6 Hz, 4H), 6.23 (s, 3H), 6.09 (d, J=2.5 Hz, 1H), 5.19-4.98 (m, 8H), 2.61 (s, 3H), 2.55 (s, 8H). MS (ESI): m/z [M+H]⁺ calcd for C₂₂H₁₈BrFN₃O₂S: 487.4, found: 486.3, 488.2.

### Benzyl 4-(2-bromo-4-fluorophenyl)-6-(morpholinomethyl)-2-(thiazol-2-yl)-1,4-dihydro-Pyrimidine-5-carboxylate (12)

To a solution of 11 (1.5 g, 3.1 mmol) in dichloroethane (15 mL) and acetic acid (1.5 mL) at 50° C. was added A-bromosuccinimide (0.55 g, 3.1 mmol). The mixture was stirred at 50° C. for 30 min then poured into a saturated solution of sodium thiosulfate (50 mL). The aqueous phase was extracted with dichloromethane (3×50 mL) and the combined organic layers were washed with a saturated solution of sodium carbonate (2×50 mL), dried over sodium sulfate and concentrated in vacuo. The residue was purified by flash chromatography (hexanes/EtOAc) to give the desired brominated intermediate. To a solution a this intermediate in DMF (5 mL) were added morpholine (0.41 mL, 4.6 mmol) and triethylamine (1.26 mL, 9.3 mmol). The mixture was stirred at room temperature for 18 h then poured into a saturated solution of ammonium chloride (50 mL). The aqueous layer was extracted with ethyl acetate (3×50 mL) and the combined organic phases were dried over sodium sulfate. After concentration in vacuo, the residue was purified by flash chromatography (hexanes/EtOAc) to yield compound 12 (0.75 g, 1.3 mmol, 43%) as a yellow foam. ¹H NMR (400 MHz, Chloroform-d) δ 9.73 (s, 1H), 7.87 (d, J=3.1 Hz, 1H), 7.46 (d, J=3.1 Hz, 1H), 7.35-7.22 (m, 5H), 7.11 (dd, J=6.6, 2.9 Hz, 2H), 6.95 (td, J=8.3, 2.6 Hz, 1H), 6.25 (s, 1H), 5.15-4.98 (m, 2H), 4.12-3.87 (m, 2H), 3.84 (dd, J=5.7, 3.7 Hz, 4H), 2.63 (t, J=4.7 Hz, 4H). ¹⁹F NMR (377 MHz, chloroform-d) 8-114.77.

### 4-(2-bromo-4-fluorophenyl)-6-(morpholinomethyl)-2-(thiazol-2-yl)-1,4-dihydropyrimidine-5-carboxylic acid (13)

To a solution of 12 (0.9 g, 1.6 mmol) in dichloromethane (30 mL) was added boron trichloride (1M in dichloromethane, 6.3 mL, 6.3 mmol) at −78° C. The reaction mixture was stirred for 2 h at room temperature and then quenched with MeOH (2 mL). After concentration in vacuo, the resulting solid was washed with diethyl ether to yield compound 13 as a yellow solid (0.5 g, 1.0 mmol, 66%). ¹H NMR (400 MHz, Chloroform-d) δ 7.89 (d, J=3.1 Hz, 1H), 7.57 (d, J=3.1 Hz, 1H), 7.42-7.30 (m, 2H), 7.01 (dd, J=8.9, 6.4 Hz, 1H), 6.15 (s, 1H), 4.61 (d, J=14.8 Hz, 1H), 4.38 (d, J=14.8 Hz, 1H), 4.09 (brs, 5H), 3.72 (brs, 2H), 3.19 (brs, 2H). MS (ESI): m/z [M+H]⁺ calcd for C₁₉H₁₉BrFN₄O₃S: 481.4, found: 481.3, 483.2.

### 4-((6-(2-Bromo-4-fluorophenyl)-5-iodo-2-(thiazol-2-yl)-3,6-dihydropyrimidin-4-yl)methyl)morpholine (14)

A suspension of carboxylic acid 13 (346 mg, 1.0 mmol), sodium iodide (750 mg, 5.0 mmol) and sodium carbonate (106 mg, 1.0 mmol) in water (8 mL) and methanol (8 mL) was stirred and sonicated until a clear, colorless solution was obtained. To this solution, protected from light, was added OXONE (431 mg, 0.7 mmol). After stirring for 20 min at room temperature, a saturated solution of sodium thiosulfate (50 mL) was added. The reaction mixture was then extracted with ethyl acetate (3×25 mL); the combined organic layers were dried over sodium sulfate and concentrated under vacuum. Purification of the residue by flash chromatography on silica gel (hexanes/ethyl acetate: 8/2) gave 14 (290 mg, 68% yield) as a yellow solid. ¹H NMR (400 MHz, Chloroform-d) δ 8.62 (s, 1H), 7.84 (d, J=3.0 Hz, 1H), 7.42 (d, J=3.1 Hz, 1H), 7.41-7.29 (m, 2H), 7.02 (td, J=8.3, 2.7 Hz, 1H), 5.99 (s, 1H), 3.78 (t, J=4.6 Hz, 4H), 3.41-3.28 (m, 2H), 2.55 (d, J=4.9 Hz, 4H). ¹⁹F NMR (377 MHz, Chloroform-d) δ−113.92 (q, J=7.5 Hz).

### Benzyl 4-(2-bromo-4-fluorophenyl)-5-iodo-6-(morpholinomethyl)-2-(thiazol-2-yl(pyrimidine-1(4H)-carboxylate (15)

To a solution of 5-iodo-dihydropyrimidine 14 (0.15 g, 0.26 mmol) in THF (10 mL) was added KHMDS (1M in THF, 0.53 mL, 0.53 mmol) and benzyl chloroformate (0.57 mL, 0.39 mmol) at 0° C. The mixture was stirred for 1 h at 0° C. and then for 15 h at room temperature. The reaction mixture was quenched with a saturated solution of ammonium chloride (50 mL) and extracted with ethyl acetate (3×50 mL). The combined organic layers were dried over sodium sulfate, concentrated in vacuo and the residue was purified by flash chromatography on silica gel (hexanes/ethyl acetate: 8/2) to give product 15 as a yellow solid (0.11 g, 0.16 mmol, 59%). ¹H NMR (400 MHz, Chloroform-d) δ 7.85 (d, J=3.2 Hz, 1H), 7.41 (d, J=3.1 Hz, 1H), 7.40-7.33 (m, 3H), 7.33-7.24 (m, 2H), 7.10-7.03 (m, 2H), 6.93 (td, J=8.3, 2.6 Hz, 1H), 5.77 (s, 1H), 5.10 (dd, J=15.3, 3.0 Hz, 2H), 4.03-3.88 (m, 2H), 3.76-3.66 (m, 4H), 2.51 (t, J=4.6 Hz, 4H). MS (ESI): m/z [M+H]⁺ calcd for C₃₁H₂₉BrFN₄O₃S: 636.5, found: 635.4, 637.4.

### 4-((6-(2-Bromo-4-fluorophenyl)-5-(cyclopropylethynyl)-2-(thiazol-2-yl)-3,6-dihydropyrimidin-4-yl)methyl)morpholine (17)

Through a solution of 15 (80 mg, 114 μmol), copper iodide (2 mg, 11 μmol) and triethylamine (47 μL, 344 μmol) in DMF (2 mL) was bubbled argon for 5 minutes. After addition of bis(triphenylphosphine) palladium dichloride (16 mg, 22 μmol), the reaction was heated at 90° C. under microwave irradiation for 30 minutes. The reaction was then diluted with ethyl acetate (50 mL) and washed with a saturated solution of ammonium chloride (50 mL). The organic phase was dried over sodium sulfate and concentrated under reduced pressure. The residue was purified by flash chromatography on silica gel (hexanes/ethyl acetate) to give compound 16 (0.02, 31 μmol, 27%). To a solution of intermediate 16 (40 mg, 63 μmol) in MeOH (2 mL) and THF (2 mL) was added a 20% aqueous solution of potassium hydroxide (2 mL). The reaction mixture was stirred for 1 h at room temperature, neutralized to pH ˜ 7 with a saturated solution of ammonium chloride and extracted with ethyl acetate (3×50 mL). The combined organic layers were dried over sodium sulfate and concentrated in vacuo. Purification of the residue by flash chromatography on silica gel (hexanes/ethyl acetate: 7/3) gave compound 17 (31 mg, 61 μmol, 98%). ¹H NMR (400 MHz, Chloroform-d) δ 8.47 (s, 1H), 7.85 (d, J=3.2 Hz, 1H), 7.43 (d, J=3.2 Hz, 1H), 7.39-7.34 (m, 1H), 7.29 (dd, J=8.4, 2.6 Hz, 1H), 7.01 (td, J=8.3, 2.7 Hz, 1H), 5.77 (s, 1H), 3.75 (t, J=4.6 Hz, 4H), 3.42-3.30 (m, 2H), 2.51 (q, J=4.1 Hz, 4H), 1.31-1.20 (m, 1H), 0.78-0.67 (m, 2H), 0.64-0.57 (m, 1H), 0.55-0.47 (m, 1H). MS (ESI): m/z [M+H]⁺ calcd for C₂₃H₂₃BrFN₄OS: 502.4, found: 501.3, 503.3.

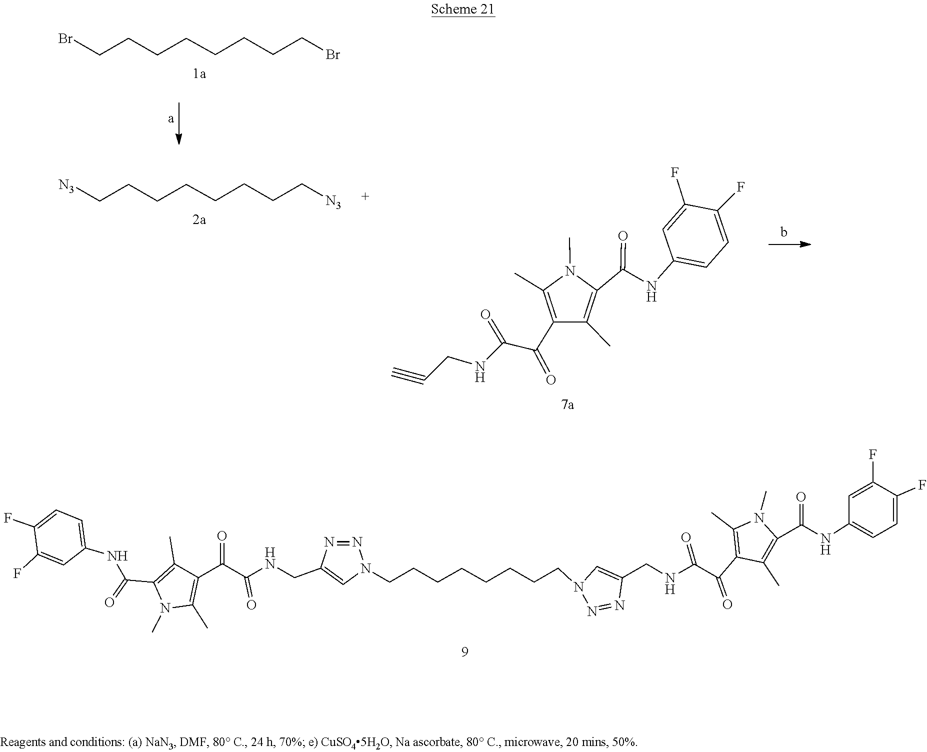 [SMILES: BrCCCCCCCCBr ; [N-]=[N+]=NCCCCCCCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCCCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### 1,8-Diazidooctane (2a)

To a solution of compound 1a (544 mg, 2 mmol) in DMF (3 mL) was added NaN₃ (400 mg, 6.2 mmol, 3.1 eq). The resulting mixture was stirred at 80° C. for 24 h and was quenched with cold water. The water layer was then extracted with ethyl acetate (3×20 mL). The organic layers were finally combined, washed with water, brine, and dried over anhydrous Na₂SO₄, and concentrated under reduced pressure. The residue was purified by flash chromatography (Hexane/Ethyl acetate 30:0 to 3:1) to afford product 2a (275 mg, 70%). ¹H NMR (400 MHz, CDCl₃) δ 3.28-3.25 (m, 4H), 1.64-1.56 (m, 4H), 1.39-1.31 (m, 8H).

### 4,4′-(2,2′-(((Octane-1,8-diylbis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (9)

To a mixture of compound 2a (8 mg, 0.13 mmol) and compound 7a (37 mg, 0.10 mmol) in H₂O/CH₃CN (0.3 mL/0.5 mL) was added CuSO₄.5H₂O (6 mg) and Na ascorbate (12 mg) under Ar atmosphere. The resulting mixture was stirred at 80° C. for 20 min under microwave irradiation. The mixture was diluted with 20 mL of cold H₂O and extracted with ethyl acetate (3×30 mL). The organic layers were combined, washed with water, brine, and dried over anhydrous Na₂SO₄, and concentrated under reduced pressure. The residue was purified by flash chromatography (DCM/MeOH, from 50:1 to 5:1) to afford product 9 (19 mg, 50%). ¹H NMR (400 MHz, CDCl₃) δ 8.86 (s, 2H), 8.09 (s, 2H), 7.75-7.70 (m, 2H), 7.60 (s, 2H), 7.34-7.32 (m, 2H), 7.11-7.04 (m, 2H), 4.45 (s, 4H), 4.27 (t, J=6.2 Hz, 4H), 3.57 (s, 6H), 2.25 (s, 6H), 2.15 (s, 6H), 1.80 (t, J=6.6 Hz, 4H), 1.23-1.19 (m, 8H). ¹⁹F NMR (377 MHz, CDCl₃) δ−135.95 (t, J=12.25 Hz), δ−142.57 (t, J=11.08 Hz). MS (ESI): m/z [M+H]⁺ calcd for C₄₆H₅₁F₄N₁₂O₆: 944.0, found: 943.7.

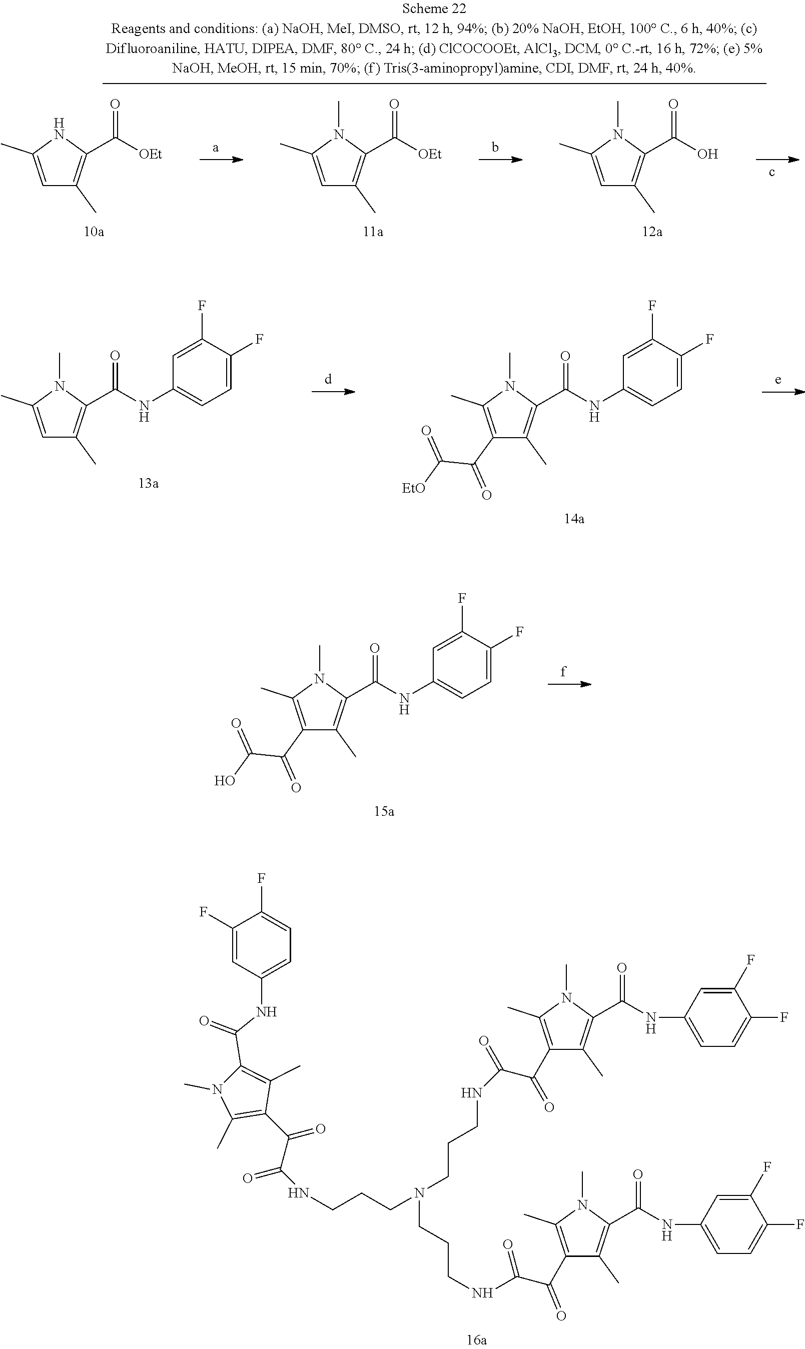

### Ethyl 1,3,5-trimethyl-1H-pyrrole-2-carboxylate (11a)

To a solution of 10a (10.4 g, 62.2 mmol) in anhydrous DMSO (120 mL) was added KOH (10.5 g, 186.6 mmol). The resulting mixture was stirred at rt for 45 min. Mel (26.5 g, 186.6 mmol) was added slowly to keep the temperature under 25° C. (ice bath control). After addition of Mel, the ice bath was removed to allow the reaction to be stirred at rt. for 6 h. Cold water with ice (400 mL) was added and the mixture was extracted with Et₂O (4×150 mL). Combined organic layers were dried over Na₂SO₄ and concentrated under reduced pressure. The residue was purified by flash chromatography using Hexane/EtOAc (20:1 to 3:1) to afford 11a (83%, 9.4 g). ¹H NMR (400 MHz, CDCl₃) δ 5.76 (s, 1H), 4.28 (dd, J=14.2 Hz, J=7.1 Hz, 2H), 3.76 (s, 3H), 2.29 (s, 3H), 2.19 (s, 3H), 1.35 (t, J=7.1 Hz, 3H). MS (ESI): m/z [M+H]⁺ calcd for C₁₀H₁₆NO₂: 182.2, found: 182.5.

### 1,3,5-Trimethyl-1H-pyrrole-2-carboxylic acid (12a)

To a solution of 11a (3 g, 72 mmol) in EtOH (100 mL) was added NaOH 20% (70 mL). The reaction was heated at 100° C. for 6 h. EtOH was evaporated under vacuum and the mixture was washed with DCM (3×30 mL). The aqueous layer was carefully acidified to pH 3-4 with 1M HCl. The mixture was extracted with DCM (3×30 mL). Combined organic layers were dried over MgSO₄ and concentrated in vacuo. The resulting solid was washed with cold Et₂O to afford 12a in 61% yield (6.7 g) as a pink solid. ¹H NMR (400 MHz, DMSO-d₆) δ 11.88 (s, 1H), 5.75 (s, 1H), 3.68 (s, 3H), 2.19 (s, 3H), 2.15 (s, 3H). MS (ESI): m/z [M+H]⁺ calcd for C₈H₁₂NO₂: 154.1, found: 154.5.

### N-(3,4-Difluorophenyl)-1,3,5-trimethyl-1H-pyrrole-2-carboxamide (13a)

To a solution of 12a (2.9 g, 18.9 mmol) in DMF (20 mL) were added 3,4-difluoroaniline (4.5 mL, 22.7 mmol), HATU (8.6 g, 22.7 mmol) and DIPEA (6.6 mL, 37.8 mmol) at 0° C. The mixture was heated at 60° C. for 2 days. The reaction mixture was then diluted with EtOAC and washed with 1M HCl, water and brine. The organic layers was dried over Na₂SO₄ and concentrated in vacuo. The residue was purified by silica gel column chromatography using Hexane/EtOAc (8:2) to afford 13a in 37% yield (1.85 g). ¹H NMR (400 MHz, CDCl₃) δ 7.76-7.65 (m, 1H), 7.19-7.07 (m, 2H), 5.80 (s, 1H), 3.77 (s, 3H), 2.38 (s, 3H), 2.24 (s, 3H). MS (ESI): m/z [M+H]⁺ calcd for C₁₄H₁₄F₂N₂O: 264.1, found: 265.5.

### 2-(5-((3,4-Difluorophenyl) carbamoyl)-1,2,4-trimethyl-1H-pyrrol-3-yl)-2-oxoacetic acid (15a)

To a solution of 13a (860 mg, 3.26 mmol) in DCM (30 mL) were added ethyl oxalylchloride (980 μL, 8.80 mmol) and AlCl₃ (1.08 g, 8.15 mmol) at 0° C. The mixture was stirred at room temperature for 16 h and poured into crushed ice. The mixture was extracted with DCM and combined organic layers were filtered on Celite. The filtrate was concentrated and the resulting residue was used in the next step without further purification. To a solution of crude 14a in EtOH was added NaOH 10% (25 mL). The mixture was stirred for 1 h at room temperature. EtOH was evaporated under vacuum and the mixture was extracted with EtOAc (3×10 mL). The aqueous layer was acidified with 1M HCl. The mixture was extracted with EtOAc (3×10 mL). Combined organic layers were dried over MgSO₄ and concentrated in vacuo. The resulting solid was washed with Et₂O to afford 15a in 59% yield (646 mg) over two steps. ¹H NMR (400 MHz, DMSO-d₆) δ 14.13 (s, 1H), 10.46 (s, 1H), 8.04-7.71 (m, 1H), 7.59-7.28 (m, 2H), 3.60 (s, 3H), 2.46 (s, 3H), 2.26 (s, 3H). MS (ESI): m/z [M+H]⁺ calcd for C₁₆H₁₅F₂N₂O₄: 337.1, found: 337.5.

### 4,4′,4″-(2,2′,2″-((Nitrilotris(propane-3,1-diyl))tris(azanediyl))tris(2-oxoacetyl))tris(N-(3,4-difluorophenyl)-1,3,5-trimethyl-1H-pyrrole-2-carboxamide) (16a)

To a solution of 15a (75 mg, 0.22 mmol) in anhydrous DMF (5 mL) was added CDI (72 mg, 0.45 mmol) at room temperature. After 15 min, tri (3-aminopropyl) amine (25 mg, 0.13 mmol) was added and the mixture was stirred at rt for 24 h. The reaction mixture was quenched with cold H₂O (20 mL) and extracted with CHCl₃ (3×30 mL). The organic layer was dried over Na₂SO₄ and concentrated in vacuo. The residue was purified by flash chromatography using DCM/MeOH (30:1-5:1) to afford compound 16a in 40% yield (34 mg). ¹H NMR (400 MHz, DMSO-d₆) δ 10.32 (s, 3H), 8.62 (t, J=5.4 Hz, 3H), 7.82-7.76 (m, 3H), 7.36-7.30 (m, 6H), 3.50 (s, 9H), 3.17-3.12 (m, 6H), 2.39-2.36 (m, 6H), 2.30 (s, 9H), 2.15 (s, 9H), 1.58-1.55 (m, 6H). ¹⁹F NMR (377 MHz, DMSO-d₆) δ−137.22 (m), δ−144.30 (m). MS (ESI): m/z [M+H]⁺ calcd for C₅₇H₆₁F₆N₁₀O₉: 1144.2, found: 1144.5.

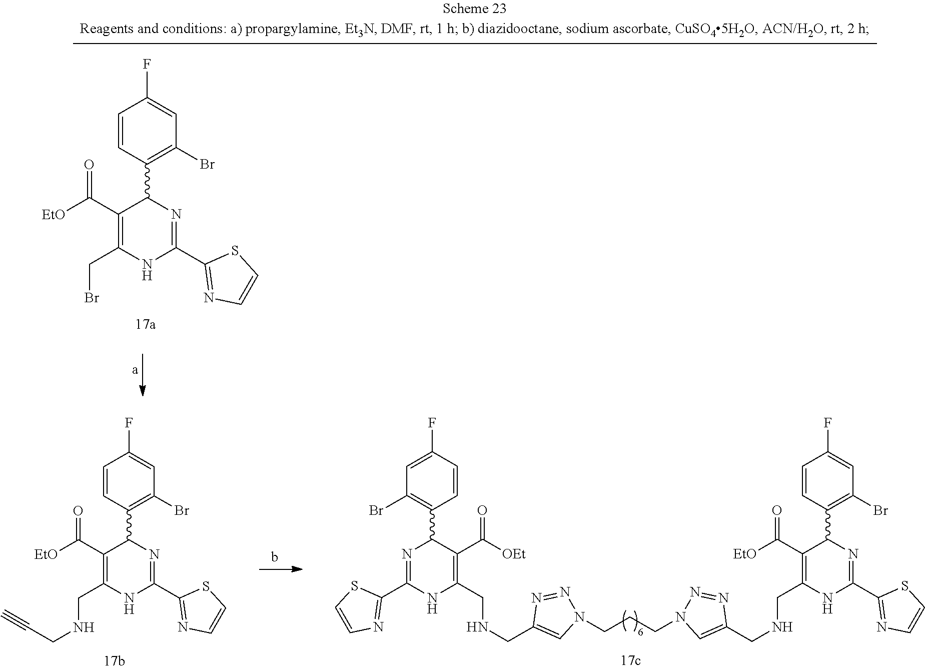

### Ethyl 4-(2-bromo-4-fluorophenyl)-6-((prop-2-yn-1-ylamino)methyl)-2-(thiazol-2-yl)-1,4-dihydropyrimidine-5-carboxylate (17b)

Propargylamine (0.09 mL, 1.34 mmol) and trimethylamine (0.25 mL, 1.78 mmol) were added to a solution of compound 17a (450 mg, 0.89 mmol) in DMF (15 mL). The mixture was stirred at room temperature for 1 h then poured into water and extracted 3 times with AcOEt. The combined organic phase was dried over Na₂SO₄, filtered and concentrated in vacuo. Purification by column chromatography (6:4 Hex:AcOEt) afforded compound 17b (190 mg, 85%). ¹H NMR (400 MHz, CDCl₃) δ 9.52 (s, 1H), 7.84 (d, J=3.1 Hz, 1H), 7.42 (d, J=3.1 Hz, 1H), 7.34-7.28 (m, 2H), 6.95 (td, J=8.3, 2.6 Hz, 1H), 6.19 (s, 1H), 4.34 (d, J=17.7 Hz, 1H), 4.24 (d, J=17.8 Hz, 1H), 4.11-4.00 (m, 2H), 3.53 (d, J=2.4 Hz, 2H), 2.28 (t, J=2.4 Hz, 1H), 1.79 (s, 1H), 1.14 (t, J=7.1 Hz, 3H). ¹³C NMR (101 MHz, CDCl₃) S 166.2, 163.1, 162.6, 160.1, 147.2, 144.2, 143.1, 140.0, 130.6 (d, J=8.5 Hz), 123.0, 120.0 (d, J=24.2 Hz), 114.9 (d, J=20.8 Hz), 97.33, 81.16, 72.29, 59.84, 58.73, 46.78, 38.42, 14.17. ¹⁹F NMR (377 MHz, CDCl₃) δ−113.5.

### Diethyl 6,6′-((((octane-1,8-diylbis(1H-1,2,3-triazole-1,4-diyl))bis(methylene))bis-(azanediyl))bis(methylene))bis(4-(2-bromo-4-fluorophenyl)-2-(thiazol-2-yl)-1,4-dihydropyrimidine-5-carboxylate) (17c)

A mixture of compound 17b (136 mg, 0.30 mmol), 1,8-diazidooctane (23 mg, 0.12 mmol), CuSO₄.5H₂O (90 mg, 0.36 mmol), sodium ascorbate (142 mg, 0.72 mmol) in acetonitrile (8 mL) and H₂O (6 mL) was stirred at room temperature for 2 h. The mixture was diluted with DCM and washed twice with a solution of NH₄OH/NH₄Cl pH 9. The organic phase was dried over Na₂SO₄, filtered and concentrated in vacuo. Purification by column chromatography (95:5 DCM:MeOH) afforded compound 17c (22 mg, 17%) as a mixture of 2 isomers (ratio 85:15). NMR of the major compound: ¹H NMR (400 MHz, CDCl₃) δ 9.73 (s, 1H), 7.90-7.77 (m, 2H), 7.64 (s, 1H), 7.59-7.41 (m, 4H), 7.37-7.20 (m, 4H), 7.08-6.92 (2H), 6.16 (s, 2H), 4.82 (d, J=15.4 Hz, 1H), 4.60 (d, J=15.3 Hz, 1H), 4.38-4.12 (m, 6H), 4.12-3.94 (m, 4H), 3.72 (q, J=7.0 Hz, 2H), 1.99-1.70 (m, 6H), 1.38-1.18 (m, 11H), 1.12 (t, J=7.1 Hz, 3H). ¹³C NMR (101 MHz, CDCl₃) S 169.2, 166.3, 160.2, 151.3, 143.9, 143.1, 131.5, 130.8, 124.5, 123.2, 122.4, 120.8, 120.0, 115.7, 115.2, 109.6, 59.9, 58.9, 58.6, 52.4, 51.8, 50.4, 47.6, 45.0, 37.4, 30.3, 28.8, 28.8, 26.5, 14.3, 14.3. ¹⁹F NMR (377 MHz, CDCl₃) δ−110.8, −113.5.

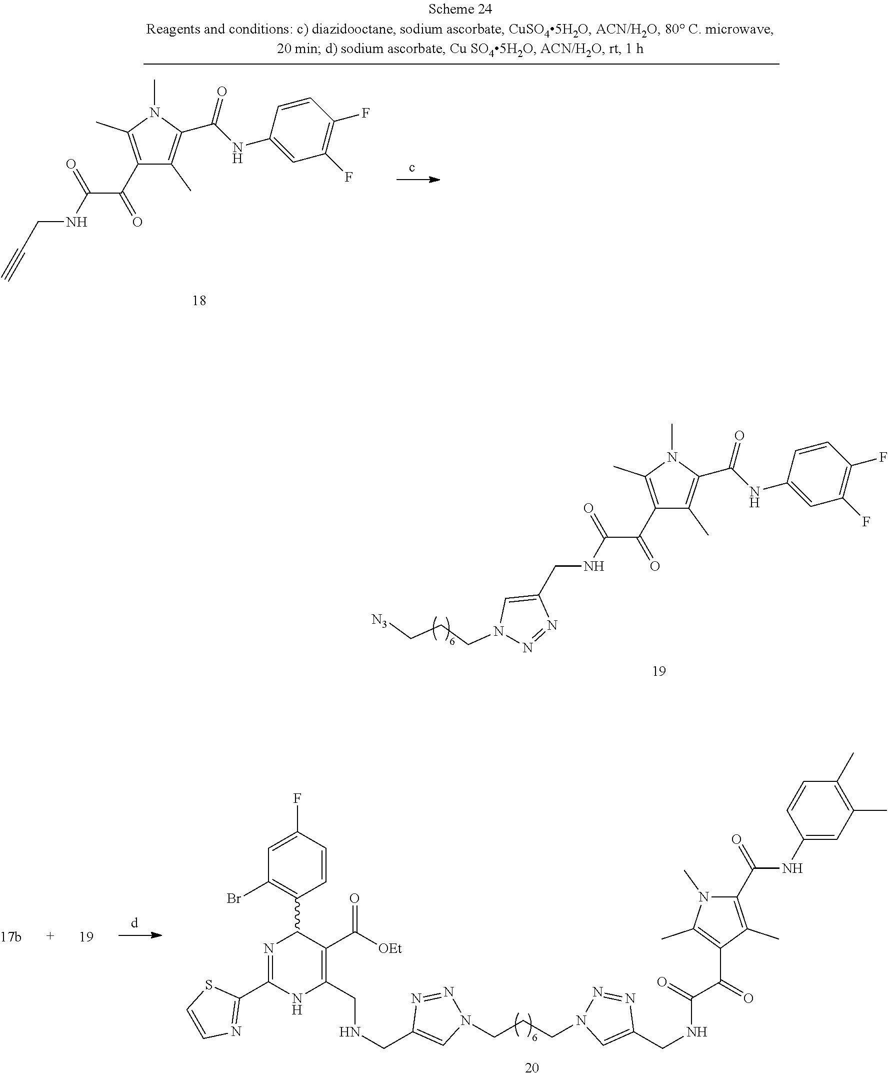

### 4-(2-(((1-(8-Azidooctyl)-1H-1,2,3-triazol-4-yl)methyl)amino)-2-oxoacetyl)-N-(3,4-difluorophenyl)-1,3,5-trimethyl-1H-pyrrole-2-carboxamide (19)

A mixture of 18 (40 mg, 0.10 mmol), 1,8-diazidooctane (30 mg, 0.15 mmol), CuSO₄.5H₂O (4 mg), sodium ascorbate (8 mg) in acetonitrile (1 mL) and H₂O (0.5 mL) was stirred at 80° C. for 20 min under microwave irradiation. The mixture was diluted with AcOEt and washed with a saturated solution of NaCl. The organic phase was dried over Na₂SO₄, filtered and concentrated in vacuo. Purification by column chromatography (98:2 DCM:MeOH) afforded 19 (24 mg, 42%). ¹H NMR (400 MHz, CDCl₃) δ 7.91 (s, 1H), 7.78-7.68 (m, 1H), 7.60-7.54 (m, 1H), 7.53-7.46 (m, 1H), 7.24-7.08 (m, 2H), 4.64-4.53 (m, 2H), 4.38-4.26 (m, 2H), 3.68 (s, 3H), 3.31-3.19 (m, 2H), 2.36 (s, 3H), 2.32 (s, 3H), 1.96-1.81 (m, 2H), 1.73 (s, 1H), 1.64-1.51 (m, 2H), 1.42-1.22 (m, 8H). ¹³C NMR (101 MHz, CDCl₃) δ 186.7, 164.7, 160.2, 143.8, 142.0, 126.2, 123.5, 122.3, 117.9, 117.3, 115.6, 109.9, 109.7, 51.5, 50.5, 35.1, 32.5, 30.3, 29.0, 28.9, 28.9, 26.7, 26.5, 12.5, 12.2. ¹⁹F NMR (377 MHz, CDCl₃) δ−135.4 to −135.5 (m), −142.2 to −142.3 (m). LC-MS (ESI) m/z: 570.5 [M+H]⁺.

### Ethyl 4-(2-bromo-4-fluorophenyl)-6-((((1-(8-(4-((2-(5-((3,4-difluorophenyl)-carbamoyl)-1,2,4-trimethyl-1H-pyrrol-3-yl)-2-oxoacelamido)methyl)-1H-1,2,3-triazol-1-yl)octyl)-1H-1,2,3-triazol-4-yl)methyl)amino)methyl)-2-(thiazol-2-yl)-1,4-dihydropyrimidine-5-carboxylate. (20)

A mixture of compound 17b (19 mg, 0.04 mmol), compound 19 (24 mg, 0.04 mmol), CuSO₄.5H₂O (42 mg, 0.17 mmol), sodium ascorbate (50 mg, 0.25 mmol) in acetonitrile (2 mL) and H₂O (1.5 mL) was stirred at room temperature for 1 h. The mixture was diluted with DCM and washed twice with a solution of NH₄OH/NH₄Cl pH 9. The organic phase was dried over Na₂SO₄, filtered and concentrated in vacuo. Purification by column chromatography (95:5 DCM:MeOH) afforded compound 20 as a yellowish solid (19 mg, 43%). ¹H NMR (400 MHz, CDCl₃) δ 9.74 (s, 1H), 8.03-7.97 (m, 1H), 7.86-7.81 (m, 1H), 7.77-7.70 (m, 1H), 7.65 (s, 1H), 7.63-7.59 (m, 1H), 7.57 (s, 1H), 7.47-7.42 (m, 1H), 7.33-7.28 (m, 1H), 7.25-7.18 (m, 2H), 7.17-7.08 (m, 1H), 7.01-6.93 (m, 1H), 6.12 (s, 1H), 4.65-4.60 (m, 2H), 4.35-4.10 (m, 6H), 4.04-3.95 (m, 4H), 3.67 (s, 3H), 2.36 (s, 3H), 2.30 (s, 3H), 1.91-1.76 (m, 4H), 1.32-1.17 (m, 8H), 1.10 (t, J=7.1 Hz, 3H). ¹³C NMR (101 MHz, CDCl₃) δ 187.4, 186.6, 173.8, 166.2, 164.7, 163.2, 160.1, 146.1, 143.8, 143.0, 141.9, 134.29, 130.7, 130.7, 126.1, 123.4, 123.1, 122.2, 122.0, 120.1, 117.7, 117.4, 117.2, 115.6, 109.8, 109.6, 97.0, 59.8, 58.7, 50.3, 50.2, 47.5, 46.1, 44.9, 35.07, 32.3, 30.1, 30.0, 28.5, 28.4, 26.1, 26.1, 14.2, 12.3, 12.2. ¹⁹F NMR (377 MHz, CDCl₃) δ−110.8, −113.4, −135.6, −142.3.

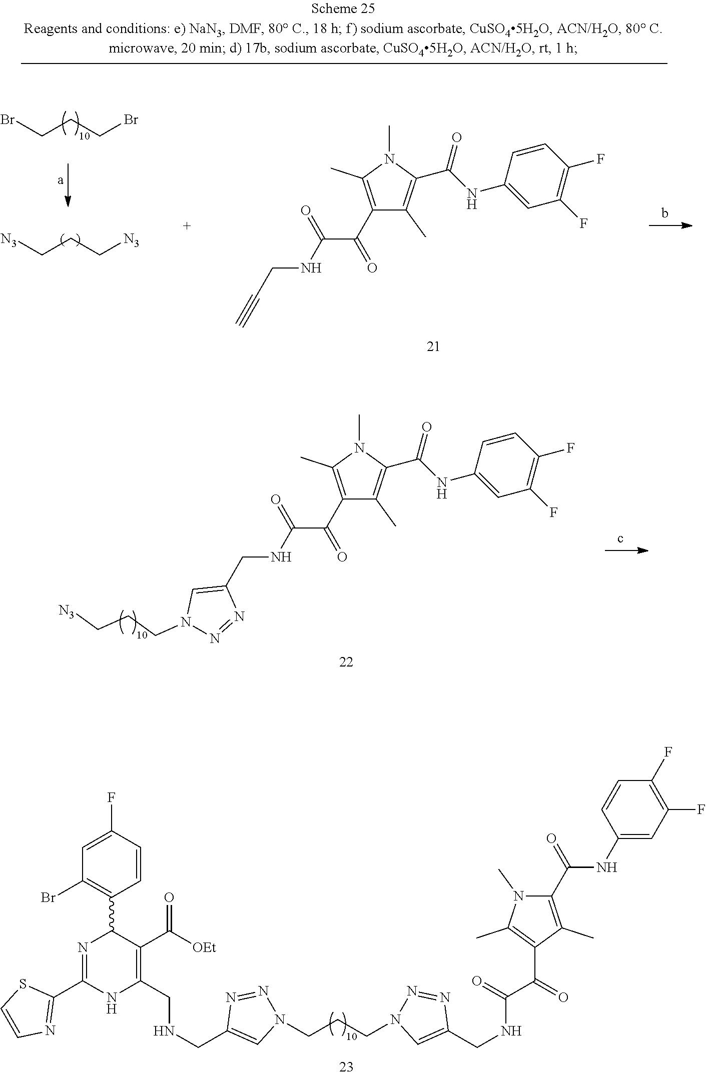

### 1,12-Diazidododecane

Sodium azide (590 mg, 9.1 mmol) was added to a solution of 1,12-dibromododecane (600 mg, 1.83 mmol) in DMF (4 mL) under nitrogen atmosphere. The mixture was stirred at 80° C. overnight. Water was added and extracted three times with hexane. The combined organic phases were dried over Na₂SO₄, filtered and concentrated in vacuo to obtain 1,12-diazidododecane as a colorless liquid (420 mg, 91%). ¹H NMR (400 MHz, CDCl₃) δ3.24 (t, J=7.0 Hz, 4H), 1.64-1.54 (m, 4H), 1.42-1.22 (m, 16H). ¹³C NMR (101 MHz, CDCl₃) δ51.4, 29.5, 29.4, 29.1, 28.9, 26.7.

### 4-(2-(((1-(12-Azidododecyl)-1H-1,2,3-triazol-4-yl)methyl)amino)-2-oxoacetyl)-N-(3,4-difluorophenyl)-1,3,5-trimethyl-1H-pyrrole-2-carboxamide (22)

A mixture of 21 (50 mg, 0.13 mmol), 1,12-diazidododecane (68 mg, 0.27 mmol), CuSO₄.5H₂O (4 mg), sodium ascorbate (8 mg) in acetonitrile (1 mL) and H₂O (0.5 mL) was stirred at 80° C. for 20 min under microwave irradiation. The mixture was diluted with AcOEt and washed with a saturated solution of NaCl. The organic phase was dried over Na₂SO₄, filtered and concentrated in vacuo. Purification by column chromatography (98:2 DCM:MeOH) afforded 22 as an oil (44 mg, 54%). ¹H NMR (400 MHz, CDCl₃) S 8.26 (s, 1H), 7.74 (ddd, J=12.1, 7.2, 2.4 Hz, 1H), 7.67-7.61 (m, 1H), 7.58 (s, 1H), 7.26-7.23 (m, 1H), 7.12 (q, J=8.9 Hz, 1H), 4.54 (d, J=5.6 Hz, 2H), 4.31 (t, J=7.3 Hz, 2H), 3.66 (s, 3H), 3.25 (t, J=7.0 Hz, 2H), 2.33 (s, 3H), 2.28 (s, 3H), 1.91-1.82 (m, 2H), 1.64-1.55 (m, 2H), 1.39-1.21 (m, 16H). ¹³C NMR (101 MHz, CDCl₃) δ 186.7, 165.0, 160.2, 151.3 (d, J=13.2 Hz), 148.9 (d, J=13.1 Hz), 148.2 (d, J=12.8 Hz), 145.8 (d, J=12.7 Hz), 143.7, 141.8, 134.4 (dd, J=8.7, 3.2 Hz), 126.3, 123.4, 122.3, 117.6, 117.2 (d, J=18.1 Hz), 115.6 (dd, J=5.8, 3.6 Hz), 109.7 (d, J=21.8 Hz), 51.5, 50.5, 34.9, 32.3, 30.3, 29.4, 29.4, 29.4, 29.3, 29.1, 29.0, 28.8, 26.7, 26.5, 12.2, 12.1. ¹⁹F NMR (377 MHz, CDCl₃) δ−135.56 to −135.73 (m), −142.32 to −142.48 (m). LC-MS (ESI) m/z: 626.4 [M+H]⁺.

### Ethyl 4-(2-bromo-4-fluorophenyl)-6-((((1-(12-(4-((2-(5-((3,4-difluorophenyl)-carbamoyl)-1,2,4-trimethyl-1H-pyrrol-3-yl)-2-oxoacetamido)methyl)-1H-1,2,3-triazol-1-yl)dodecyl)-1H-1,2,3-triazol-4-yl)methyl)amino)methyl)-2-(thiazol-2-yl)-1,4-dihydropyrimidine-5-carboxylate (23)

A mixture of compound 17b (31 mg, 0.07 mmol), compound 22 (43 mg, 0.07 mmol), CuSO₄.5H₂O (70 mg, 0.28 mmol), sodium ascorbate (83 mg, 0.42 mmol) in acetonitrile (4 mL) and H₂O (3 mL) was stirred at room temperature for 1 h. The mixture was diluted with DCM and washed twice with a solution of NH₄OH/NH₄Cl (pH 9). The organic phase was dried over Na₂SO₄, filtered and concentrated in vacuo. Purification by column chromatography (96:4 DCM:MeOH) afforded compound 23 as a yellowish solid (15 mg, 19%). 2 isomers are observed by NMR (ratio 9:1), only the major one is described: ¹H NMR (400 MHz, CDCl₃) δ 9.74 (s, 1H), 7.91 (s, 1H), 7.83 (d, J=3.1 Hz, 1H), 7.73 (ddd, J=12.2, 7.2, 2.5 Hz, 1H), 7.65 (s, 1H), 7.56 (s, 1H), 7.52-7.47 (m, 1H), 7.47-7.42 (m, 1H), 7.30 (dd, J=8.4, 2.6 Hz, 1H), 7.22-7.17 (m, 1H), 7.17-7.09 (m, 1H), 7.01-6.93 (m, 1H), 6.14 (s, 1H), 4.61 (d, J=5.9 Hz, 2H), 4.35-4.28 (m, 4H), 4.27-4.12 (m, 2H), 4.06-3.94 (m, 4H), 3.68 (s, 3H), 2.36 (s, 3H), 2.31 (s, 3H), 1.92-1.82 (m, 4H), 1.32-1.19 (m, 16H), 1.11 (t, J=7.1 Hz, 3H). ¹³C NMR (101 MHz, CDCl₃) δ 186.6, 164.5, 160.1, 143.7, 141.9, 134.3, 130.6, 126.1, 123.4, 122.2, 121.9, 117.8, 117.3 (d, J=18.4 Hz), 115.4, 114.9, 109.7 (d, J=21.8 Hz), 50.4, 50.4, 35.0, 32.4, 30.2, 29.3, 29.3, 29.2, 28.9, 28.9, 26.4, 26.4, 14.2, 12.4, 12.2. ¹⁹F NMR (377 MHz, CDCl₃) δ−110.8, −113.5, −135.4 to −135.5 (m, F), −142.2 to −142.3 (m, F).

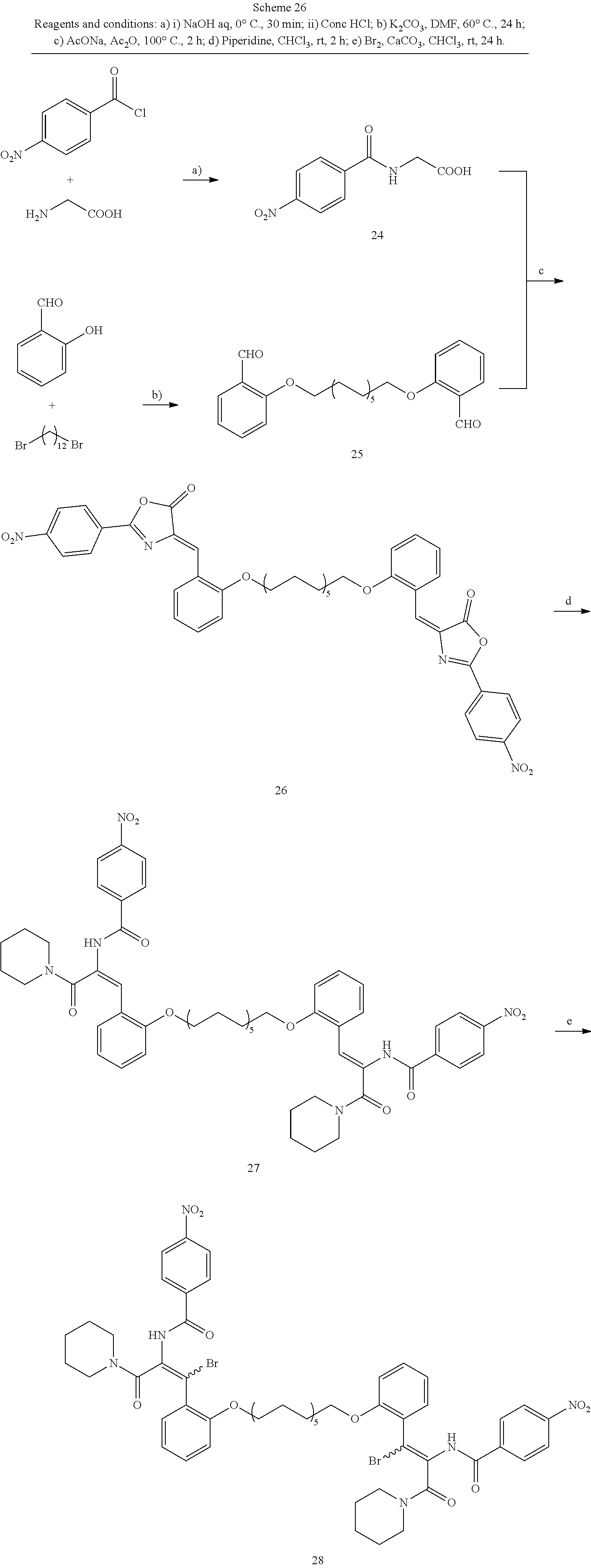

### 2-(4-Nitrobenzamido)acetic acid (24)

To a solution of glycine (0.963 g, 12.8 mmol) in 10 mL of a 10% sodium hydroxide solution was added 4-nitrobenzoyl chloride (2 g, 10.7 mmol). The reaction was stirred for 30 min at 0° C. and then acidified by adding 1 M HCl solution. The precipitated solid was filtered off and the solid was recrystallized from water to afford 24 as a yellow solid (1.8 g, 75%); ¹H NMR (400 MHz, DMSO-d₆): δ 12.72 (s, 1H), 9.21 (t, J=5.9 Hz, 1H), 8.33 (d, J=8.9 Hz, 2H), 8.10 (d, J=8.9 Hz, 2H), 3.98 (d, J=5.9 Hz, 2H); ¹³C NMR (101 MHz, DMSO): δ 171.1, 165.0, 149.2, 139.5, 128.8, 123.7, 41.4, 40.1.

### 2,2′-(Dodecane-1,12-diylbis(oxy))dibenzaldehyde (25)

A solution of 1,12-dibromododecane (1.34 g, 4.09 mmol), salicylaldehyde (1 g, 8.18 mmol) and K₂CO₃ (1.13 g, 8.18 mmol) in 12 mL of DMF was stirred for 24 h at 60° C. The mixture was quenched with water. After extraction with AcOEt, the combined organic layers were dried over MgSO₄ and evaporated. The crude residue was purified via column chromatography on silica gel (hexanes/EtOAc 95:5) to afford 25 as a white solid (750 mg, 56%); ³H NMR (400 MHz, CDCl₃): δ10.52 (d, J=1.8 Hz, 2H), 7.83 (dd, J=7.3, 1.8 Hz, 2H), 7.53 (ddd, J=8.4, 7.3, 1.9 Hz, 2H), 7.02-6.96 (m, 4H), 4.07 (t, J=6.4 Hz, 4H), 1.88-1.83 (m, 4H), 1.54-1.43 (m, 4H), 1.37-1.30 (m, 13H); ¹³C NMR (101 MHz, CDCl₃): δ190.0, 161.7, 136.0, 128.3, 125.0, 120.8, 112.6, 68.7, 29.7, 29.4, 29.2, 26.2; SM (IS): m/z=411 [M+1];

### (4Z,4′E)-4,4′-(((Dodecane-1,12-diylbis(oxy))bis(2,1-phenylene))bis(methanylylidene))bis(2-(4-nitrophenyl)oxazol-5(4H)-one) (26)

A mixture of 25 (0.552 g, 1.34 mmol), 2-(4-nitrobenzamido)acetic acid (24) (0.652 mg, 2.68 mmol), sodium acetate (0.22 g, 2.69 mmol) and acetic anhydride (0.6 mL) was heated for 2 h at 100° C. After cooling down the reaction to room temperature, the precipitate was triturated with ethanol, washed with water, and then dried under vacuum to afford 26 as a yellow solid (0.8 g, 76%); ³H NMR (400 MHz, CDCl₃): δ8.82 (d, J=7.6 Hz, 2H), 8.34 (q, J=8.7 Hz, 8H), 7.98 (s, 1H), 7.44 (t, J=7.6 Hz, 2H), 7.08 (t, J=7.6 Hz, 2H), 6.93 (d, J=8.4 Hz, 2H), 4.06 (t, J=6.5 Hz, 4H), 2.08-1.71 (m, 4H), 1.71-1.36 (m, 4H), 1.50-1.34 (m, 13H); ¹³C NMR (101 MHz, CDCl₃): δ167.1, 160.9, 159.5, 150.3, 134.0, 133.2, 131.7, 131.6, 129.1, 129.1, 124.2, 122.5, 121.0, 112.0, 69.0, 29.6, 29.4, 29.2, 26.2. SM (IS): m/z=787 [M+1]; HRMS

### N,N′-((1E,1′E)-((Dodecane-1,12-diylbis(oxy))bis(2,1-phenylene))bis(3-oxo-3-(piperidin-1-yl)prop-1-ene-2,1-diyl))bis(4-nitrobenzamide) (27)

To a solution of 26 (0.8 g, 1.01 mmol) in chloroform (3 ml), was added dropwise a solution of piperidine (0.2 ml, 2.035 mmol) in chloroform (2 ml). The reaction was stirred for 1 h at rt. After removal of the volatiles under vacuum, the crude residue was purified via column chromatography on silica gel (DCM/MeOH 99:1 to 95:5) to afford 27 as a yellow solid (710 mg, 73%); ¹H NMR (400 MHz, CDCl₃); δ 10.10 (s, 2H), 8.10 (d, J=8.9 Hz, 4H), 7.98 (d, J=8.9 Hz, 4H), 7.47 (s, 1H), 7.32-7.27 (m, 3H), 7.02-6.94 (m, 4H), 6.31 (s, 2H), 3.98 (t, J=6.6 Hz, 4H), 3.77 (s, 4H), 3.49 (d, J=6.4 Hz, 4H), 1.66 (dt, J=18.8, 6.7 Hz, 12H), 1.36 (q, J=7.1 Hz, 5H), 1.29-1.17 (m, 15H); ¹³C NMR (101 MHz, CDCl₃): δ 167.8, 163.8, 155.9, 149.5, 138.6, 130.1, 129.7, 128.8, 123.5, 123.3, 121.0, 117.6, 113.1, 69.5, 49.3, 43.4, 29.6, 29.4, 29.3, 26.1, 25.8, 25.5, 24.8; SM (IS): m/z=957 [M+1]; HRMS

### N,N′-((Dodecane-1,12-diylbis(oxy))bis(2,1-phenylene))bis(1-bromo-3-oxo-3-(piperidin-1-yl)prop-1-ene-2,1-diyl))bis(4-nitrobenzamide) (28)

To a solution of 27 (0.3 g, 0.313 mmol) in chloroform (1 ml), was added dropwise a solution of Br₂ (0.1 mg, 0.626 mmol) in chloroform (1 ml). The reaction was stirred for 24 h at rt. After removal of the volatiles under vacuum, the crude residue was purified via column chromatography on silica gel (DCM/Hexane/MeOH 80/20/1 to 80/20/5) to afford 28 as a yellow solid (40 mg, 9%); ¹H NMR (400 MHz, CDCl₃): 8.44 (s, 2H), 8.33 (d, J=8.9 Hz, 4H), 8.08 (d, J=8.9 Hz, 4H), 7.35-7.26 (m, 4H), 6.96-6.88 (m, 4H), 4.01 (dt, J=6.3, 3.9 Hz, 4H), 3.55-3.45 (m, 2H), 3.35-3.23 (m, 4H), 3.16 (t, J=10.6 Hz, 2H), 1.80 (p, J=6.3 Hz, 4H), 1.66 (dt, J=18.8, 6.3 Hz, 8H), 1.41 (q, J=5.5, 3.9 Hz, 5H), 1.30-1.21 (m, 15H); ¹³C NMR (101 MHz, CDCl₃): δ 162.2, 157.2, 150.2, 138.4, 131.5, 131.2, 130.7, 128.8, 124.1, 120.3, 111.5, 110.2, 68.3, 47.8, 42.7, 29.7, 29.4, 29.3, 26.3, 25.2, 25.0, 24.3; SM (IS): m/z=1115 [M+1]; HRMS

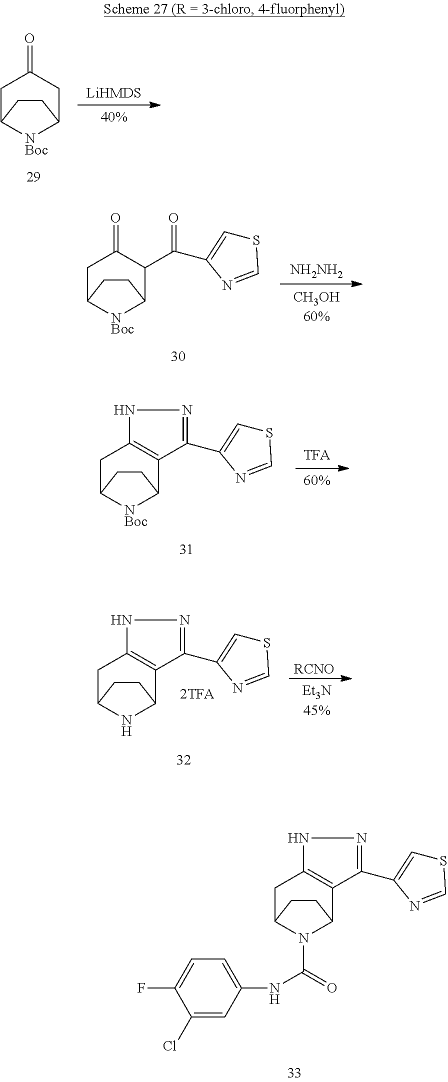

### (Tert-butyl(1S,5R)-3-oxo-2-(thiazole-4-carbonyl)-8-azabicyclo[3,2,1]octane-8-carboxylate) (30)

To a solution of tert-butyl (1R,5S)-3-oxo-8-azabicyclo[3.2.1]octane-8-carboxylate 29 (1.05 g, 4.66 mmol) in THF (14 mL) at −75° C. was added LiHMDS (1.5 M in THF, 4.6 mL, 6.9 mmol). After 30 min at this temperature, thiazole-4-carbonyl chloride (0.7 g, 4.74 mmol) was added to the solution. The reaction mixture was stirred at −75° C. for 2 h, quenched with a saturated solution of ammonium chloride (20 mL), extracted with ethyl acetate (10 mL×3). The combined organic layers were dried over sodium sulfate, concentrated, and purified via column chromatography on silica gel (hexane/ethyl acetate, 100/30 to 100:/50) to give 30 as a white solid (0.75 g, 48%). ¹H-NMR (CDCl₃, 400 MHz): δ 8.86 (s, 1H), 8.22 (d, J=2.0 Hz, 1H), 6.04 (s, 1H), 4.44 (s, 1H), 3.01 (s, 1H), 2.40 and 2.35 (2s, 1H), 2.22-2.32 (m, 2H), 2.02-2.04 (m, 1H), 1.70-1.74 (m, 1H), 1.25-1.52 (m, 10H). ¹³C-NMR (CDCl₃, 400 MHz): 197.8, 169.8, 168.7, 154.6, 152.47, 125.5, 114.0, 79.7, 55.6, 52.5, 51.5, 44.0, 34.8, 29.4, 28.3. LC-MS m/z (M+H)⁺: 337.4.

### (Tert-butyl(4S,7R)-3-(thiazol-4-yl)-1,4,5,6,7,8-hexahydro-4,7-epiminocyclohenta[c]pyrazole-9-carboxylate) (31)

To a solution of 30 (200 mg, 0.59 mmol) in MeOH was added hydrazine monohydrate (0.05 mL). The reaction was stirred at room temperature overnight and concentrated under vacuum. The residue was purified via column chromatography on silica gel (dichloromethane:methanol v/v; 100:1 to 100:10) to give 31 as a white solid (170 mg, 86%). ¹H-NMR (CDCl₃, 400 MHz): δ 8.97 (s, 1H), 7.47 (s, 1H), 5.36 (m, 1H), 4.55 (m, 1H), 3.24-3.38 (m, 1H), 2.59 (m, 1H), 2.15-2.23 (m, 1H), 1.88 (t, J=10 Hz, 1H), 1.67 (m, 1H), 1.35-1.44 (m, 10H). ¹³C-NMR (CDCl₃, 100 MHz): δ 171.1, 153.5, 146.5, 144.2, 132.7, 118.4, 113.0, 79.7, 51.4, 34.7, 30.9, 29.2, 28.4. LC-MS m/z (M+H)⁺: 333.4.

### ((4S,7R)—N-(3-Chloro-4-fluorophenyl)-3-(thiazol-4-yl)-1,4,5,6,7,8-hexahydro-4,7-epiminocyclo hepta[c]pyrazole-9-carboxamide) (33)

To a solution of 31 (200 mg, 0.6 mmol) in dichloromethane (3 mL) was added TFA (0.5 mL). The reaction mixture was stirred at room temperature overnight and then concentrated under vacuum. The residue was co-evaporated with ethyl acetate (3 mL), washed by ether (2 mL×2) to obtain 32 as a white solid (0.23 g, 85%). To a solution of 32 (0.23 g, 0.5 mmol) in dichloromethane (2 mL) was added triethylamine (0.28 mL, 2 mmol) and 3-chloro-4-fluorophenyl isocyanate (0.09 g, 0.52 mmol). The reaction mixture was stirred at room temperature overnight and then poured into water (3 mL). The mixture was extracted with ethyl acetate (5 mL×3). The combined organic layers were washed with water and a saturated solution of sodium chloride then dried over sodium sulfate and concentrated under vacuum. The residue was purified via column chromatography on silica gel (dichloromethane:methanol (v/v)=100:1 to 100:10) to give 33 as a white solid (0.13 g, 65%). ¹H-NMR (DMSO-d₆+D₂O, 400 MHz): δ 9.18 and 9.22 (2s, 1H), 7.83 and 7.90 (2s, 1H), 7.69 (d, J=4.8 Hz, 1H), 7.35 (d, J=7.6 Hz, 1H), 7.20 (t, J=8.8 Hz, 1H), 5.62 (s, 1H), 4.72 (s, 1H), 3.21 (m, 1H), 2.51 (m, 2H), 2.28 (m, 1H), 2.10 (m, 1H), 1.82 (m, 1H), 1.67 (m, 1H). ¹⁹F-NMR (DMSO-d₆, 376 MHz): δ 125.4. ¹³C-NMR (DMSO-d₆, 100 MHz): δ 154.6, 153.7, 151.0, 145.8, 141.1, 137.8, 136.6, 131.1, 120.9, 119.7, 118.7, 116.7, 113.8, 52.1, 51.5, 35.1, 31.6, 29.1. LC-MS m/z (M+H)⁺: 404.3

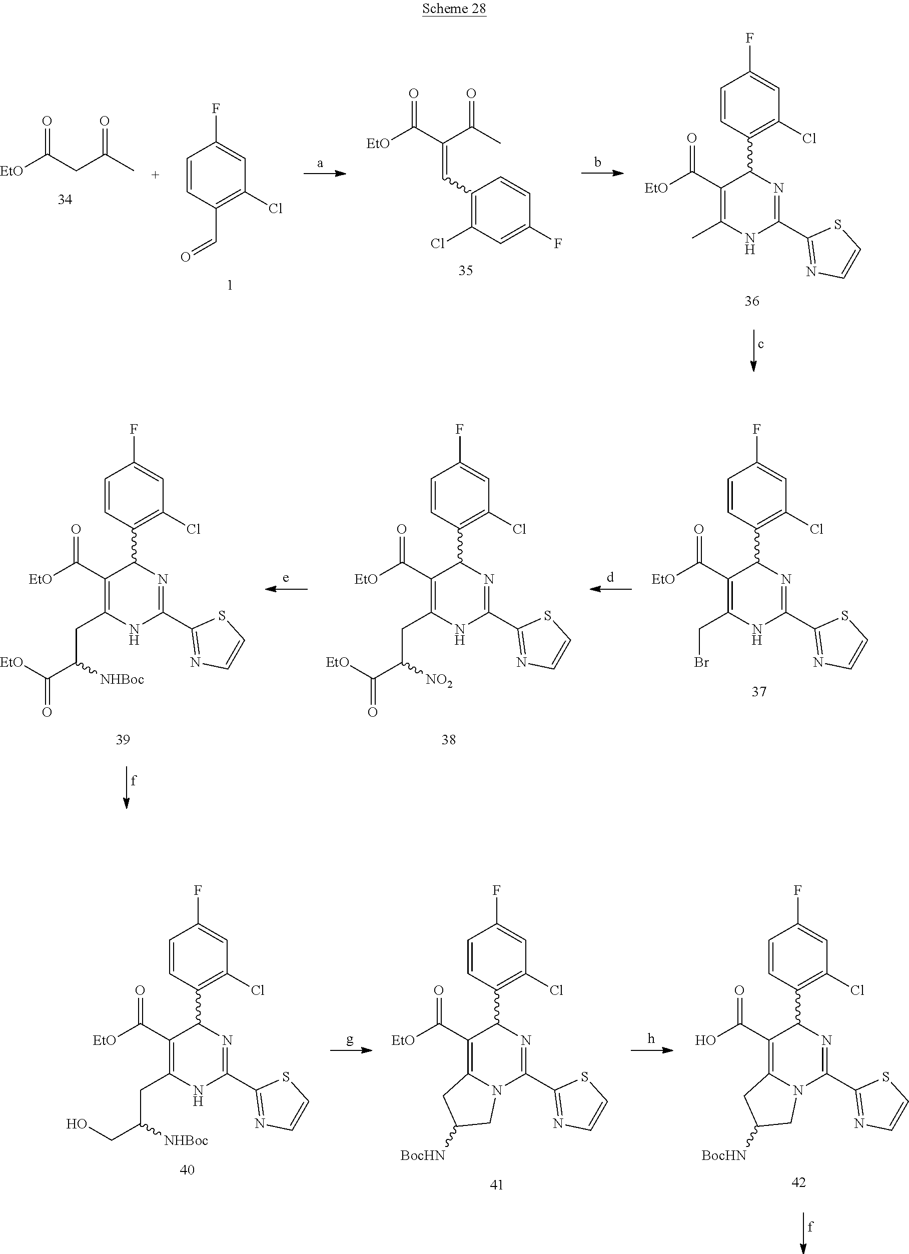

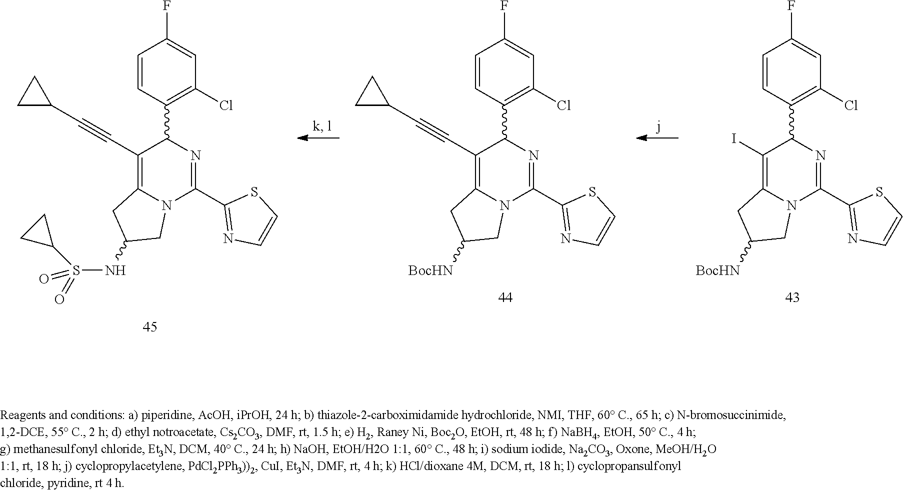 [SMILES: O=S(=O)(NC1CC2=C(C#CC3CC3)C(c3ccc(F)cc3Cl)N=C(c3nccs3)N2C1)C1CC1 ; CC(C)(C)OC(=O)NC1CC2=C(C#CC3CC3)C(c3ccc(F)cc3Cl)N=C(c3nccs3)N2C1 ; CC(C)(C)OC(=O)NC1CC2=C(I)C(c3ccc(F)cc3Cl)N=C(c3nccs3)N2C1]

### Ethyl 2-(2-chloro-4-fluorobenzylidene)-3-oxobutanoate (35)

To a solution of 2-chloro-4-fluorobenzaldehyde (5 g, 31.5 mmol) in isopropanol (50 mL) were added ethyl 3-oxobutanoate (4.83 mL, 37.9 mmol), piperidine (0.62 mL, 6.3 mmol) and acetic acid (0.9 mL, 15.8 mmol). The mixture was stirred at room temperature for 24 h before being concentrated in vacuo. The residue was then diluted with AcOEt (100 mL) and washed with a saturated solution of Na₂CO₃. The organic layer was dried over Na₂SO₄, filtered and concentrated in vacuo. Purification by column chromatography (9:1 Hex:AcOEt) afforded 35 as a 6:4 mixture of isomers (6.56 g, 77%). ¹H NMR (400 MHz, CDCl₃) δ 7.87 (s, 0.4H), 7.80 (s, 0.6H), 7.46 (dd, J=8.7, 5.9 Hz, 0.6H), 7.35 (dd, J=8.7, 5.9 Hz, 0.4H), 7.24-7.18 (m, 1H), 7.03-6.94 (m, 1H), 4.32 (q, J=6.9 Hz, 0.8H), 4.25 (q, J=7.1 Hz, 1.2H), 2.46 (s, 1.8H), 2.26 (s, 1.2H), 1.35 (t, J=7.1 Hz, 1.2H), 1.21 (t, J=7.1 Hz, 1.8H). ¹³C NMR (101 MHz, CDCl₃) δ 202.1, 194.5, 166.8, 164.6, 164.5, 163.9, 162.1, 161.9, 137.0, 136.6, 136.4, 135.9 (d, J=10.5 Hz), 135.6 (d, J=10.4 Hz), 131.5 (d, J=9.1 Hz), 130.7 (d, J=9.1 Hz), 128.3 (d, J=3.8 Hz), 128.0 (d, J=3.7 Hz), 117.5 (d, J=24.9 Hz), 117.5 (d, J=24.9 Hz), 114.6 (d, J=21.4 Hz), 114.5 (d, J=21.5 Hz), 61.8, 61.8, 31.3, 26.8, 14.1, 13.9. ¹⁹F NMR (377 MHz, CDCl₃): δ−107.3 to −107.4 (m, F), −107.7 to −107.7 (m, F). HRMS (ESI) m/z calcd for C₁₃H₁₃ClFO₃ [M+H]⁺: 271.0459; found 271.0526.

### Ethyl 4-(2-chloro-4-fluorophenyl)-6-methyl-2-(thiazol-2-yl)-1,4-dihydropyrimidine-5-carboxylate (36)

A mixture of compound 35 (6.56 g, 24.2 mmol), thiazole-2-carboximidamide hydrochloride (4.74 g, 29.1 mmol) and A-methylimidazole (3.84 mL, 48.4 mmol) in THF (70 mL) under nitrogen atmosphere, was stirred at 60° C. for 65 h. The mixture was then poured into an aqueous solution of NH₄Cl (200 mL) and extracted with AcOEt (3×50 mL). The combined organic layers were dried over Na₂SO₄, filtered and concentrated in vacuo. Purification by column chromatography (7:3 Hex:AcOEt) afforded compound 36 as a yellow solid (8.56 g, 93%). ¹H NMR (400 MHz, CDCl₃) δ 7.83 (d, J=3.1 Hz, 0.25H), 7.80 (d, J=3.1 Hz, 0.75H), 7.50 (d, J=3.1 Hz, 0.25H), 7.43 (d, J=3.1 Hz, 0.75H), 7.37-7.32 (m, 1H), 7.15-7.10 (m, 1H), 6.97-6.88 (m, 1H), 6.20 (s, 0.75H), 6.08 (d, J=2.4 Hz, 0.25H), 4.09-4.00 (m, 2H), 2.57 (s, 0.75H), 2.51 (s, 2.25H), 1.13 (t, J=7.1 Hz, 3H). ¹³C NMR (101 MHz, CDCl₃) δ 166.3, 166.1, 163.2, 162.8, 162.7, 162.5, 160.7, 160.2, 157.7, 149.2, 145.1, 143.9, 143.8, 142.8, 138.3 (d, J=3.5 Hz), 136.7 (d, J=3.5 Hz), 133.7 (d, J=10.3 Hz), 132.5 (d, J=10.5 Hz), 130.7 (d, J=8.9 Hz), 130.6 (d, J=9.0 Hz), 124.2, 123.2, 117.0 (d, J=24.8 Hz), 116.8 (d, J=24.5 Hz), 114.8 (d, J=21.0 Hz), 114.3 (d, J=20.9 Hz), 104.6, 98.9, 60.1, 59.9, 56.2, 49.4, 23.2, 18.7, 14.1. ¹⁹F NMR (377 MHz, CDCl₃) δ−111.6 to −111.7 (m, F), −113.4 to −113.5 (m, F). HRMS (ESI) m/z calcd for C₁₇H₁₆ClFN₃O₂S [M+H]⁺: 380.0558; found 380.0621.

### Ethyl 6-(bromomethyl)-4-(2-chloro-4-fluorophenyl)-2-(thiazol-2-yl)-1,4-dihydropyrimidine-5-carboxylate (37)

Compound 36 (8.56 g, 22.5 mmol) was dissolved in a solution of 1,2-DCE (50 mL) and acetic acid (35 mL) at 55° C. A solution of N-bromosuccinimide (4.41 g, 24.8 mmol) in 1,2-DCE (150 mL) and acetic acid (35 mL) was added dropwise over 1.5 h to the solution at 55° C. After addition, the mixture was stirred for an additional 30 min before being cooled to room temperature. The mixture was then poured into a saturated solution of Na₂CO₃ (300 mL) and extracted with DCM (3×50 mL). The combined organic phase was dried over Na₂SO₄, filtered and concentrated in vacuo. Purification by column chromatography (9:1 Hex:AcOEt) afforded 37 as a yellow oil (6.73 g, 65%). ¹H NMR (400 MHz, CDCl₃) δ 8.23 (s, 0.3H), 7.84 (d, J=3.1 Hz, 1H), 7.54 (d, J=3.1 Hz, 0.7H), 7.48 (s, 0.7H), 7.46 (d, J=3.2 Hz, 0.3H), 7.44-7.34 (m, 1H), 7.14 (dd, J=8.3, 2.5 Hz, 1H), 7.01-6.90 (m, 1H), 6.20 (s, 0.3H), 6.11 (d, J=2.5 Hz, 0.7H), 4.98-4.90 (m, 1H), 4.75 (d, J=11.3 Hz, 0.3H), 4.59 (d, J=8.4 Hz, 0.7H), 4.17-4.04 (m, 2H), 1.19-1.12 (m, 3H). ¹³C NMR (101 MHz, CDCl₃) δ 165.3, 164.7, 163.3, 162.0, 160.8, 155.7, 150.3, 143.9, 143.0 (d, J=11.9 Hz), 136.2, 132.5 (d, J=10.5 Hz), 130.5 (d, J=9.2 Hz), 124.6, 123.4, 117.1 (d, J=24.8 Hz), 115.1 (d, J=21.1 Hz), 114.5 (d, J=21.4 Hz), 106.0, 60.7, 60.6, 56.1, 49.2, 31.8, 26.1, 14.0. ¹⁹F NMR (377 MHz, CDCl₃) δ−110.94 to −111.0 (m, F), −112.7 to −112.8 (m, F). HRMS (ESI) m/z calcd for C₁₇H₁₅BrClFN₃CO₂S [M+H]⁺: 457.9663; found 457.9726.

### Ethyl 4-(2-chloro-4-fluorophenyl)-6-(3-ethoxy-2-nitro-3-oxopropyl)-2-(thiazol-2-yl)-1,4-dihydropyrimidine-5-carboxylate (38)

Cesium carbonate (14.2 g, 43.5 mmol) was added to a solution of 37 (6.60 g, 14.4 mmol) and ethyl nitroacetate (3.2 mL, 28.8 mmol) in DMF (50 mL). The mixture was stirred at room temperature for 1.5 h then poured into water (500 mL) and extracted with DCM (3×100 mL). The combined organic layers were dried over Na₂SO₄, filtered and concentrated in vacuo. Purification by column chromatography (9:1 Hex:AcOEt) afforded 38 as a yellow oil (5.19 g, 71%). NMR spectra show 2 isomers (5:5 ratio). ³H NMR (400 MHz, CDCl₃) δ 7.82 (d, J=3.1 Hz, 1H), 7.56-7.49 (m, 2H), 7.39-7.32 (m, 1H), 7.17-7.11 (m, 1H), 6.99 (qd, J=7.8, 2.6 Hz, 1H), 6.12 (d, J=10.5 Hz, 1H), 6.01-5.93 (m, 1H), 4.38-4.16 (m, 3H), 4.08 (q, J=7.1 Hz, 2H), 3.82 (dd, J=17.6, 4.8 Hz, 0.5H), 3.66 (dd, J=17.5, 4.4 Hz, 0.5H), 1.33 (t, J=7.2 Hz, 3H), 1.13 (t, J=7.1 Hz, 3H). ¹³C NMR (101 MHz, CDCl₃) δ 165.3, 165.2, 163.3, 161.9, 160.8, 153.8, 153.5, 149.6, 143.9, 136.3, 132.5, 130.8 (t, J=8.9 Hz), 125.0, 124.9, 117.3, 117.1 (d, J=24.7 Hz), 117.0 (d, J=24.9 Hz), 115.1 (t, J=22 Hz), 104.9, 85.4, 77.4, 63.0, 60.5, 49.4, 35.0, 35.0, 14.0, 14.0. ¹⁹F NMR (377 MHz, CDCl₃) δ−110.95 to −111.03 (m, F). HRMS (ESI) m/z calcd for C₂₁H₂₁ClFN₄O₆S [M+H]⁺: 511.0776; found 511.0841.

### Ethyl 6-(2-((tert-butoxycarbonyl)amino)-3-ethoxy-3-oxopropyl)-4-(2-chloro-4-fluoro-phenyl)-2-(thiazol-2-yl)-1,4-dihydropyrimidine-5-carboxylate (39)

A commercially available solution of Raney nickel in water (5 mL) was washed three times with EtOH. The Raney nickel was then suspended in EtOH (50 mL) before addition of a solution of 38 (5.19 g, 10.2 mmol) in EtOH (150 mL) and di-tert-butyl dicarbonate (4.45 g, 20.4 mmol). The mixture was stirred at room temperature for 48 h under hydrogen (1 atm). The mixture was then filtered over celite and concentrated in vacuo. Purification by column chromatography (7:3 Hex:AcOEt) afforded 39 as a yellow oil (5.46 g, 92%). ¹H NMR (400 MHz, CDCl₃) δ 7.86-7.80 (m, 1H), 7.55-7.33 (m, 3H), 7.16-7.09 (m, 1H), 7.01-6.88 (m, 1H), 6.21 (d, J=12.4 Hz, 0.3H), 6.13-6.08 (m, 0.9H), 6.01 (d, J=8.4 Hz, 0.3H), 4.75-4.65 (m, 0.5H), 4.56 (d, J=17.2 Hz, 0.3H), 4.45 (d, J=17.2 Hz, 0.3H), 4.39-4.30 (m, 0.5H), 4.26-4.15 (m, 1.75H), 4.15-4.00 (m, 2.8H), 3.70 (dd, J=14.6, 7.4 Hz, 0.3H), 3.61 (dd, J=14.9, 7.4 Hz, 0.3H), 3.38 (dd, J=15.0, 4.2 Hz, 0.3H), 3.18 (dd, J=14.6, 4.5 Hz, 0.3H), 1.56-1.39 (m, 9H), 1.30-1.23 (m, 3H), 1.18-1.09 (m, 3H). ¹³C NMR (101 MHz, CDCl₃) δ 172.5, 172.3, 171.5, 171.4, 171.1, 165.8, 165.7, 165.3, 163.2, 162.5, 162.27, 162.2, 162.1, 160.8, 156.9, 156.2, 155.8, 151.1, 149.5, 149.3, 149.1, 143.9, 142.9, 136.5 (d, J=8.5 Hz), 133.6 (d, J=10.5 Hz), 132.5 (d, J=10.2 Hz), 131.0 (d, J=8.4 Hz), 130.8 (d, J=10.9 Hz), 124.7 (d, J=10.2 Hz), 123.2, 117.1, 117.1, 116.9, 116.8, 115.1, 114.9, 106.42, 106.2, 104.4, 79.3, 62.3, 61.9, 61.1, 60.4, 56.2, 56.0, 52.6, 52.6, 49.6, 49.4, 36.2, 36.0, 32.9, 28.4, 28.3, 27.7, 14.2. ¹⁹F NMR (377 MHz, CDCl₃) δ−111.13 to −111.45 (m, F), −113.22 to −113.33 (m, F). HRMS (ESI) m/z calcd for C₂₆H₃₁ClFN₄O₆S [M+H]⁺: 581.1559; found 581.1623.

### Ethyl 6-(2-((tert-butoxycarbonyl)amino)-3-hydroxypropyl)-4-(2-chloro-4-fluorophenyl)-2-(thiazol-2-yl)-1,4-dihydropyrimidine-5-carboxylate (40)

Sodium borohydride (1.39 g, 36.9 mmol) was added to a solution of 39 (5.36 g, 9.2 mmol) in ethanol (150 mL) under nitrogen atmosphere. The mixture was stirred at 50° C. for 4 h before being concentrated in vacuo. The residue was dissolved in ethyl acetate (100 mL) and washed with water (200 mL) and brine (200 mL). The organic layer was dried over Na₂SO₄, filtered and concentrated in vacuo. Purification by column chromatography (10:0 to 0:10 Hex:AcOEt) afforded 40 as a yellow oil (3.17 g, 64%). ¹H NMR (400 MHz, CDCl₃) δ 7.86-7.79 (m, 1H), 7.57-7.49 (m, 1H), 7.44-7.40 (m, 0.5H), 7.40-7.31 (m, 1H), 7.17-7.09 (m, 1H), 7.01-6.87 (m, 1H), 6.22 (s, 0.25H), 6.17 (s, 0.2H), 6.11 (d, J=2.4 Hz, 0.2H), 6.09 (d, J=2.5 Hz, 0.25H), 5.92 (d, J=7.4 Hz, 0.2H), 5.80 (d, J=7.4 Hz, 0.25H), 5.67 (d, J=7.2 Hz, 0.2H), 5.51 (d, J=9.3 Hz, 0.25H), 4.24-3.83 (m, 3.7H), 3.83-3.60 (m, 2.3H), 3.47 (dd, J=13.2, 8.9 Hz, 0.3H), 3.41-3.27 (m, 0.7H), 3.11-2.98 (m, 0.5H), 2.93 (dd, J=12.5, 6.6 Hz, 0.25H), 2.77 (dd, J=13.2, 5.9 Hz, 0.25H), 2.43 (s, 0.2H), 1.44 (s, 9H), 1.16-1.08 (m, 3H). ¹³C NMR (101 MHz, CDCl₃) δ 163.3, 162.8, 162.7, 162.5, 161.9, 161.8, 160.8, 160.3, 158.6, 158.1, 157.0, 156.7, 156.1, 154.8, 151.0, 149.3, 149.2, 148.3, 147.5, 146.6, 145.8, 144.0, 144.0, 143.3, 142.9, 142.0, 141.9, 138.4, 136.5, 136.4, 133.6, 133.5, 132.6, 131.0, 130.6, 130.6, 130.5, 130.5, 124.6, 124.5, 123.1, 117.3, 117.3, 117.1, 117.0, 117.0, 116.8, 116.7, 115.1 (d, J=21.9 Hz), 114.5 (d, J=20.8 Hz), 106.7 (d, J=16.1 Hz), 79.8, 79.8, 79.1, 79.1, 65.5, 65.4, 65.3, 65.2, 63.5, 63.2, 63.1, 62.8, 60.8, 60.8, 60.6, 60.6, 60.4, 59.6, 58.0, 57.9, 56.4, 55.9, 53.4, 52.2, 52.1, 52.1, 51.5, 51.5, 49.7, 49.6, 36.0, 36.0, 35.8, 32.9, 32.2, 28.5, 28.4, 28.4. ¹⁹F NMR (377 MHz, CDCl₃) δ−110.8 to −111.0 (m, F), −113.0 to −113.0 (m, F), −113.1 to −113.2 (m, F). HRMS (ESI) m/z calcd for C₂₄H₂₉ClFN₄O₅S [M+H]⁺: 539.1453; found 539.1518.

### Ethyl 6-((tert-butoxycarbonyl)amino)-3-(2-chloro-4-fluorophenyl)-1-(thiazol-2-yl)-3,5,6,7-tetrahydropyrrolo[1,2-c]pyrimidine-4-carboxylate (41)

Methanesulfonyl chloride (0.91 mL, 11.8 mmol) was added to a solution of 39 (3.17 g, 5.9 mmol) and trimethylamine (1.6 mL, 5.9 mmol) in dichloromethane (150 mL) under nitrogen atmosphere. The mixture was stirred at 40° C. for 24 h to reach completion. The mixture was then poured into an aqueous solution of NH₄Cl (200 mL) and extracted with DCM (2×100 mL). The combined organic layers were dried over Na₂SO₄, filtered and concentrated in vacuo. Purification by column chromatography (7:3 Hex:AcOEt) afforded 41 as a yellow solid (2.00 g, 65%). ³H NMR (400 MHz, CDCl₃) δ 7.81 (d, J=3.2 Hz, 0.6H), 7.80 (d, J=3.2 Hz, 0.4H), 7.37 (d, J=3.2 Hz, 0.6H), 7.36 (d, J=3.2 Hz, 0.4H), 7.32 (dd, J=8.6, 6.1 Hz, 0.6H), 7.28-7.23 (m, 0.4H), 7.16-7.10 (m, 1H), 6.97-6.89 (m, 1H), 6.18 (s, 0.6H), 6.17 (s, 0.4H), 4.87-4.78 (m, 1H), 4.50-4.30 (m, 3H), 4.09-4.01 (m, 2H), 3.56 (dd, J=18.0, 7.2 Hz, 0.4H), 3.41-3.34 (m, 0.6H), 3.28 (dd, J=18.4, 6.3 Hz, 0.6H), 3.08 (dd, J=18.0, 6.1 Hz, 0.4H), 1.47 (s, 5.4H), 1.46 (s, 3.6H), 1.15 (t, J=7.1 Hz, 1.8H), 1.13 (t, J=7.1 Hz, 1.2H). ¹³C NMR (101 MHz, CDCl₃) δ 166.0, 166.0, 164.3, 164.2, 162.7, 162.7, 160.3, 160.2, 155.2, 155.2, 151.9, 151.5, 144.7, 144.2, 143.2, 143.1, 138.2 (d, J=3.4 Hz), 138.0 (d, J=3.5 Hz), 133.9 (dd, J=12.4, 10.5 Hz), 130.8 (t, J=93 Hz), 122.80, 117.2 (d, J=10.4 Hz), 117.0 (d, J=10.4 Hz), 114.3, 114.1, 96.5, 96.3, 80.2, 60.0, 59.9, 57.9, 56.6, 56.4, 56.2, 48.0, 37.6, 37.1, 28.4, 28.4, 14.2, 14.2. ¹⁹F NMR (377 MHz, CDCl₃) δ−113.3 to −113.5 (m, F). HRMS (ESI) m/z calcd for C₂₄H₂₇ClFN₄O₄S [M+H]⁺: 521.1347; found 521.1413.

### 6-((tert-Butoxycarbonyl)amino)-3-(2-chloro-4-fluorophenyl)-1-(thiazol-2-yl)-3,5,6,7-tetrahydropyrrolo[1,2-c]pyrimidine-4-carboxylic acid (42)

A solution of sodium hydroxide (456 mg, 11.4 mmol) in water (50 mL) was added to a solution of 41 (2.00 g, 3.8 mmol) in ethanol (150 mL). The mixture was stirred at 60° C. for 48 h then poured into an aqueous solution of ammonium chloride (200 mL) and extracted with ethyl acetate (3×50 mL). The combined layers were dried over Na₂SO₄, filtered and concentrated in vacuo. Purification by column chromatography (6:4 to 0:10 Hex:AcOEt) afforded 42 as an orange solid (1.48 g, 79%). ¹H NMR (400 MHz, CDCl₃) δ 7.82-7.77 (m, 1H), 7.38-7.34 (m, 1H), 7.33-7.27 (m, 0.45H), 7.24-7.17 (m, 0.55), 7.15-7.09 (m, 1H), 6.96-6.87 (m, 1H), 6.14 (s, 0.55H), 6.11 (s, 0.45H), 4.98-4.80 (m, 1H), 4.48-4.17 (m, 3H), 3.56-2.99 (m, 2H), 1.46 (s, 5H), 1.45 (s, 4H). ¹³C NMR (101 MHz, CDCl₃) δ 164.1, 144.6, 144.1, 143.2, 143.2, 137.1, 134.1, 130.5, 123.0, 117.5, 117.2, 114.2 (d, J=7.7 Hz), 114.0 (d, J=7.5 Hz), 94.7, 58.1, 56.4, 56.1, 56.1, 47.8, 37.4, 28.4, 28.4, 14.2. ¹⁹F NMR (377 MHz, CDCl₃) δ−113.3. HRMS (ESI) m/z calcd for C₂₃H₂₃ClFN₄O₄S [M+H]⁺: 493.1034; found 493.1099.

### tert-Butyl (3-(2-chloro-4-fluorophenyl)-4-iodo-1-(thiazol-2-yl)-3,5,6,7-tetrahydropyrrolo-[1,2-c]pyrimidin-6-yl)carbamate (43)

Sodium iodide (2.25 g, 15.0 mmol) was added to a solution of 42 (1.48 g, 3.0 mmol) and Na₂CO₃ (350 mg, 3.3 mmol) in methanol (70 mL) and water (70 mL). The flask was protected from light with aluminium foil, then Oxone® (1.94 g, 3.15 mmol) was added to the mixture and the reaction was stirred for 18 h. The mixture was poured into a saturated aqueous solution of Na₂S₂O₃ (200 mL) and extracted with ethyl acetate (3×100 mL). The combined organic layers were dried over Na₂SO₄, filtered and concentrated in vacuo. Purification by column chromatography (6:4 Hex:AcOEt) afforded 43 as a yellow solid (926 mg, 54%). ¹H NMR (400 MHz, CDCl₃) δ 7.80 (d, J=3.2 Hz, 1H), 7.41-7.34 (m, 2H), 7.17-7.11 (m, 1H), 7.04-6.96 (m, 1H), 6.01 (s, 1H), 4.86-4.76 (m, 1H), 4.58-4.49 (m, 0.5H), 4.49-4.44 (m, 1H), 4.44-4.30 (m, 1.5H), 3.04-2.93 (m, 1H), 2.66-2.56 (m, 1H), 1.50-1.43 (m, 9H). ¹³C NMR (101 MHz, CDCl₃) δ 164.4, 163.0, 163.2, 160.5, 160.5, 155.1, 155.1, 145.3, 144.9, 143.2, 143.2, 141.5, 141.4, 137.3, 137.2, 136.9, 136.9, 134.0 (d, J=8.6 Hz), 133.9 (d, J=8.6 Hz), 131.5 (d, J=1.9 Hz), 131.4 (d, J=1.7 Hz), 122.5, 117.2 (d, J=20.5 Hz), 117.0 (d, J=20.1 Hz), 114.6, 114.4, 80.2, 65.5, 65.1, 64.7, 58.7, 57.8, 47.2, 40.6, 40.4, 28.4, 28.4. ¹⁹F NMR (377 MHz, CDCl₃) δ−112.6 to −112.7 (m, F). HRMS (ESI) m/z calcd for C₂₁H₂₂ClFIN₄O₂S [M+H]⁺: 575.0102; found 575.0169.

### tert-Butyl(3-(2-chloro-4-fluorophenyl)-4-(cyclopropylethynyl)-1-(thiazol-2-yl)-3,5,6,7-tetrahydropyrrolo[1,2-c]pyrimidin-6-yl)carbamate (44)

To a solution of 43 (100 mg, 0.17 mmol) in DMF (5 mL) under nitrogen atmosphere, were added cyclopropylacetylene (22 μL, 0.26 mmol), trimethylamine (0.07 mL, 0.51 mmol), copper iodine (2 mg, 0.01 mmol) and PdCl₂(PPh₃)₂ (21 mg, 0.03 mmol). The mixture was stirred 4 h at room temperature before being quenched by addition of an aqueous solution of NH₄Cl (50 mL). The solution was extracted with ethyl acetate (3×20 mL), and the combined organic layers were dried over Na₂SO₄, filtered and concentrated in vacuo. Purification by column chromatography (8:2 Hex:AcOEt) afforded 44 as a yellow oil (69 mg, 79%). ³H NMR (400 MHz, CDCl₃) δ 7.84-7.79 (m, 1H), 7.39-7.32 (m, 2H), 7.14-7.08 (m, 1H), 7.02-6.96 (m, 1H), 5.82 (s, 1H), 4.86-4.70 (m, 1H), 4.45-4.27 (m, 3H), 3.05-2.93 (m, 1H), 2.70-2.59 (m, 1H), 1.46 (s, 9H), 1.31-1.18 (m, 1H), 0.78-0.66 (m, 2H), 0.63-0.45 (m, 2H). ¹³C NMR (101 MHz, CDCl₃) δ 164.9, 164.9, 162.8, 162.8, 160.4, 160.3, 155.2, 145.4, 143.3, 142.6, 138.1, 138.0, 133.8, 133.7, 131.4 (d, J=8.9 Hz), 131.2 (d, J=8.9 Hz), 122.6, 122.5, 116.8 (d, J=24.6 Hz), 116.4 (d, J=24.6 Hz), 114.4 (d, J=20.9 Hz), 114.4 (d, J=20.9 Hz), 96.8, 96.7, 90.7, 90.5, 80.2, 71.9, 71.8, 59.4, 58.7, 57.8, 57.7, 48.1, 35.8, 28.5, 14.3, 8.8, 8.8, 0.5, 0.5. ¹⁹F NMR (377 MHz, CDCl₃) δ−113.8 to −113.9 (m, F), −114.0 to −114.0 (m, F). HRMS (ESI) m/z calcd for C₂₆H₂₇ClFN₄O₂S [M+H]⁺: 513.1449; found 513.1516.

### N-(3-(2-Chloro-4-fluorophenyl)-4-(cyclopropylethynyl)-1-(thiazol-2-yl)-3,5,6,7-tetrahydro-pyrrolo[1,2-c]pyrimidin-6-yl)cyclopropanesulfonamide (45)

A solution of HCl in dioxane (4M, 1.5 mL) was added to a solution of 44 (69 mg, 0.13 mmol) in DCM (10 mL) at 0° C. The mixture was then stirred at room temperature for 18 h before being concentrated in vacuo. To a solution of the crude compound (LC-MS (ESI) m/z: 413 [M+H]⁺) in pyridine (2 mL) under nitrogen atmosphere, was added cyclopropanesulfonyl chloride (20 μL, 0.2 mmol). The mixture was stirred at room temperature for 2 h. Cyclopropanesulfonyl chloride (20 μL, 0.2 mmol) was added again every 2 h over a 4 h time period in order to reach completion of the reaction. The mixture was then poured into an aqueous saturated solution of Na₂CC)₃ (30 mL) and extracted with DCM (3×10 mL). The combined organic layers were washed with water (50 mL) and brine (50 mL) then dried over Na₂SO₄, filtered and concentrated in vacuo. Purification by column chromatography (8:2 Hex:AcOEt) afforded 45 (25 mg, 37%). ¹H NMR (400 MHz, CDCl₃) δ 7.82 (d, J=3.2 Hz, 1H), 7.40-7.31 (m, 2H), 7.11 (dd, J=8.6, 2.6 Hz, 1H), 7.00 (dt, J=8.3, 2.6 Hz, 1H), 5.81 (s, 1H), 4.81 (d, J=7.7 Hz, 1H), 4.49 (dd, J=11.7, 5.7 Hz, 1H), 4.44 (dd, J=12.1, 4.0 Hz, 1H), 4.25-4.16 (m, 1H), 3.07 (dd, J=16.3, 7.3 Hz, 1H), 2.75 (dd, J=16.9, 4.5 Hz, 1H), 2.50-2.41 (m, 1H), 1.23-1.16 (m, 3H), 1.06-1.00 (m, 2H), 0.77-0.70 (m, 2H), 0.62-0.49 (m, 2H). ¹³C NMR (101 MHz, CDCl₃) δ 164.7, 162.8, 160.4, 145.1, 143.3, 141.7, 137.8 (d, J=3.4 Hz), 133.8 (d, J=10.4 Hz), 131.4 (d, J=8.8 Hz), 122.7, 116.8, 116.6, 114.5, 114.3, 97.1, 91.0, 71.6, 59.2, 57.8, 50.9, 36.4, 31.4, 8.8, 6.0, 5.9, 0.5. ¹⁹F NMR (377 MHz, CDCl₃) δ−113.62 (q, J=1.9 Hz). HRMS (ESI) m/z calcd for C₂₄H₂₃ClFN₄O₂S₂ [M+H]⁺: 517.0857; found 517.0922.

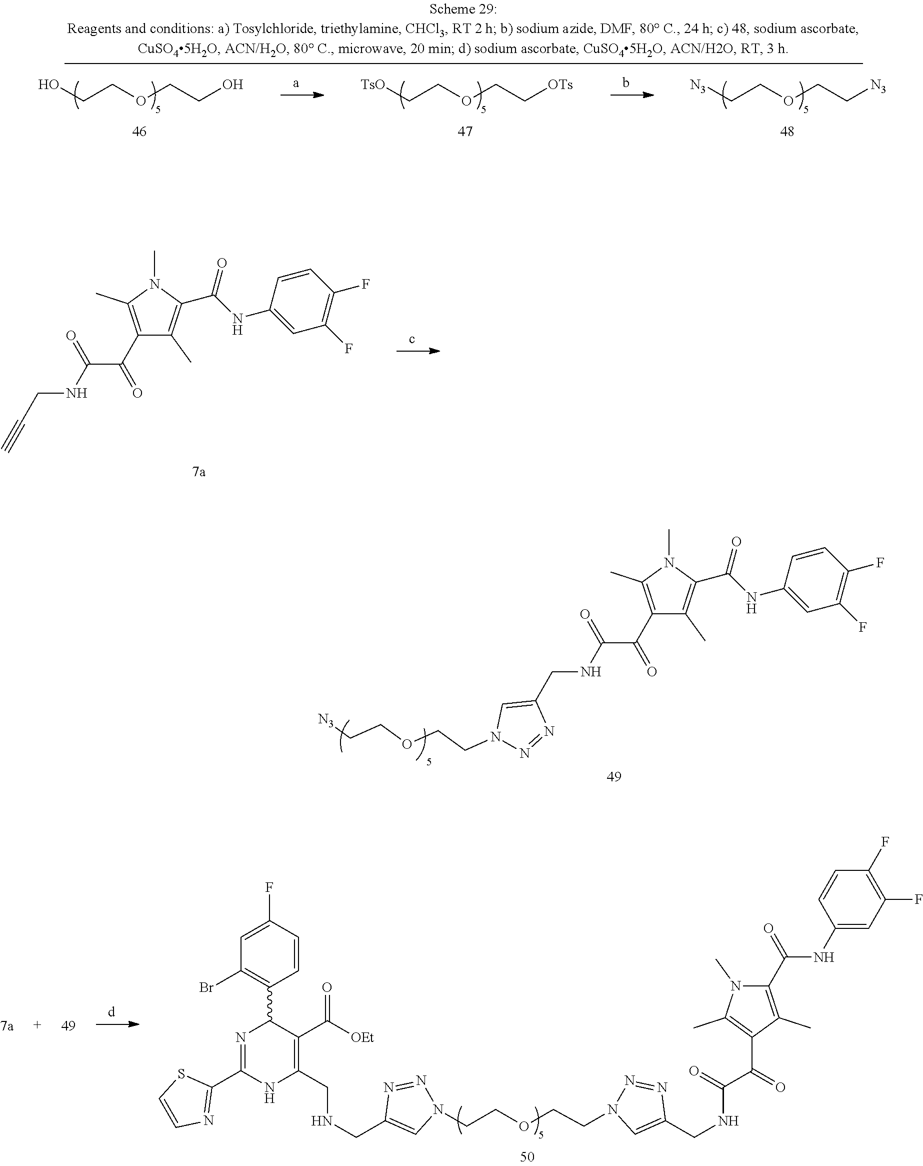

### 3,6,9,12,15-Pentaoxahentadecane-1,17-diyl bis(4-methylbenzenesulfonate) (47)

Triethylamine (0.92 mL, 6.4 mmol) and tosylchloride (0.91 g, 4.8 mmol) were added to a solution of 3,6,9,12,15-pentaoxaheptadecane-1,17-diol 46 (0.4 mL, 1.6 mmol) in chloroform (10 mL) at 0° C. The mixture was then stirred at room temperature for 2 h, quenched with a saturated solution of Na₂CO₃ (100 mL) then extracted with DCM (2×50 mL). The combined organic layers were dried over Na₂SO₄, filtered and concentrated in vacuo. Purification by column chromatography (7:3 hexane:AcOEt) afforded 47 as an oil (870 mg, 92%). ¹H NMR (400 MHz, CDCl₃) δ7.79 (d, J=8.3 Hz, 4H), 7.34 (d, J=8.1 Hz, 4H), 4.17-4.13 (m, 4H), 3.70-3.66 (m, 4H), 3.62 (s, 8H), 3.58 (s, 8H), 2.45 (s, 6H). ¹³C NMR (101 MHz, CDCl₃) δ 144.8, 133.0, 129.8, 128.0, 70.7, 70.6, 70.5, 70.5, 69.3, 68.7, 21.6.

### 1,17-Diazido-3,6,9,12,15-pentaoxaheptadecane (48)

Sodium azide (385 mg, 5.9 mmol) was added to a solution of 47 (870 mg, 1.47 mmol) in DMF (8 mL) under nitrogen atmosphere. The mixture was stirred at 80° C. for 24 h. Water (100 mL) was added then extracted with hexane (3×30 mL). The combined organic layers were dried over Na₂SO₄, filtered and concentrated in vacuo to obtain 48 (430 mg, 88%). ³H NMR (400 MHz, CDCl₃) δ) δ 3.70-3.64 (m, 20H), 3.41-3.37 (m, 4H). ¹³C NMR (101 MHz, CDCl₃) δ 70.4, 70.4, 70.3, 69.8, 50.5. LC-MS (ESI) m/z: 333.5 [M+H]⁺.

### 4-(2-(((1-(17-Azido-3,6,9,12,15-pentaoxaheptadecyl)-1H-1,2,3-triazol-4-yl)methyl)amino)-2-oxoacetyl)-N-(3,4-difluorophenyl)-1,3,5-trimethyl-1H-pyrrole-2-carboxamide (49)

A mixture of 7a (50 mg, 0.13 mmol), 48 (95 mg, 0.26 mmol), CuSO₄.5H₂O (4 mg), sodium ascorbate (8 mg) in acetonitrile (1 mL) and H₂O (0.5 mL) was stirred at 80° C. for 20 min under microwave irradiations. The mixture was diluted with ethyl acetate and washed with a saturated solution of NaCl. The organic layer was dried over Na₂SO₄, filtered and concentrated in vacuo. Purification by column chromatography (98:2 DCM:MeOH) afforded 49 (36 mg, 40%). ³H NMR (400 MHz, CDCl₃) δ 8.06 (s, 1H), 7.80 (s, 1H), 7.72 (ddd, J=12.2, 7.2, 2.4 Hz, 1H), 7.57 (s, 1H), 7.26-7.20 (m, 1H), 7.13 (q, J=8.8 Hz, 1H), 4.64-4.56 (m, 2H), 4.53 (t, J=4.9 Hz, 2H), 3.85 (t, J=4.0 Hz, 2H), 3.70-3.56 (m, 20H), 3.37 (t, J=6.0 Hz, 2H), 2.36 (s, 3H), 2.29 (s, 3H). ¹³C NMR (101 MHz, CDCl₃) δ 186.8, 164.8, 160.2, 151.3 (d, J=13.2 Hz), 148.9 (d, J=13.0 Hz), 148.2 (d, J=12.9 Hz), 145.8 (d, J=12.8 Hz), 141.9, 134.4 (dd, J=8.6, 3.1 Hz), 126.1, 123.7, 123.4, 117.7, 117.3 (d, J=18.0 Hz), 115.6 (dd, J=5.8, 3.5 Hz), 109.8 (d, J=21.8 Hz), 70.6, 70.6, 70.5, 70.5, 70.4, 70.0, 69.3, 50.7, 50.4, 34.9, 32.3, 12.3, 12.0. ¹⁹F NMR (377 MHz, CDCl₃) δ−135.6 to −135.7 (m, F), −142.4 to −142.5 (m, F). HRMS (ESI) m/z calcd for C₃₁H₄₂F₂N₉O₈ [M+H]⁺: 706.3046; found 706.3102.

### Ethyl 4-(2-bromo-4-fluorophenyl)-6-((((1-(17-(4-((2-(5-((3,4-difluorophenyl)carbamoyl)-1,2,4-trimethyl-1H-pyrrol-3-yl)-2-oxoacetamido)methyl)-1H-1,2,3-triazol-1-yl)-3,6,9,12,15-pentaoxaheptadecyl)-1H-1,2,3-triazol-4-yl)methyl)amino)methyl)-2-(thiazol-2-yl)-1,4-dihydropyrimidine-5-carboxylate (50)

A mixture of 7a (22 mg, 0.05 mmol), 49 (36 mg, 0.05 mmol), CuSO₄.5H₂O (50 mg, 0.2 mmol), sodium ascorbate (60 mg, 0.3 mmol) in acetonitrile (4 mL) and H₂O (3 mL) was stirred at room temperature for 3 h. The mixture was diluted with DCM and washed twice with a solution of NH₄OH/NH₄Cl pH 9. The organic phase was dried over Na₂SO₄, filtered and concentrated in vacuo. Purification by column chromatography (96:4 DCM:MeOH) afforded 50 (10 mg, 17%). ¹H NMR (400 MHz, CDCl₃) δ 9.81 (s, 0.5H), 8.22-8.15 (m, 1H), 7.86-7.83 (m, 1.4H), 7.82 (s, 0.4H), 7.79 (s, 0.6H), 7.77-7.71 (m, 1H), 7.69 (s, 0.4H), 7.64-7.59 (m, 0.6H), 7.58-7.50 (m, 1H), 7.43 (d, J=3.1 Hz, 0.6H), 7.33-7.20 (m, 2.6H), 7.17-7.07 (m, 1H), 7.02 (dt, J=8.2, 2.5 Hz, 0.4H), 6.96 (dt, J=8.3, 2.6 Hz), 6.14-6.08 (m, 1H), 4.79-4.72 (m, 0.4H), 4.66-4.60 (m, 1.6H), 4.55-4.46 (m, 4H), 4.27-4.19 (m, 1H), 4.16-4.07 (m, 1.4H), 4.03-3.96 (m, 0.6H), 3.91 (s, 1H), 3.88-3.79 (m, 4H), 3.75-3.69 (m, 0.6H), 3.67 (s, 3H), 3.64-3.51 (m, 16H), 2.37 (s, 1.8H), 2.36 (s, 1.2H), 2.31 (s, 1,2H), 2.26 (s, 1,8H), 1.29-1.22 (m, 1.2H), 1.10 (t, J=7.1 Hz, 1.8H). ¹³C NMR (101 MHz, CDCl₃) δ 186.8, 169.1, 166.1, 164.8, 164.7, 163.1, 162.2, 160.2, 160.2, 160.1, 160.1, 151.1, 147.8, 145.9, 144.3, 143.8, 143.7, 143.6, 143.2, 141.8, 141.8, 140.1, 140.0, 134.5, 131.4 (d, J=9.0 Hz), 130.7 (d, J=8.6 Hz), 126.2, 126.1, 124.3, 123.6, 123.6, 123.3, 123.3, 123.1, 123.1, 120.5 (d, J=24.7 Hz), 120.0 (d, J=24.2 Hz), 117.7, 117.7, 117.3, 117.3, 117.1, 117.1, 115.6, 115.5, 115.1, 114.9, 109.9, 109.8, 109.7, 109.3, 97.1, 70.5, 70.4, 69.5, 69.4, 60.4, 59.8, 58.6, 52.1, 51.6, 50.3, 50.2, 47.4, 44.8, 37.1, 35.0, 32.3, 29.7, 14.2, 14.1, 12.3, 12.2, 12.1, 12.1. ¹⁹F NMR (377 MHz, CDCl₃) δ−110.7 (q, J=7.5 Hz, 0.4F), −113.4 (q, J=8.1 Hz, 0.6F), −135.6 to −135.7 (m, F), −142.4 to −142.7 (m, F). HRMS (ESI) m/z calcd for C₅₁H₆₀BrF₃N₁₃O₁₀S [M+H]⁺: 1182.3364; found 1182.3429.

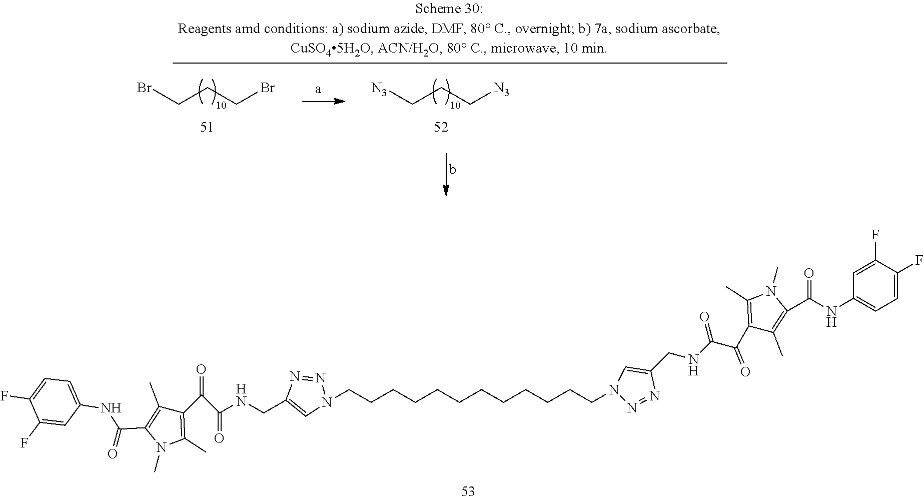

### 1,10-Diazidododecane (52)

Sodium azide (260 mg) was added to a solution of 1,10-dibromododecane 51 (340 mg) in DMF (2 mL) under nitrogen atmosphere. The mixture was stirred at 80° C. overnight. Water was added and extracted three times with hexane. The combined organic phases were dried over Na₂SO₄, filtered and concentrated in vacuo to obtain 1,10-diazidododecane 52.

### 4,4′-(2,2′-(((Dodecane-1,12-diylbis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (53)

7a (92 mg, 0.25 mmol), 1,12-diazidododecane 52 (25 mg, 0.1 mmol), CuSO₄.5H₂O (4 mg), sodium ascorbate (8 mg) in acetonitrile (0.5 mL) and H₂O (0.3 mL) was stirred at 80° C. for 10 min under microwave irradiation. The mixture was diluted with AcOEt and washed with a saturated solution of NaCl. The organic phase was dried over Na₂SO₄, filtered and concentrated in vacuo. Purification by column chromatography (98:2 DCM:MeOH) afforded 53 (55 mg). ¹H NMR (400 MHz, CDCl₃) δ 8.69 (s, 2H), 7.88 (s, 2H), 7.78-7.73 (m, 2H), 7.61 (s, 2H), 7.34-7.31 (m, 2H), 7.14-7.07 (m, 2H), 4.48 (s, 4H), 4.30 (t, J=7.2 Hz, 4H), 3.62 (s, 6H), 2.29 (s, 6H), 2.21 (s, 6H), 1.85 (t, J=6.4 Hz, 4H), 1.27-1.21 (m, 16H); ¹⁹F NMR (377 MHz, CDCl₃) δ−135.8-−135.9 (m), δ−142.5-−142.6 (m); MS (ESI): m/z [M+H]⁺ calcd for C₅₀H₅₉F₄N₁₂O₆: 999.5, found: 999.6.

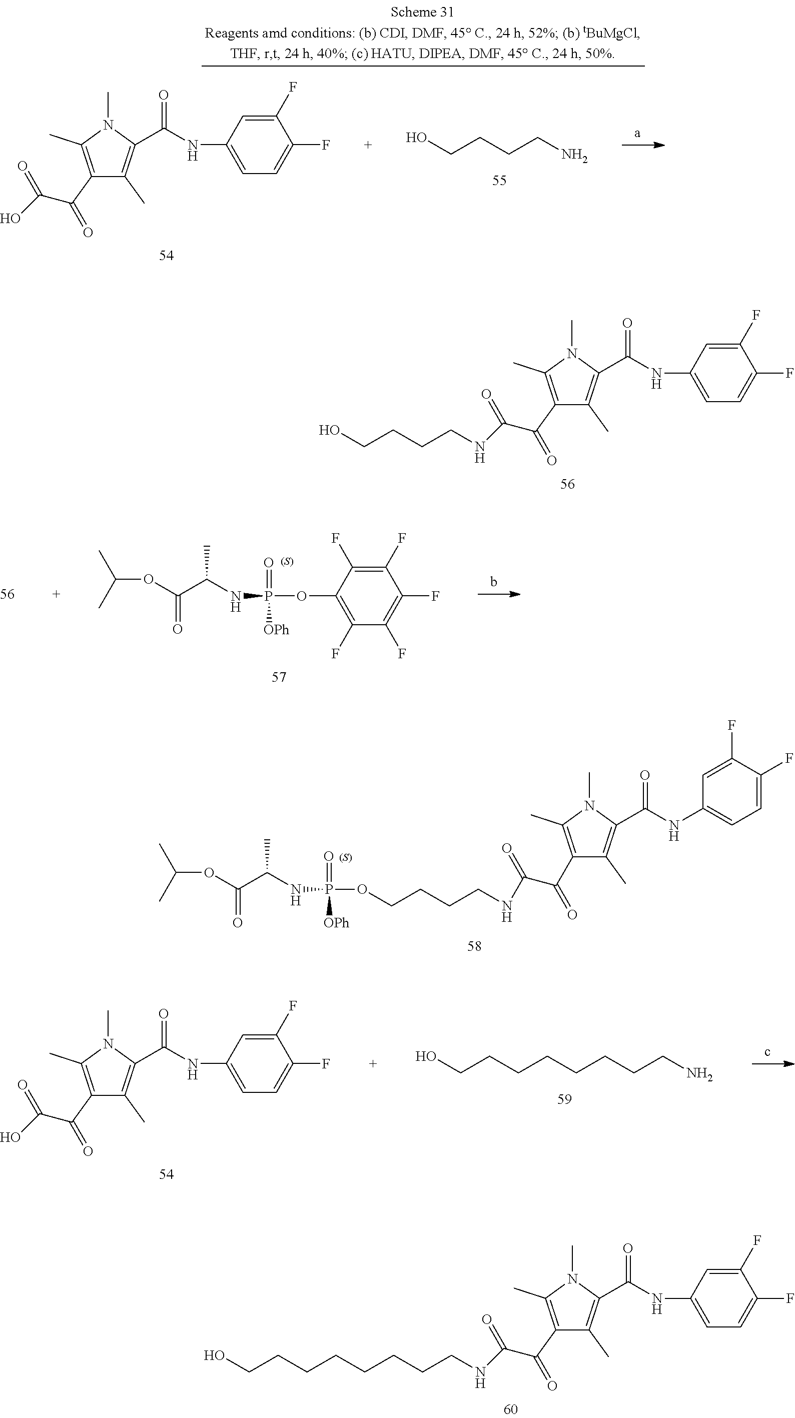

### N-(3,4-Difluorophenyl)-4-(2-((4-hydroxybutyl)amino)-2-oxoacetyl)-1,3,5-trimethyl-1H-pyrrole-2-carboxamide (56)

To a solution of 2-(5-((3,4-difluorophenyl) carbamoyl)-1,2,4-trimethyl-1H-pyrrol-3-yl)-2-oxoacetic acid 54 (75 mg, 0.22 mmol) in DMF (4 mL) was added CDI (90 mg, 0.56 mmol) and 4-amino-butan-1-ol 55 (50 mg, 0.56 mmol). After stirring at r.t. for 24 h, the reaction mixture was poured into a saturated NH₄Cl (20 mL) and extracted with CHCl₃ (3×50 mL). The organic layers were combined and dried over Na₂SO₄, concentrated in vacuo and the residue was purified by Combi-flash chromatography to yield N-(3,4-difluorophenyl)-4-(2-((4-hydroxybutyl)amino)-2-oxoacetyl)-1,3,5-trimethyl-1H-pyrrole-2-carboxamide 56 (69 mg, 52% yield). ¹H NMR (400 MHz, DMSO-d₆) δ 10.34 (s, 1H), 8.60 (t, J=5.8 Hz, 1H), 7.82-7.77 (m, 1H), 7.38-7.33 (m, 2H), 4.37 (t, J=5.1 Hz, 1H), 3.51 (s, 3H), 3.34-3.33 (m, 2H), 3.11-3.09 (m, 2H), 2.32 (s, 3H), 2.15 (s, 3H), 1.47-1.36 (m, 4H); MS (ESI): m/z [M+H]⁺ calcd for C₂₀H₂₄F₂N₃O₄: 408.4, found: 408.4.

### Isopropyl ((S)-(4-(2-(5-((3,4-difluorophenyl)carbamoyl)-1,2,4-trimethyl-1H-pyrrol-3-yl)-2-oxoacetamido)butoxy)(phenoxy)phosphoryl)-L-alaninate (58)

To a stirred mixture of N-(3,4-difluorophenyl)-4-(2-(4-hydroxybutylamino)-2-oxoacetyl)-1,3,5-trimethyl-1H-pyrrole-2-carboxamide 56 (10 mg, 0.025 mmol) in dry THF (2 mL) was added a 1.0 M solution of tert-butylmagnesium chloride in THF (74 μL, 0.074 mmol) at 0° C. The resulting mixture was stirred at r.t. for 30 min. The reaction mixture was cooled to 0° C., and then was added a solution of (S)-2-[(S)-(2, 3, 4, 5, 6-pentafluorophenoxy)-phenoxyphosphorylamino]propionic acid isopropyl ester 57 (17 mg, 0.037 mmol) in THF (1.0 mL) over a period of 2 min. The mixture was stirred at r.t for 24 h, cooled to 0° C., and then quenched with a saturated solution of NH₄Cl (0.1 mL). The solvent was evaporated and the residue was purified by Combi-flash chromatography (DCM/MeOH=30:1 to 10:1) to afford 58 (6.6 mg, 40% yield) as a foam. ¹H NMR (400 MHz, MeOH-d₄) δ 7.83-7.77 (m, 1H), 7.38-7.34 (m, 3H), 7.27-7.17 (m, 4H), 5.02-4.95 (m, 2H), 4.18-4.11 (m, 2H), 3.92-3.88 (m, 1H), 3.67 (s, 3H), 3.38-3.34 (m, 1H), 2.46 (s, 3H), 2.34 (s, 3H), 1.78-1.69 (m, 4H), 1.36-1.31 (m, 3H), 1.26-1.23 (m, 6H). MS (ESI): m/z [M+H]⁺ calcd for C₃₂H₄₀F₂N₄O₈P: 677.7, found: 677.5.

### N-(3,4-Difluorophenyl)-4-(2-(8-hydroxyoctylamino)-2-oxoacetyl)-1,3,5-trimethyl-1H-pyrrole-2-carboxamide (60)

To a solution of 2-(5-((3,4-difluorophenyl) carbamoyl)-1,2,4-trimethyl-1H-pyrrol-3-yl)-2-oxoacetic acid 54 (100 mg, 0.30 mmol) in DMF (4 mL) was added HATU (283 mg, 0.74 mmol), 8-amino-1-octanol 59 (86 mg) and DIPEA (208 μL, 0.59 mmol). After stirring at 45° C. for 24 h, the reaction mixture was poured into a saturated NH₄Cl (30 mL) and extracted with EtOAc (3×50 mL). The organic layers was combined and dried over Na₂SO₄, concentrated in vacuo and the residue was purified by Combi-flash chromatography (DCM/MeOH=100:1 to 10:1) to yield 60 (69 mg, 50% yield). ¹H NMR (400 MHz, MeOH-di) δ 7.83-7.78 (m, 1H), 7.36-7.33 (m, 1H), 7.30-7.23 (m, 1H), 3.67 (s, 3H), 3.57-3.54 (m, 2H), 3.35-3.31 (m, 2H), 2.46 (s, 3H), 2.34 (s, 3H), 1.66-1.59 (m, 2H), 1.57-1.52 (m, 2H), 1.38 (brs, 8H); MS (ESI): m/z [M+H]⁺ calcd for C₂₄H₃₂F₂N₃O₄: 464.5, found: 464.5.

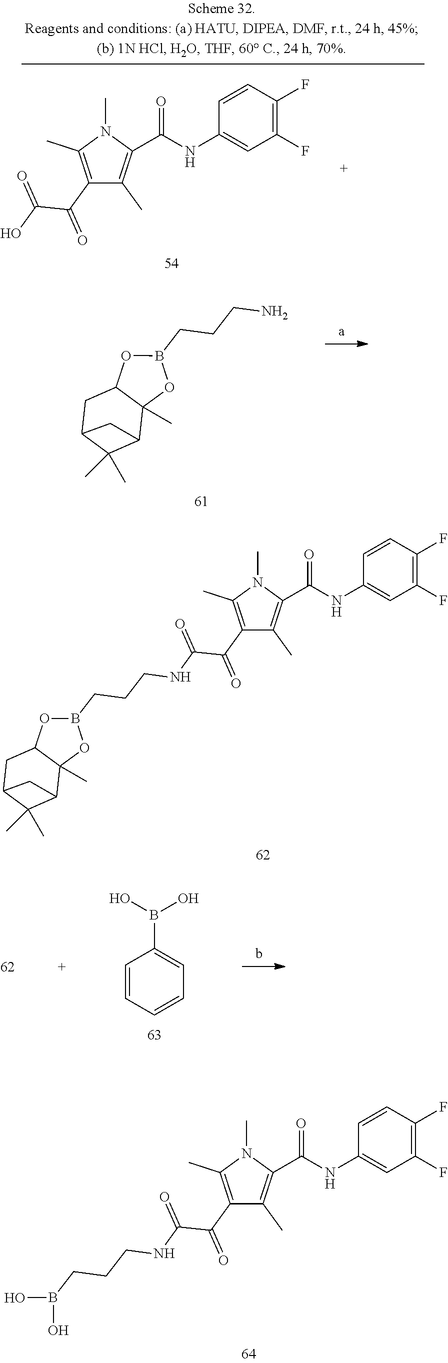 [SMILES: Cc1c(C(=O)C(=O)O)c(C)n(C)c1C(=O)Nc1ccc(F)c(F)c1 ; CC1(C)C2CC3OB(CCCN)OC3(C)C1C2 ; Cc1c(C(=O)C(=O)NCCCB2OC3CC4CC(C4(C)C)C3(C)O2)c(C)n(C)c1C(=O)Nc1ccc(F)c(F)c1 ; OB(O)c1ccccc1 ; Cc1c(C(=O)C(=O)NCCCB(O)O)c(C)n(C)c1C(=O)Nc1ccc(F)c(F)c1]

### N-(3,4-difluorophenyl)-1,3,5-trimethyl-4-(2-oxo-2-((3-(3a,5,5-trimethylhexahydro-4,6-methanobenzo[d][1,3,2]dioxaborol-2-yl)propyl)amino)acetyl)-1H-pyrrole-2-carboxamide (62)

To a solution of 2-(5-((3,4-difluorophenyl) carbamoyl)-1,2,4-trimethyl-1H-pyrrol-3-yl)-2-oxoacetic acid 54 (600 mg, 1.78 mmol) in DMF (40 mL) was added HATU (1.70 g, 4.46 mmol), DIPEA (1.25 mL, 7.14 mmol) and 3-(3a,5,5-trimethylhexahydro-4,6-methanobenzo[d][1,3,2]dioxaborol-2-yl)propan-1-amine 61 (508 mg, 2.14 mmol). After stirring at r.t for 24 h, the reaction mixture was poured into a saturated NH₄Cl (30 mL) and extracted with EtOAc (3×50 mL). The organic layers was combined and dried over Na₂SO₄, concentrated in vacuo and the residue was purified by Combi-flash chromatography (Hexane/EtOAc=10:1 to 1:3) to yield N-(3,4-difluorophenyl)-1,3,5-trimethyl-4-(2-oxo-2-((3-(3a,5,5-trimethylhexahydro-4,6-methanobenzo[d][1,3,2]dioxaborol-2-yl)propyl)amino)acetyl)-1H-pyrrole-2-carboxamide 62 (446 mg, 45% yield). ¹H NMR (400 MHz, CH₃COCH₃-d₆) δ 9.52 (s, 1H), 8.00-7.95 (m, 1H), 7.81-7.85 (m, 1H), 7.53-7.49 (m, 1H), 7.34-7.27 (m, 1H), 4.29 (dd, J=8.7 Hz, 1.92 Hz, 1H), 3.67 (s, 3H), 3.32 (dd, J=13.7 Hz, 6.5 Hz, 2H), 2.39 (s, 3H), 2.39-2.31 (m, 1H), 2.27 (s, 3H), 2.22-2.18 (m, 1H), 2.00-1.97 (m, 1H), 1.88-1.86 (m, 1H), 1.81-1.76 (m, 1H), 1.72-1.69 (m, 2H), 1.35 (s, 3H), 1.27 (s, 3H), 1.11 (d, J=10.8 Hz, 1H), 0.86-0.82 (m, 5H). HRMS (ESI): m/z [M+H]⁺ calcd. For C₂₉H₃₇BF₂N₃O₅: 556.27943, Found: 556.2786.

### (3-(2-(5-((3,4-Difluorophenyl)carbamoyl)-1,2,4-trimethyl-1H-pyrrol-3-yl)-2-oxoacetamido)propyl)boronic acid (64)

To a solution of N-(3,4-difluorophenyl)-1,3,5-trimethyl-4-(2-oxo-2-((3-(3a,5,5-trimethylhexahydro-4,6-methanobenzo[d][1,3,2]dioxaborol-2-yl)propyl)amino)acetyl)-1H-pyrrole-2-carboxamide 62 (30 mg, 0.054 mmol) and phenyl boronic acid (13 mg, 0.108 mmol) in Et₂O (2 mL) and H₂O (2 mL) was added 1N HCl (0.5 mL), the resulting mixture was stirred at 50° C. for 48 h, then concentrated in vacuo and the residue was purified by Combi-flash chromatography (DCM/MeOH=100:1 to 5:1) to yield (3-(2-(5-((3,4-difluorophenyl)carbamoyl)-1,2,4-trimethyl-1H-pyrrol-3-yl)-2-oxoacetamido)propyl)boronic acid 64 (16 mg, 70% yield). ¹H NMR (400 MHz, CH₃COCH₆) δ 9.48 (s, 1H), 8.01-7.96 (m, 1H), 7.81-7.78 (m, 1H), 7.53-7.49 (m, 1H), 7.36-7.29 (m, 1H), 6.77 (s, 2H), 3.68 (s, 3H), 3.34-3.29 (m, 2H), 2.42 (s, 3H), 2.28 (s, 3H), 1.73-1.69 (m, 2H), 0.79 (t, J=8.0 Hz, 2H); MS (ESI): m/z [M+H]⁺ calcd for C₁₉H₂₃BF₂N₃O₅: 422.2, found: 422.3.

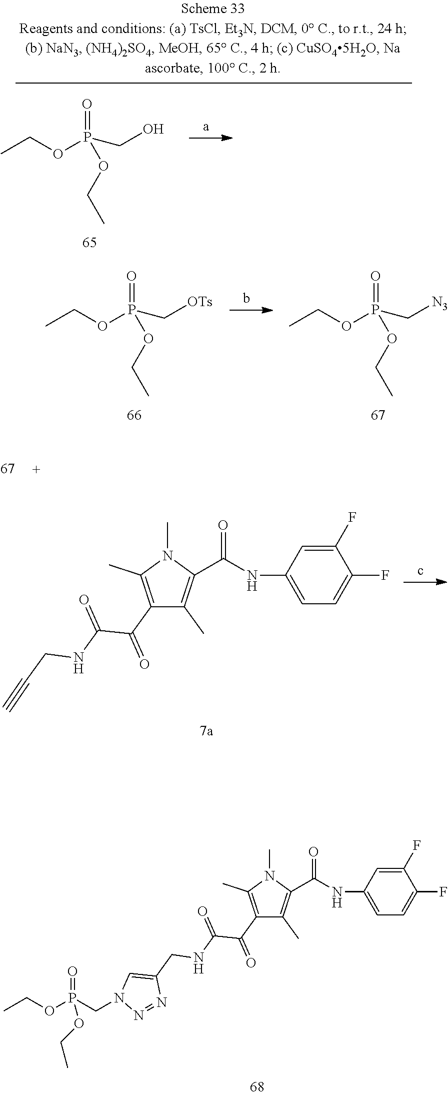

### (Diethoxyphosphoryl) methyl 4-methylbenzenesulfonate (66)

To a solution of compound 65 (3.0 g, 17.8 mmol, 1 eq), Et₃N (5.0 mL, 35.6 mmol, 2 eq) in DCM (90 mL) at 0° C. was added TsCl (3.4 g, 17.8 mmol, 1.0 eq). The resulting mixture was stirred at r.t. for 24 h and was quenched with cold saturated Na₂S₂O₃. The water layer was then extracted with DCM (3×80 mL). The organic layers were finally combined, washed with water, brine, and dried over anhydrous Na₂SO₄, and concentrated under reduced pressure. The residue was purified by flash chromatography (DCM/Ethyl acetate 3:1 to 1:2) to afford product 66 (4.0 g, 70%). ¹H NMR (400 MHz, CDCl₃) δ7.80 (d, J=8.0 Hz, 2H), 7.37 (d, J=8.0 Hz, 2H), 4.20-4.13 (m, 6H), 2.46 (s, 3H), 1.32 (t, J=7.0 Hz, 6H).

### Diethyl(azidomethyl) phosphonate (67)

To a solution of compound 66 (2.4 g, 7.45 mmol, 1.0 eq) and (NH₄)₂SO₄ (1.97 g, 14.89, 2.0 eq) in MeOH (50 mL) was added NaN₃ (0.97 g, 14.89 mmol, 2.0 eq). The resulting mixture was stirred at 65° C. for 24 h and was quenched with cold H₂O (100 mL). The water layer was then extracted with EtOAc (2×200 mL). The organic layers were finally combined, washed with water, brine, and dried over anhydrous Na₂SO₄, and concentrated under reduced pressure. The residue was purified by flash chromatography (DCM/Ethyl acetate 10:1 to 1:4) to afford product 67 (1.18 g, 82%). ¹H NMR (400 MHz, CDCl₃) δ 4.25-4.18 (m, 4H), 3.48 (d, J=11.6 Hz, 2H), 1.38 (t, J=6.8 Hz, 6H).

### Diethyl((4-((2-(5-((3,4-difluorophenyl)carbamoyl)-1,2,4-trimethyl-1H-pyrrol-3-yl)-2-oxoacetamido) methyl)-1H-1,2,3-triazol-1-yl) methyl) phosphonate (68)

To a mixture of compound 67 (103 mg, 0.54 mmol, 2.0 eq) and compound 7a (100 mg, 0.27 mmol, 1.0 eq) in H₂O/CH₃CN (3 mL/6.0 mL) was added CuSO₄.5H₂O (15 mg) and Na ascorbate (30 mg) under Ar atmosphere. The resulting mixture was stirred at 100° C. overnight. The mixture was diluted with 20 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 68 (138 mg, 91%). ¹H NMR (400 MHz, DMSO-d₆) δ10.42 (s, 1H), 9.25 (t, J=5.6 Hz, 1H), 7.94 (s, 1H), 7.90-7.85 (m, 1H), 7.44-7.38 (m, 2H), 5.10 (d, J=13.0 Hz, 2H), 4.48 (d, J=5.6 Hz, 2H), 4.10-4.03 (m, 4H), 3.59 (s, 3H), 2.36 (s, 3H) 2.20 (s, 3H), 1.21 (t, J=7.0 Hz, 6H). ¹⁹F NMR (377 MHz, DMSO-d₆) δ−137.2 to −137.3 (m, 1F), −144.2 to −144.3 (m, 1F). HRMS (ESI) m/z calcd for C₂₄H₃₀F₂N₆O₆P [M+H]⁺: 567.1933; found 567.1918.

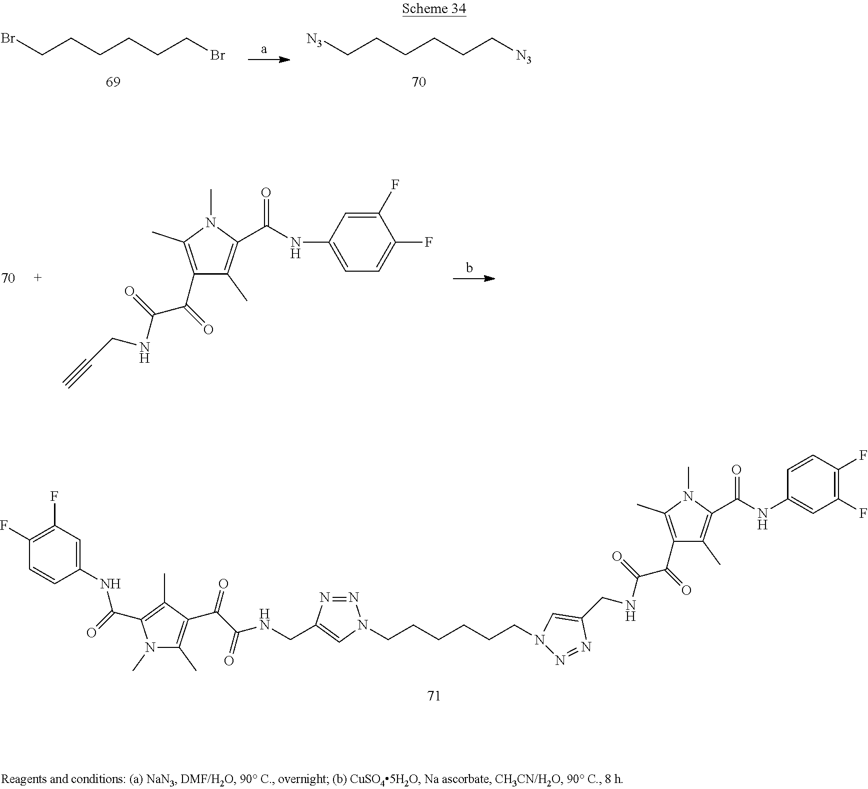 [SMILES: BrCCCCCCBr ; [N-]=[N+]=NCCCCCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### 1,6-Diazidohexane (70)

To a solution of compound 69 (2.5 g, 10.25 mmol, 1.0 eq) in DMF/H₂O (54 mL/6 mL) was added NaN₃ (1.3 g, 20.49 mmol, 2.0 eq). The resulting mixture was stirred at 90° C. overnight and was quenched with cold H₂O (50 mL). The water layer was then extracted with EtOAc (2×100 mL). The organic layers were finally combined, washed with water, brine, and dried over anhydrous Na₂SO₄, and concentrated under reduced pressure. The residue was purified by flash chromatography (Hexane/Ethyl acetate 10:1 to 1:4) to afford product 70 (1.4 g, 82%). ¹H NMR (400 MHz, DMSO-d₆) δ 3.33 (d, J=6.8 Hz, 4H), 1.58-1.51 (m, 4H), 1.39-1.33 (m, 4H).

### 4,4′-(2,2′-(((Hexane-1,6-diylbis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (71)

To a mixture of compound 70 (100 mg, 0.27 mmol, 1.0 eq) and compound 7a (22.5 mg, 0.13 mmol, 0.5 eq) in H₂O/CH₃CN (3 mL/5 mL) was added CuSO₄.5H₂O (24 mg) and Na ascorbate (48 mg) under Ar atmosphere. The resulting mixture was stirred at 90° C. for 8 h. The mixture was diluted with 40 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 71 (62 mg, 51%). ¹H NMR (400 MHz, DMSO-d₆) δ 10.42 (s, 2H), 9.18 (t, J=5.6 Hz, 2H), 7.99 (s, 2H), 7.90-7.84 (m, 2H), 7.46-7.38 (m, 4H), 4.44 (d, J=5.6 Hz, 4H), 4.31 (t, J=7.0 Hz, 4H), 3.58 (s, 6H), 2.18 (s, 6H), 2.34 (s, 6H). 1.77 (t, J=6.6 Hz, 4H), 1.23 (s, 4H). ¹⁹F NMR (377 MHz, DMSO-d₆) δ−137.1 to −137.2 (m, 1F), −144.2 to −144.4 (m, 1F). LCMS (ESI) m/z calcd for C₄₄H₄₇F₄N₁₂O₆ [M+H]⁺: 915.9; found 915.2.

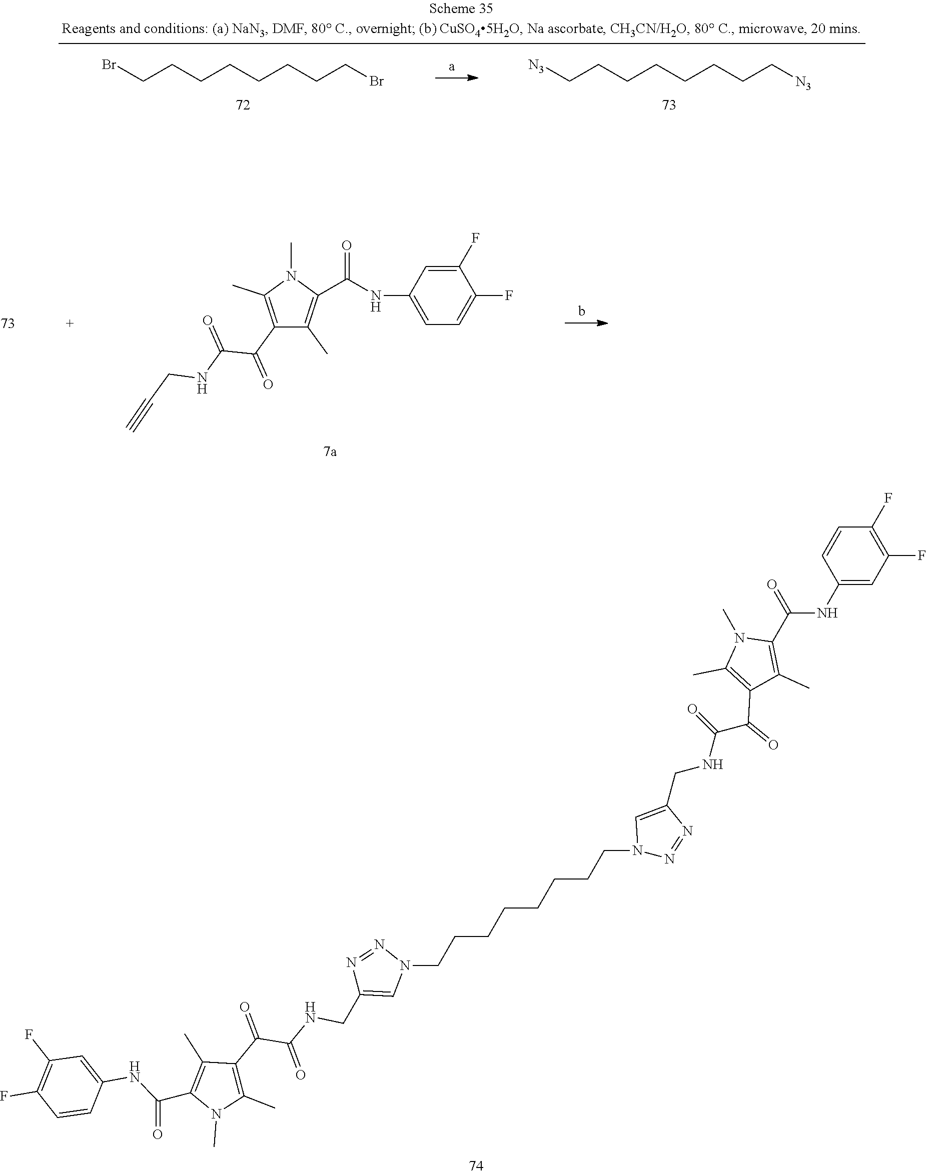 [SMILES: BrCCCCCCCCBr ; [N-]=[N+]=NCCCCCCCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCCCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### 1,8-Diazidooctane (73)

To a solution of compound 72 (544 mg, 2.0 mmol, 1.0 eq) in DMF (9 mL) was added NaN₃ (400 mg, 6.2 mmol, 3.1 eq). The resulting mixture was stirred at 80° C. overnight and was quenched with cold H₂O (50 mL). The water layer was then extracted with EtOAc (2×50 mL). The organic layers were finally combined, washed with water, brine, and dried over anhydrous Na₂SO₄, and concentrated under reduced pressure. The residue was purified by flash chromatography (Hexane/Ethyl acetate 10:1 to 1:4) to afford product 73 (275 mg, 70%). ¹H NMR (400 MHz, CDCl₃) δ 3.26 (t, J=6.8 Hz, 4H), 1.64-1.57 (m, 4H), 1.39-1.31 (m, 8H).

### 4,4′-(2,2′-(((Octane-1,8-diylbis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (74)

To a mixture of compound 73 (8 mg, 0.04 mmol, 0.4 eq) and compound 7a (38 mg, 0.1 mmol, 1.0 eq) in H₂O/CH₃CN (0.6 mL/1 mL) was added CuSO₄.5H₂O (6 mg) and Na ascorbate (12 mg) under Ar atmosphere. The resulting mixture was stirred at 80° C., microwave for 20 mins. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 74 (19 mg, 50%). ¹H NMR (400 MHz, CDCl₃) δ 8.87 (s, 2H), 8.09 (s, 2H), 7.75-7.70 (m, 2H), 7.61 (s, 2H), 7.33 (d, J=8.0 Hz, 2H), 7.11-7.04 (m, 2H), 4.45 (s, 4H), 4.27 (t, J=6.2 Hz, 4H), 3.57 (s, 6H), 2.25 (s, 6H), 2.15 (s, 6H), 8.09 (t, J=6.6 Hz, 4H), 1.23-1.19 (m, 8H). ¹⁹F NMR (377 MHz, CDCl₃) δ−135.9 to −136.0 (m, 1F), −142.5 to −142.6 (m, 1F). LCMS (ESI) m/z calcd for C₄₆H₅₁F₄N₁₂O₆[M+H]⁺: 944.0; found: 943.7.

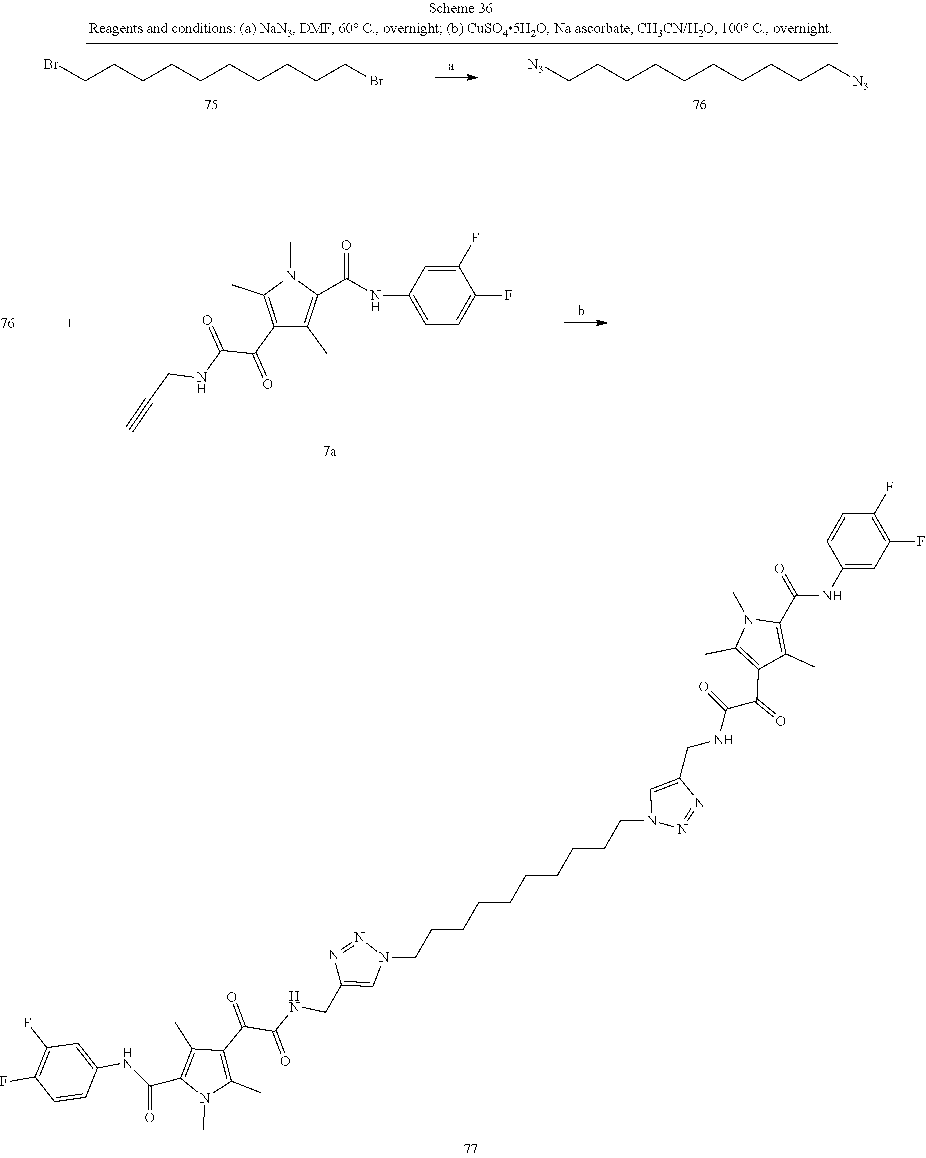 [SMILES: BrCCCCCCCCCCBr ; [N-]=[N+]=NCCCCCCCCCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCCCCCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### 1,10-Diazidodecane (76)

To a solution of compound 75 (2.5 g, 8.33 mmol, 1.0 eq) in DMF (100 mL) was added NaN₃ (1.6 g, 24.99 mmol, 3.0 eq). The resulting mixture was stirred at 60° C. overnight and was quenched with cold H₂O (50 mL). The water layer was then extracted with EtOAc (2×100 mL). The organic layers were finally combined, washed with water, brine, and dried over anhydrous Na₂SO₄, and concentrated under reduced pressure. The residue was purified by flash chromatography (Hexane/Ethyl acetate 20:1 to 1:4) to afford product 76 (1.68 g, 90%). ¹H NMR (400 MHz, CDCl₃) δ 3.31 (t, J=6.8 Hz, 4H), 1.57-1.50 (m, 4H), 1.34-1.28 (m, 12H).

### 4,4′-(2,2′-(((decane-1,10-diylbis(1H-1,2,3-triazole-1,4-diyl)) bis(methylene)) bis(azanediyl)) bis (2-oxoacetyl))bis(N-(3,4-difluorophenyl)-1,3,5-trimethyl-1H-pyrrole-2-carboxamide) (77)

To a mixture of compound 76 (30 mg, 0.134 mmol, 0.5 eq) and compound 7a (100 mg, 0.268 mmol, 1.0 eq) in H₂O/CH₃CN (5 mL/10 mL) was added CuSO₄.5H₂O (20 mg) and Na ascorbate (40 mg) under Ar atmosphere. The resulting mixture was stirred at 100° C. overnight. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 77 (52 mg, 40%). ¹H NMR (400 MHz, CDCl₃) δ 8.66 (s, 2H), 7.99 (s, 2H), 7.75-7.66 (m, 4H), 7.31-7.30 (m, 2H), 7.11-7.05 (m, 2H), 4.51 (s, 4H), 4.30 (s, 4H), 3.59 (s, 6H), 2.27 (s, 6H), 2.18 (s, 6H), 1.83 (s, 4H), 1.24-1.19 (m, 12H). ¹⁹F NMR (377 MHz, CDCl₃) δ−135.8 to −135.9 (m, 1F), −142.5 to −142.6 (m, 1F). LCMS (ESI) m/z calcd for C₄₈H₅₅F₁₂N₁₂O₆ [M+H]⁺: 972.0; found 971.3.

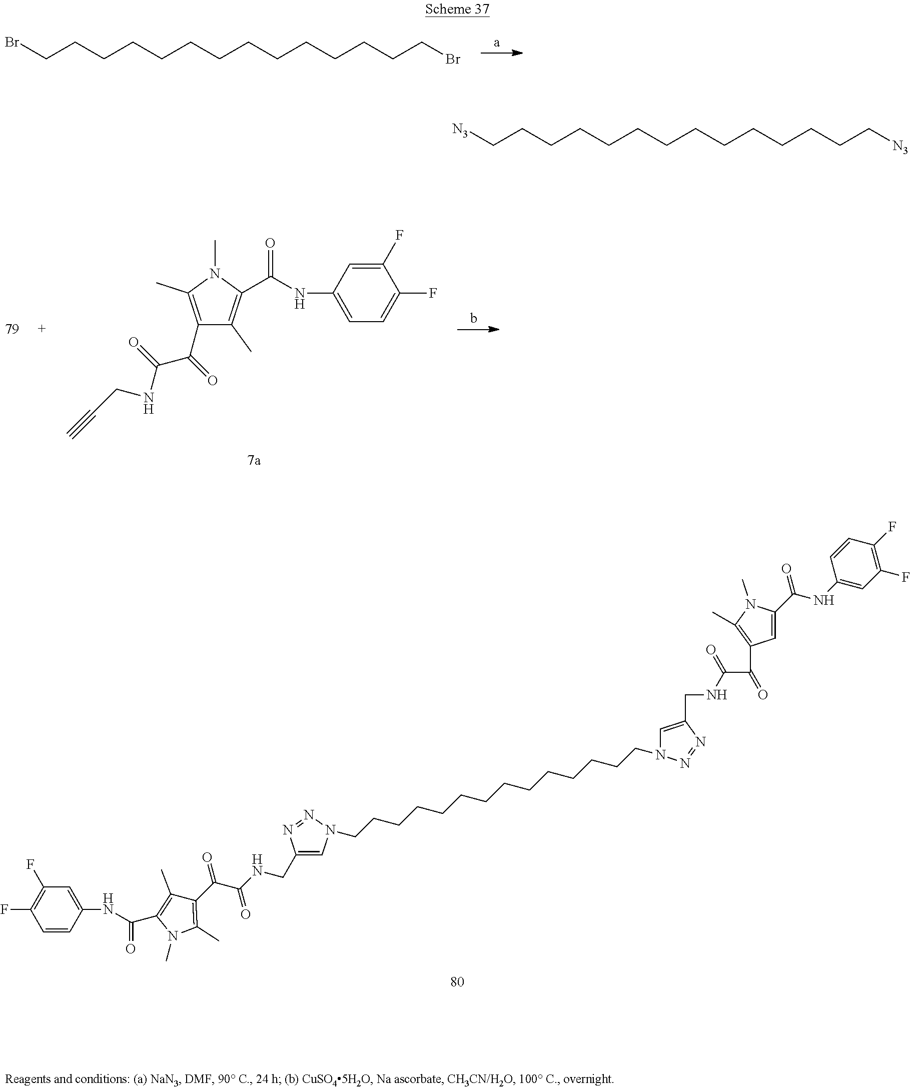 [SMILES: BrCCCCCCCCCCCCCCBr ; [N-]=[N+]=NCCCCCCCCCCCCCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCCCCCCCCCCCCCn3cc(CNC(=O)C(=O)c4cc(C(=O)Nc5ccc(F)c(F)c5)n(C)c4C)nn3)nn2)c(C)n(C)c1C(=O)Nc1ccc(F)c(F)c1]

### 1,14-Diazidotetradecane (79)

To a solution of compound 78 (500 mg, 1.40 mmol, 1.0 eq) in DMF (20 mL) was added NaN₃ (274 mg, 4.21 mmol, 3.0 eq). The resulting mixture was stirred at 90° C. for 24 h and was quenched with cold H₂O (40 mL). The water layer was then extracted with EtOAc (2×50 mL). The organic layers were finally combined, washed with water, brine, and dried over anhydrous Na₂SO₄, and concentrated under reduced pressure. The residue was purified by flash chromatography (Hexane/Ethyl acetate 20:1 to 1:4) to afford product 79 (339 mg, 86%). ¹H NMR (400 MHz, CDCl₃) δ 3.26 (t, J=7.2 Hz, 4H), 1.62-1.56 (m, 4H), 1.38-1.27 (m, 20H).

### 4,4′-(2,2′-(((Tetradecane-1,14-diylbis(1H-1,2,3-triazole-1,4-diyl))bis(methylene)) bis(azanediyl)) bis(2-oxoacetyl))bis(N-(3,4-difluorophenyl)-1,3,5-trimethyl-1H-pyrrole-2-carboxamide) (80)

To a mixture of compound 79 (30 mg, 0.107 mmol, 0.5 eq) and compound 7a (80 mg, 0.214 mmol, 1.0 eq) in H₂O/CH₃CN (8 mL/12 mL) was added CuSO₄.5H₂O (20 mg) and Na ascorbate (40 mg) under Ar atmosphere. The resulting mixture was stirred at 100° C. overnight. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 80 (55 mg, 50%). ¹H NMR (400 MHz, CDCl₃) δ 8.08 (s, 2H), 7.76-7.71 (m, 2H), 7.57 (s, 2H), 7.54 (t, J=5.9 Hz, 2H), 7.24-7.22 (m, 2H), 7.22-7.09 (m, 2H), 4.56 (d, J=5.9 Hz, 4H), 4.32 (t, J=7.2 Hz, 4H), 3.67 (s, 6H), 2.34 (s, 6H), 2.29 (s, 6H), 1.87 (t, J=7.2 Hz, 4H), 1.29-1.22 (m, 20H). ¹⁹F NMR (377 MHz, CDCl₃) δ−135.8 to −135.9 (m, 1F), −142.5 to −142.6 (m, 1F). LCMS (ESI) m/z calcd for C₅₂H₆₃F₄N₁₂O₆ [M+H]⁺: 1028.1; found 1027.3.

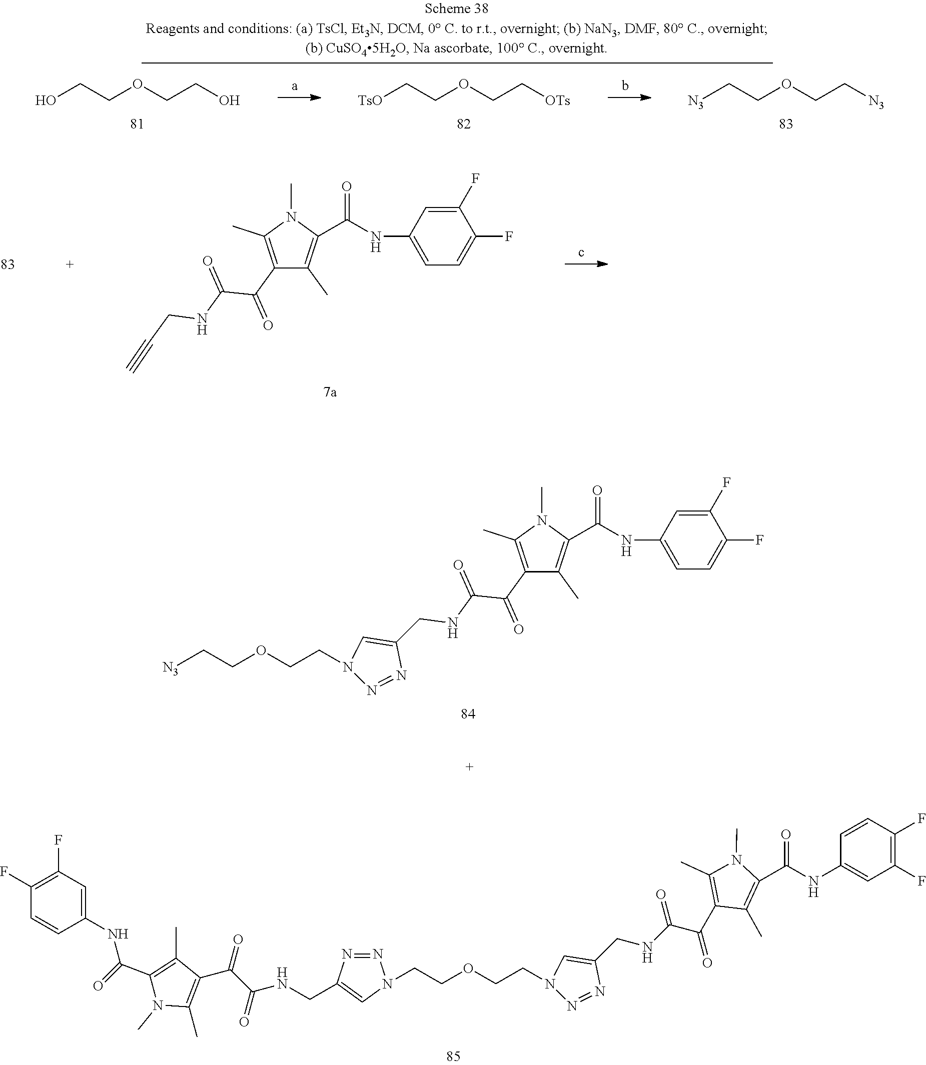 [SMILES: OCCOCCO ; Cc1ccc(S(=O)(=O)OCCOCCOS(=O)(=O)c2ccc(C)cc2)cc1 ; [N-]=[N+]=NCCOCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCOCCN=[N+]=[N-])nn2)c(C)n(C)c1C(=O)Nc1ccc(F)c(F)c1 ; Cc1c(C(=O)C(=O)NCc2cn(CCOCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### 1-Azido-2-(2-azidoethoxy) ethane (83)

To a solution of compound 81 (2.1 g, 19.79 mmol, 1 eq), Et₃N (8.3 mL, 59.37 mmol, 3 eq) in DCM (60 mL) at 0° C. was added TsCl (8.3 g, 43.54 mmol, 2.2 eq). The resulting mixture was stirred at r.t. for 24 h and was quenched with cold saturated solution of Na₂S₂O₃. The water layer was then extracted with DCM (3×100 mL). The organic layers were finally combined, washed with water, brine, and dried over anhydrous Na₂SO₄, and concentrated under reduced pressure to give crude compound 82 (10 g). To a solution of compound 82 (7.5 g. 18.09 mmol, 1 eq) in DMF (150 mL) was added NaN₃ (2.6 g, 39.8 mmol, 2.2 eq). The resulting mixture was stirred at 80° C. overnight and was quenched with cold FLO (100 mL). The water layer was then extracted with EtOAc (2×200 mL). The organic layers were finally combined, washed with water, brine, dried over anhydrous Na₂SO₄, and concentrated under reduced pressure. The residue was purified by flash chromatography (Hexane/Ethyl acetate 20:1 to 1:1) to afford product 83 (1.2 g, 52% over two steps). ¹H NMR (400 MHz, CD₃COCD₃) δ 3.73-3.71 (m, 4H), 3.43 (t, J=7.2 Hz, 4H).

### 4-(2-(((1-(2-(2-Azidoethoxy)ethyl)-1H-1,2,3-triazol-4-yl)methyl)amino)-2-oxoacetyl)-N-(3,4-difluorophenyl)-1,3,5-trimethyl-1H-pyrrole-2-carboxamide (84) and 4,4′-(2,2′-((((oxybis(ethane-2,1-diyl))bis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (85)

To a mixture of compound 83 (16.7 mg, 0.107 mmol, 0.5 eq) and compound 7a (80 mg, 0.214 mmol, 1.0 eq) in H₂O/CH₃CN (8 mL/12 mL) was added CuSO₄.5H₂O (20 mg) and Na ascorbate (40 mg) under Ar atmosphere. The resulting mixture was stirred at 100° C. overnight. The mixture was diluted with 30 mL of cold H₂O and extracted with ethyl acetate (3×50 mL). The organic layers were combined, washed with water, brine, dried over anhydrous Na₂SO₄, and concentrated under reduced pressure. The residue was purified by flash chromatography (DCM/MeOH, from 100:1 to 5:1) to afford product 84 (28 mg, 25%) ¹H NMR (400 MHz, DMSO-d₆) δ 10.42 (s, 1H), 9.20 (t, J=5.8 Hz 1H), 7.98 (s, 1H), 7.91-7.85 (m, 1H), 7.47-7.38 (m, 2H), 4.56 (t, J=5.2 Hz, 2H), 4.46 (d, J=5.7 Hz, 2H), 3.85 (t, J=5.2 Hz, 2H), 3.59 (t, J=4.4 Hz, 5H), 3.39-3.36 (m, 2H), 2.36 (s, 3H), 2.20 (s, 3H). ¹⁹F NMR (377 MHz, DMSO-d₆) δ−137.1 to −137.3 (m, 1F), −144.2 to −144.4 (m, 1F). LCMS (ESI) m/z calcd for C₂₃H₂₆F₂N₉O₄ [M+H]⁺: 530.5; found 530.1; and product 85 (58 mg, 60%). ¹H NMR (400 MHz, DMSO-d₆) δ 10.41 (s, 2H), 9.19 (t, J=5.7 Hz, 2H), 7.91 (s, 2H), 7.90-7.84 (m, 2H), 7.45-7.38 (m, 4H), 4.50 (t, J=5.0 Hz, 4H), 4.45 (d, J=5.7 Hz, 4H), 3.79 (t, J=5.2 Hz, 4H), 3.58 (s, 6H), 2.34 (s, 6H), 2.20 (s, 6H). ¹⁹F NMR (377 MHz, DMSO-d₆) δ−137.1 to −137.2 (m, 1F), −144.2 to −144.4 (m, 1F). LCMS (ESI) m/z calcd for C₄₂H₄₃F₄N₁₂O₇ [M+H]⁺: 903.9; found 903.1.

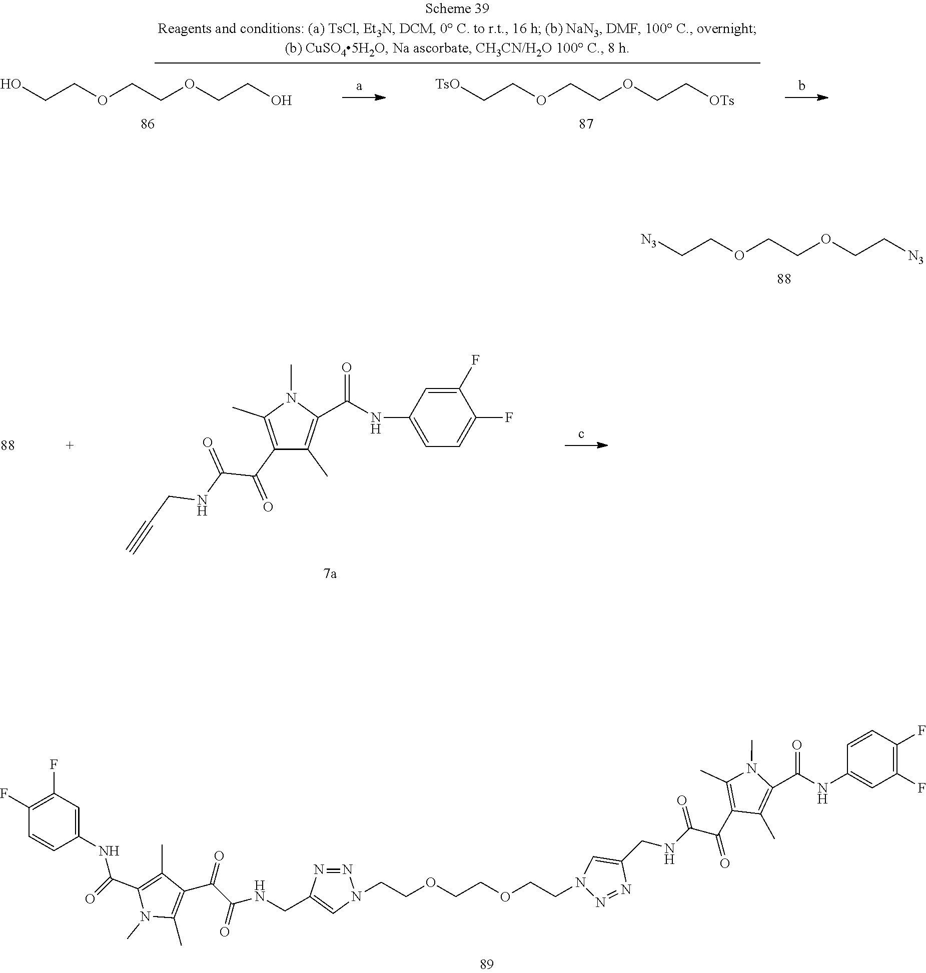 [SMILES: OCCOCCOCCO ; Cc1ccc(S(=O)(=O)OCCOCCOCCOS(=O)(=O)c2ccc(C)cc2)cc1 ; [N-]=[N+]=NCCOCCOCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCOCCOCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### (Ethane-1,2-diylbis (oxy)) bis (ethane-2,1-diyl) bis (4-methylbenzenesulfonate) (87)

To a solution of compound 86 (2.0 g, 13.3 mmol, 1 eq), Et₃N (4.6 mL, 33.3 mmol, 2.5 eq) in DCM (50 mL) at 0° C. was added TsCl (5.6 g, 29.3 mmol, 2.2 eq). The resulting mixture was stirred at r.t. for 16 h and was quenched with a cold saturated solution of Na₂S₂O₃. The water layer was then extracted with DCM (3×100 mL). The organic layers were finally combined, washed with water, brine, and dried over anhydrous Na₂SO₄, and concentrated under reduced pressure. The residue was purified by flash chromatography (DCM/Ethyl acetate 3:1 to 1:2) to afford product 87 (5.2 g, 85%).

### 1,2-bis (2-Azidoethoxy)ethane (88)

To a solution of compound 87 (5.8 g, 12.65 mmol, 1.0 eq) in DMF (120 mL) was added NaN₃ (1.64 g, 25.30 mmol, 2.0 eq). The resulting mixture was stirred at 100° C. overnight and was quenched with cold H₂O (40 mL). The water layer was then extracted with EtOAc (2×100 mL). The organic layers were finally combined, washed with water, brine, and dried over anhydrous Na₂SO₄, and concentrated under reduced pressure. The residue was purified by flash chromatography (Hexane/Ethyl acetate 20:1 to 1:4) to afford product 88 (2.0 g, 80%). ¹H NMR (400 MHz, CDCl₃) δ 3.71-3.68 (m, 8H), 3.39 (t, J=5.2 Hz, 4H).

### 4,4′-(2,2′-(((((Ethane-1,2-diylbis(oxy))bis(ethane-2,1-diyl))bis(1H-1,2,3-triazole-1,4-diyl))bis (methylene))bis(azanediyl))bis(2-oxoacetyl))bis(N-(3,4-difluorophenyl)-1,3,5-trimethyl-1H-pyrrole-2-carboxamide) (89)

To a mixture of compound 88 (13.4 mg, 0.067 mmol, 0.5 eq) and compound 7a (50 mg, 0.134 mmol, 1.0 eq) in H₂O/CH₃CN (4 mL/6 mL) was added CuSO₄.5H₂O (15 mg) and Na ascorbate (30 mg) under Ar atmosphere. The resulting mixture was stirred at 100° C. for 8 h. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 89 (39 mg, 61%). ¹H NMR (400 MHz, DMSO-d₆) δ10.42 (s, 2H), 9.21 (t, J=5.8 Hz, 2H), 7.96 (s, 2H), 7.90-7.85 (m, 2H), 7.46-7.38 (m, 4H), 4.50 (d, J=5.1 Hz, 4H), 4.45 (d, J=5.7 Hz, 4H), 3.76 (t, J=5.1 Hz, 4H), 3.58 (s, 6H), 3.48 (s, 4H), 2.35 (s, 6H), 2.19 (s, 6H). ¹⁹F NMR (377 MHz, DMSO-d₆) δ−137.1 to −137.2 (m, 1F), −144.2 to −144.4 (m, 1F). HRMS (ESI) m/z calcd for C₄₄H₄₇F₄N₁₂O₈ [M+H]⁺: 947.3576; found 947.3555.

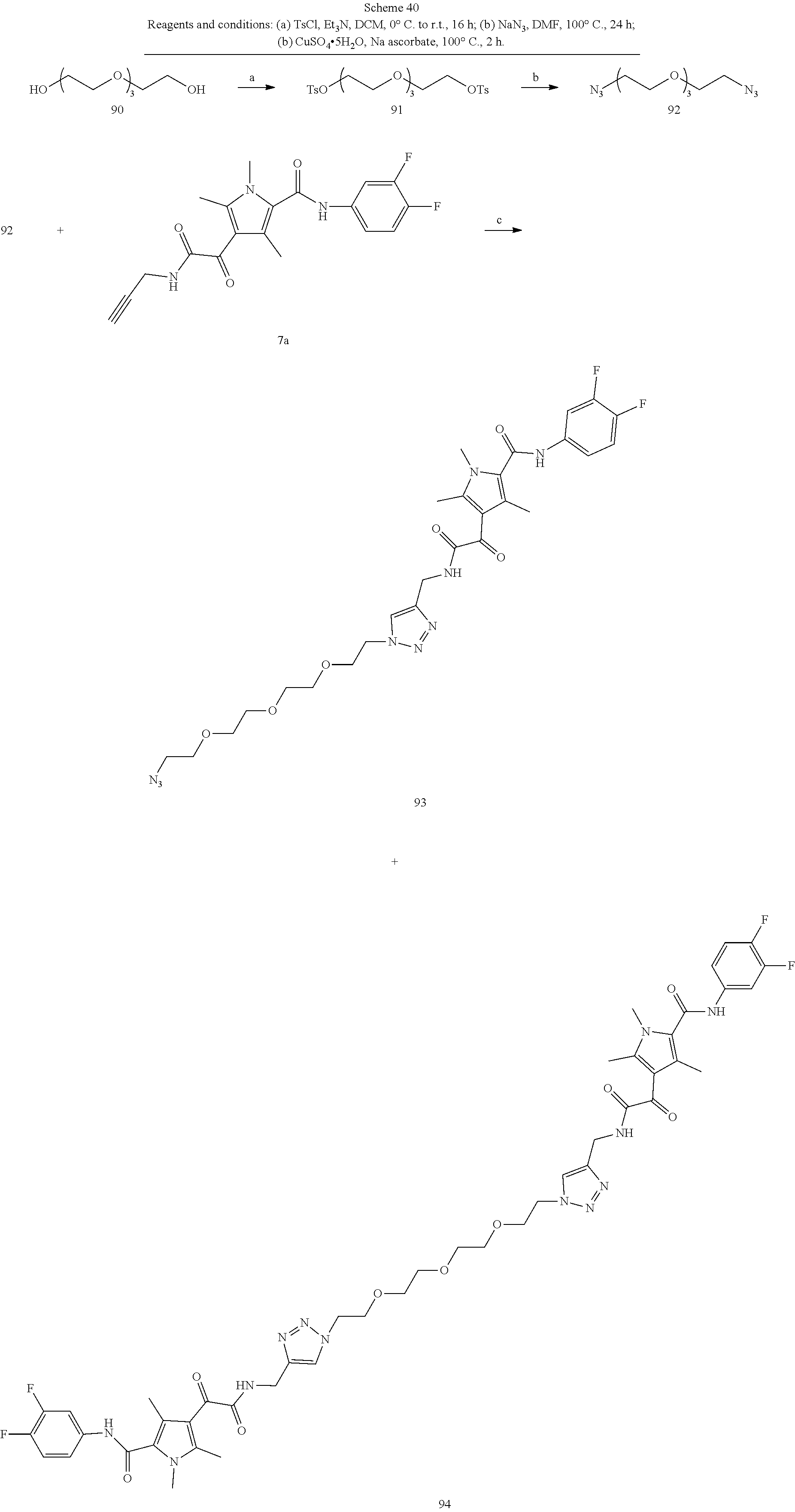

### ((Oxybis(ethane-2,1-diyl))bis(oxy))bis(ethane-2,1-diyl) bis(4-methylbenzenesulfonate) (92)

To a solution of compound 90 (2.0 g, 10.30 mmol, 1 eq), Et₃N (3.6 mL, 25.74 mmol, 2.5 eq) in DCM (50 mL) at 0° C. was added TsCl (4.3 g, 22.65 mmol, 2.2 eq). The resulting mixture was stirred at r.t. for 16 h and was quenched with a cold solution of saturated Na₂S₂O₃. The water layer was then extracted with DCM (3×100 mL). The organic layers were finally combined, washed with water, brine, and dried over anhydrous Na₂SO₄, and concentrated under reduced pressure. The residue was purified by flash chromatography (DCM/Ethyl acetate 3:1 to 1:2) to afford product 91 (4.7 g, 90%). To a solution of compound 91 (4.5 g, 8.95 mmol, 1.0 eq) in DMF (100 mL) was added NaN₃ (1.16 g, 17.91 mmol, 2.0 eq). The resulting mixture was stirred at 100° C. for 24 h and was quenched with cold H₂O (40 mL). The water layer was then extracted with EtOAc (2×100 mL). The organic layers were finally combined, washed with water, brine, and dried over anhydrous Na₂SO₄, and concentrated under reduced pressure. The residue was purified by flash chromatography (Hexane/Ethyl acetate 20:1 to 1:4) to afford product 92 (2.0 g, 80%). ¹H NMR (400 MHz, CDCl₃) δ 3.72-3.62 (m, 12H), 3.39 (t, J=6.4 Hz, 4H).

### 4-(2-(((1-(2-(2-(2-(2-Azidoethoxy)ethoxy)ethoxy)ethyl)-1H-1,2,3-triazol-4-yl)methyl)amino)-2-oxoacetyl)-N-(3,4-difluorophenyl)-1,3,5-trimethyl-1H-pyrrole-2-carboxamide (93) and 4,4′-(2,2′-((((((oxybis(ethane-2,1-diyl))bis(oxy))bis(ethane-2,1-diyl))bis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (94)

To a mixture of compound 92 (16.4 mg, 0.067 mmol, 0.5 eq) and compound 7a (50 mg, 0.134 mmol, 1.0 eq) in H₂O/CH₃CN (4 mL/6 mL) was added CuSO₄.5H₂O (20 mg) and Na ascorbate (40 mg) under Ar atmosphere. The resulting mixture was stirred at 100° C. for 2 h. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 93 (17 mg, 20%). ¹H NMR (400 MHz, DMSO-d₆) δ10.42 (s, 1H), 9.20 (t, J=5.8 Hz, 1H), 7.97 (s, 1H), 7.90-7.85 (m, 1H), 7.45-7.38 (m, 2H), 4.52 (t, J=5.1 Hz, 2H), 4.45 (d, J=5.8 Hz, 2H), 3.81 (t, J=5.3 Hz, 2H), 3.60-3.57 (m, 5H), 3.55-3.48 (m, 8H), 3.39-3.36 (m, 2H), 2.35 (s, 3H), 2.19 (s, 3H). ¹⁹F NMR (377 MHz, DMSO-d₆) δ−137.2 to −137.3 (m, 1F), −144.3 to −144.4 (m, 1F). HRMS (ESI) m/z calcd for C₂₇H₃₄F₂N₉O₆ [M+H]⁺: 618.2600; found 618.2587. and product 94 (41 mg, 61%). ¹H NMR (400 MHz, DMSO-d₆) δ10.42 (s, 2H), 9.19 (t, J=5.8 Hz, 2H), 7.96 (s, 2H), 7.90-7.84 (m, 2H), 7.45-7.38 (m, 4H), 4.51 (t, J=5.1 Hz, 4H), 4.44 (d, J=5.8 Hz, 4H), 3.79 (t, J=5.2 Hz, 4H), 3.58 (s, 6H), 3.52-3.49 (m, 4H), 3.45-3.43 (m, 4H), 2.34 (s, 6H), 2.19 (s, 6H). ¹⁹F NMR (377 MHz, DMSO-d₆) δ−137.1 to −137.2 (m, 1F), −144.3 to −144.4 (m, 1F). HRMS (ESI) m/z calcd for C₄₆H₅₁F₄N₁₂O₉ [M+H]⁺: 991.3838; found 991.3814.

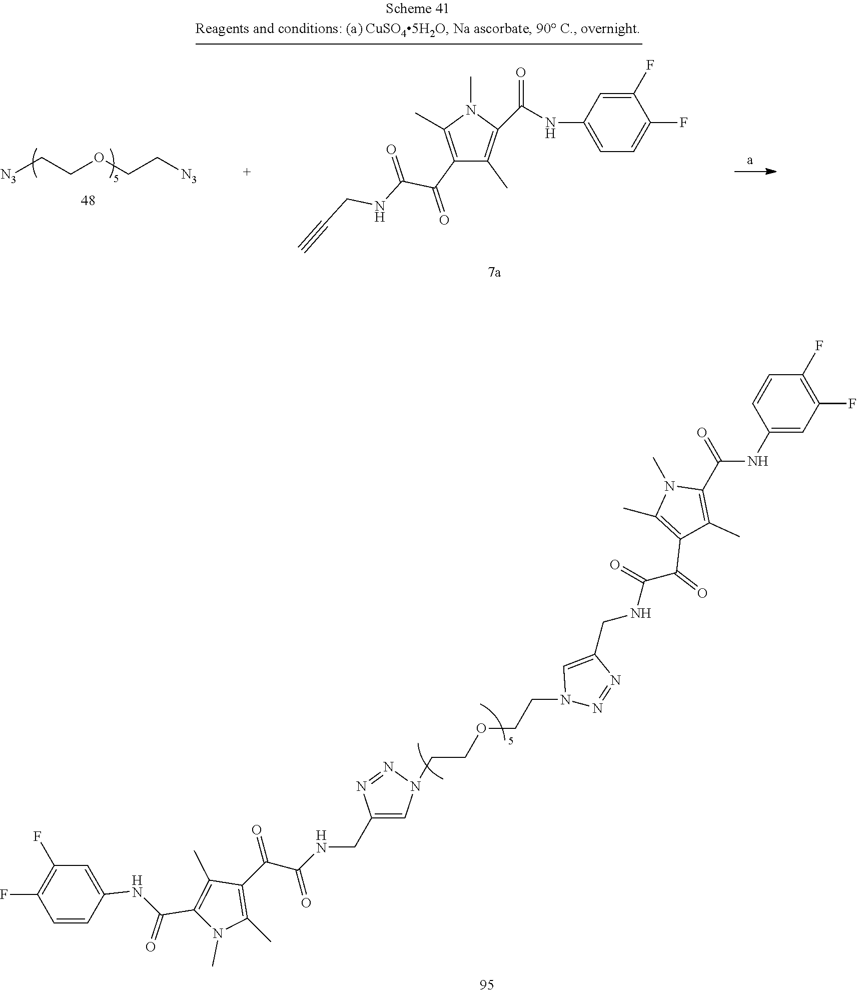

### 4,4′-(2,2′-((((3,6,9,12,15-Pentaoxaheptadecane-1,17-diyl)bis(1H-1,2,3-triazole-1,4-diyl)) bis (methylene))bis(azanediyl))bis(2-oxoacetyl))bis(N-(3,4-difluorophenyl)-1,3,5-trimethyl-1H-pyrrole-2-carboxamide) (95)

To a mixture of compound 48 (45 mg, 0.134 mmol, 0.5 eq) and compound 7a (100 mg, 0.268 mmol, 1.0 eq) in H₂O/CH₃CN (6 mL/10 mL) was added CuSO₄.5H₂O (28 mg) and Na ascorbate (60 mg) under Ar atmosphere. The resulting mixture was stirred at 90° C. overnight. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 95 (65 mg, 45%). ¹H NMR (400 MHz, CDCl₃) δ 8.72 (s, 2H), 7.93 (t, J=5.7 Hz, 2H), 7.81 (s, 2H), 7.76-7.71 (m, 2H), 7.33-7.28 (m, 2H), 7.13-7.06 (m, 2H), 4.48 (t, J=3.9 Hz, 8H), 3.81 (t, J=4.8 Hz, 4H), 3.59-3.54 (m, 22H), 2.29 (s, 6H), 2.18 (s, 6H). ¹⁹F NMR (377 MHz, CDCl₃) δ−135.9 to −136.0 (m, 1F), −142.5 to −142.7 (m, 1F). LCMS (ESI) m/z calcd for C₅₀H₅₉F₄N₁₂O₁₁ [M+H]⁺: 1080.1; found 1079.4.

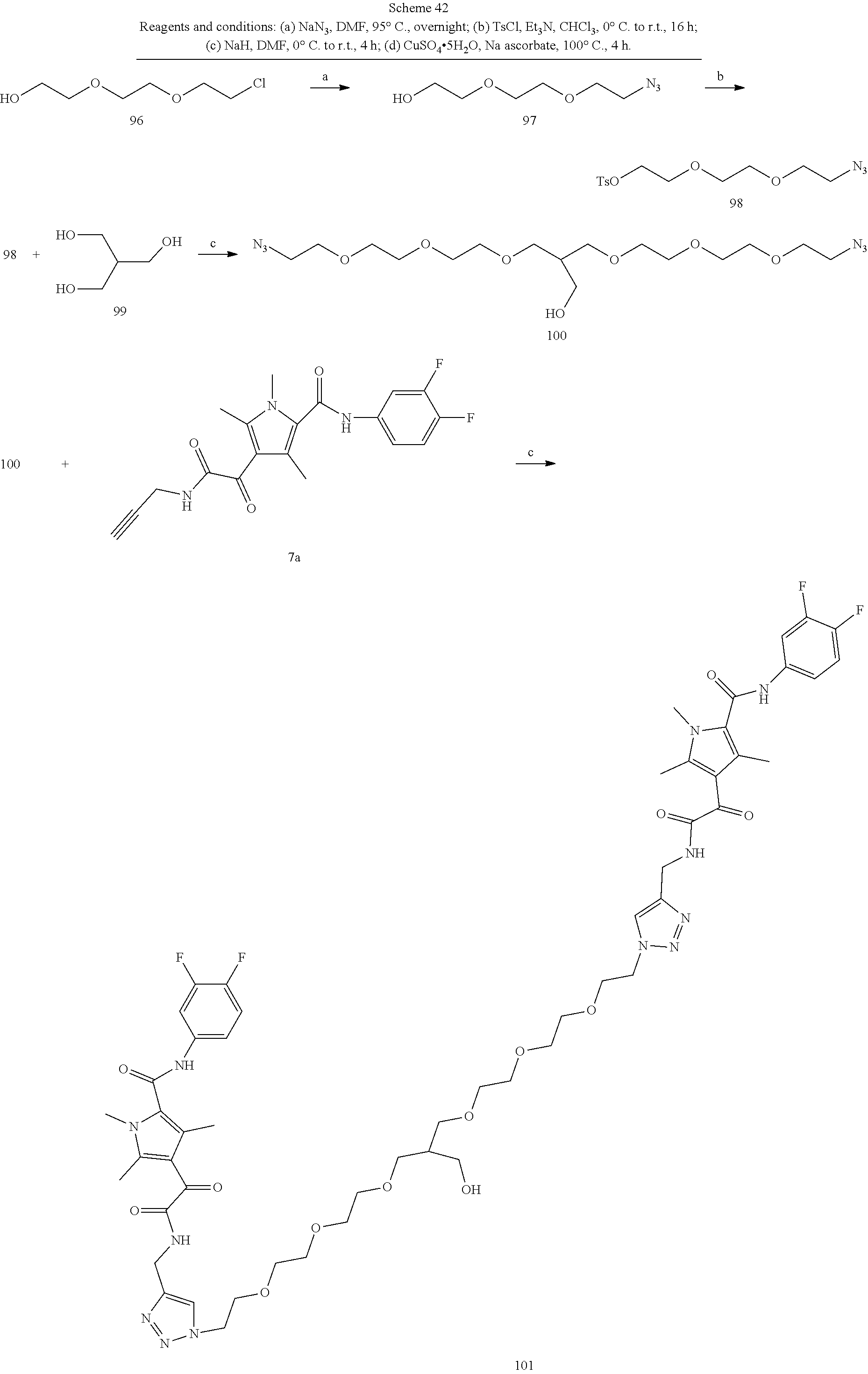

### 2-(2-(2-azidoethoxy) ethyl) ethan-1-ol (97)

To a solution of compound 96 (2.5 g, 14.8 mmol, 1.0 eq) in DMF/H₂O (60 mL/1.5 mL) was added NaN₃ (1.0 g, 15.4 mmol, 1.05 eq). The resulting mixture was stirred at 95° C. overnight and was quenched with cold H₂O (50 mL). The water layer was then extracted with EtOAc (2×100 mL). The organic layers were finally combined, washed with water, brine, and dried over anhydrous Na₂SO₄, and concentrated under reduced pressure. The residue was purified by flash chromatography (Hexane/Ethyl acetate 10:1 to 1:4) to afford product 97 (2.5 g, 95%). ¹H NMR (400 MHz, CDCl₃) δ 3.76-3.72 (m, 2H), 3.71-3.67 (m, 6H), 3.63-3.61 (m, 2H), 3.41 (t, J=5.2 Hz, 2H), 2.86-2.85 (m, 1H).

### 2-(2-(2-Azidoethoxy)ethoxy)ethyl 4-methylbenzenesulfonate (98)

To a solution of compound 97 (1.8 g, 10.28 mmol, 1 eq), Et₃N (2.9 mL, 30.83 mmol, 2.0 eq) in DCM (60 mL) at 0° C. was added TsCl (2.4 g, 12.33 mmol, 1.2 eq). The resulting mixture was stirred at r.t. for 16 h and was quenched with cold saturated Na₂S₂O₃. The water layer was then extracted with DCM (3×100 mL). The organic layers were finally combined, washed with water, brine, and dried over anhydrous Na₂SO₄, and concentrated under reduced pressure. The residue was purified by flash chromatography (DCM/Ethyl acetate 3:1 to 1:2) to afford product 98 (3.8 g, 90%). H NMR (400 MHz, CDCl₃) δ 7.80 (d, J=8.4 Hz, 2H), 7.35 (d, J=8.4 Hz, 2H), 4.18-4.15 (m, 2H), 3.71-3.69 (m, 2H), 3.66-3.63 (m, 2H), 3.60 (s, 4H), 3.37 (t, J=5.2 Hz, 2H), 2.45 (s, 3H).

### 3-(2-(2-(2-Azidoethoxy) ethoxy)ethyl)-2-((2-(2-(2-azidoethoxy)ethyl)ethyl)methyl)propan-1-ol (100)

Compound 99 (200 mg, 1.88 mmol, 1.0 eq) was dissolved in anhydrous DMF (30 mL) and NaH (60% in oil, 407 mg, 10.18 mmol, 5.4 eq) in powder form was added portion wise to the reaction under Ar. The reaction was stirred for 30 minutes and then compound 98 (1.86 g, 5.65 mmol, 3.0 eq) was added dropwise. After completion of the reaction, the mixture was filtered and the organic phase was quenched with cold H₂O and ice with caution. The water layer was then extracted with EtOAc (3×100 mL). The organic layers were finally combined, washed with water, brine, and dried over anhydrous Na₂SO₄, and concentrated under reduced pressure. The residue was purified by flash chromatography (DCM/Ethyl acetate 3:1 to 1:10) to afford product 100 (3.8 g, 90%). H NMR (400 MHz, CD₃COCD₃) δ 3.69-3.68 (m, 4H), 3.64-3.58 (m, 15H), 3.56-3.51 (m, 9H), 3.39 (t, J=5.0 Hz, 4H). LCMS (ESI) m/z calcd for C₁₆H₃₃N₆O₇ [M+H]⁺: 421.5; found 421.4.

### 4,4′-(2,2′-((((11-(Hydroxymethyl)-3,6,9,13,16,19-hexaoxahenicosane-1,21-diyl)bis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (101)

To a mixture of compound 100 (30 mg, 0.071 mmol, 1.0 eq) and compound 7a (58 mg, 0.156 mmol, 3.0 eq) in H₂O/CH₃CN (3 mL/3 mL) was added CuSO₄.5H₂O (15 mg) and Na ascorbate (30 mg) under Ar atmosphere. The resulting mixture was stirred at 100° C. for 4 h. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 101 (33 mg, 40%). ¹H NMR (400 MHz, DMSO-d₆) δ10.41 (s, 2H), 9.19 (t, J=5.8 Hz, 2H), 7.97 (s, 2H), 7.90-7.84 (m, 2H), 7.45-7.38 (m, 4H), 4.52 (t, J=5.1 Hz, 4H), 4.44 (d, J=5.8 Hz, 4H), 3.80 (t, J=5.2 Hz, 4H), 3.58 (s, 6H), 3.53-3.43 (m, 15H), 3.41-3.35 (m, 8H), 2.35 (s, 6H), 2.19 (s, 6H), 1.89-1.83 (m, 1H). ¹⁹F NMR (377 MHz, DMSO-d₆) δ−137.1 to −137.2 (m, 1F), −144.2 to −144.4 (m, 1F). HRMS (ESI) m/z calcd for C₅₄H₆₇F₄N₁₂O₁₃ [M+H]⁺: 1167.4887; found 1167.4870.

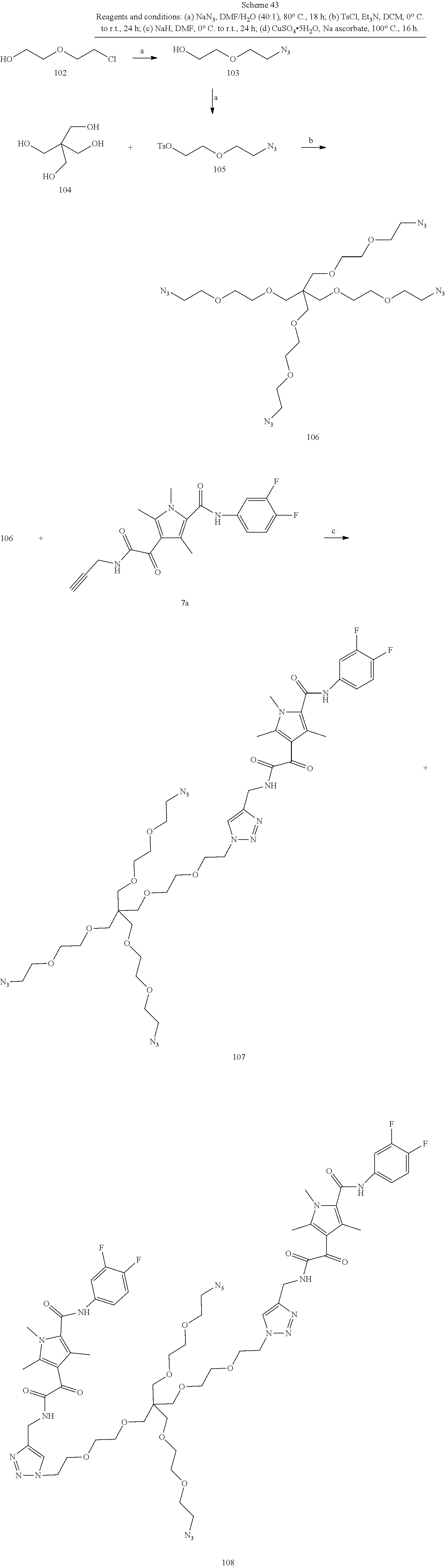

### 2-(2-Azidoethoxy) ethan-1-ol (103)

To a solution of compound 102 (2.5 g, 20.0 mmol, 1.0 eq) in DMF/H₂O (60 mL/1.5 mL) was added NaN₃ (1.4 g, 21.1 mmol, 1.05 eq). The resulting mixture was stirred at 80° C. overnight and was quenched with cold H₂O (50 mL). The water layer was then extracted with EtOAc (3×50 mL). The organic layers were finally combined, washed with water, brine, and dried over anhydrous Na₂SO₄, and concentrated under reduced pressure. The residue was purified by flash chromatography (Hexane/Ethyl acetate 10:1 to 1:4) to afford product 103 (2.3 g, 88%). ¹H NMR (400 MHz, CDCl₃) δ 3.78-3.75 (m, 2H), 3.74-3.69 (m, 2H), 3.63-3.61 (m, 2H), 3.42 (t, J=5.1 Hz, 2H), 2.77 (t, J=6.0 Hz, 1H).

### 2-(2-Azidoethoxy) ethyl 4-methylbenzenesulfonate (105)

To a solution of compound 103 (1.4 g, 10.7 mmol, 1 eq), Et₃N (3.0 mL, 21.4 mmol, 2.0 eq) in DCM (60 mL) at 0° C. was added TsCl (2.4 g, 12.8 mmol, 1.2 eq). The resulting mixture was stirred at r.t. for 24 h and was quenched with cold saturated Na₂S₂O₃. The water layer was then extracted with DCM (3×50 mL). The organic layers were finally combined, washed with water, brine, and dried over anhydrous Na₂SO₄, and concentrated under reduced pressure. The residue was purified by flash chromatography (DCM/Ethyl acetate 3:1 to 1:2) to afford product 105 (2.7 g, 90%). H NMR (400 MHz, CDCl₃) δ 7.81 (d, J=8.3 Hz, 2H), 7.35 (d, J=8.0 Hz, 2H), 4.18-4.16 (m, 2H), 3.71-3.69 (m, 2H), 3.62-3.59 (m, 2H), 3.32 (t, J=5.1 Hz, 2H), 2.45 (s, 3H).

### 1,15-Diazido-8,8-bis((2-(2-azidoethoxy)ethoxy)methyl)-3,6,10,13-tetraoxapentadecane (106)

Compound 104 (260 mg, 1.91 mmol, 1.0 eq) was dissolved in anhydrous DMF (25 mL) and NaH (60% in oil, 367 mg, 9.2 mmol, 4.8 eq) in powder form was added portionwise to the reaction under Ar. The reaction was stirred for 30 minutes and then compound 105 (2.18 g, 7.64 mmol, 4.0 eq) was added dropwise. After the reaction was complete and the mixture was filtered and the organic phase was quenched with cold H₂O and ice with caution. The water layer was then extracted with EtOAc (3×100 mL). The organic layers were finally combined, washed with water, brine, and dried over anhydrous Na₂SO₄, and concentrated under reduced pressure. The residue was purified by flash chromatography (DCM/Ethyl acetate 3:1 to 1:10) to afford product 106 (910 mg, 81%). ¹H NMR (400 MHz, CDCl₃) δ 3.69-3.66 (m, 8H), 3.64-3.62 (m, 8H), 3.59-3.56 (m, 8H), 3.47 (s, 8H), 3.74 (t, J=5.2 Hz, 8H).

### 4-(2-(((1-(15-azido-8,8-bis((2-(2-azidoethoxy)ethoxy)methyl)-3,6,10,13-tetraoxapentadecyl)-1H-1,2,3-triazol-4-yl)methyl)amino)-2-oxoacetyl)-N-(3,4-difluorophenyl)-1,3,5-trimethyl-1H-pyrrole-2-carboxamide (107) and 4,4′-(2,2′-((((8,8-bis((2-(2-azidoethoxy)ethoxy)methyl)-3,6,10,13-tetraoxapentadecane-1,15-diyl)bis(1H-1,2,3-triazole-1,4-diyl))bis(methylene))bis(azanediyl))bis(2-oxoacetyl))bis(N-(3,4-difluorophenyl)-1,3,5-trimethyl-1H-pyrrole-2-carboxamide) (108)

To a mixture of compound 106 (426 mg, 0.723 mmol, 4.5 eq) and compound 7a (60 mg, 0.161 mmol, 1.0 eq) in H₂O/CH₃CN (4 mL/4 mL) was added CuSO₄.5H₂O (20 mg) and Na ascorbate (40 mg) under Ar atmosphere. The resulting mixture was stirred at 100° C. for 16 h. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 107 (46 mg, 30%) and product 108 (48 mg, 45%). For compound 107: ¹H NMR (400 MHz, DMSO-d₆) δ 10.41 (s, 1H), 9.19 (t, J=5.7 Hz, 1H), 7.96 (s, 1H), 7.91-7.85 (m, 2H), 4.53 (t, J=5.2 Hz, 2H), 4.46 (d, J=5.7 Hz, 2H), 3.84-3.82 (m, 2H), 3.63-3.44 (m, 26H), 3.39-3.33 (m, 14H), 2.36 (s, 3H), 2.20 (s, 3H). ¹⁹F NMR (377 MHz, DMSO-d₆) δ−137.2 to −137.3 (m, 1F), −144.3 to −144.4 (m, 1F). HRMS (ESI) m/z calcd for C₄₀H₅₈F₂N₁₅O₁₁[M+H]⁺: 962.4408; found 962.4393. For compound 108: ¹H NMR (400 MHz, DMSO-d₆) δ 10.42 (s, 2H), 9.20 (t, J=5.7 Hz, 2H), 7.97 (s, 2H), 7.91-7.86 (m, 2H), 7.48-7.37 (m, 4H), 4.53 (t, J=5.1 Hz, 4H), 4.46 (d, J=18.6 Hz, 4H), 3.83 (t, J=5.2 Hz, 4H), 3.63-3.60 (m, 10H), 3.56-3.51 (m, 16H), 3.50-3.31 (m, 12H), 2.36 (s, 6H), 2.21 (s, 6H). ¹⁹F NMR (377 MHz, DMSO-d₆) δ−137.1 to −137.2 (m, 1F), −144.2 to −144.4 (m, 1F). HRMS (ESI) m/z calcd for C₅₈H₇₅F₄N₁₈O₁₄[M+H]⁺: 1335.5646; found 1335.5639.

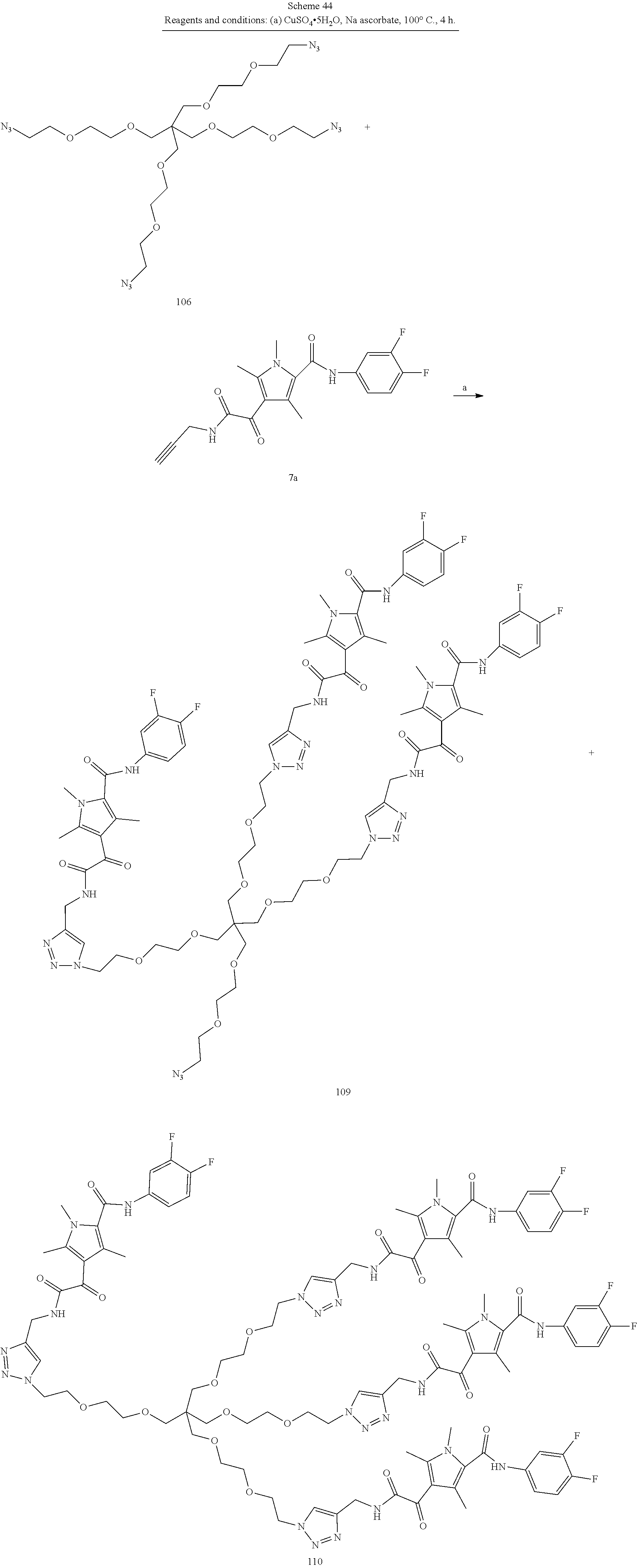

### Trimer (109) and Tetramer (110)

To a mixture of compound 106 (32 mg, 0.0544 mmol, 1 eq) and compound 7a (162 mg, 0.4349 mmol, 8.0 eq) in H₂O/CH₃CN (4 mL/4 mL) was added CuSO₄.5H₂O (20 mg) and Na ascorbate (40 mg) under Ar atmosphere. The resulting mixture was stirred at 100° C. for 4 h. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 109 (Trimer, 33 mg, 35%) and product 110 (Tetramer, 52 mg, 46%). For trimer 109: ¹H NMR (400 MHz, DMSO-d₆) δ10.42 (s, 3H), 9.21 (t, J=5.7 Hz, 3H), 7.98 (s, 3H), 7.91-7.86 (m, 3H), 7.48-7.37 (m, 6H), 4.53 (t, J=5.0 Hz, 6H), 4.47 (d, J=5.6 Hz, 6H), 3.83 (t, J=5.1 Hz, 6H), 3.62-3.60 (s, 10H), 3.56-3.44 (m, 17H), 3.38 (dd, J=4.2 Hz, 9.3 Hz, 3H), 3.31-3.29 (m, 7H), 2.37 (s, 9H), 2.21 (s, 9H). ¹⁹F NMR (377 MHz, DMSO-d₆) δ−137.1 to −137.2 (m, 1F), −144.2 to −144.3 (m, 1F). HRMS (ESI) m/z calcd for C₇₈H₉₂F₆N₂₁O₁₇[M+H]⁺: 1708.6884; found 1708.6865. For tetramer 110: ¹H NMR (400 MHz, DMSO-d₆) δ 10.41 (s, 4H), 9.19 (t, J=5.7 Hz, 4H), 7.95 (s, 4H), 7.89-7.84 (m, 4H), 7.45-7.37 (m, 8H), 4.50 (t, J=5.1 Hz, 8H), 4.44 (d, J=5.7 Hz, 8H), 3.80 (t, J=5.2 Hz, 8H), 3.57 (s, 12H), 3.50-3.48 (m, 8H), 3.42-3.39 (m, 8H), 3.24 (s, 8H), 2.34 (s, 12H), 2.18 (s, 12H). ¹⁹F NMR (377 MHz, DMSO-d₆) δ−137.1 to −137.2 (m, 1F), −144.2 to −144.4 (m, 1F). HRMS (ESI) m/z calcd for C₉₇H₁₀₉F₈N₂₄O₂₀[M+H]⁺: 2081.8122; found [M+H]²⁺: 1041.9089.

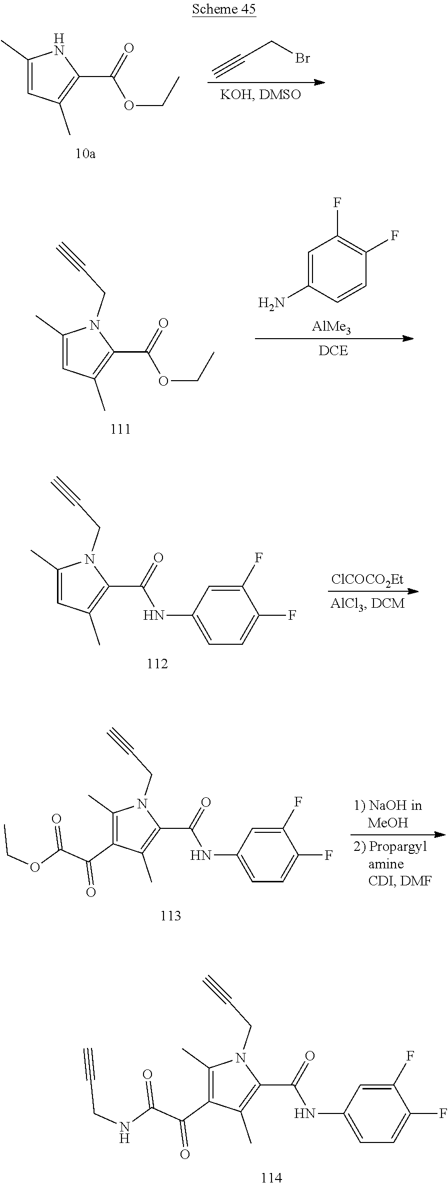

### Ethyl 3,5-dimethyl-1-(prop-2-yn-1-yl)-1H-pyrrole-2-carboxylate (111)

A solution of ethyl 3,5-dimethyl-1H-pyrrole-2-carboxylate (10a) (1.76 g, 10.5 mmol, 1 equiv.) in DMSO (10 mL) was treated with KOH (1.46 g, 26.0 mmol, 2.5 equiv.) and propargyl bromide (80% in toluene, 12.0 mL). The solution was stirred at ambient temperature for 24 hours after which time the solution was poured into water (150 mL) and then extracted three times with EtOAc (50 mL). The combined organic phase was dried over anhydrous Na₂SO₄, filtered and concentrated in vacuo. The residue was purified via SiO₂ chromatography using hexane:EtOAc (90:10) as eluent to give ethyl 3,5-dimethyl-1-(prop-2-yn-1-yl)-1H-pyrrole-2-carboxylate 111 (64.9% yield) as a white solid. ¹H NMR (CDCl₃) δ ppm 5.79 (s, 1H), 5.10 (d, J=2.4 Hz, 2H), 4.29 (q, J=7.2 Hz, 2H), 2.28 (s, 3H), 2.27 (s, 3H), 2.23 (t, J=2.4 Hz, 1H), 1.35 (t, J=7.2 Hz, 3H). ¹³C NMR (CDCl₃) δ 161.97, 135.50, 118.16, 111.46, 79.33, 71.50, 59.51, 34.46, 14.15, 14.33, 12.12. LC-MS (ES⁺) m/z 206.3 [M+H],

### N-(3,4-Difluorophenyl)-3,5-dimethyl-1-(prop-2-yn-1-yl)-1H-pyrrole-2-carboxamide 112

A solution of ethyl 3,5-dimethyl-1-(prop-2-yn-1-yl)-1H-pyrrole-2-carboxylate (111) (1.37 g, 6.67 mmol, 1 equiv.) and 3,4-difluoroaniline (1.40 mL, 13.7 mmol, 2 equiv.) in dichloroethane (40 mL) was cooled to 0° C. and was treated with trimethyl aluminum (2.0 M in toluene) (6.70 mL, 13.4 mmol, 2 equiv.). The solution was allowed to slowly come to ambient temperature over 2 hours and then heated to reflux under nitrogen for 24 hours after which time the solution was poured into water (150 mL) and extracted three times with dichloromethane (50 mL). The combined organic phase was dried over anhydrous Na₂SO₄, filtered and concentrated in vacuo. The residue was purified via SiO₂ chromatography using hexane:EtOAc (80:20) as eluent to give N-(3,4-difluorophenyl)-3,5-dimethyl-1-(prop-2-yn-1-yl)-1H-pyrrole-2-carboxamide 112 (89.3% yield) as an off white solid. ¹H NMR (CDCl₃) δ ppm 7.72-7.67 (m, 1H), 7.37 (s, 1H), 7.10-7.07 (m, 2H), 5.81 (s, 1H), 5.07 (d, J=2.4 Hz, 2H), 2.34 (s, 3H), 2.30 (s, 3H), 2.27 (t, J=2.4 Hz, 1H). ¹⁹F NMR (CDCl₃) δ ppm −135.79 (d, J=24 Hz, 1F), −143.23 (d, J=24 Hz, 1F). ¹³C NMR (CDCl₃) δ ppm 160.31, 151.46, 151.33, 149.00, 148.87, 148.06, 147.93, 145.63, 145.50, 134.95, 134.73, 134.70, 134.64, 134.61, 123.37, 121.91, 117.25, 117.08, 115.32, 111.21, 109.74, 109.52, 79.24, 71.94, 34.24, 13.85, 11.99. LC-MS (ES⁺) m/z 289.3 [M+H],

### Ethyl 2-(5-((3,4-difluorophenyl)carbamoyl)-2,4-dimethyl-1-(prop-2-yn-1-yl)-1H-pyrrol-3-yl)-2-oxoacetate 113

A solution of N-(3,4-difluorophenyl)-3,5-dimethyl-1-(prop-2-yn-1-yl)-1H-pyrrole-2-carboxamide (112) (2.05 g, 7.11 mmol, 1 equiv.) and aluminum trichloride (2.85 g, 21.4 mmol, 3 equiv.) in DCM (70 mL) was cooled to 0° C. and ClCOCO₂Et (2.38 mL, 21.3 mmol, 3 equiv.) was added dropwise over 30 min. The solution was allowed to slowly come to ambient temperature and was stirred under nitrogen for 24 hours. The solution was then poured over cold water (200 mL). The aqueous phase was extracted three time with DCM (50 mL). The combined organic phase was dried over anhydrous Na₂SO₄, filtered and concentrated in vacuo. The residue was purified via SiO₂ chromatography using hexane:EtOAc (80:20) as eluent to give ethyl 2-(5-((3,4-difluorophenyl)carbamoyl)-2,4-dimethyl-1-(prop-2-yn-1-yl)-1H-pyrrol-3-yl)-2-oxoacetate 113 (66.0% yield) as a white solid. ³H NMR (CDCl₃) δ ppm 7.82 (s, 1H), 7.73 (dd, J=7.2, 2.4 Hz, 1H), 7.22-7.10 (m, 2H), 4.99 (d, J=2.4 Hz, 2H), 2.52 (s, 3H), 2.36 (s, 4H), 1.39 (t, J=7.2 Hz, 3H). ¹⁹F NMR (CDCl₃) δ ppm −135.29 (d, J=24 Hz, 1F), −141.80 (d, J=24 Hz, 1F). ¹³C NMR (CDCl₃) δ ppm 165.55, 159.69, 151.46, 151.33, 149.00, 148.87, 148.54, 148.41, 146.09, 145.97, 142.34, 134.03, 134.00, 133.94, 133.91, 125.33, 123.33, 117.46, 117.28, 117.14, 115.69, 115.66, 115.63, 115.60, 109.99, 109.77, 73.56, 62.31, 34.35, 13.97, 11.67, 11.62. LC-MS (ES⁺) m/z 389.4 [M+H],

### N-(3,4-Difluorophenyl)-3,5-dimethyl-4-(2-oxo-2-(prop-2-yn-1-ylamino)acetyl)-1-(prop-2-yn-1-yl)-1H-pyrrole-2-carboxamide (114)

A solution of ethyl 2-(5-((3,4-difluorophenyl)carbamoyl)-2,4-dimethyl-1-(prop-2-yn-1-yl)-1H-pyrrol-3-yl)-2-oxoacetate (113) (0.780 g, 2.00 mmol, 1 equiv.) in MeOH (10 mL) and THF (2 mL) was cooled to 0° C. and 5% aqueous NaOH (10 mL) was added slowly. The solution was stirred at 0° C. for 20 min. after which time the solution was acidified to pH 4-5 by the slow addition of 1N HCl. The solution was partitioned between water (50 mL) and EtOAc (25 mL). The aqueous phase was extracted two times with EtOAc (25 mL). The combined organic phase was dried over anhydrous Na₂SO₄, filtered and concentrated in vacuo. The residue was dissolved in DMF (20 mL) and CDI (1.62 g, 9.99 mmol, 10 equiv.) was added. After stirring the solution for 30 min. under nitrogen at ambient temperature, propargyl amine (0.380 mL, 5.93 mmol, 3 equiv.) was added and the reaction was stirred for 24 hours. The solution was then poured over water (150 mL) and extracted tree times with EtOAc (30 mL). The combined organic phase was dried over anhydrous Na₂SO₄, filtered and concentrated in vacuo. The residue was purified via SiO₂ chromatography using hexane:EtOAc (65:35) as eluent to give N-(3,4-difluorophenyl)-3,5-dimethyl-4-(2-oxo-2-(prop-2-yn-1-ylamino)acetyl)-1-(prop-2-yn-1-yl)-1H-pyrrole-2-carboxamide 114 (35% yield) as a white solid. ¹H NMR (DMSO-d⁶) δ ppm 10.53 (s, 1H), 9.21 (t, J=5.6 Hz, 1H), 7.90-7.85 (m, 1H), 7.47-7.42 (m, 2H), 5.03 (d, J=2 Hz, 2), 4.02 (dd, J=2.4, 5.6 Hz, 2H), 3.42 (t, J=2.4 Hz, 1H), 3.2 (t, J=2.4 Hz, 1H), 2.47 (s, 3H), 2.26 (s, 3H). ¹⁹F NMR (DMSO-d⁶) δ ppm −137.15 (d, J=28 Hz, 1F), −144.14 (d, J=24 Hz, 1F). ¹³C NMR (DMSO-d⁶) δ ppm 187.93, 167.31, 160.30, 150.68, 150.55, 148.26, 148.13, 147.33, 147.19, 144.91, 144.79, 141.23, 136.28, 136.20, 126.17, 123.69, 118.05, 117.87, 117.26, 116.51, 109.29, 109.08, 80.50, 78.99, 76.07, 73.86, 34.06, 28.12, 11.97, 11.69. LC-MS (ES⁺) m/z 398.3 [M+H],

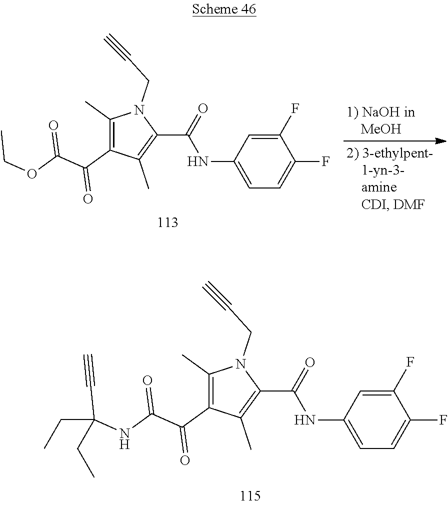 [SMILES: C#CCn1c(C)c(C(=O)C(=O)OCC)c(C)c1C(=O)Nc1ccc(F)c(F)c1 ; C#CCn1c(C)c(C(=O)C(=O)NC(C#C)(CC)CC)c(C)c1C(=O)Nc1ccc(F)c(F)c1]

### N-(3,4-Difluorophenyl)-4-(2-((3-ethylpent-1-yn-3-yl)amino)-2-oxoacetyl)-3,5-dimethyl-1-(prop-2-yn-1-yl)-1H-pyrrole-2-carboxamide 115

A solution of ethyl 2-(5-((3,4-difluorophenyl)carbamoyl)-2,4-dimethyl-1-(prop-2-yn-1-yl)-1H-pyrrol-3-yl)-2-oxoacetate (113) (0.450 g, 1.16 mml, 1 equiv.) in MeOH (11 mL) and THF (1 mL) was cooled to 0° C. and 5% aqueous NaOH (11 mL) was added slowly. The solution was stirred at 0° C. for 20 min after which time the solution was acidified to pH 4-5 by the slow addition of 1N HCl. The solution was partitioned between water (100 mL) and EtOAc (50 mL). The aqueous phase was extracted two times with EtOAc (25 mL). The combined organic phase was dried over anhydrous Na₂SO₄, filtered and concentrated in vacuo. The residue was then dissolved in DMF (11 mL) and treated with HATU (1.33 g, 3.50 mmol, 3 equiv.) and DIPEA (0.610 mL, 3.50 mmol, 3 equiv.) and stirred at ambient temperature under nitrogen for 30 min after which time 3-ethylpent-1-yn-3-amine (0.520 g, 4.68 mmol, 4 equiv.) was added and the reaction was continued for 2 hours. The solution was then heated to 65° C. and stirred under nitrogen for 24 hours. After which time the solution was poured over water (150 mL) and extracted tree times with EtOAc (30 mL). The combined organic phase was dried over anhydrous Na₂SO₄, filtered and concentrated in vacuo. The residue was purified via SiO₂ chromatography using hexane:EtOAc (65:35) as eluent to give N-(3,4-difluorophenyl)-4-(2-((3-ethylpent-1-yn-3-yl)amino)-2-oxoacetyl)-3,5-dimethyl-1-(prop-2-yn-1-yl)-1H-pyrrole-2-carboxamide 115 (35.7% yield) as a white solid. ¹H NMR (CDCl₃) δ ppm 7.73-7.69 (m, 1H), 7.65 (s, 1H), 7.17-7.09 (m, 2H), 6.88 (s, 1H), 5.01 (d, J=2.4 Hz, 2H), 0.45 (s, 3H), 2.43 (s, 1H), 2.37 (s, 3H), 2.31 (t, J=2.4 Hz, 1H), 2.21-2.13 (m, 2H), 1.96-1.87 (m, 2H), 1.04 (t, J=7.2 Hz, 6H). ¹⁹F NMR (CDCl₃) δ ppm-135.31 (d, J=24 Hz, 1F), −147.04 (d, J=20 Hz, 1F). ¹³C NMR (CDCl₃) δ ppm 187.17, 162.41, 159.87, 151.47, 151.34, 149.01, 148.88, 148.46, 146.01, 145.88, 141.95, 134.12, 134.03, 124.68, 124.30, 118.41, 117.43, 117.25, 115.53, 115.49, 115.44, 109.90, 109.68, 83.89, 73.21, 72.40, 57.24, 34.29, 30.35, 12.61, 12.00, 8.57. LC-MS (ES⁺) m/z 454.2 [M+H],

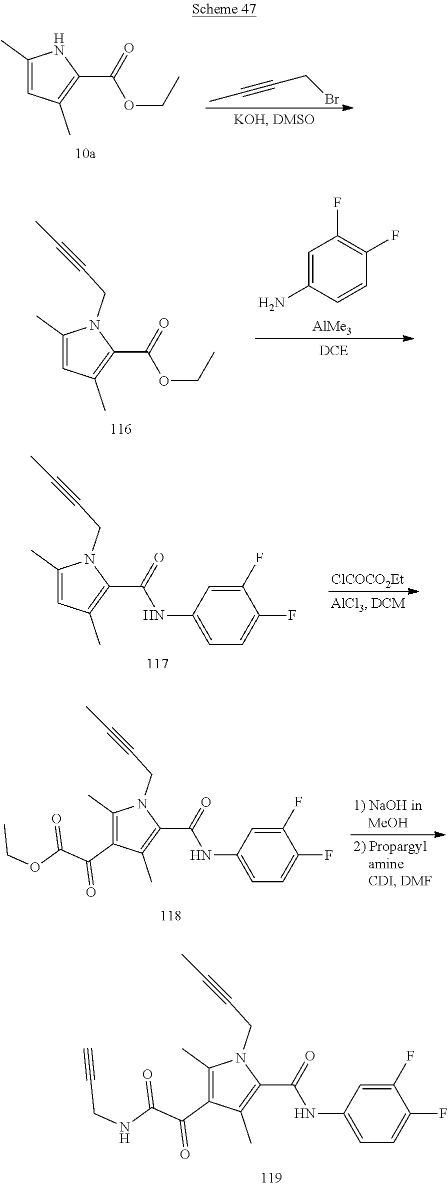

### Ethyl 1-(but-2-yn-1-yl)-3,5-dimethyl-1H-pyrrole-2-carboxylate 116

A solution of ethyl 3,5-dimethyl-1H-pyrrole-2-carboxylate 10a (1.03 g, 6.16 mmol, 1 equiv.) in DMSO (6 mL) was treated with KOH (0.710 g, 12.7 mmol, 2 equiv.) and 1-bromobut-2-yne (1.60 mL, 18.2 mmol, 3 equiv.). The solution was stirred for 24 hours after which time the solution was poured into water (150 mL) and then extracted three times with ethyl acetate (50 mL). The combined organic phase was dried over anhydrous Na₂SO₄, filtered and concentrated in vacuo. The residue was purified via SiO₂ chromatography using hexane:EtOAc (95:5) as eluent to give ethyl 1-(but-2-yn-1-yl)-3,5-dimethyl-1H-pyrrole-2-carboxylate 116 (84.4% yield) as a white solid. ³H NMR (CDCl₃) δ ppm 5.75 (s, 1H), 5.03 (q, J=2.4 Hz, 2H), 4.28 (q, J=7.2 Hz, 2H), 2.27 (s, 3H), 2.25 (s, 3H), 1.74 (t, J=2.4 Hz, 3H), 1.34 (t, J=7.2 Hz, 3H). ¹³C NMR (CDCl₃) δ ppm 161.91, 135.46, 129.93, 118.02, 111.17, 78.95, 74.68, 59.35, 34.75, 14.43, 14.33, 12.05, 3.46. LC-MS (ES⁺) m/z 220.3 [M+H],

### 1-(But-2-yn-1-yl)-N-(3,4-difluorophenyl)-3,5-dimethyl-1H-pyrrole-2-carboxamide 117

A solution of ethyl 1-(but-2-yn-1-yl)-3,5-dimethyl-1H-pyrrole-2-carboxylate (116) (1.14 g, 5.20 mmol, 1 equiv.) and 3,4-difluoroaniline (1.00 mL, 9.76 mmol, 2 equiv.) in dichloroethane (50 mL) was cooled to 0° C. and was treated with trimethyl aluminum (2.0 M in toluene) (5.20 mL, 10.4 mmol, 2 equiv.). The solution was allowed to slowly come to ambient temperature over 2 hours and then heated to reflux under nitrogen for 24 hours after which time the solution was poured into water (150 mL) and extracted three times with dichloromethane (50 mL). The combined organic phase was dried over anhydrous Na₂SO₄, filtered and concentrated in vacuo. The residue was purified via SiO₂ chromatography using hexane:EtOAc (85:15) as eluent to give 1-(but-2-yn-1-yl)-N-(3,4-difluorophenyl)-3,5-dimethyl-1H-pyrrole-2-carboxamide 117 (64.9% yield) as a white solid. ³H NMR (CDCl₃) δ ppm 7.70-7.69 (m, 1H), 7.44 (s, 1H), 7.11-7.05 (m, 2H), 5.79 (s, 1H), 4.97 (d, J=2.4 Hz, 2H), 2.33 (s, 3H), 2.30 (s, 3H), 1.78 (t, J=2.4 Hz, 3H). ¹⁹F NMR (CDCl₃) δ ppm −135.87 (d, J=20 Hz, 1F), −143.43 (d, J=24 Hz, 1F). ¹³C NMR (CDCl₃) δ ppm 160.35, 151.45, 151.32, 149.00, 148.87, 147.98, 147.85, 134.86, 123.32, 122.01, 117.21, 117.04, 115.15, 110.90, 109.63, 109.41, 79.86, 74.56, 34.66, 14.19, 12.02, 3.64. LC-MS (ES⁺) m/z 303.3 [M+H],

### Ethyl 2-(1-(but-2-yn-1-yl)-5-((3,4-difluorophenyl)carbamoyl)-2,4-dimethyl-1H-pyrrol-3-yl)-2-oxoacetate 118

A solution of 1-(but-2-yn-1-yl)-N-(3,4-difluorophenyl)-3,5-dimethyl-1H-pyrrole-2-carboxamide (117) (1.02 g, 3.37 mmol, 1 equiv.) and aluminum trichloride (1.35 g, 10.1 mmol, 3 equiv.) in DCM (70 mL) was cooled to 0° C. and ClCOCO₂Et (1.10 mL, 9.83 mmol, 3 equiv.) was added dropwise over 30 min. The solution was allowed to slowly come to ambient temperature and was stirred under nitrogen for 24 hours. The solution was then poured over cold water (200 mL). The aqueous phase was extracted three time with DCM (50 mL). The combined organic phase was dried over anhydrous Na₂SO₄, filtered and concentrated in vacuo. The residue was purified via SiO₂ chromatography using hexane:EtOAc (75:25) as eluent to give ethyl 2-(1-(but-2-yn-1-yl)-5-((3,4-difluorophenyl)carbamoyl)-2,4-dimethyl-1H-pyrrol-3-yl)-2-oxoacetate 118 (53.7% yield) as an off white solid. ¹H NMR (CDCl₃) δ ppm 8.13 (s, 1H), 7.73 (dd, J=2.4, 7.2 Hz, 1H), 7.29-7.21 (m, 1H), 7.13 (q, J=8.8 Hz, 1H), 4.86 (d, J=2.4 Hz, 2H), 4.36 (q, J=7.2 Hz, 2H), 2.49 (s, 3H), 2.31 (s, 3H), 1.78 (t, J=2.4 Hz, 3H), 1.37 (t, J=7.2 Hz, 3H). ¹⁹F NMR (CDCl₃) δ ppm-135.55 (d, J=24 Hz, 1F), −142.14 (d, J=24 Hz, 1F). ¹³C NMR (CDCl₃) δ ppm. 183.36, 165.74, 159.87, 151.37, 151.24, 148.92, 148.78, 148.39, 148.27, 145.95, 145.82, 142.27, 134.29, 135.26, 134.20, 125.63, 123.65, 117.36, 117.18, 116.75, 115.68, 115.64, 115.62, 115.59, 109.88, 109.66, 81.68, 72.81, 62.25, 34.94, 13.92, 11.59, 11.47, 3.52.

### 1-(But-2-yn-1-yl)-N-(3,4-difluorophenyl)-3,5-dimethyl-4-(2-oxo-2-(prop-2-yn-1-ylamino)acetyl)-1H-pyrrole-2-carboxamide 119

A solution of ethyl 2-(1-(but-2-yn-1-yl)-5-((3,4-difluorophenyl)carbamoyl)-2,4-dimethyl-1H-pyrrol-3-yl)-2-oxoacetate (118) (0.730 g, 1.81 mml, 1 equiv.) in MeOH (18 mL) and THF (4 mL) was cooled to 0° C. and 5% aqueous NaOH (18 mL) was added slowly. The solution was stirred at 0° C. for 20 min. after which time the solution was acidified to pH 4-5 by the slow addition of 1N HCl. The solution was partitioned between water (100 mL) and EtOAc (50 mL). The aqueous phase was extracted two times with EtOAc (25 mL). The combined organic phase was dried over anhydrous Na₂SO₄, filtered and concentrated in vacuo. The residue was then dissolved in DMF (20 mL) and CDI (1.50 g, 9.25 mmol, 5 equiv.) was added. After stirring the solution for 30 min. under nitrogen at ambient temperature, propargyl amine (0.350 mL, 5.46 mmol, 3 equiv.) was added and the reaction was continued for 24 hours. The solution was then poured over water (150 mL) and extracted tree times with EtOAc (30 mL). The combined organic phase was dried over anhydrous Na₂SO₄, filtered and concentrated in vacuo. The residue was purified via SiO₂ chromatography using hexane:EtOAc (65:35) as eluent to give 1-(but-2-yn-1-yl)-N-(3,4-difluorophenyl)-3,5-dimethyl-4-(2-oxo-2-(prop-2-yn-1-ylamino)acetyl)-1H-pyrrole-2-carboxamide 119 (38.6% yield) as a white solid. ¹H NMR (DMSO-d⁶) δ ppm 10.53 (s, 1H), 9.19 (t, J=5.6 Hz, 1H), 7.81 (dd, J=2.4, 7.6 Hz, 1H), 7.45-7.39 (m, 2H), 4.95 (d, J=2.4 Hz, 2H), 4.01 (dd, J=2.4, 5.6 Hz, 2H), 3.2 (t, J=2.4 Hz, 1H), 2.50 (s, 3H), 2.24 (s, 3H), 1.75 (s, 3H). ¹⁹F NMR (DMSO-d⁶) δ ppm −137.14 (d, J=24 Hz, 1F), −141.19 (d, J=24 Hz, 1F). ¹³C NMR (DMSO-d⁶) δ ppm 187.92, 167.35, 160.30, 150.68, 150.55, 148.26, 148.13, 147.28, 147.15, 144.87, 144.74, 141.11, 136.36, 136.33, 136.27, 136.24, 126.22, 123.49, 118.08, 117.90, 117.15, 116.49, 116.46, 109.21, 109.00, 81.25, 80.53, 74.43, 73.90, 34.37, 28.10, 11.95, 11.73, 3.45. LC-MS (ES⁺) m/z 412.3 [M+H],

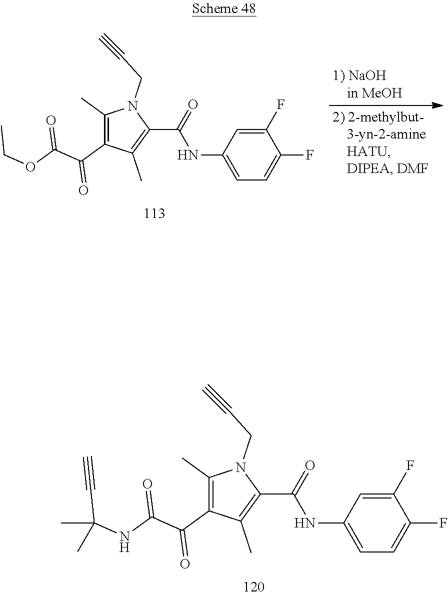 [SMILES: C#CCn1c(C)c(C(=O)C(=O)OCC)c(C)c1C(=O)Nc1ccc(F)c(F)c1 ; C#CCn1c(C)c(C(=O)C(=O)NC(C)(C)C#C)c(C)c1C(=O)Nc1ccc(F)c(F)c1]

### N-(3,4-Difluorophenyl)-3,5-dimethyl-4-(2-((2-methylbut-3-yn-2-yl)amino)-2-oxoacetyl)-1-(prop-2-yn-1-yl)-1H-pyrrole-2-carboxamide 120

A solution of ethyl 2-(5-((3,4-difluorophenyl)carbamoyl)-2,4-dimethyl-1-(prop-2-yn-1-yl)-1N-pyrrol-3-yl)-2-oxoacetate (113) (0.500 g, 1.28 mml, 1 equiv.) in MeOH (10 mL) was cooled to 0° C. and 5% aqueous NaOH (10 mL) was added slowly. The solution was stirred at 0° C. for 20 min. after which time the solution was acidified to pH 4-5 by the slow addition of 1N HCl. The solution was partitioned between water (100 mL) and EtOAc (50 mL). The aqueous phase was extracted two times with EtOAc (25 mL). The combined organic phase was dried over anhydrous Na₂SO₄, filtered and concentrated in vacuo. The residue was dissolved in DMF (12 mL) and treated with HATU (4.34 g, 11.4 mmol, 3 equiv.) and DIPEA (0.670 mL, 3.85 mmol, 3 equiv.) and stirred at ambient temperature under nitrogen. After 30 minutes, 2-methylbut-3-yn-2-amine (0.430 g, 5.17 mmol, 4 equiv.) was added and the reaction was stirred for 2 hours at this temperature. The solution was then heated to 65° C. and stirred under nitrogen for 24 hours. The solution was then poured in water (150 mL) and extracted tree times with EtOAc (30 mL). The combined organic phase was dried over anhydrous Na₂SO₄, filtered and concentrated in vacuo. The residue was purified via SiO₂ chromatography using hexane:EtOAc (65:35) as eluent to give N-(3,4-difluorophenyl)-3,5-dimethyl-4-(2-((2-methylbut-3-yn-2-yl)amino)-2-oxoacetyl)-1-(prop-2-yn-1-yl)-1H-pyrrole-2-carboxamide 120 (32.3% yield) as a white solid. ¹H NMR (DMSO-d⁶) δ ppm 10.53 (s, 1H), 8.83 (s, 1H), 7.89 (dd, J=2.4, 7.6 Hz, 1H), 7.49-7.43 (m, 2H), 5.05 (d, J=2.0 Hz, 2H), 3.41 (t, J=2.4 Hz, 1H), 3.22 (s, 3H), 2.31 (s, 3H), 1.59 (s, 6H). ¹⁹F NMR (DMSO-d⁶) δ ppm-137.13 (d, J=28 Hz, 1F), −144.14 (d, J=24 Hz, 1F). ¹³C NMR (DMSO-d⁶) δ ppm 187.96, 167.05, 160.34, 150.69, 150.56, 148.27, 148.14, 147.31, 147.18, 144.90, 144.77, 141.21, 136.35, 136.32, 136.26, 136.23, 126.07, 123.85, 118.00, 117.82, 117.28, 116.47, 109.27, 109.05, 87.28, 79.04, 76.02, 72.05, 46.84, 34.02, 29.16, 12.15, 11.75. LC-MS (ES⁺) m/z 426.3 [M+H],

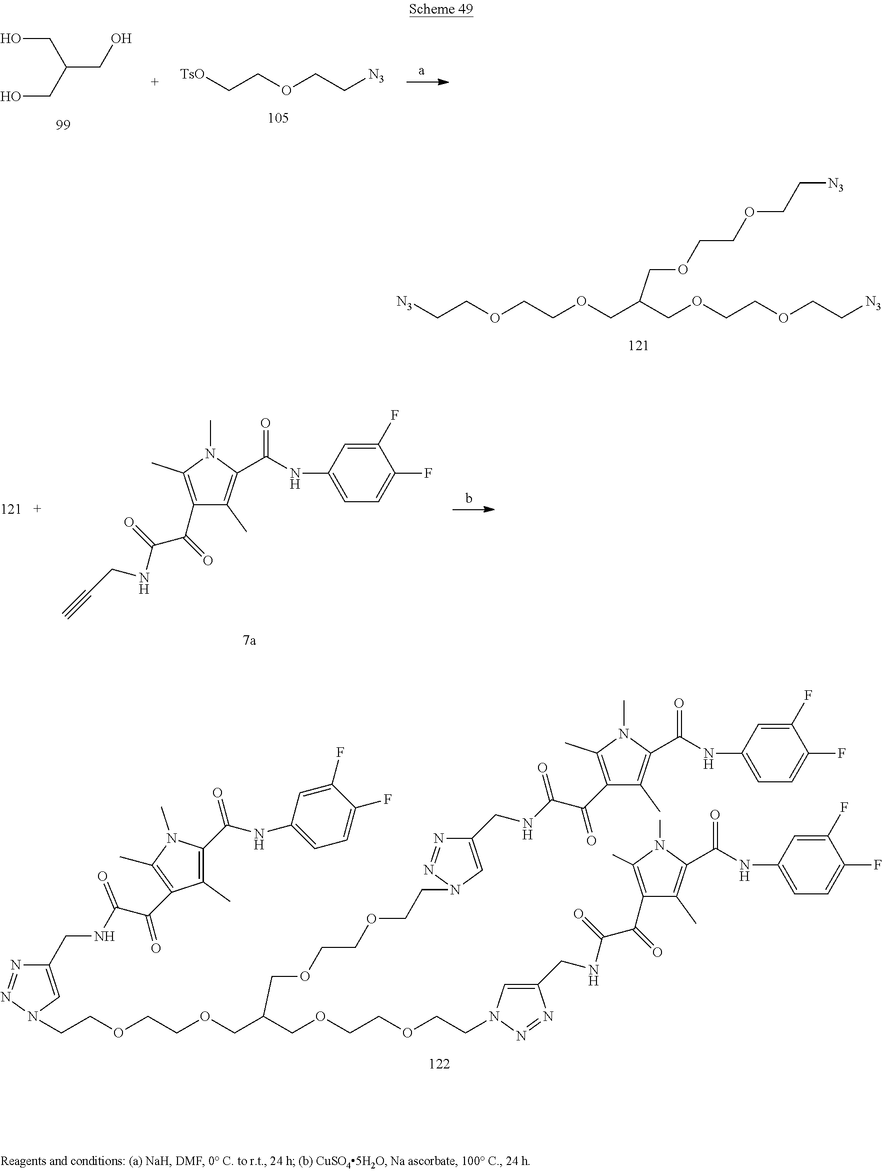 [SMILES: OCC(CO)CO ; Cc1ccc(S(=O)(=O)OCCOCCN=[N+]=[N-])cc1 ; [N-]=[N+]=NCCOCCOCC(COCCOCCN=[N+]=[N-])COCCOCCN=[N+]=[N-] ; C#CCNC(=O)C(=O)c1c(C)c(C(=O)Nc2ccc(F)c(F)c2)n(C)c1C ; Cc1c(C(=O)C(=O)NCc2cn(CCOCCOCC(COCCOCCn3cc(CNC(=O)C(=O)c4c(C)c(C(=O)Nc5ccc(F)c(F)c5)n(C)c4C)nn3)COCCOCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1]

### 1,3-Bis(2-(2-azidoethoxy)ethoxy)-2-((2-(2-azidoethoxy)ethoxy)methyl)propane (121)

Compound 99 (315 mg, 2.97 mmol, 1.0 eq) was dissolved in anhydrous DMF (30 mL) and NaH (60% in oil, 428 mg, 10.69 mmol, 3.6 eq) in powder form was added portionwise to the reaction under Argon. The reaction was stirred for 30 minutes and then compound 105 (3.39 g, 11.87 mmol, 4.0 eq) was added dropwise. The reaction was stirred at r.t. for 24 h and the mixture was quenched with cold H₂O and ice (100 mL) at 0° C. with caution. The resulting mixture was filtered and the water phase was then extracted with EtOAc (3×100 mL). The organic layers were finally combined, washed with water, brine, and dried over anhydrous Na₂SO₄, and concentrated under reduced pressure. The residue was purified by flash chromatography (DCM/Ethyl acetate 3:1 to 1:10) to afford product 121 (952 mg, 72%). ¹H NMR (400 MHz, CDCl₃) δ 3.69-3.63 (m, 12H), 3.60-3.58 (m, 6H), 3.52 (d, J=6.0 Hz, 6H), 3.38 (t, J=5.2 Hz, 6H), 2.25-2.19 (m, 1H). LCMS (ESI) m/z calcd for C₁₆H₃₂N₉O₆ [M+H]⁺: 446.5; found 446.3.

### Trimer (122)

To a mixture of compound 121 (32 mg, 0.045 mmol, 1 eq) and compound 7a (100 mg, 0.2678 mmol, 6.0 eq) in H₂O/CH₃CN (4 mL/4 mL) was added CuSO₄.5H₂O (20 mg) and Na ascorbate (40 mg) under Argon atmosphere. The resulting mixture was stirred at 100° C. for 24 h. The mixture was diluted with 30 mL of cold H₂O and extracted with ethyl acetate (3×50 mL). The organic layers were combined, washed with water, brine, and dried over anhydrous Na₂SO₄, and concentrated under reduced pressure. The residue was purified by flash chromatography (DCM/MeOH, from 100:1 to 5:1) to afford product 112 (Trimer, 36 mg, 51%)¹H NMR (400 MHz, DMSO-d₆) δ10.42 (s, 3H), 9.19 (t, J=5.6 Hz, 3H), 7.97 (s, 3H), 7.90-7.85 (m, 3H), 7.45-7.40 (m, 6H), 4.52 (t, J=5.2 Hz, 6H), 4.45 (d, J=5.6 Hz, 6H), 3.80 (t, J=5.2 Hz, 6H), 3.58 (s, 9H), 3.52-3.50 (m, 6H), 3.44-3.41 (m, 6H), 3.73-3.32 (m, 6H), 2.35 (s, 9H), 2.19 (s, 9H), 2.03-1.93 (m, 1H). ¹⁹F NMR (377 MHz, DMSO-d₆) δ−137.1 to −137.2 (m, 1F), −144.2 to −144.3 (m, 1F). HRMS (ESI) m/z calcd for C₇₃H₈₃F₆N₁₈O₁₅[M+H]⁺: 1566.5; found 1565.9.

### Example 2

Cellular Toxicity Assays

The toxicity of the compounds was assessed in Vero, human PBM, CEM (human lymphoblastoid), MT-2, and HepG2 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide was included as positive cytotoxic control, and untreated cells exposed to solvent were included as negative controls. The cytotoxicity IC₅₀ was obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11). The results are shown in Table 1 below:

TABLE 1
Cytotoxicity, CC₅₀, μM (% inhibition)

Cytotoxicity; CC₅₀ (μM)

| Compound | PBM | CEM | VERO | HepG2 |
|---|---|---|---|---|
| 8 | 62 | 20 | 49 | 36 |
| 17 | 19 | 17 | 23 | 81 |
| 28 | >100 | >100 | >100 | >100 |
| 33 | 32 | 32 | 61 | 19 |
| 17c | 11 | 7.1 | >100 | 38.5 |
| 16a | >100 | >100 | 9 | >100 |
| 20 | 33 | >100 | >100 | >100 |
| 23 | >100 | 14 | >100 | 63 |
| 9 | 26.4 | >100 | >100 | >100 |
| 45 | 75 | 24 | >100 | >100 |
| 50 | 99 | 27 | >100 | >100 |
| 53 | 35 | 45 | >100 | >100 |
| 58 | 7 | 10 | 12 | 29 |
| 60 | 36 | 4 | 14 | 63 |
| 62 | 13 | 4 | 7 | 6 |
| 64 | 62 | 26 | 76 | 98 |
| 68 | 70 | | | >100 |
| 71 | >100 | | | >100 |
| 74 | >100 | | | >100 |
| 77 | >100 | | | >100 |
| 80 | >100 | | | >100 |
| 89 | 68 | | | >100 |
| 94 | >100 | | | >100 |
| 101 | >100 | | | >100 |
| 108 | >100 | | | >100 |
| 109 | >100 | | | >100 |
| 110 | >100 | | | >100 |
| 114 | >100 | | | >100 |
| 115 | 45 | | | 35 |
| 119 | >100 | | | >100 |
| 120 | >100 | | | >100 |

### Example 3

Mitochondrial Toxicity Assays in HepG2 Cells:

i) Effect of Compounds on Cell Growth and Lactic Acid Production: The effect on the growth of HepG2 cells was determined by incubating cells in the presence of 0 μM, 0.1 μM, 1 μM, 10 μM and 100 μM drug. Cells (5×10⁴ per well) were plated into 12-well cell culture clusters in minimum essential medium with nonessential amino acids supplemented with 10% fetal bovine serum, 1% sodium pyruvate, and 1% penicillin/streptomycin and incubated for 4 days at 37° C. At the end of the incubation period the cell number was determined using a hemocytometer. Also taught by Pan-Zhou X-R, Cui L, Zhou X-J, Sommadossi J-P, Darley-Usmer V M. “Differential effects of antiretroviral nucleoside analogs on mitochondrial function in HepG2 cells,” Antimicrob. Agents Chemother. 2000; 44: 496-503.

To measure the effects of the compounds on lactic acid production, HepG2 cells from a stock culture were diluted and plated in 12-well culture plates at 2.5×10⁴ cells per well. Various concentrations (0 μM, 0.1 μM, 1 μM, 10 μM and 100 μM) of compound were added, and the cultures were incubated at 37° C. in a humidified 5% CO₂ atmosphere for 4 days. At day 4, the number of cells in each well was determined and the culture medium collected. The culture medium was then filtered, and the lactic acid content in the medium was determined using a colorimetric lactic acid assay (Sigma-Aldrich). Since lactic acid product can be considered a marker for impaired mitochondrial function, elevated levels of lactic acid production detected in cells grown in the presence of test compounds would indicate a drug-induced cytotoxic effect.

ii) Effect on Compounds on Mitochondrial DNA Synthesis: a real-time PCR assay to accurately quantify mitochondrial DNA content has been developed (see Stuyver L J, Lostia S, Adams M, Mathew J S, Pai B S, Grier J, Tharnish P M, Choi Y, Chong Y, Choo H, Chu C K, Otto M J, Schinazi R F. Antiviral activities and cellular toxicities of modified 2′,3′-dideoxy-2′,3′-didehydrocytidine analogs. Antimicrob. Agents Chemother. 2002; 46: 3854-60). This assay was used in all studies described in this application that determine the effect of compounds on mitochondrial DNA content. In this assay, low-passage-number HepG2 cells were seeded at 5,000 cells/well in collagen-coated 96-well plates. Test compounds were added to the medium to obtain final concentrations of 0 μM, 0.1 μM, 10 μM and 100 μM. On culture day 7, cellular nucleic acids were prepared by using commercially available columns (RNeasy 96 kit; Qiagen). These kits co-purify RNA and DNA, and hence, total nucleic acids are eluted from the columns. The mitochondrial cytochrome c oxidase subunit II (COXII) gene and the ß-actin or rRNA gene were amplified from 5 μl of the eluted nucleic acids using a multiplex Q-PCR protocol with suitable primers and probes for both target and reference amplifications. For COXII the following sense, probe and antisense primers were used, respectively: 5′-TGCCCGCCATCATCCTA-3′, 5′-tetrachloro-6-carboxyfluorescein-TCCTCATCGCCCTCCCATCCC-TAMRA-3′ and 5′-CGTCTGTTATGTAAAGGATGCGT-3′. For exon 3 of the ß-actin gene (GenBank accession number E01094) the sense, probe, and antisense primers are 5′-GCGCGGCTACAGCTTCA-3′, 5′-6-FAMCACCACGGCCGAGCGGGATAMRA-3′ and 5′-TCTCCTTAATGTCACGCACGAT-3′, respectively. The primers and probes for the rRNA gene are commercially available from Applied Biosystems. Since equal amplification efficiencies are obtained for all genes, the comparative CT method was used to investigate potential inhibition of mitochondrial DNA synthesis. The comparative CT method uses arithmetic formulas in which the amount of target (COXII gene) is normalized to the amount of an endogenous reference (the ß-actin or rRNA gene) and is relative to a calibrator (a control with no drug at day 7). The arithmetic formula for this approach is given by 2−ΔΔCT, where ΔΔCT is (CT for average target test sample−CT for target control)−(CT for average reference test −CT for reference control) (see Johnson M R, K Wang, J B Smith, M J Heslin, R B Diasio. Quantitation of dihydropyrimidine dehydrogenase expression by real-time reverse transcription polymerase chain reaction. Anal. Biochem. 2000; 278:175-184). A decrease in mitochondrial DNA content in cells grown in the presence of drug indicated mitochondrial toxicity.

### Example 4

Mitochondrial Toxicity Assays in Neuro2A Cells

To estimate the potential of the compounds of this invention to cause neuronal toxicity, mouse Neuro2A cells (American Type Culture Collection 131) can be used as a model system (see Ray A S, Hernandez-Santiago B I, Mathew J S, Murakami E, Bozeman C, Xie M Y, Dutschman G E, Gullen E, Yang Z, Hurwitz S, Cheng Y C, Chu C K, McClure H, Schinazi R F, Anderson K S. Mechanism of anti-human immunodeficiency virus activity of beta-D-6-cyclopropylamino-2′,3′-didehydro-2′,3′-dideoxyguanosine. Antimicrob. Agents Chemother. 2005, 49, 1994-2001). The concentrations necessary to inhibit cell growth by 50% (CC₅₀) can be measured using the 3-(4,5-dimethyl-thiazol-2-yl)-2,5-diphenyltetrazolium bromide dye-based assay, as described. Perturbations in cellular lactic acid and mitochondrial DNA levels at defined concentrations of drug can be performed as described above. ddC and AZT can be used as control nucleoside analogs.

### Example 5

Assay for Bone Marrow Cytotoxicity Primary human bone marrow mononuclear cells can be obtained commercially from Cambrex Bioscience (Walkersville, Md.). CFU-GM assays is carried out using a bilayer soft agar in the presence of 50 units/mL human recombinant granulocyte/macrophage colony-stimulating factor, while BFU-E assays used a ethylcellulose matrix containing 1 unit/mL erythropoietin (see Sommadossi J P, Carlisle R. Toxicity of 3′-azido-3′-deoxythymidine and 9-(1,3-dihydroxy-2-propoxymethyl) guanine for normal human hepatopoietic progenitor cells in vitro. Antimicrob. Agents Chemother. 1987; 31: 452-454; Sommadossi, J P, Schinazi, R F, Chu, C K, and Xie, M Y. Comparison of cytotoxicity of the (−) and (+) enantiomer of 2′,3′-dideoxy-3′-thiacytidine in normal human bone marrow progenitor cells. Biochem. Pharmacol. 1992; 44:1921-1925). Each experiment can be performed in duplicate in cells from three different donors. AZT is used as a positive control. Cells can be incubated in the presence of the compound for 14-18 days at 37° C. with 5% CO₂, and colonies of greater than 50 cells can be counted using an inverted microscope to determine the IC₅₀. The 50% inhibitory concentration (IC₅₀) can be obtained by least-squares linear regression analysis of the logarithm of drug concentration versus BFU-E survival fractions. Statistical analysis can be performed with Student's t test for independent non-paired samples.

### Example 6

Anti-HBV Assay

The anti-HBV activity of the compounds was determined by treating the AD-38 cell line carrying wild type HBV under the control of tetracycline (see Ladner S. K., Otto M. J., Barker C. S., Zaifert K., Wang G. H., Guo J. T., Seeger C. & King R. W. Antimicrob. Agents Chemother. 1997, 41, 1715-20). Removal of tetracycline from the medium [Tet (−)] results in the production of HBV. The levels of HBV in the culture supernatant fluids from cells treated with the compounds were compared with that of the untreated controls. Control cultures with tetracycline [Tet (+)] were also maintained to determine the basal levels of HBV expression. 3TC was included as positive control.

The median effective concentrations (EC₅₀) ranges of several of the compounds described herein against HBV are shown in Table 3:

TABLE 2
Anti-HBV activity

| | EC₅₀ | EC₉₀ |
|---|---|---|
| 8 | B | B |
| 17 | C | B |
| 28 | >A | |
| 33 | B | >A |
| 17c | A | A |
| 16a | B | >A |
| 20 | C | A |
| 23 | C | B |
| 9 | B | A |
| 45 | C | B |
| 50 | B | A |
| 53 | E | C |
| 58 | B | A |
| 60 | C | B |
| 62 | C | C |
| 64 | C | C |
| 68 | B | |
| 71 | B | A |
| 74 | E | D |
| 77 | D | B |
| 80 | D | |
| 85 | >A | |
| 89 | A | |
| 94 | D | |
| 95 | C | |
| 101 | C | |
| 108 | C | B |
| 109 | B | A |
| 110 | B | A |
| 114 | E | |
| 115 | E | |
| 119 | C | |
| 120 | F | D |
| 122 | >B | |
| 3TC | B | A |

A = 1-10 μM
B = 0.1-1 μM
C = 0.01-0.1 μM
D = 0.001-0.01 μM
E = 0.0001-0.001 μM
F = 0.00001-0.0001 μM

### Example 7

Production of secreted HBeAg is predominantly cccDNA-dependent in HepAD38 cells and therefore can serve as a surrogate marker for cccDNA (Ladner, S. K., Otto, M. J., Barker, C. S., Zaifert, K., Wang, G. H., Guo, J. T., Seeger, C., King, R. W. Antimicrob Agents Chemother 1997, 41, 1715-1720; Zhou T, Guo H, Guo J T, Cuconati A, Mehta A, Block™. Antiviral Res. 2006; 72 (2): 116-24.). The effect on the levels of cccDNA formation was assessed using a cell-based assay that measures HBV e antigen (HBeAg) as a cccDNA-dependent marker in the HepAD38 system. HepAD38 cells were seeded at 50,000 cells/well in collagen-coated 96-well plates with DMEM/F12 medium (Life Technologies) supplemented with 10% heat-inactivated fetal bovine serum. Cells were treated with 0.3 μg/ml tetracycline as needed. Test compounds and controls were added to cells to a final concentration of 10 μM or in a dose response manner ranging from 0.001 to 10 μM. Medium and test compounds were replenished every 5 days in culture. Supernatants were harvested at day-14, clarified by centrifugation at 5000 rpm for 5 min, and stored at −70° C. until use. ELISA—Culture medium was diluted 1:15 in DMEM/F12 and the levels of HBeAg secreted in the culture medium were measured by using HBeAg ELISA kit (BioChain Institute Inc. Hayward, Calif.) according to the manufacturer's protocol. The concentration of compound that reduced levels of secreted HBeAg by 50% (EC₅₀) was determined by linear regression.

### Example 8

TABLE 3
Computer-simulated combination index (CI) of drug-drug combinations at
50, 75, 90, and 95% inhibition of HBV replication in HepAD38 cells.

| | | | Combination index | | | | CI | |
|---|---|---|---|---|---|---|---|---|
| | EC₅₀, | EC₉₀, | (CI) at inhibition of | | | | value, | |
| Compound | nM | nM | 50% | 75% | 90% | 95% | WT | Summary |
| ETV | 0.04 | 12.9 | — | — | — | — | — | — |
| 53 | 0.02 | 5.29 | — | — | — | — | — | — |
| ETV + 53 | 0.01 | 0.99 | 0.9 | 0.5 | 0.3 | 0.2 | 0.3 | Synergism |

### Combinations at Ratio 1:1

Combination index (CI) values were determined for a mutually exclusive interaction using CalcuSyn program; CI<1, =1 or >1 indicates synergism, additive effect, and antagonism, respectively. **CI_{WT}, weighted average CI value was assigned as CI₅₀+2CI₇₅+3CI₉₀, +4CI₉₅]/10 (Chou T C, 2006).

### Example 9

Antiviral Activity in HepG2-NTCP Infectious System: HepG2-NTCP (Clone 7) cells were pretreated with antivirals for 30 min prior infection for 24 h with stock concentrated virus produced from HepAD38 at a dose of ˜300 virus genome equivalent (VGE) per cell in the presence of media containing 4% polyethylene glycol 8000 (PEG) and 2% DMSO. After 24 h, virus inoculum with or without antiviral agents were washed out from cells and media in the presence of 4% PEG and 2% DMSO, with or without antiviral agents were replenished every two days until day 11. Cells and supernatants were harvested and tested for intracellular HBV DNA, pregenomic (pg) RNA, surface RNA, and secreted HBeAg (ELISA).

| | | | | HBV RNA | |
|---|---|---|---|---|---|
| | HBeAg | HBV DNA/rDNA | | EC₅₀, nM | |
| Cpd | EC₅₀, nM | EC₅₀ (nM) | CC₅₀ (nM) | pgRNA | sRNA |
| 114 | 487.9 | 0.6 | >1000 | 79.6 | 67.8 |
| Entecavir | >1000 | <1 | >1000 | >1000 | >1000 |
| Myrcludex | 6.5 | 7.8 | >1000 | 4.3 | 5.1 |

### Example 10

Glutathione levels (GSH) were measured in Huh7 cells after 22 h treatment with 10 μM CAMs (GSH-Glo™ Glutathione Assay—Promega). Treatment of Huh7 with 10 μM positive control, L-buthionine-sulfoximine, reduced cellular GSH levels by 58% (data not shown); Samples were tested in triplicates.

| | GSH levels |
|---|---|
| | relative to untreated control |
| Cpd | (% inhibition at 10 μM) |
| 114 | >10 μM (27) |
| 115 | >10 μM (<1) |

Various publications are cited herein, the disclosures of which are incorporated by reference in their entireties for all purposes.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

We claim:

1. A compound of the formula: 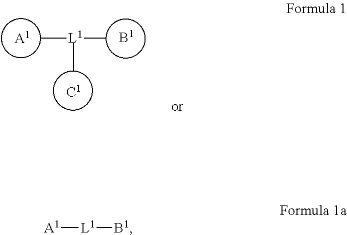
wherein:
A¹, B¹ and C¹ are, independently, an HBV capsid assembly effector,
each A¹, and B¹, and C¹ may be the same or different;
L¹ is a chemical linker selected from the group consisting of —W¹—(C₃₋₆ alkyl-O)ₛ—C₁₋₆ alkyl-W¹—; —W¹—(CH₂—CH₂—O)ₜ—C₁₋₆ alkyl-W¹—; —W¹—((CH₂—CH₂—O)ₛ)ₘ—; W¹—C₁₋₃₀ alkyl-W¹—; W¹—(C₃₋₆ alkyl-O)ₛ—C₁₋₆ alkyl-X—(C₃₋₆ alkyl-O)ₛ—C₁₋₆ alkyl-W¹; —W¹(CH₂—CH₂—O)ₜ—C₁₋₆ alkyl-X—(CH₂—CH₂—O)ₜ—C₁₋₆ alkyl-W¹—C₁₋₃₀ alkyl-X—C₁₋₃₀ alkyl-W¹—; 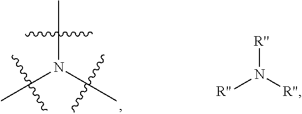
NR′—; —N(R′)C(O)—; —C(O)N(R′); —N(R′)SO₂—; —SO₂N(R′); —O—; —C(O)—; —OC(O)—; —C(O)O—; —S—; —S(O)—; —SO₂—; —C(═S)—; —C(═NR′); —Si(R⁷)(R⁸)—, —Si(R⁷)(R⁸)—O—, —O—Si(R⁷)(R⁸)—, —NR′—; —N(R′)C(O)—; —C(O)N(R′)—; —N(R′)SO₂—; —SO₂N(R′); —O—; —C(O)—; —OC(O)—; —C(O)O—; —S—; —S(O)—; —SO₂—; —C(═S)—; —C(═NR′)—; an optionally substituted unsaturated or partially saturated aliphatic moiety, an optionally substituted unsaturated or partially saturated cycloalkyl moiety; an optionally substituted unsaturated or partially saturated heteroaliphatic moiety; an optionally substituted unsaturated or partially saturated heterocycloalkyl moiety; optionally substituted aryl, optionally substituted heteroaryl, and a covalently bonded combination thereof;
wherein X is Si(R⁷)(R⁸)—, —Si(R⁷)(R⁸)—O—, —O—Si(R⁷)(R⁸)—, —NR′—; —N(R′)C(O)—; —C(O)N(R′)—; —N(R′)SO₂—; —SO₂N(R′); —O—; —C(O)—; —OC(O)—; —C(O)O—; —S—; —S(O)—; —SO₂—; —C(═S)—; or —C(═NR′)—;
wherein R″ is, independently, H, alkylene, arylene, —C(O)—, —(C₃₋₆ alkyl-O)ₛ—C₁₋₆ alkyl-, —(CH₂—CH₂—O)ₛ—; C(O)N(R′)—; —SO₂N(R′)—; —C(O)—; —OC(O)—; —C(O)O—; —S(O)—; —SO₂—; —C(═S)—; —C(═NR′)—; or an optionally substituted unsaturated or partially saturated aliphatic moiety or an optionally substituted unsaturated or partially saturated heteroaliphatic moiety; wherein only one R″ can be H,
wherein the optional substituents for the heteroaliphatic, aliphatic, aryl, and heteroaryl moieties are selected from the group consisting of halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, phosphonyl, phosphinyl, phosphoryl, phosphine, thioester, thioether, oxime, hydrazine, carbamate, silyl ether, boronic ester, boronic acid, phosphonic acid, phosphonamidate, and phosphonate,
W¹ is, independently for each occurrence, aryl, heteroaryl, CHR′, NR′, N, O or S;
s and m are integers from 1 to 15;
t is an integer from 3 to 15;
R′ is H, C₁₋₆ alkyl, optionally unsaturated C₃₋₈ cycloalkyl, C₁₋₆ haloalkyl, C₂₋₈ alkoxyalkyl, C₂₋₆ alkenyl, C₂₋₆ alkynyl, aryl, heteroaryl, alkylaryl, or arylalkyl,
R⁷ and R⁸ are, independently, —OH, optionally unsaturated —O—C₁₋₆ alkyl, C₁₋₆ alkyl, C₂₋₆ alkenyl, C₂₋₆ alkynyl, optionally unsaturated C₃₋₈ cycloalkyl, aryl, heteroaryl, alkylaryl, or arylalkyl, and
alternatively, R⁷ and R⁸, together with the silicon to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, cyano, oxo and OH;
or an enantiomer, diasteroisomer, tautomer, pharmaceutically acceptable salt or prodrug thereof.

2. A compound of the following formula: 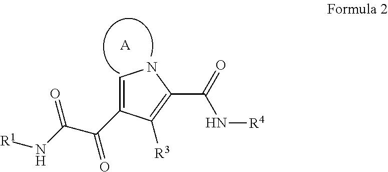
or an enantiomer, a diasteroisomer, a tautomer, a pharmaceutically acceptable salt or prodrug thereof,
wherein:
R¹ is C₂₋₆ alkenyl optionally substituted with one or more R^{a} or C₂₋₆ alkynyl optionally substituted with one or more R^{a},
R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —OR′, —C(O)OR′, oxo, —SF₅, 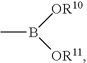
N(R′)S(O)₂R′, —S—R′; S(O)₂R′, S(O)₂N(R′)₂, 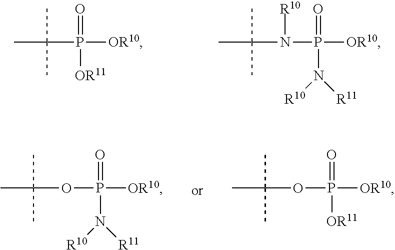
R^{b} is independently C₁₋₆ alkyl optionally substituted with cyano N₃, —O—R′, —C(O)O—R′, oxo, —SF₅, 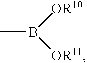
N(R′)S(O)₂R′, —S—R′, S(O)₂R′, S(O)₂N(R′)₂, 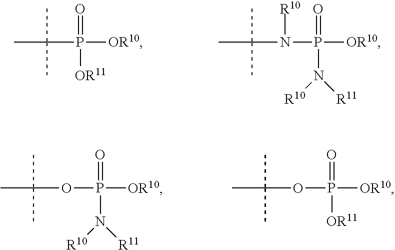
or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;
R^{d} is, independently, cyclopropanyl, alkylamino carbonyl, amino carbonyl, alkyl carbonyl, cyclopropanyloxy, halocyclopropanyl, or halocyclopropanyloxy;
A is a pyrrolidine or a 5-7 membered bicyclic heterocycle having one nitrogen, optionally substituted with one or more R²;
R² is, independently, hydrogen, halogen, OH, C₁₋₆ alkyl, C₂₋₆ alkene, C₂₋₆ alkyne, haloalkyl, cycloalkyl, CN, amino, alkoxy, alkylsulfonylamino, azido or cycloalkyloxyl;
R³ is hydrogen, halogen, C₁₋₆ alkyl, haloalkyl, alkyloxyl, haloalkyloxyl or cycloalkyl,
R⁴ is an C₆₋₁₀ aryl optionally substituted with 1 to 5 R^{4a} or a 5-12 membered heteroaryl having one or more heteroatoms selected from the group consisting of N, O, and S, optionally substituted with one or more R^{4b};
R^{4a} and R^{4b} are, independently, H, CN, halogen, —O—C₁₋₆ alkyl, C₁₋₆ alkyl, C₂₋₆ alkenyl, C₂₋₆ alkynyl, C₃₋₆ cycloalkyl, alkylamino carbonyl, amino carbonyl, alkyl carbonyl, cyclopropanyloxy, halocyclopropanyl, or halocyclopropanyloxy;
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms; and
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

3. A compound of the following formula: 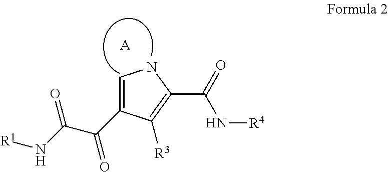
or an enantiomer, a diasteroisomer, a tautomer, a pharmaceutically acceptable salt or prodrug thereof,
wherein:
R¹ is C₁₋₆ alkyl substituted with one or more R^{a}, C₃₋₆ cycloalkyl substituted with one or more R^{b}, or monocyclic or bicyclic heterocyclyl containing one or more heteroatoms selected from the group consisting of N, O, and S, optionally substituted with one or more R^{c};
R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —OR′, —C(O)O—R′, oxo, —SF₅, 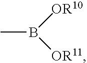
N(R′)S(O)₂R′, —S—R′; S(O)₂R′, S(O)₂N(R′)₂, 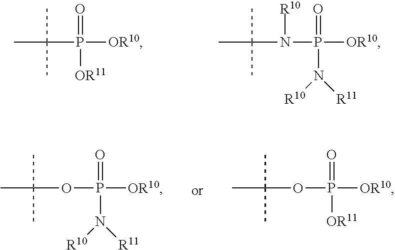
R^{b} is independently C₁₋₆ alkyl optionally substituted with cyano, N₃, —O—R′, —C(O)O—R′, oxo, —SF₅, 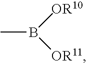
N(R′)S(O)₂R′, —S—R′, S(O)₂R′, S(O)₂N(R′)₂, 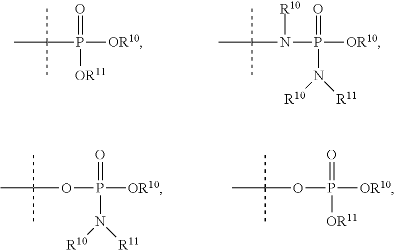
or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;
R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅, 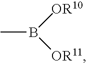
N(R′)S(O)₂R′, —S—R′, S(O)₂R′, S(O)₂N(R′)₂, 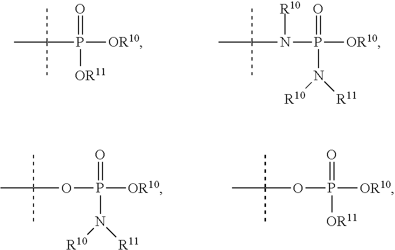
or —C(O)N(R′)₂,
R^{d} is, independently, cyclopropanyl, alkylamino carbonyl, amino carbonyl, alkyl carbonyl, cyclopropanyloxy, halocyclopropanyl, or halocyclopropanyloxy;
A is a pyrrolidine or a 5-7 membered bicyclic heterocycle having one nitrogen, optionally substituted with one or more R²;
R² is, independently, hydrogen, halogen, OH, C₁₋₆ alkyl, C₂₋₆ alkene, C₂₋₆ alkyne, haloalkyl, cycloalkyl, CN, amino, alkoxy, alkylsulfonylamino, azido or cycloalkyloxyl;
R³ is hydrogen, halogen, C₁₋₆ alkyl, haloalkyl, alkyloxyl, haloalkyloxyl or cycloalkyl,
R⁴ is an C₆₋₁₀ aryl optionally substituted with 1 to 5 R^{4a} or a 5-12 membered heteroaryl having one or more heteroatoms selected from the group consisting of N, O, and S, optionally substituted with one or more R^{4b};
R^{4a} and R^{4b} are, independently, H, CN, halogen, —O—C₁₋₆ alkyl, C₁₋₆ alkyl, C₂₋₆ alkenyl, C₂₋₆ alkynyl, C₃₋₆ cycloalkyl, alkylamino carbonyl, amino carbonyl, alkyl carbonyl, cyclopropanyloxy, halocyclopropanyl, or halocyclopropanyloxy;
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms; and
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
wherein at least one of R², R′, R^{a}, or R^{c} is C₂₋₆ alkenyl or C₂₋₆ alkynyl, or
wherein when R¹ is C₁₋₆ alkyl, R^{a} is not OR¹, or
wherein when R¹ is cycloalkyl, R^{b} is not unsubstituted C₁₋₆ alkyl or unsubstituted heteroaryl.

4. The compound of claim 3, wherein R³ is haloalkyl, alkyloxyl, haloalkyloxyl, or cycloalkyl.

5. The compound of claim 3, wherein R¹ is C₁₋₆ alkyl, and
R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —C(O)O—R′, oxo, —SF₅, 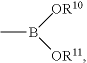
N(R′)S(O)₂R′, —S—R′; S(O)₂R′, S(O)₂N(R′)₂, 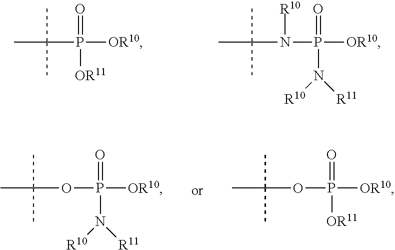
R^{b} is independently C₁₋₆ alkyl optionally substituted with cyano, N₃, —O—R′, —C(O)O—R′, oxo, —SF₅, 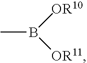
N(R′)S(O)₂R′, —S—R′, S(O)₂R′, S(O)₂N(R′)₂, 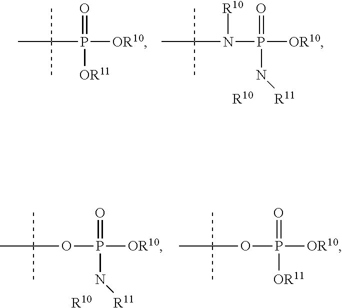
or C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl.

6. The compound of claim 3, wherein R¹ is cycloalkyl, and
R^{b} is C₁₋₆ alkyl substituted with cyano, N₃, —O—R′, —C(O)O—R′, oxo, —SF₅, 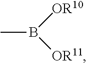
N(R′)S(O)₂R′, —S—R′, S(O)₂R′, S(O)₂N(R′)₂, 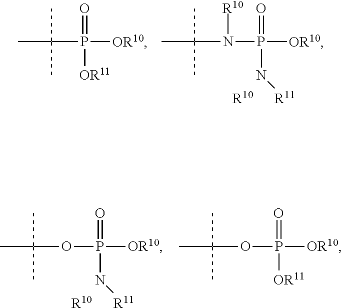
or C₅₋₈ heteroaryl containing one or more heteroatoms selected from the group consisting of N, O, and S, substituted with one or more R^{d}, cycloalkylalkynyl, cycloalkyloxyl, or halocycloalkylalkyl.

7. The compound of claim 3, wherein at least one of R², R′, R^{a}, or R^{c} is C₂₋₆ alkenyl or C₂₋₆ alkynyl

8. A compound of the following formula: 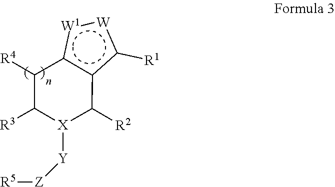
or an enantiomer, a diasteroisomer, a tautomer, a pharmaceutically acceptable salt or prodrug thereof, wherein:
W¹ and W are each independently selected from the group consisting of N, NR^{m}, CR^{m}, O and S, with the proviso that when one of W and W¹ is O or S, the other is CR^{m}; and wherein when one of W¹ or W is N, the other is NR^{m},
X is N or CRⁿ
Y is a bond, —C(O)— or —SO₂—;
Z is NH or —(CR⁶R⁷)ₘ—NH—;
n is 0, 1, 2 or 3,
m is 0, 1, 2, 3 or 4,
R^{m} and Rⁿ are, independently, H, C₁₋₆ alkyl, C₂₋₆ alkynyl, or C₂₋₆ alkenyl;
R¹ is C₂₋₆ alkynyl, optionally substituted with one or more R^{z},
R^{z} is C₁₋₆ alkyl, C₁₋₆ haloalkyl, C₁₋₆ alkyl-OH, aryl, heteroaryl, C₃₋₆ cycloalkyl, C₃₋₆ halocycloalkyl, C₃₋₆ heterocyclyl, CN, —C(O)R′, —C(O)O—R′, 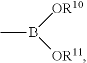
N(R′)S(O)₂R′, —S—R′; S(O)₂R′, S(O)₂N(R′)₂, 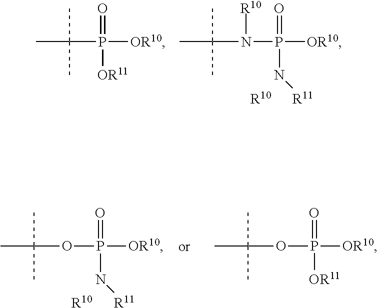
R², R³ and R⁴ are independently selected from the group consisting of H, OH, halo, C₁₋₆ alkyl, C₁₋₆ haloalkyl, O—C₁₋₆ alkyl, —O—C₁₋₆ haloalkyl, C₂₋₆ alkynyl, C₂₋₆ alkenyl, C₃₋₆ cycloalkyl, C₃₋₆ halocycloalkyl, C₃₋₆ heterocyclyl, —CN, O—C₃₋₆ cycloalkyl, and O—C₃₋₆ halocycloalkyl;
alternatively, R² and R³ can come together to form a 5-7 heterocyclic ring containing 1 to 3 heteroatoms selected from the group consisting of N, O and S,
alternatively, R² and R⁴ can come together to form a C₃₋₆ cycloalkyl ring or a 3-7 heterocyclic ring containing 1 to 3 heteroatoms selected from the group consisting of N, O and S,
R⁵ is C₁₋₆ alkyl, optionally substituted with one or more R^{a}, C₂₋₆ alkenyl, optionally substituted with one or more R^{a}, C₂₋₆ alkynyl, optionally substituted with one or more R^{a}, C₃₋₆ cycloalkyl optionally substituted with one or more R^{b}, or monocyclic or bicyclic heterocyclyl containing one or more heteroatoms selected from the group consisting of N, O, and S, optionally substituted with one or more R^{c};
R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;
R⁷ is selected from the group consisting of H, C₁-C₆ alkyl, and C₁-C₆ alkyl-OH:
R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —O—R′, —C(O)O—R′, oxo, —SF₅, 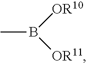
N(R′)S(O)₂R′, —S—R′; S(O)₂R′, S(O)₂N(R′)₂, 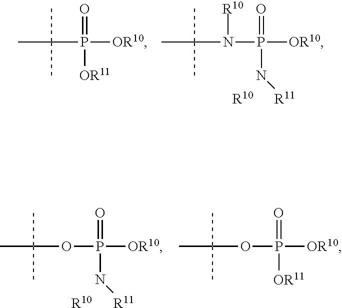
R^{b} is independently C₁₋₆ alkyl optionally substituted with cyano N₃, —O—R′, —C(O)O—R′, oxo, —SF₅, 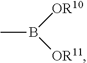
N(R′)S(O)₂R′, —S—R′, S(O)₂R′, S(O)₂N(R′)₂; 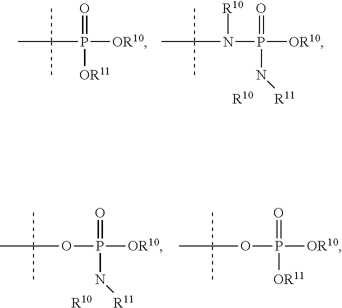
C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;
R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅, 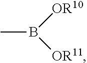
N(R′)S(O)₂R′, —S—R′, S(O)₂R′, S(O)₂N(R′)₂, 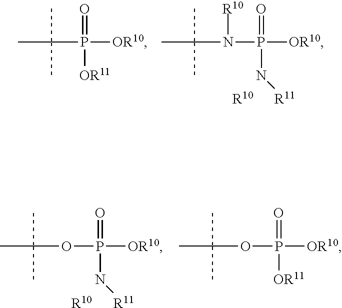
—C(O)N(R′)₂,
R^{d} is independently cyclopropanyl, alkylamino carbonyl, amino carbonyl, alkyl carbonyl, cyclopropanyloxy, halocyclopropanyl, halocyclopropanyloxy;
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;
alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

9. A compound of the following formula: 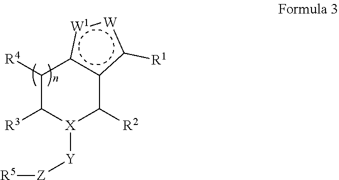
or an enantiomer, a diasteroisomer, a tautomer, a pharmaceutically acceptable salt or prodrug thereof, wherein:
W¹ and W are each independently selected from the group consisting of N, NR^{m}, CR^{m}, O and S, with the proviso that when one of W and W¹ is O or S, the other is CR^{m}; and wherein when one of W¹ or W is N, the other is NR^{m},
X is N or CRⁿ
Y is a bond, —C(O)— or —SO₂—;
Z is NH or —(CR⁶R⁷)ₘ—NH—;
n is 0, 1, 2 or 3,
m is 0, 1, 2, 3 or 4,
R^{m} and Rⁿ are, independently, H, C₁₋₆ alkyl, C₂₋₆ alkynyl, or C₂₋₆ alkenyl;
R¹ is C₂₋₆ alkenyl substituted with one or more R^{y}, C₃₋₈-cycloalkyl substituted with one or more R^{y}, C₃₋₆ heterocyclyl substituted with one or more R^{y}, or C₁₋₆ alkyl substituted with one or more R^{y},
R^{y} is C₂₋₆ alkynyl, C₂₋₆ alkenyl, aryl, heteroaryl, C₃₋₆ cycloalkyl, C₃₋₆halocycloalkyl, C₃₋₆ heterocyclyl, —CN, —C(O)R′, —C(O)O—R′, 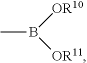
—N(R′)S(O)₂R′, —SR′; —S(O)₂R′, —S(O)₂N(R′)₂, 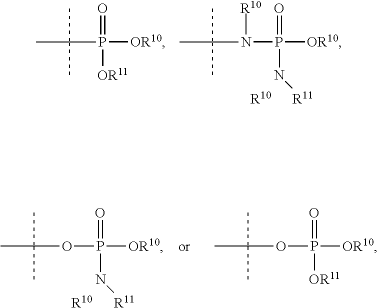
R², R³ and R⁴ are independently selected from the group consisting of H, OH, halo, C₁₋₆ alkyl, C₁₋₆haloalkyl, O—C₁₋₆ alkyl, —O—C₁₋₆haloalkyl, C₂₋₆alkynyl, C₂₋₆alkenyl, C₃₋₆cycloalkyl, C₃₋₆ halocycloalkyl, C₃₋₆ heterocyclyl, —CN, O—C₃₋₆ cycloalkyl, and O—C₃₋₆halocycloalkyl;
alternatively, R² and R³ can come together to form a 5-7 heterocyclic ring containing 1 to 3 heteroatoms selected from the group consisting of N, O and S,
alternatively, R² and R⁴ can come together to form a C₃₋₆ cycloalkyl ring or a 3-7 heterocyclic ring containing 1 to 3 heteroatoms selected from the group consisting of N, O and S,
R⁵ is C₁₋₆ alkyl, optionally substituted with one or more R^{a}, C₂₋₆ alkenyl, optionally substituted with one or more R^{a}, C₂₋₆ alkynyl, optionally substituted with one or more R^{a}, C₃₋₆ cycloalkyl optionally substituted with one or more R^{b}, or monocyclic or bicyclic heterocyclyl containing one or more heteroatoms selected from the group consisting of N, O, and S, optionally substituted with one or more R^{c};
R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;
R⁷ is selected from the group consisting of H, C₁-C₆-alkyl, and C₁-C₆-alkyl-OH;
R^{a} is C₁₋₆ alkyl optionally substituted with one or more R′, C₂₋₆ alkenyl optionally substituted with one or more R′, C₂₋₆ alkynyl optionally substituted with one or more R′, C₃₋₆ cycloalkyl optionally substituted with one or more R^{b}, N₃, —O—R′, —C(O)O—R′, oxo, —SF₅, 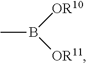
N(R′)S(O)₂R′, —S—R′; S(O)₂R′, S(O)₂N(R′)₂, 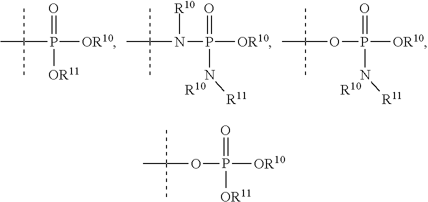
R^{b} is independently C₁₋₆ alkyl optionally substituted with cyano N₃, —O—R′, —C(O)O—R′, oxo, —SF₅, 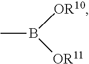
N(R′)S(O)₂R′, —S—R′, S(O)₂R′, S(O)₂N(R′)₂, 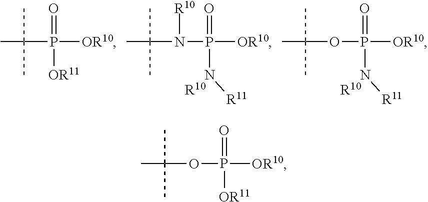
C₅₋₈ heteroaryl containing one or more heteroatoms selected from the group consisting of N, O, and S, optionally substituted with one or more R^{d}, cycloalkylalkynyl, cycloalkyloxyl, or halocycloalkylalkyl;
R^{c} is, independently, C₂₋₆ alkenyl optionally substituted with one or more R^{a}, C₂₋₆ alkynyl optionally substituted with one or more R^{a}, C₃₋₆ cycloalkyl optionally substituted with one or more R^{b}, N₃, —O—R′, —SF₅, 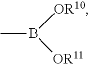
N(R′)S(O)₂R′, —S—R′, S(O)₂R′, S(O)₂N(R′)₂, 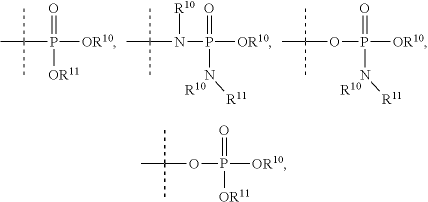
—C(O)N(R′)₂,
R^{d} is independently cyclopropanyl, alkylamino carbonyl, amino carbonyl, alkyl carbonyl, cyclopropanyloxy, halocyclopropanyl, halocyclopropanyloxy;
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;
alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

10. A compound of the following formula: 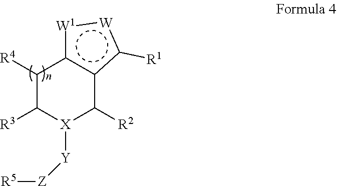
or an enantiomer, a diasteroisomer, a tautomer, a pharmaceutically acceptable salt or prodrug thereof, wherein:
W¹ and W are each, independently, N, NR^{m}, CR^{m}, O or S; with the proviso that when one of W and W¹ is O or S, the other is CR^{m}, and wherein when one of W¹ or W is N, the other is NR^{m},
X is NR^{m} or CRⁿ,
Y is a bond, —C(O)— or —SO₂—;
Z is NH or —(CR⁶R⁷)ₘ—NH—;
n is 0, 1, 2 or 3,
m is 0, 1, 2, 3 or 4,
R^{m} and Rⁿ are, independently, H, C₁₋₆ alkyl, C₂₋₆ alkynyl, or C₂₋₆ alkenyl;
R², R³ and R⁴ are, independently, —O—C₁₋₆ haloalkyl, C₂₋₆ alkynyl, C₂₋₆ alkenyl, C₃₋₆ cycloalkyl, C₃₋₆ halocycloalkyl, C₃₋₆ heterocyclyl, —CN, —O—C₃₋₆ cycloalkyl, or —O—C₃₋₆ halocycloalkyl;
alternatively, R² and R³ can come together to form a 5-7 heterocyclic ring containing 1 to 3 heteroatoms selected from the group consisting of N, O and S;
alternatively, R² and R⁴ can come together to form a C₃₋₆ cycloalkyl ring or a 3-7 heterocyclic ring containing 1 to 3 heteroatoms selected from the group consisting of N, O and S;
R¹ is C₃₋₆ cycloalkyl, C₃₋₆ heterocyclyl, C₁₋₆ alkyl, halo, C₂₋₆ alkynyl optionally substituted with one or more R^{z}, C₂₋₆ alkenyl optionally substituted with one or more R^{y}, C₃₋₈-cycloalkyl optionally substituted with one or more R^{y}, or C₃₋₆ heterocyclyl, optionally substituted with one or more R^{y}, or C₁₋₆ alkyl optionally substituted with one or more R^{y},
R^{z} is C₁₋₆ alkyl, C₁₋₆ haloalkyl, C₁₋₆ alkyl-OH, C₂₋₆ alkynyl, C₂₋₆ alkenyl, aryl, heteroaryl, C₃₋₆ cycloalkyl, C₃₋₆halocycloalkyl, C₃₋₆ heterocyclyl, —CN, —C(O)R′, —C(O)O—R′, 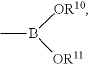
—N(R′)S(O)₂R′, —S—R′; —S(O)₂R′, —S(O)₂N(R′)₂, 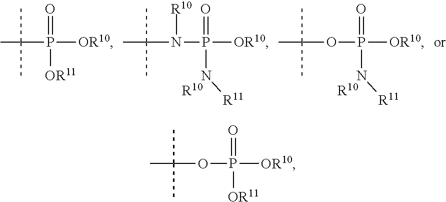
R^{y} is C₁₋₆ alkyl, C₁₋₆haloalkyl, C₁₋₆ alkyl-OH, C₂₋₆ alkynyl, C₂₋₆ alkenyl, aryl, heteroaryl, C₃₋₆ cycloalkyl, C₃₋₆halocycloalkyl, C₃₋₆ heterocyclyl, —CN, —C(O)R′, —C(O)O—R′, 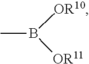
—N(R′)S(O)₂R′, —SR′; —S(O)₂R′, —S(O)₂N(R′)₂, 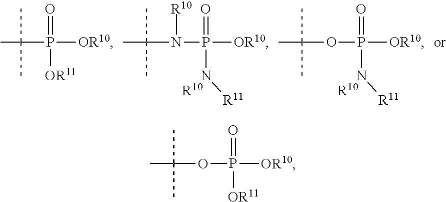
R⁵ is C₁₋₆ alkyl, optionally substituted with one or more R^{a}, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{b}, or a monocyclic or bicyclic heterocyclyl containing one or more heteroatoms selected from the group consisting of N, O, and S, optionally substituted with one or more R^{c};
R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;
R⁷ is selected from the group consisting of H, C₁-C₆-alkyl, and C₁-C₆ alkyl-OH:
R^{a} is C₁₋₆ alkyl, optionally substituted with one or more R′, C₂₋₆ alkenyl, optionally substituted with one or more R′, C₂₋₆ alkynyl, optionally substituted with one or more R′, or C₃₋₆ cycloalkyl, optionally substituted with one or more R^{b}, —N₃, —OR′, —C(O)O—R′, oxo, —SF₅, 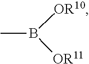
N(R′)S(O)₂R′, —S—R′; S(O)₂R′, S(O)₂N(R′)₂, 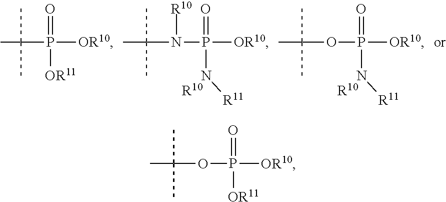
R^{b} is independently C₁₋₆ alkyl, optionally substituted with cyano, —N₃, —OR′, —R′, oxo, —SF₅, 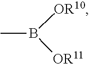
—N(R′)S(O)₂R′, —SR′, —S(O)₂R′, —S(O)₂N(R′)₂, 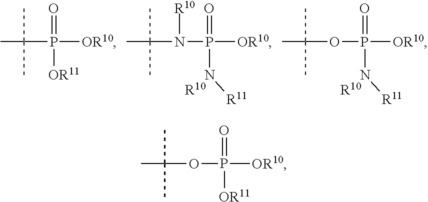
or C₅₋₈ heteroaryl containing one or more heteroatoms selected from the group consisting of N, O, and S, optionally substituted with one or more R^{d}, cycloalkylalkynyl, cycloalkyloxyl, halocycloalkylalkyl;
R^{c} is, independently, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{b}, —N₃, —OR′, —SF₅, 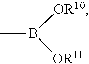
—N(R′)S(O)₂R′, —SR′, S(O)₂R′, —S(O)₂N(R′)₂, 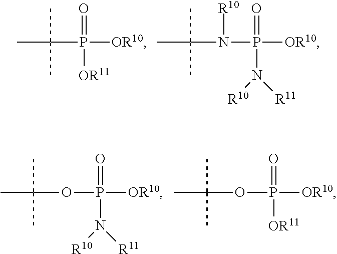
or —C(O)N(R′)₂,
R^{d} is, independently, cyclopropanyl, alkylamino carbonyl, amino carbonyl, alkyl carbonyl, cyclopropanyloxy, halocyclopropanyl, or halocyclopropanyloxy;
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate.

11. A compound of claim 10, wherein at least one of R′, R^{a}, R^{c}, R^{m}, Rⁿ, R^{y}, R^{z}, R¹, R², R³, R⁴, or R⁵ is C₂₋₆ alkynyl.

12. A compound of the following formula: 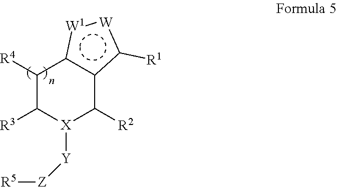
or an enantiomer, a diasteroisomer, a tautomer, a pharmaceutically acceptable salt or prodrug thereof,
W¹ and W are each independently N, NR^{m}, CR^{m}, O or S, with the proviso that when one of W and W¹ is O or S, the other is CR^{m}, and with the proviso that when one of W and W¹ is O or S, the other is CR^{m}, and wherein when one of W¹ or W is N, the other is NR^{m},
X is N or CRⁿ
Y is a bond, —C(O)—, or —SO₂—;
Z is NH or —(CR⁶R⁷)ₘ—NH—;
n is 0, 1, 2 or 3,
m is 0, 1, 2, 3 or 4
R^{m} and Rⁿ are, independently, H, C₁₋₆ alkyl, C₂₋₆ alkynyl, or C₂₋₆ alkenyl;
R⁵ is C₁₋₄ alkyl, optionally substituted with one or more R^{b}, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{a}, or monocyclic or bicyclic heterocyclyl containing one or more heteroatoms selected from the group consisting of N, O, and S, optionally substituted with one or more R^{c};
R⁶ is, at each occurrence, independently selected from the group consisting of H, —OH, halo, C₁-C₆-alkyl, C₁-C₆-haloalkyl, —O—C₁-C₆-alkyl, and C₁-C₆-alkyl-OH;
R⁷ is selected from the group consisting of H, C₁-C₆-alkyl, and C₁-C₆ alkyl-OH:
R^{a} is C₁₋₆ alkyl, optionally substituted with one or more R′, C₂₋₆ alkenyl, optionally substituted with one or more R′, C₂₋₆ alkynyl, optionally substituted with one or more R′, C₃₋₆ cycloalkyl, optionally substituted with one or more R′, N₃, —O—R′, —C(O)O—R′, oxo, —SF₅, 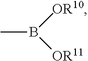
N(R′)S(O)₂R′, —S—R′; S(O)₂R′, S(O)₂N(R′)₂, 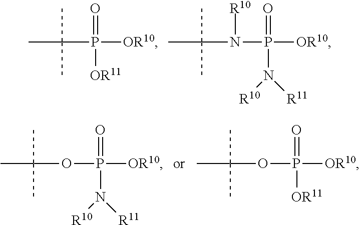
R^{b} is, independently, C₁₋₆ alkyl, optionally substituted with one or more R^{a}, —N₃, —OR′, —C(O)O—R′, oxo, —SF₅, 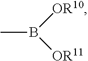
—N(R′)S(O)₂R′, —SR′, —S(O)₂R′, —S(O)₂N(R′)₂; 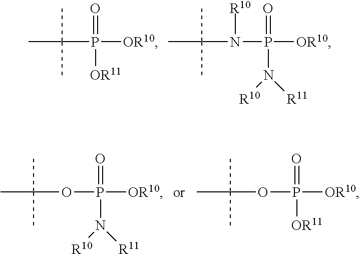
R^{c} is, independently, C₂₋₆ alkenyl, optionally substituted with one or more R^{a}, C₂₋₆ alkynyl, optionally substituted with one or more R^{a}, C₃₋₆ cycloalkyl, optionally substituted with one or more R^{b}, —N₃, —OR′, —SF₅, 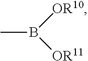
—N(R′)S(O)₂R′, —SR′, S(O)₂R′, —S(O)₂N(R′)₂, 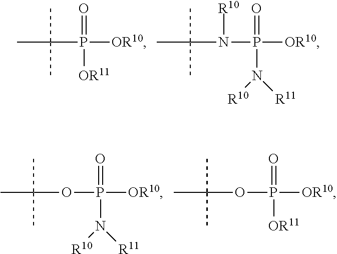
or —C(O)N(R′)₂,
R^{d} is, independently, cyclopropanyl, alkylamino carbonyl, amino carbonyl, alkyl carbonyl, cyclopropanyloxy, halocyclopropanyl, or halocyclopropanyloxy;
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S;
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatoms which are, independently N, O, or S;
alternatively, R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate;
R¹ is C₁₋₆ alkyl, halo, C₂₋₆ alkynyl optionally substituted with one or more R^{z}, C₂₋₆ alkenyl, optionally substituted with one or more R^{y}, C₃₋₈ cycloalkyl, optionally substituted with one or more R^{y}, C₃₋₆ heterocyclyl, optionally substituted with one or more R^{y}, C₁₋₆ alkyl optionally substituted with one or more R^{y},
R^{z} is C₁₋₆ alkyl, C₁₋₆ haloalkyl, C₁₋₆ hydroxyalkyl, C₂₋₆ alkynyl, C₂₋₆ alkenyl, aryl, heteroaryl, C₃₋₆ cycloalkyl, C₃₋₆halocycloalkyl, C₃₋₆ heterocyclyl, —CN, —C(O)R′, —C(O)O—R′, 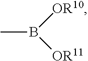
N(R′)S(O)₂R′, —S—R′; S(O)₂R′, S(O)₂N(R′)₂, 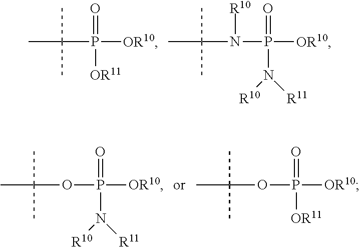
R^{y} is C₁₋₆ alkyl, C₁₋₆ haloalkyl, C₁₋₆ hydroxyalkyl, C₂₋₆ alkynyl, C₂₋₆ alkenyl, aryl, heteroaryl, C₃₋₆ cycloalkyl, C₃₋₆ halocycloalkyl, C₃₋₆ heterocyclyl, CN, —C(O)R′, —C(O)O—R′, 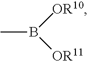
—N(R′)S(O)₂R′, —SR′; S(O)₂R′, —S(O)₂N(R′)₂, 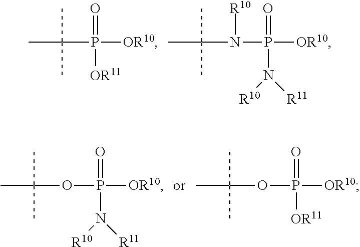
R², R³ and R⁴ are, independently, H, OH, halo, C₁₋₆ alkyl, C₁₋₆ haloalkyl, —O—C₁₋₆ alkyl, C₁₋₆ alkyl-OH, —O—C₁₋₆haloalkyl, C₂₋₆alkynyl, C₂₋₆alkenyl, C₃₋₆cycloalkyl, C₃₋₆halocycloalkyl, C₃₋₆ heterocyclyl, —CN, —O—C₃₋₆ cycloalkyl, or —O—C₃₋₆ halocycloalkyl;
alternatively, R² and R³ can come together to form a 5-7 heterocyclic ring containing 1 to 3 heteroatoms selected from the group consisting of N, O and S; and
alternatively, R² and R⁴ can come together to form a C₃₋₆ cycloalkyl ring or a 3-7 heterocyclic ring containing 1 to 3 heteroatoms selected from the group consisting of N, O and S,
wherein at least one of R′, R^{a}, R^{c}, R^{m}, Rⁿ, R^{y}, R^{z}, R¹, R², R³, R⁴, or R⁵ is C₂₋₆ alkynyl.

13. A compound of the following formula: 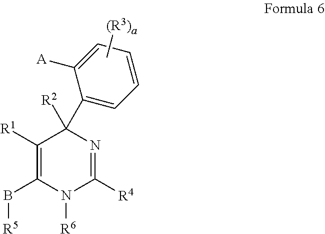
or an enantiomer, diasteroisomer, tautomer, pharmaceutically acceptable salt or prodrug thereof,
wherein:
A is H, CN, OH, C₁₋₃ alkyl, C₁₋₃ haloalkyl, C₁₋₃ hydroxyalkyl, C₁₋₃ thioalkyl or halogen;
R¹ is 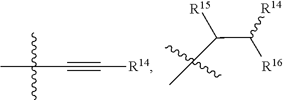
or an optionally substituted C₁₋₆ alkyl;
R² is hydrogen, deuterium or C₁₋₃ alkyl optionally substituted with one or more fluoro atoms;
or R² and A, together with the carbons to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, oxo and OH;
each R³ is, independently, H, —CN, —OH, C₁₋₃ alkyl, C₁₋₃ haloalkyl, C₁₋₃ hydroxy alkyl, C₁₋₃ thioalkyl or halogen;
R⁶ is H, C₁₋₃ alkyl, or C₁₋₃ haloalkyl,
B is —(CR⁷R^{7a})ₙ— or —C(O)—, where n is 0, 1, 2 or 3,
each a is independently 0, 1, 2, 3 or 4;
each R⁷ and R^{7a} is independently H, C₁₋₆ alkyl or C₁₋₆ haloalkyl;
alternatively, R⁷ and R^{7a} can come together with the carbon to which they are attached to form a 6-10 membered bicyclic or bridged ring, a 3 to 8 saturated ring, or a 5 membered unsaturated ring; such bicyclic, bridged, saturated and unsaturated rings optionally containing one or more additional heteroatoms, where each is, independently, O, S or N, and optionally being substituted with one or more substituents, wherein each, independently, is halogen (including F, Cl, Br, I), —CF₃, hydroxy, —N(R′)S(O)₂R′, —S(O)₂R′, —S(O)₂N(R′)₂, C₁₋₆ alkoxy, cyano, azido, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆haloalkyl, heterocyclylalkyl, or C₁₋₆ hydroxyalkyl,
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
R⁴ is an optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
wherein the substituents on the optionally substituted C₁₋₆ alkyl are, independently, halo, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate;
R⁵ is H, —O—R¹⁰, 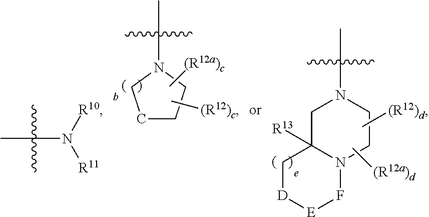
R¹⁰ and R¹¹ are, independently, H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatoms which are, independently, N, O, or S; —COOR′, —(C═O)—R′, —S(O)₂R′, —S(O)₂N(R′)₂, C(═O)NR′₂; —S(═O)₂—NR′₂; or P(═O)(OR′)₂;
R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
R¹² and R^{12a} are, independently, H, F, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(CO)—R¹⁰, —N(R′)S(O)₂R′, —S(O)₂R′, —S(O)₂N(R′)₂, —C(═O)NR¹⁰R¹¹; —NR′—S(O)₂—R¹⁰; —NR′—S(O)₂—NR¹⁰R¹¹; or —NR¹⁰—P(O)(OR¹⁰)₂,
alternatively, R¹² and R^{12a} can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
if R¹² and R^{12a} are on the same carbon, they can come together to form, with the carbon to which they are attached, —C═O;
R¹² and R^{12a} groups can optionally be substituted with one or more substituents, which substituents are, independently, halogen, C₁₋₆ haloalkyl, C₁₋₆ hydroxy alkyl, hydroxyl, carboxyl, acyl, aryl, acyloxy, amino, amido, azido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
b is 0, 1, 2 or 3,
c is independently 0, 1, 2, 3, 4 or 5,
C is O, S, NR¹⁰, —C(O), or —C(R¹²R^{12a})—,
d is 0, 1, 2 or 3,
F is sulfonyl or —C(R¹²R^{12a})—,
D is —C(R¹²R^{12a})— or —O—,
E is N—R¹⁰, —C═O, or —C(R¹²R^{12a})—,
e is 0 or 1,
R¹⁴, R¹⁵ and R¹⁶ are, independently, H, optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(C═O)—R¹⁰, —N(R′)S(O)₂R′, —S(O)₂R′, —S(O)₂N(R′)₂, —C(═O)NR¹⁰R¹¹; —NR′—S(═O)₂—R¹⁰; —NR′—S(═O)₂—NR¹⁰R¹¹; —P(═O)(OR¹⁰)₂, or —NR¹⁰—P(═O)(OR¹⁰)₂,
wherein the optional substituents for the C₁₋₆ alkyl are selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,
R¹⁴, R¹⁵ and R¹⁶ can optionally, and independently, be substituted with one or more substituents selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,
alternatively, R¹⁴ and R¹⁶ can come together to form, with the carbon to which they are attached, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring,
wherein R³ is C₁₋₃ alkyl, C₁₋₃ haloalkyl, C₁₋₃ hydroxy alkyl, or C₁₋₃ thioalkyl, or
wherein R⁴ is C₁₋₆ alkyl, C₁₋₆ haloalkyl, C₂₋₈ alkoxyalkyl, C₂₋₆ alkenyl, or C₂₋₆ alkynyl.

14. The compound of claim 13, wherein R⁴ is C₁₋₆ alkyl, C₁₋₆ haloalkyl, C₂₋₈ alkoxyalkyl, C₂₋₆ alkenyl, or C₂₋₆ alkynyl.

15. The compound of claim 14, wherein R⁴ is C₂₋₆ alkynyl.

16. The compound of claim 14, wherein R³ is C₁₋₃ alkyl, C₁₋₃ haloalkyl, C₁₋₃ hydroxyalkyl, or C₁₋₃ thioalkyl.

17. A compound of the following formula: 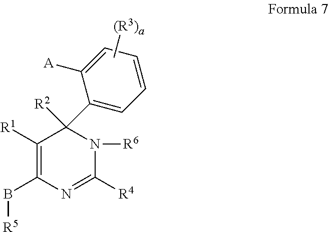
or an enantiomer, diasteroisomer, tautomer, pharmaceutically acceptable salt or prodrug thereof,
wherein:
A is H, CN, OH, C₁₋₃ alkyl, C₁₋₃ haloalkyl, C₁₋₃ hydroxyalkyl, C₁₋₃ thioalkyl or halogen;
R¹ is 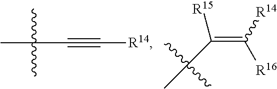
or an optionally substituted C₁₋₆ alkyl;
R² is hydrogen, deuterium or C₁₋₃ alkyl optionally substituted with one or more fluoro atoms;
or R² and A, together with the carbons to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, oxo and OH;
each R³ is, independently, H, —CN, —OH, C₁₋₃ alkyl, C₁₋₃ haloalkyl, C₁₋₃ hydroxy alkyl, C₁₋₃ thioalkyl or halogen;
R⁶ is H, C₁₋₃ alkyl, or C₁₋₃ haloalkyl,
B is —(CR⁷R^{7a})ₙ— or —C(O)—, where n is 0, 1, 2 or 3,
each a is independently 0, 1, 2, 3 or 4;
each R⁷ and R^{7a} is independently H, C₁₋₆ alkyl or C₁₋₆ haloalkyl;
alternatively, R⁷ and R^{7a} can come together with the carbon to which they are attached to form a 6-10 membered bicyclic or bridged ring, a 3 to 8 saturated ring, or a 5 membered unsaturated ring; such bicyclic, bridged, saturated and unsaturated rings optionally containing one or more additional heteroatoms, where each is, independently, O, S or N, and optionally being substituted with one or more substituents, wherein each, independently, is halogen (including F, Cl, Br, I), —CF₃, hydroxy, —N(R′)S(O)₂R′, —S(O)₂R′, —S(O)₂N(R′)₂, C₁₋₆ alkoxy, cyano, azido, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, or C₁₋₆ hydroxyalkyl,
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
R⁴ is an optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
wherein the substituents on the optionally substituted C₁₋₆ alkyl are, independently, halo, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate;
R⁵ is H, —O—R¹⁰, 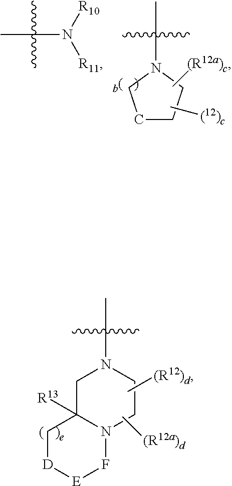
R¹⁰ and R¹¹ are, independently, H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatoms which are, independently, N, O, or S; —COOR′, —(C═O)—R′, —S(O)₂R′, —S(O)₂N(R′)₂, C(═O)NR′₂; —S(═O)₂—NR′₂; or P(═O)(OR′)₂;
R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
R¹² and R^{12a} are, independently, H, F, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(CO)—R¹⁰, —N(R′)S(O)₂R′, —S(O)₂R′, —S(O)₂N(R′)₂, —C(═O)NR¹⁰R¹¹; —NR′—S(O)₂—R¹⁰; —NR′—S(O)₂—NR¹⁰R¹¹; or —NR¹⁰—P(O)(OR¹⁰)₂, alternatively, R¹² and R^{12a} can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
if R¹² and R^{12a} are on the same carbon, they can come together to form, with the carbon to which they are attached, —C═O;
R¹² and R^{12a} groups can optionally be substituted with one or more substituents, which substituents are, independently, halogen, C₁₋₆ haloalkyl, C₁₋₆ hydroxy alkyl, hydroxyl, carboxyl, acyl, aryl, acyloxy, amino, amido, azido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
b is 0, 1, 2 or 3,
c is independently 0, 1, 2, 3, 4 or 5,
C is O, S, NR¹⁰, —C(O), or —C(R¹²R^{12a})—,
d is 0, 1, 2 or 3,
F is sulfonyl or —C(R¹²R^{12a})—,
D is —C(R¹²R^{12a})— or —O—,
E is N—R¹⁰, —C═O, or —C(R¹²R^{12a})—,
e is 0 or 1,
R¹⁴, R¹⁵ and R¹⁶ are, independently, H, optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(C═O)—R¹⁰, —N(R′)S(O)₂R′, —S(O)₂R′, —S(O)₂N(R′)₂, —C(═O)NR¹⁰R¹¹; —NR′—S(═O)₂—R¹⁰; —NR′—S(═O)₂—NR¹⁰R¹¹; —P(═O)(OR¹⁰)₂, or —NR¹⁰P—(═O)(OR¹⁰)₂,
wherein the optional substituents for the C₁₋₆ alkyl are selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,
R¹⁴, R¹⁵ and R¹⁶ can optionally, and independently, be substituted with one or more substituents selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,
alternatively, R¹⁴ and R¹⁶ can come together to form, with the carbon to which they are attached, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring.

18. A compound of the following formula: 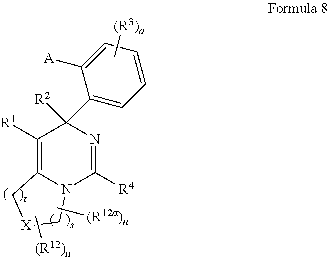
or an enantiomer, a diasteroisomer, a tautomer, a pharmaceutically acceptable salt or prodrug thereof,
wherein:
A is H, CN, OH, C₁₋₃ alkyl, C₁₋₃ haloalkyl, C₁₋₃ hydroxyalkyl, C₁₋₃ thioalkyl or halogen;
R¹ is 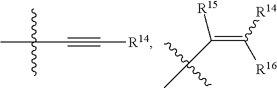
or an optionally substituted C₁₋₆ alkyl;
wherein the substituents on the optionally substituted C₁₋₆ alkyl are selected from the group consisting of halogen, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,
R² is hydrogen, deuterium or C₁₋₃ alkyl optionally substituted with one or more fluoro atoms;
or R² and A, together with the carbons to which they are attached, form a 4-7 membered ring, optionally containing one or more heteroatoms, where each is, independently, O, S or N, wherein the 4-7 membered ring is optionally substituted with one or more substituent selected from the group consisting of C₁-C₃ alkyl, halogen, oxo and OH;
each R³ is, independently, H, CN, OH, C₁₋₃ alkyl, C₁₋₃ haloalkyl, C₁₋₃ hydroxyalkyl, C₁₋₃ thioalkyl or halogen;
each a is independently 0, 1, 2, 3 or 4;
R⁴ is an optionally substituted C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
wherein the substituents on the optionally substituted C₁₋₆ alkyl are selected from the group consisting of halogen, C₁₋₆ haloalkyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, and phosphonate,
R¹² and R^{12a} are, independently, H, F, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatom which are, independently, N, O, or S; —COOR¹⁰, —(C═O)—R¹⁰, —N(R′)S(O)₂R′, —S(O)₂R′, —S(O)₂N(R′)₂, —C(═O)NR¹⁰R¹¹; —NR′—S(═O)₂—R¹⁰; —NR′—S(═O)₂—NR¹⁰R¹¹; or —NR¹⁰—P(═O)(OR¹⁰)₂,
R¹⁰ and R¹¹ are, independently, H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; a three membered ring containing zero or one heteroatoms which are, independently, N, O, or S; —COOR′, —(CO)—R′, —S(O)₂R′, —S(O)₂N(R′)₂, C(═O)NR₂; —S(O)₂—NR′₂; or P(O)(OR′)₂
R¹⁰ and R¹¹ can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
R¹⁰ and R¹¹ groups can optionally be substituted with one or more substituents, which substituents are, independently, halo, C₁₋₆ haloalkyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
alternatively, R¹² and R^{12a} can come together to form a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms;
if R¹² and R^{12a} are on the same carbon, they can come together to form, with the carbon to which they are attached, —C═O;
R¹² and R^{12a} groups can optionally be substituted with one or more substituents, which substituents are, independently, halogen, C₁₋₆ haloalkyl, C₁₋₆ hydroxy alkyl, hydroxyl, carboxyl, acyl, aryl, acyloxy, amino, amido, azido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, or phosphonate,
R′ is H, C₁₋₆ alkyl, C₁₋₆ haloalkyl, C₂₋₈ alkoxyalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatoms which are, independently, N, O, or S,
s is 0, 1, 2, or 3,
t is 1, 2, 3, or 4,
X is —C(R¹²R^{12a})—, —C(═O)N(R¹⁰)—, —N(R¹⁰)—, —C(═NR¹⁰)—, —S(═O)N(R¹⁰)—, —O—, —S—, —C(═O)O—, —C(═O)—; —C(═S)—; —S(═O)—; or —S(═O)₂—;
u is independently 0, 1, 2, 3, 4, 5, 6, or 7.

19. A compound of the following formula: 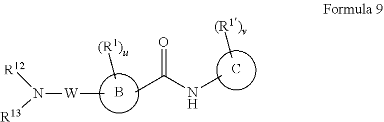
or a pharmaceutically acceptable salt or prodrug thereof, wherein
when R¹ and R^{1′} are attached to a carbon they are, independently, hydrogen, halogen, CF₃, hydroxy, SF₅, N(R′)S(O)₂R′, S(O)₂R′, S(O)₂N(R′)₂, C₁₋₆ alkoxy, cyano, C₂₋₆ alkynyl, C₃₋₆ alkoxyalkyl, alkoxycarbonyl, alkoxycarbonylalkyl, C₁₋₆ alkyl, C₁₋₆ haloalkyl, arylalkoxycarbonyl, carboxy, C₁₋₆ haloalkyl, heterocyclylalkyl, or C₁₋₆ hydroxyalkyl;
when R¹ and R^{1′} are attached to a nitrogen they are, independently, hydrogen, C₁₋₆ alkoxy, C₃₋₆ alkoxyalkyl, alkoxycarbonyl, carbonylalkyl, carbonyl aryl, C₁₋₆ alkyl, C₂₋₆ alkynyl, C₂₋₆ alkenyl, heterocyclylalkyl, C₁₋₆ hydroxyalkyl, or S(O)₂R′;
each R′ is independently H, C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, or if two R′ reside on the same nitrogen atom they can come together to form a C₃₋₆ alkyl ring optionally containing a N, O, or S; wherein the R′ groups can be substituted with one or more substituents as defined above, for example, C₁₋₆ hydroxyalkyl, aminoalkyl, and alkoxyalkyl;
u and v are independently 0, 1, 2, 3, 4 or 5;
C is phenyl, a six-membered heteroaromatic ring containing one, two, or three nitrogen atoms, a five-membered heteroaromatic ring containing one, two, or three heteroatoms, which are, independently, N, O, or S, a C₄₋₁₄ bicyclic ring; alkylheteroaryl, or alkylaryl;
B is selected from the group consisting of five-membered heteroaromatic rings containing one, two, or three heteroatoms, which are, independently, N, O, or S, six or seven-membered rings or six or seven-membered bridged or spiro-fused rings containing zero, one, or two heteroatoms which are, independently, N, O, or S, five-membered rings containing zero, one, or two heteroatoms which are, independently, N, O, or S; four-membered rings containing zero, one, or two heteroatoms which are, independently, N, O, or S,
W is 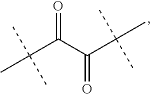
R¹² is H, C₁₋₆ alkyl, C₃₋₇ cycloalkyl, C₁₋₆ haloalkyl, C₂₋₆ alkenyl, or C₂₋₆ alkynyl,
R¹³ is C₇₋₂₀ alkyl, C₇₋₂₀ alkenyl, C₇₋₂₀ alkynyl, 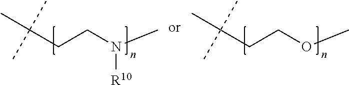
n is 1, 2, 3, 4, 5, 6, 7 or 8,
R¹³ is optionally substituted with one or more substituents each independently selected from the group consisting of hydrogen, halogen, CF₃, SF₅, hydroxy, 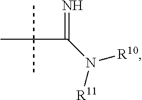
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 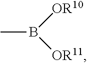
—S—R¹⁰; 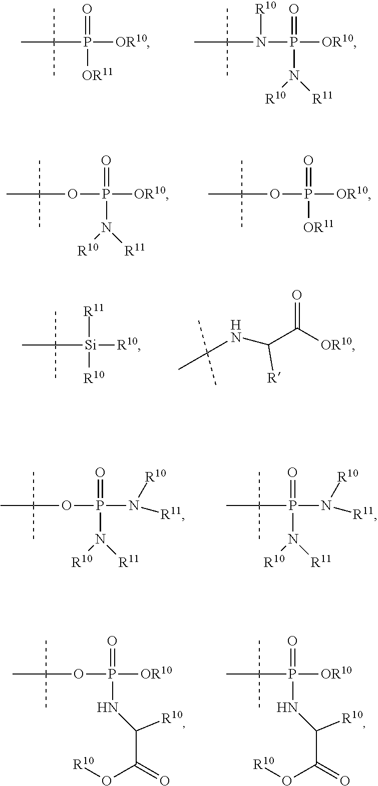
N(R¹⁰)S(O)₂R¹⁰, S(O)₂R¹⁰, S(O)₂N(R¹⁰)₂, C(O)R¹⁰, a saturated or unsaturated five or six or seven-membered ring or a six or seven-membered bridged or spiro-fused ring containing one, or two heteroatoms which are, independently, N, O, S, Se or Si, C₁₋₆ alkoxy, C₁₋₆ haloalkoxy, cyano, azido, C₂₋₆ alkynyl, C₃₋₆ alkoxyalkyl, alkoxy carbonyl, alkoxy carbonylalkyl, C₁₋₆ alkyl, cycloalkyl, arylalkoxy carbonyl, carboxyl, haloalkyl, heterocyclylalkyl, C₁₋₆ hydroxyalkyl, aryl, substituted aryl, heteroaryl, or substituted heteroaryl,
where substituents on the substituted aryl and substituted heteroaryl are selected from the group consisting of halogen, SF₅, CF₃, hydroxy, 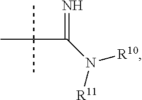
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 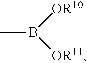
—S—R¹⁰; 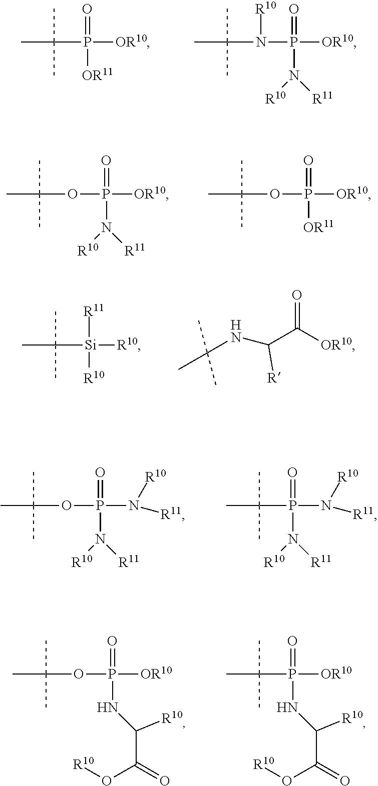
N(R¹⁰)S(O)₂R¹⁰, S(O)₂R¹⁰, S(O)₂N(R¹⁰)₂, C(O)R¹⁰, a saturated or unsaturated five or six or seven-membered ring or a six or seven-membered bridged or spiro-fused ring containing one, or two heteroatoms which are, independently, N, O, S, Se or Si, C₃-C₇ cycloalkyl, C₁₋₆ alkoxy, cyano, azido, C₂₋₆ alkynyl, C₃₋₆ alkoxyalkyl, alkoxycarbonyl, alkoxycarbonylalkyl, and C₁₋₆ alkyl;
or R¹² and R¹³, together with the nitrogen to which they are attached, form a saturated or unsaturated 4 to 7 membered ring optionally substituted with one or more substituents each independently selected from the group consisting of hydrogen, halogen, CF₃, hydroxy, 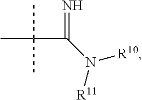
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 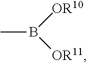
—S—R¹⁰; 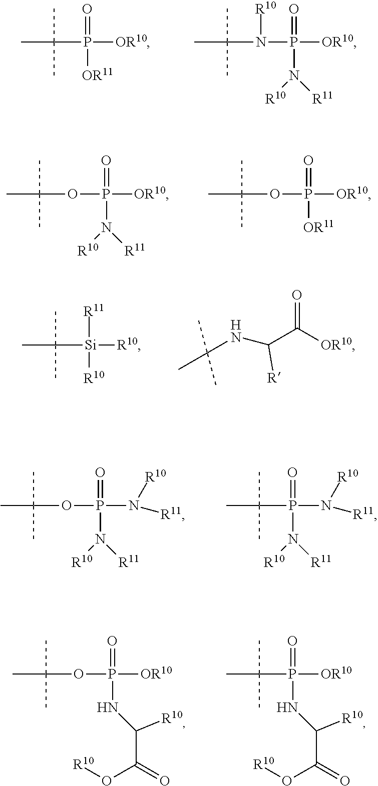
N(R¹⁰)S(O)₂R¹⁰, S(O)₂R¹⁰, S(O)₂N(R¹⁰)₂, C(O)R¹⁰, C₁₋₆ alkoxy, cyano, azido, C₂₋₆ alkynyl, C₃₋₆ alkoxyalkyl, alkoxycarbonyl, alkoxycarbonylalkyl, C₁₋₆ alkyl, arylalkoxycarbonyl, carboxy, C₁₋₆ haloalkyl, heterocyclylalkyl, and C₁₋₆ hydroxy alkyl;
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
alternatively, R¹⁰ and R¹¹ can come together to form a saturated or partially unsaturated 5 to 8 membered ring or a six-membered bridged or spiro-fused ring containing zero, one, or two heteroatoms which are, independently, N, O, or S, a seven-membered bridged or spiro-fused a saturated or partially unsaturated ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms, a saturated or partially unsaturated 6 to 12 membered hetero-bicyclic ring; a saturated or partially unsaturated 6 to 12 membered hetero-tricyclic ring; and
R¹⁰ and R¹¹ groups can optionally be substituted with one or more substituents, which substituents are, independently, halo, C₁₋₆ alkyl, C₁₋₆ haloalkyl, C₂₋₆ alkenyl, C₂₋₆ alkynyl, C₁₋₆ hydroxyalkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphoramidate or phosphonate.

20. A compound of the following formula: 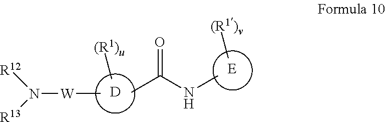
or a pharmaceutically acceptable salt or prodrug thereof, wherein:
when R¹ and R^{1′} are attached to a carbon they are, independently, hydrogen, halogen, CF₃, hydroxy, SF₅, N(R′)S(O)₂R′, S(O)₂R′, S(O)₂N(R′)₂, C₁₋₆ alkoxy, cyano, C₂₋₆ alkynyl, C₃₋₆ alkoxyalkyl, alkoxycarbonyl, alkoxycarbonylalkyl, C₁₋₆ alkyl, C₁₋₆ haloalkyl, arylalkoxycarbonyl, carboxy, C₁₋₆ haloalkyl, heterocyclylalkyl, or C₁₋₆ hydroxyalkyl;
when R¹ and R^{1′} are attached to a nitrogen they are, independently, hydrogen, C₁₋₆ alkoxy, C₃₋₆ alkoxyalkyl, alkoxycarbonyl, carbonylalkyl, carbonyl aryl, C₁₋₆ alkyl, C₂₋₆ alkynyl, C₂₋₆ alkenyl, heterocyclylalkyl, C₁₋₆ hydroxyalkyl, or S(O)₂R′;
each R′ is independently H, C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, or if two R′ reside on the same nitrogen atom they can come together to form a C₃₋₆ alkyl ring optionally containing a N, O, or S; wherein the R′ groups can be substituted with one or more substituents as defined above, for example, C₁₋₆ hydroxyalkyl, aminoalkyl, and alkoxyalkyl;
u and v are, independently, 0, 1, 2, 3, 4 or 5;
E is phenyl, a six-membered heteroaromatic ring containing one, two, or three nitrogen atoms, a five-membered heteroaromatic ring containing one, two, or three heteroatoms, which are, independently, N, O, or S, a C₄₋₁₄ bicyclic ring; alkylheteroaryl, or alkylaryl;
D is selected from the group consisting of a five-membered heteroaromatic ring containing one, two, or three heteroatoms, which are, independently, N, O, or S, a six or seven-membered ring or a six or seven-membered bridged or spiro-fused ring containing zero, one, or two heteroatoms which are, independently, N, O, or S, a five-membered ring containing zero, one, or two heteroatoms which are, independently, N, O, or S; and a four-membered ring containing zero, one, or two heteroatoms which are, independently, N, O, or S,
W is 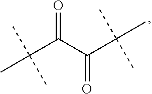
R¹² is H, C₁₋₆ alkyl, C₁₋₆ haloalkyl, C₂₋₆ alkenyl, or C₂₋₆ alkynyl,
R¹³ is C₁₋₆ alkyl, C₂₋₆ alkenyl, C₂₋₆ alkynyl, aryl, heteroaryl, alkylaryl, arylalkyl, a C₄₋₁₄ bicyclic ring; a six-membered bridged or spiro-fused ring containing zero, one, or two heteroatoms which are, independently, N, O, S, or a 3-7 membered saturated or partially unsaturated ring containing one or more heteroatoms each independently selected from the group of N, O or S,
R¹³ is optionally substituted with one or more substituents each independently selected from the group consisting of 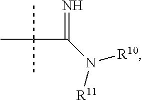
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 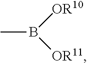
—S—R¹⁰; 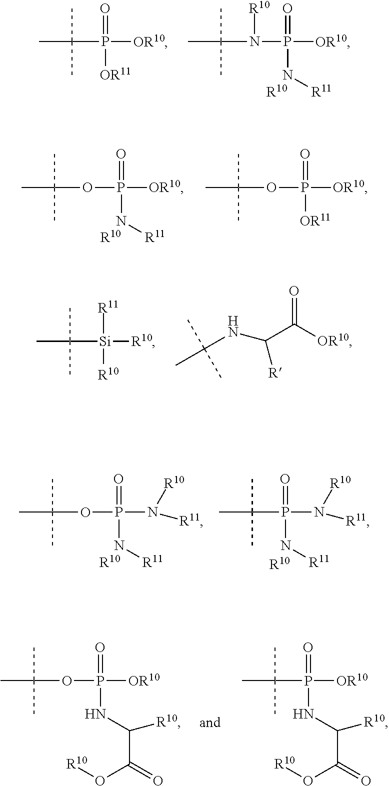
where substituents on the substituted aryl and substituted heteroaryl are selected from the group consisting of 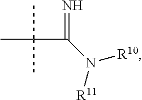
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 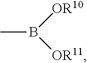
—S—R¹⁰; 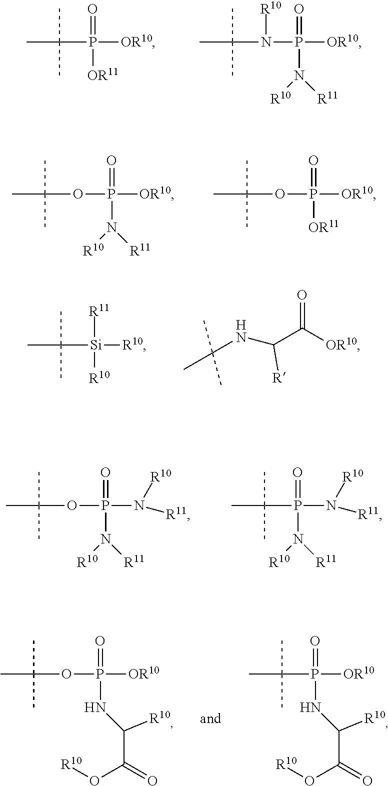
or R¹² and R¹³ together with the nitrogen to which they are attached form a saturated or unsaturated 4 to 7 membered ring optionally substituted with one or more substituents each independently selected from the group consisting of 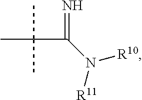
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 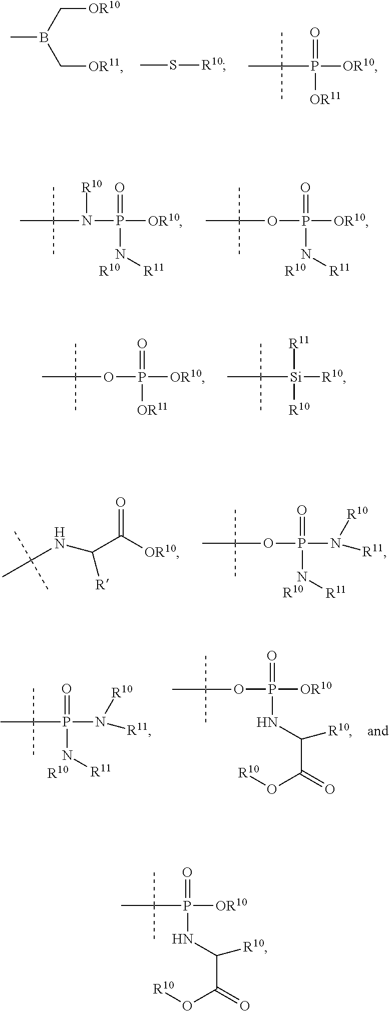
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
alternatively, R¹⁰ and R¹¹ can come together to form a saturated or partially unsaturated 5 to 8 membered ring or a six-membered bridged or spiro-fused ring containing zero, one, or two heteroatoms which are, independently, N, O, or S, a seven-membered bridged or spiro-fused a saturated or partially unsaturated ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms, a saturated or partially unsaturated 6 to 12 membered hetero-bicyclic ring; or a saturated or partially unsaturated 6 to 12 membered hetero-tricyclic ring; and
R¹⁰ and R¹¹ groups can optionally be substituted with one or more substituents, which substituents are, independently, halo, C₁₋₆ alkyl, C₃₋₆ alkyl, C₁₋₆ haloalkyl, C₂₋₆ alkenyl, C₂₋₆ alkynyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphoramidate or phosphonate.

21. The compound of claim 18, wherein R¹ is

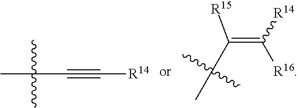

22. A compound selected from the group consisting of: 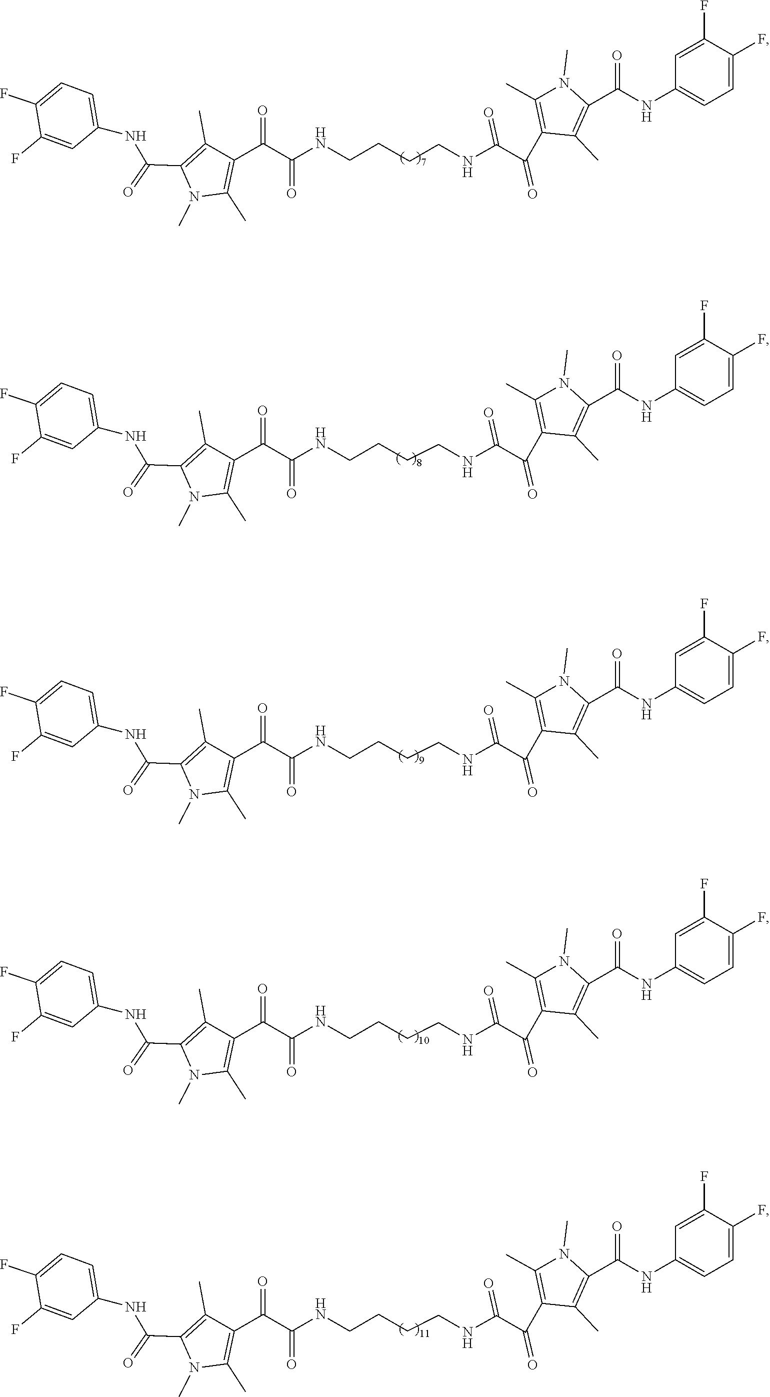 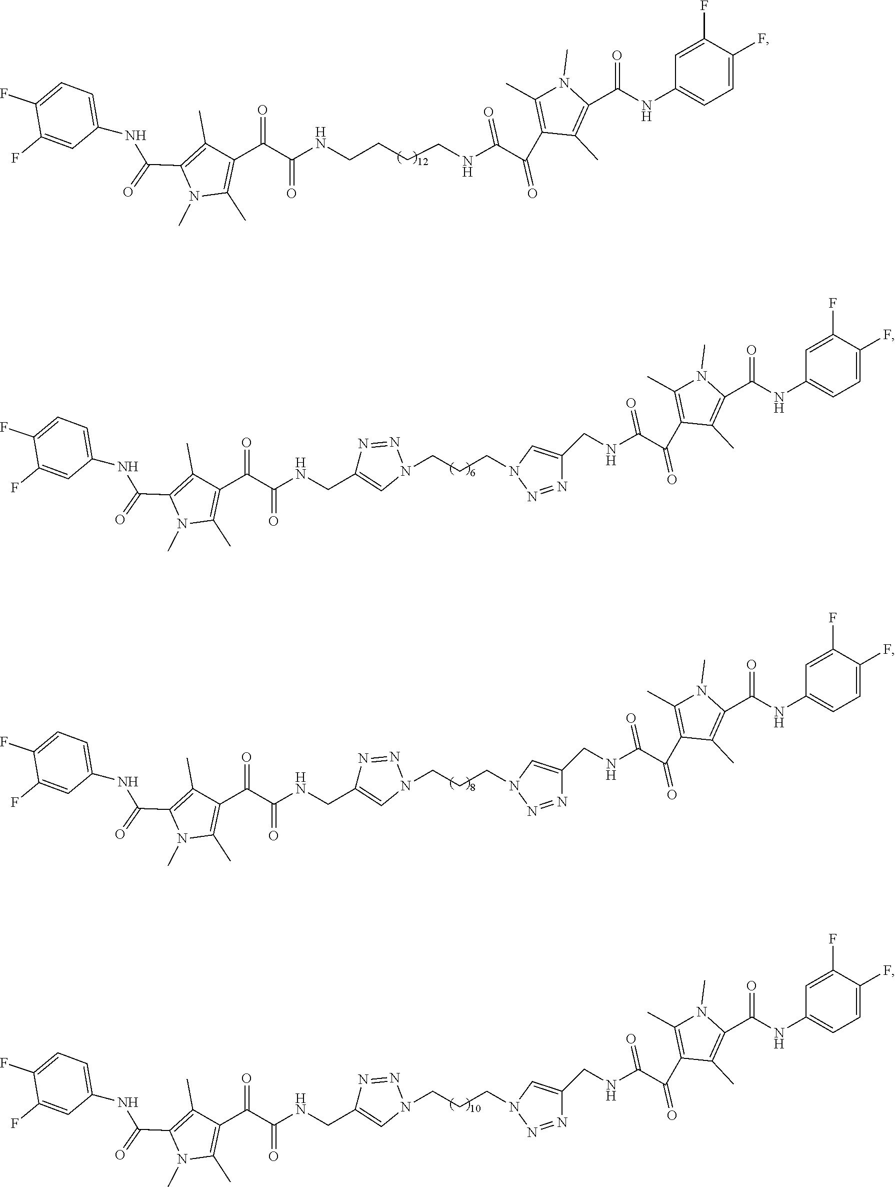 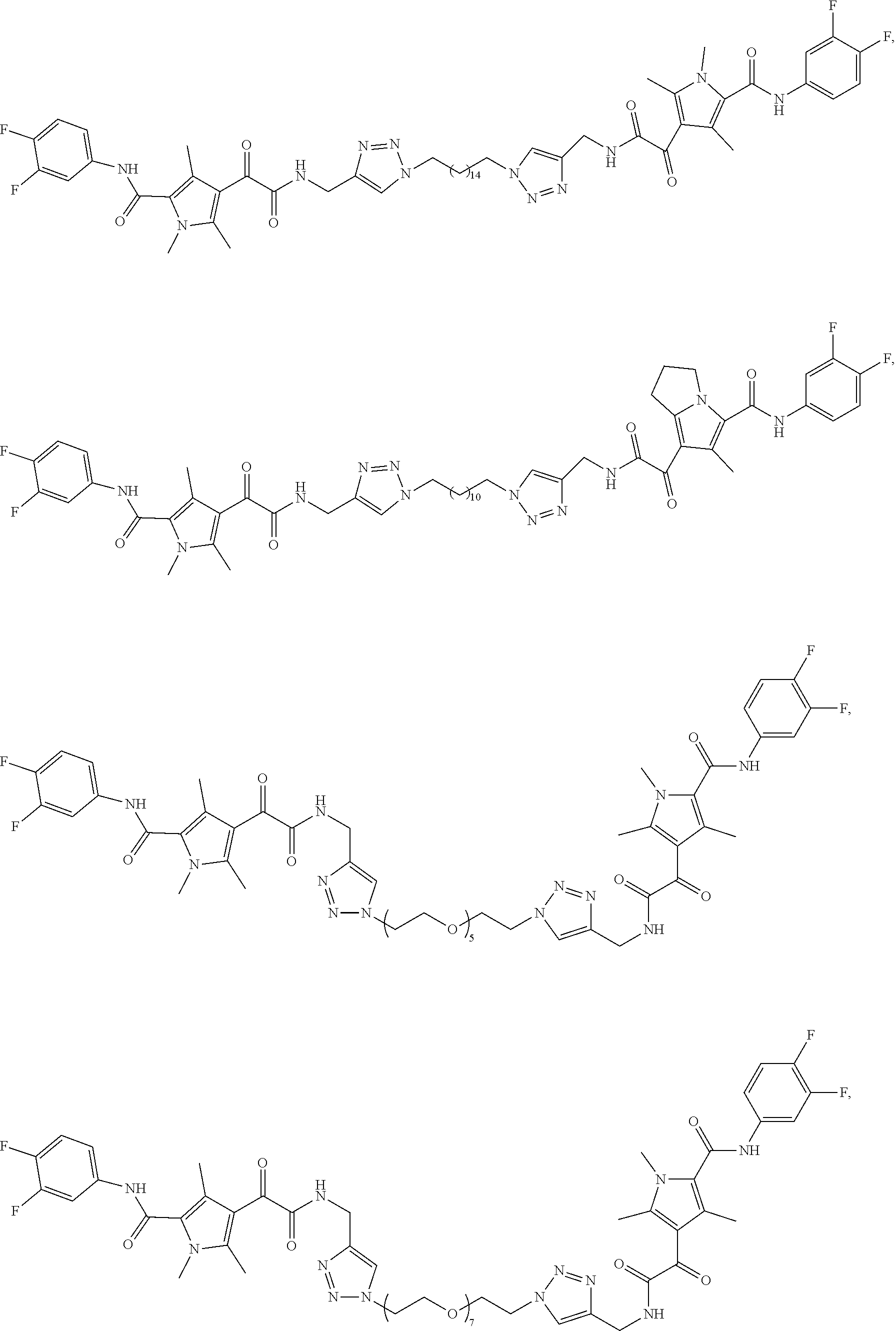 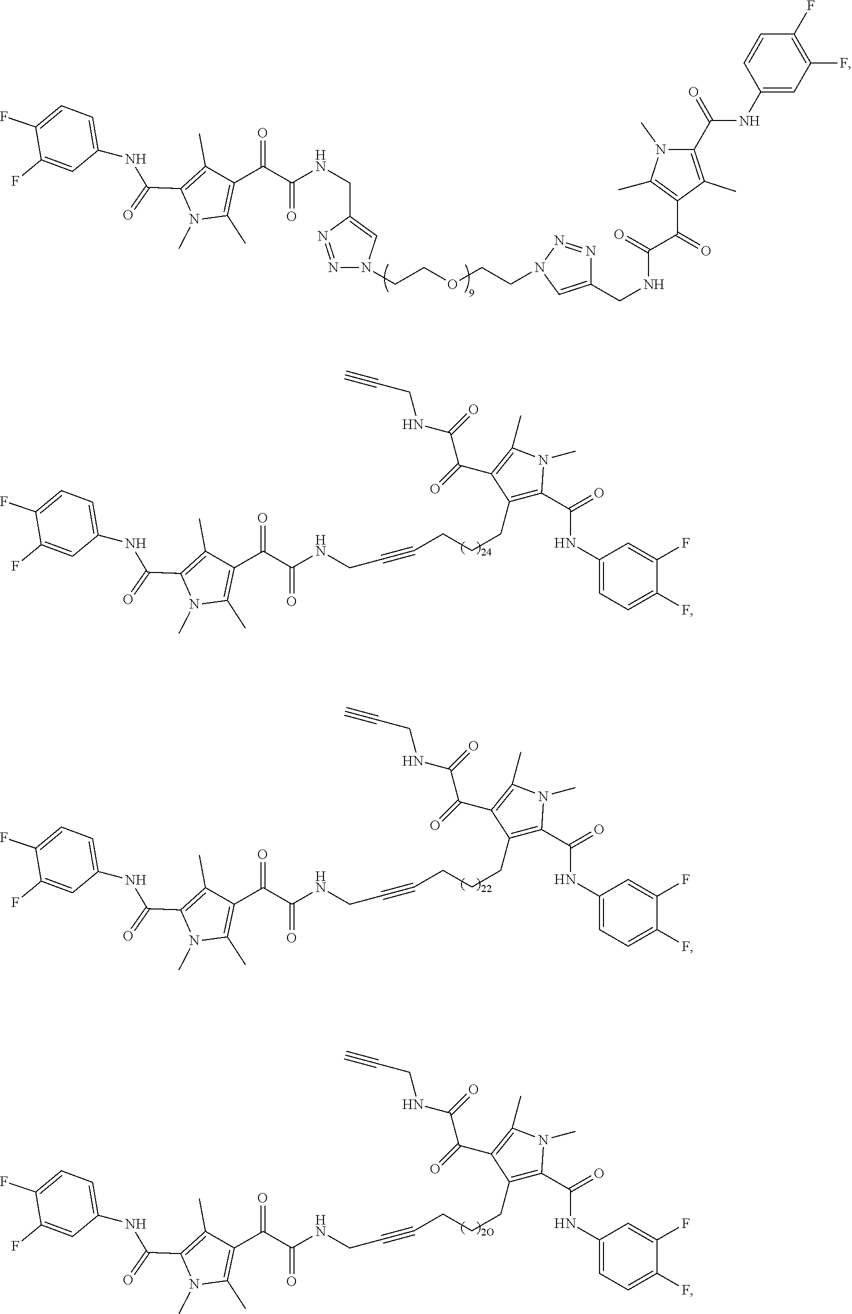 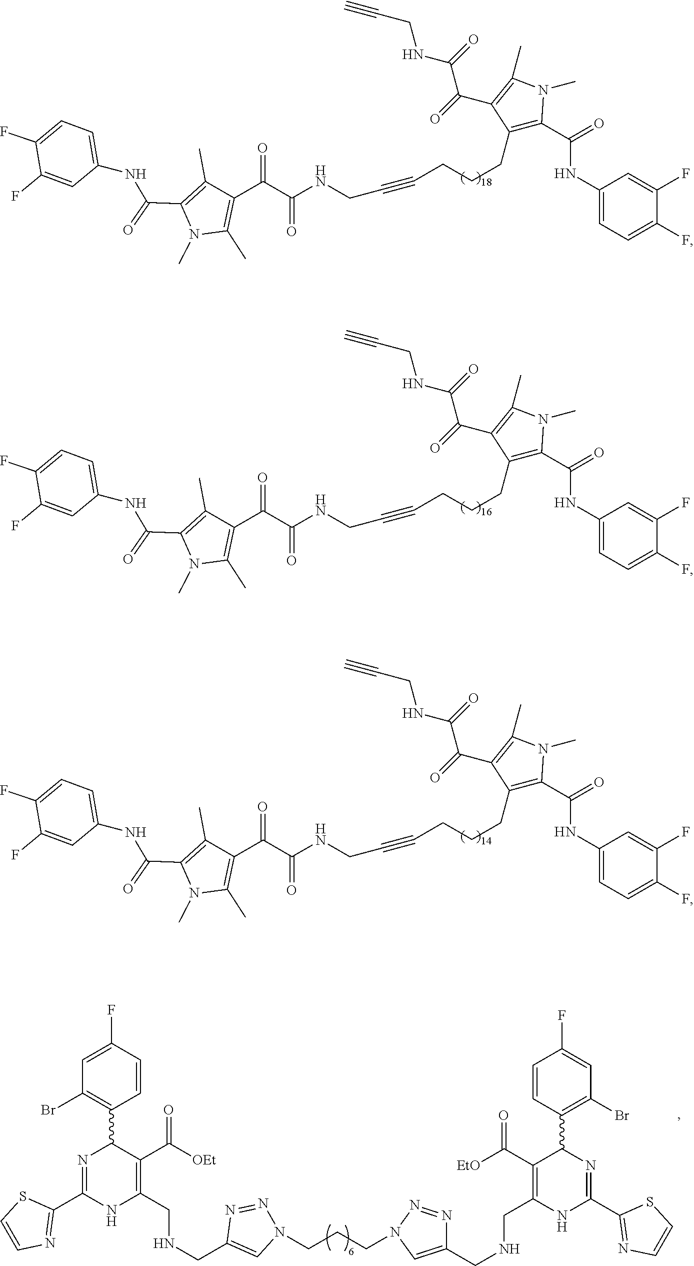 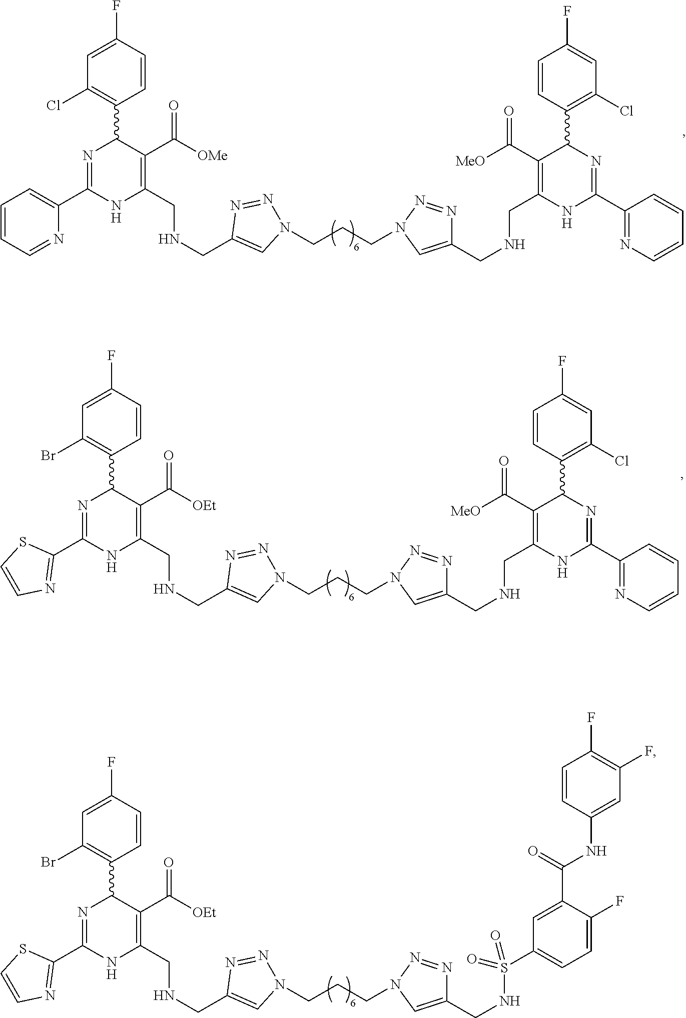 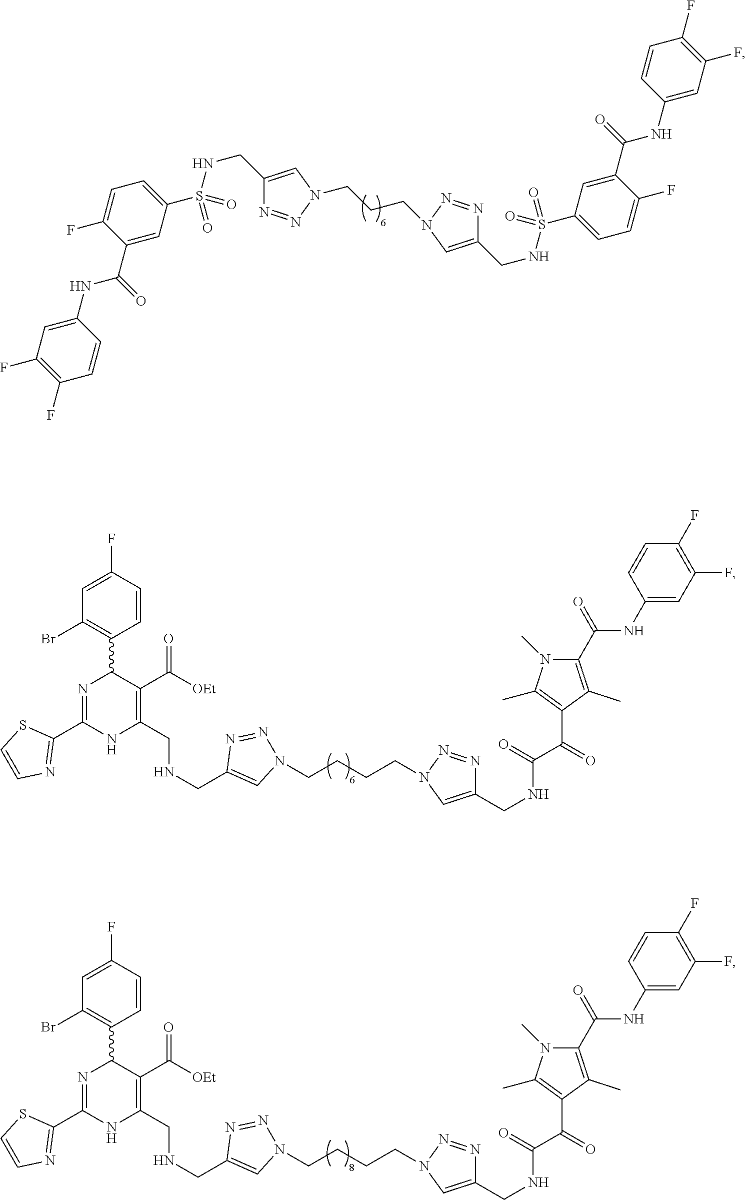 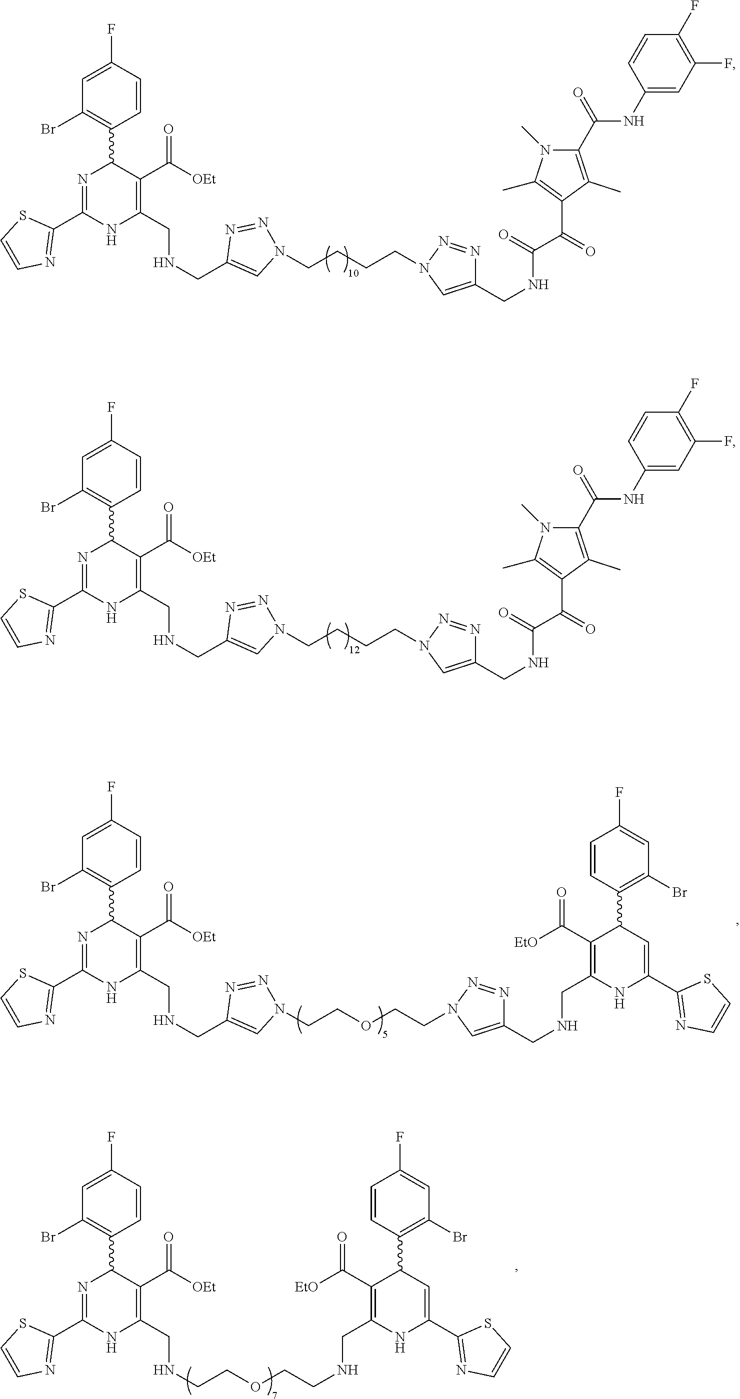 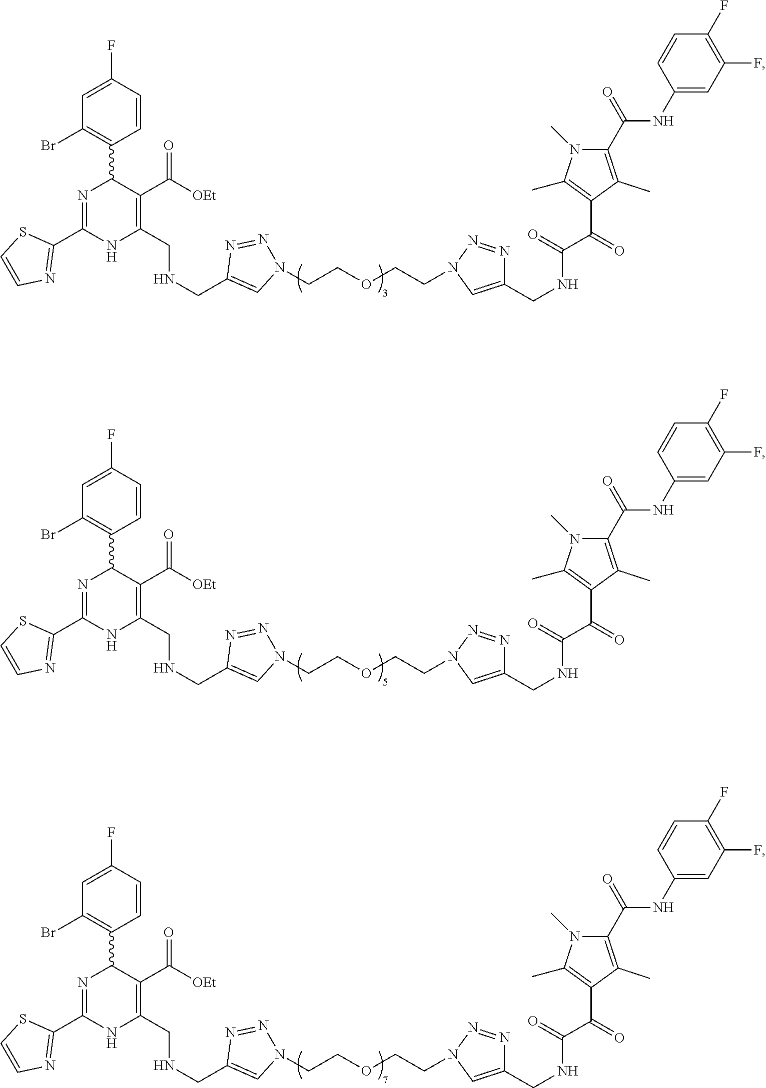 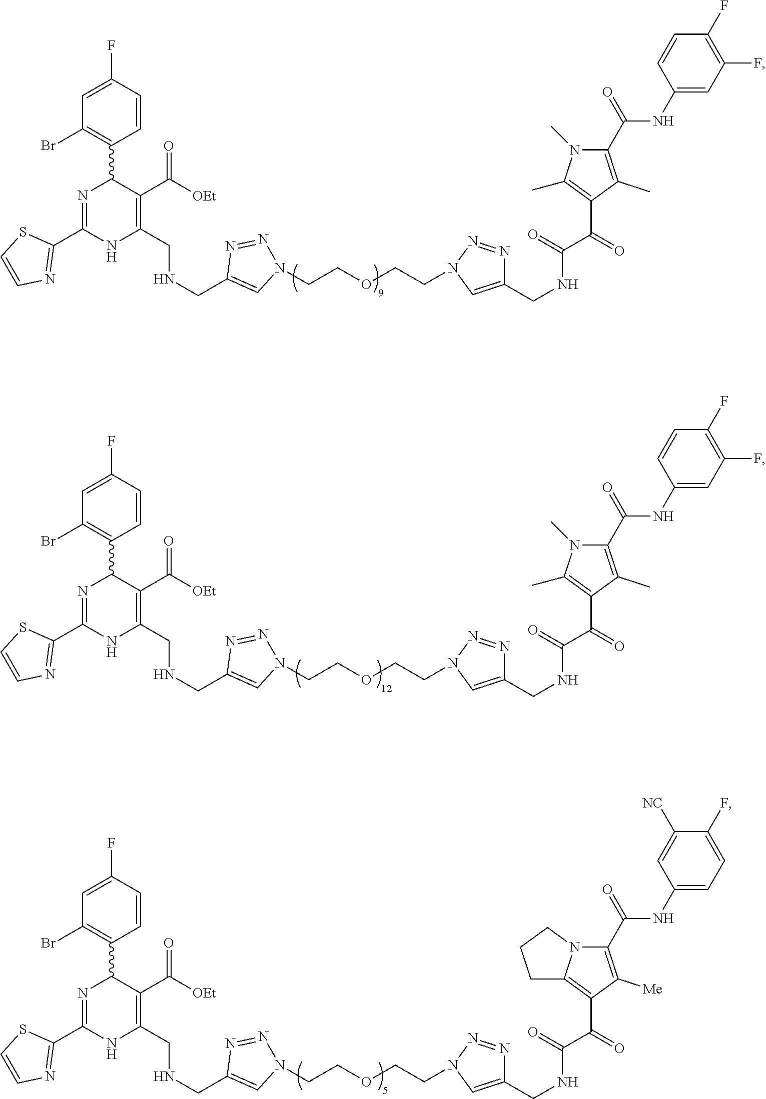 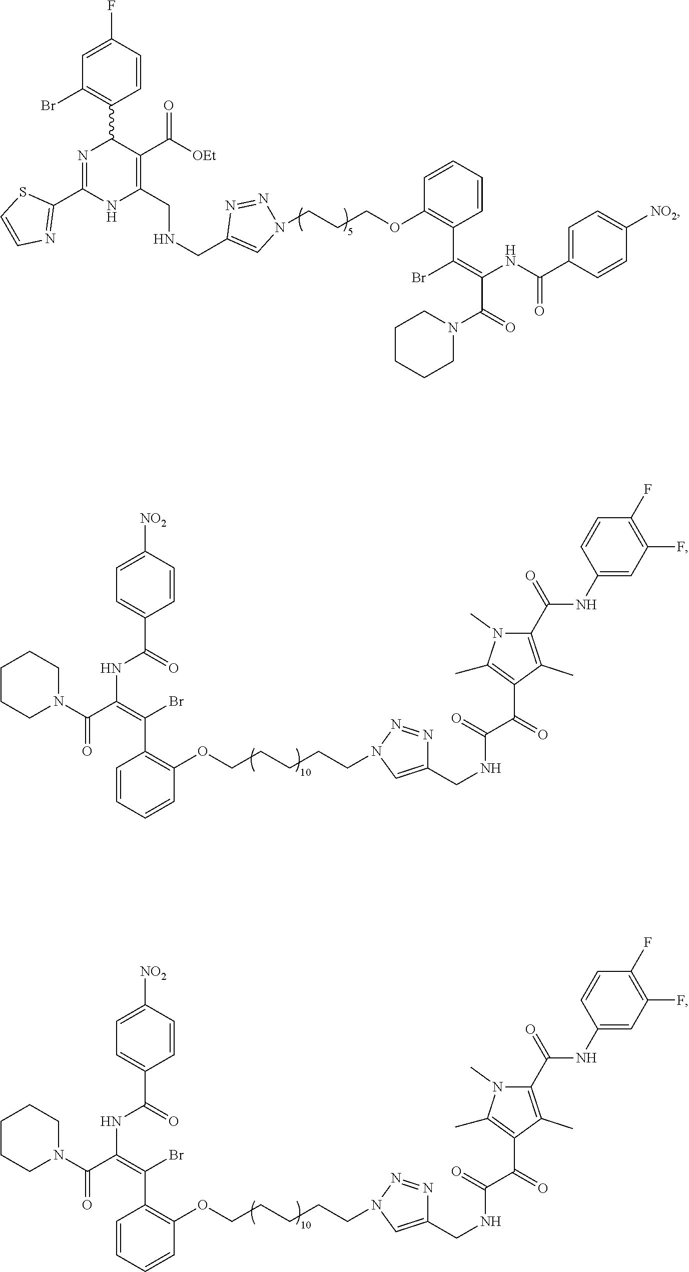 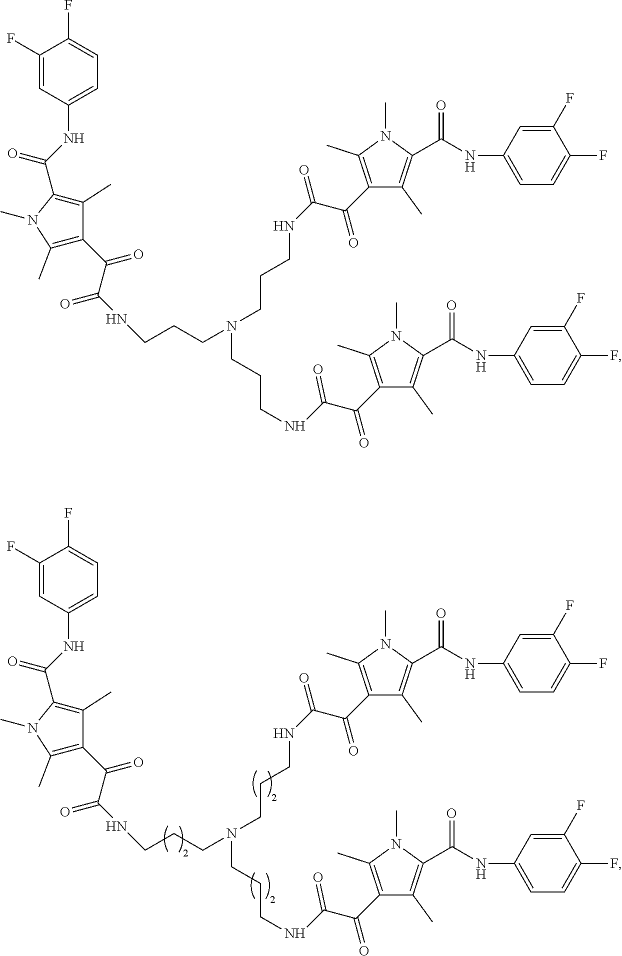 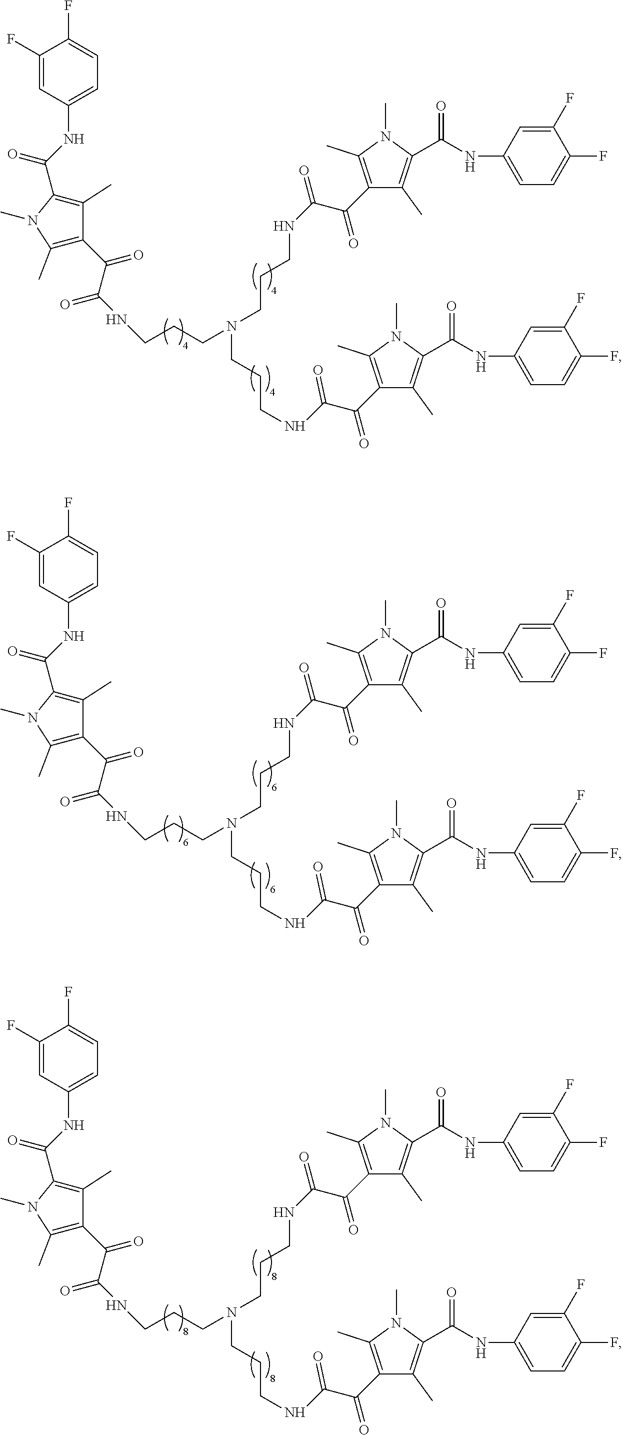 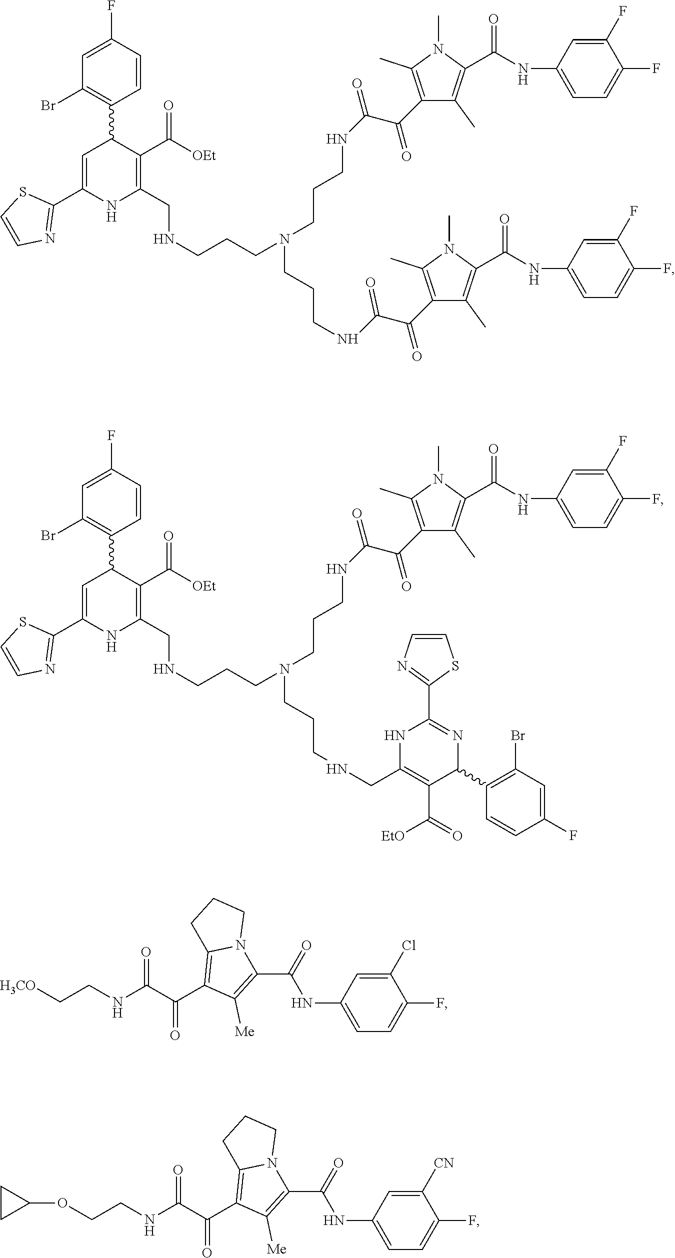 [SMILES: CCOC(=O)C1=C(CNCCCN(CCCNC(=O)C(=O)c2c(C)c(C(=O)Nc3ccc(F)c(F)c3)n(C)c2C)CCCNC(=O)C(=O)c2c(C)c(C(=O)Nc3ccc(F)c(F)c3)n(C)c2C)NC(c2nccs2)=CC1c1ccc(F)cc1Br ; CCOC(=O)C1=C(CNCCCN(CCCNCC2=C(C(=O)OCC)C(c3ccc(F)cc3Br)N=C(c3nccs3)N2)CCCNC(=O)C(=O)c2c(C)c(C(=O)Nc3ccc(F)c(F)c3)n(C)c2C)NC(c2nccs2)=CC1c1ccc(F)cc1Br ; COCCNC(=O)C(=O)c1c(C)c(C(=O)Nc2ccc(F)c(Cl)c2)n2c1CCC2 ; Cc1c(C(=O)C(=O)NCCOC2CC2)c2n(c1C(=O)Nc1ccc(F)c(C#N)c1)CCC2] 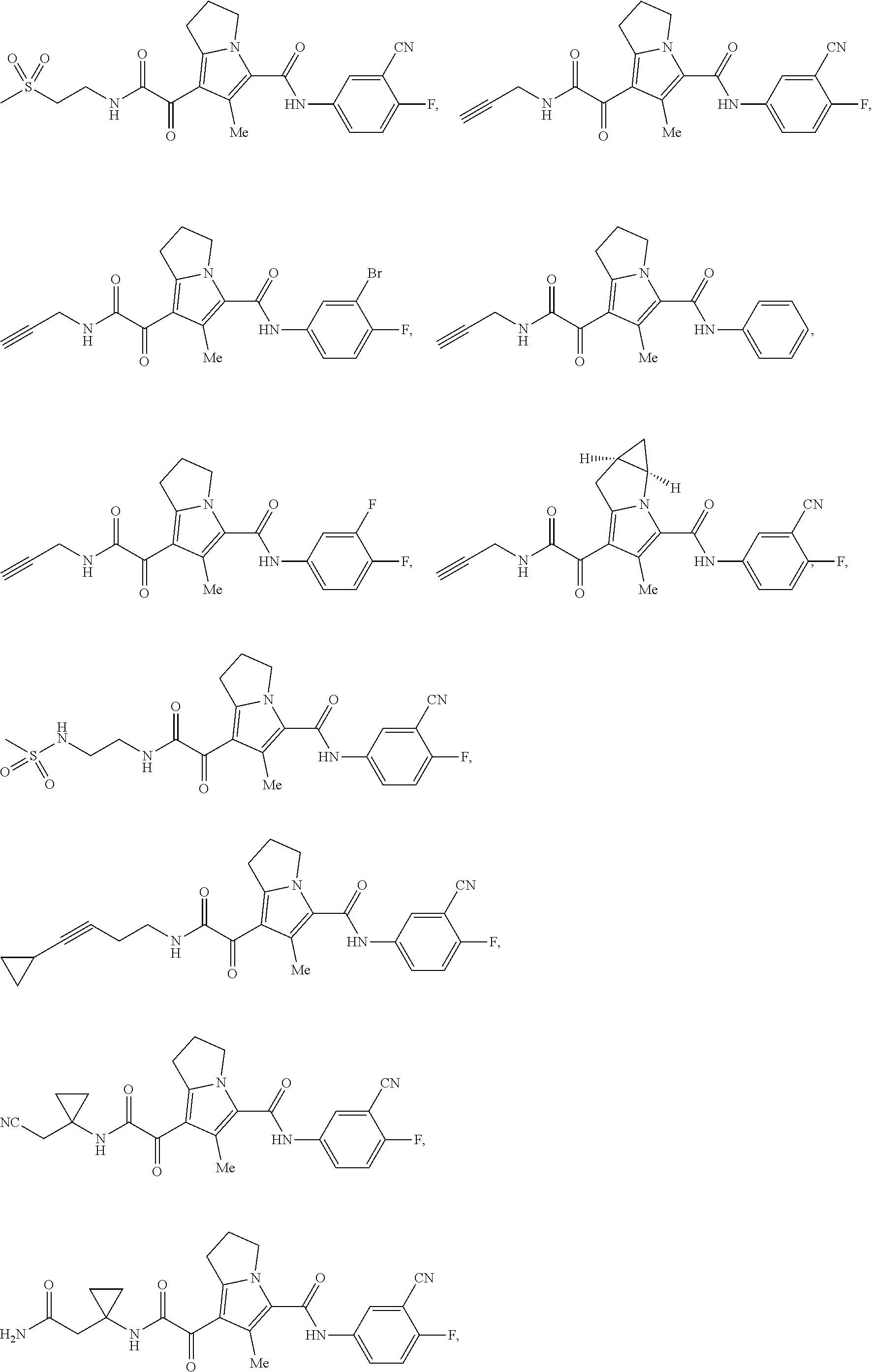 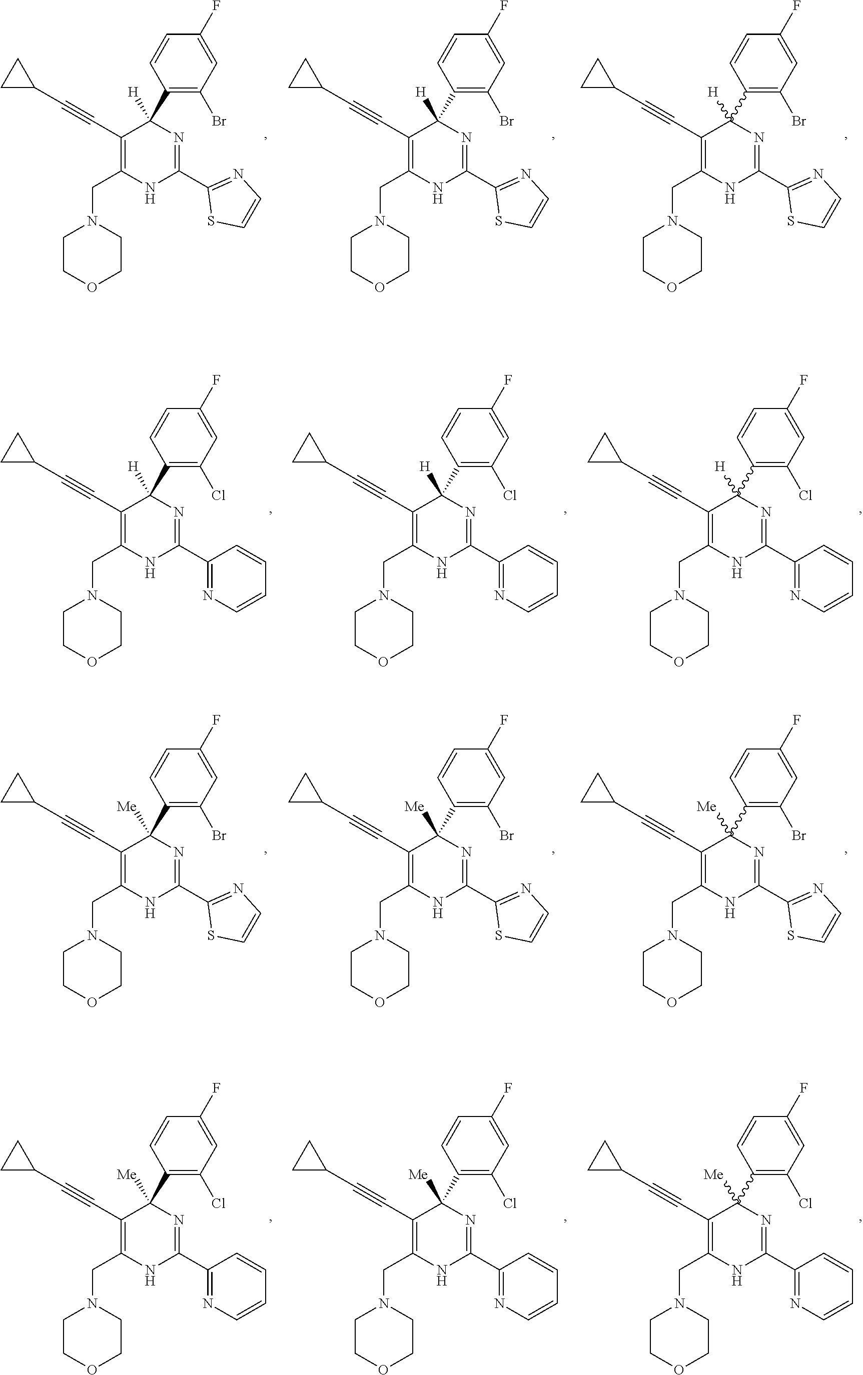 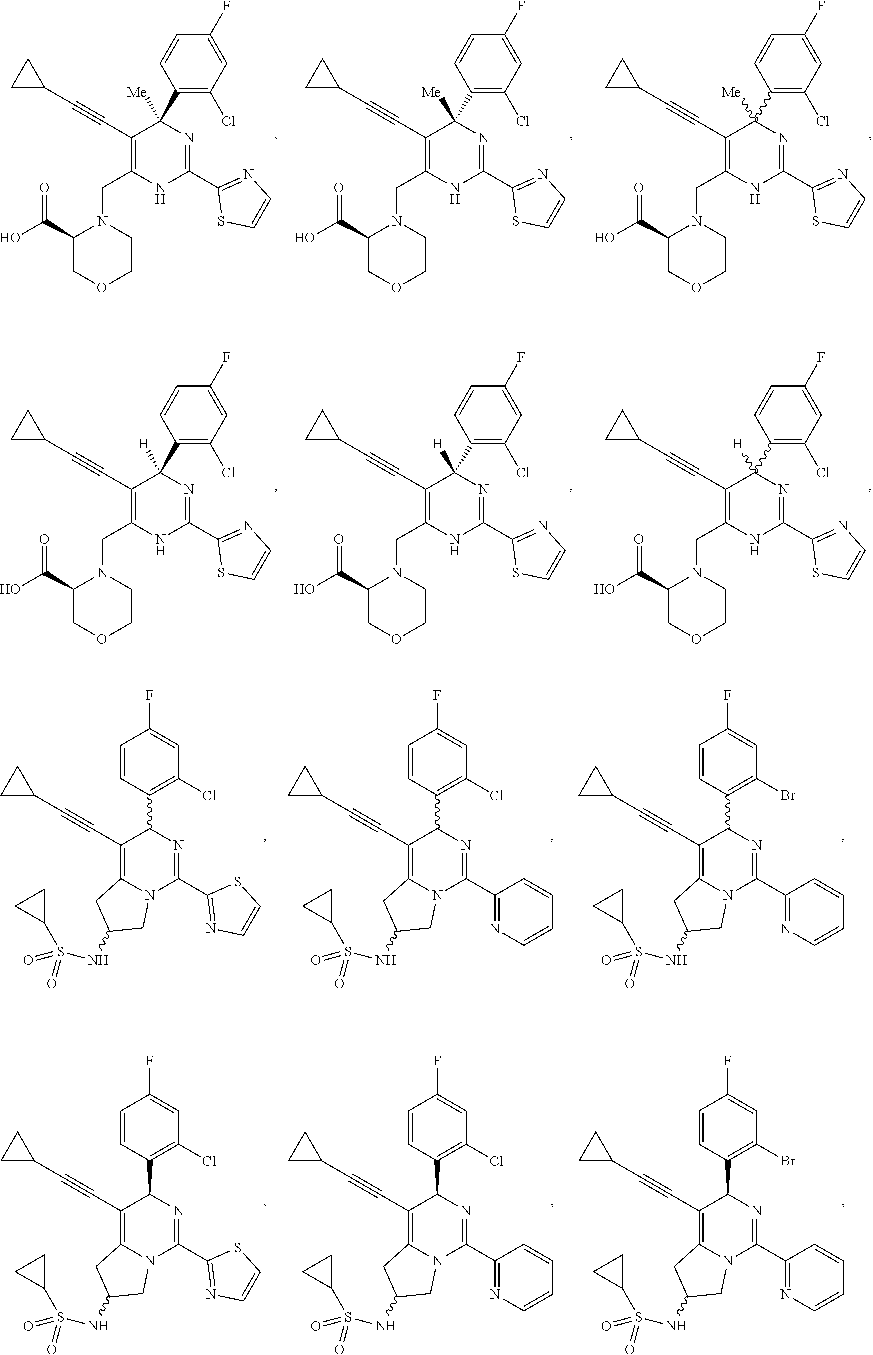 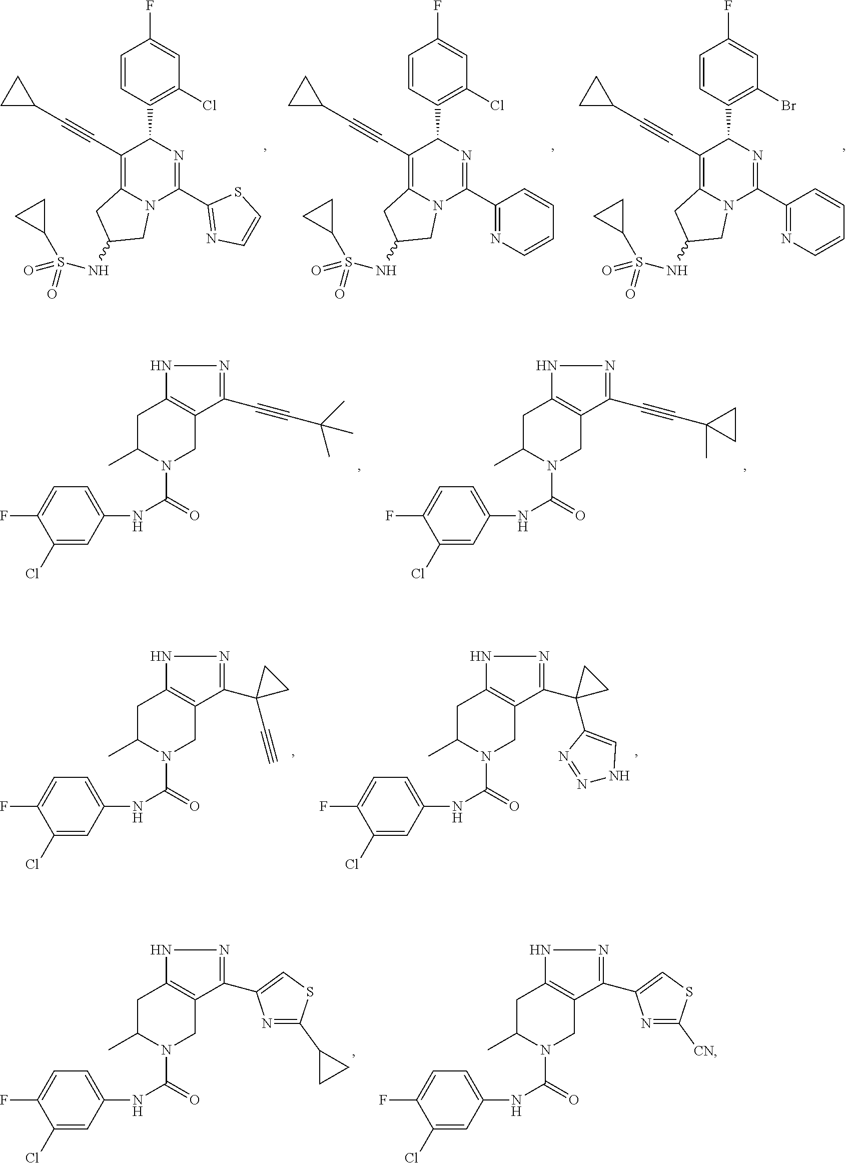 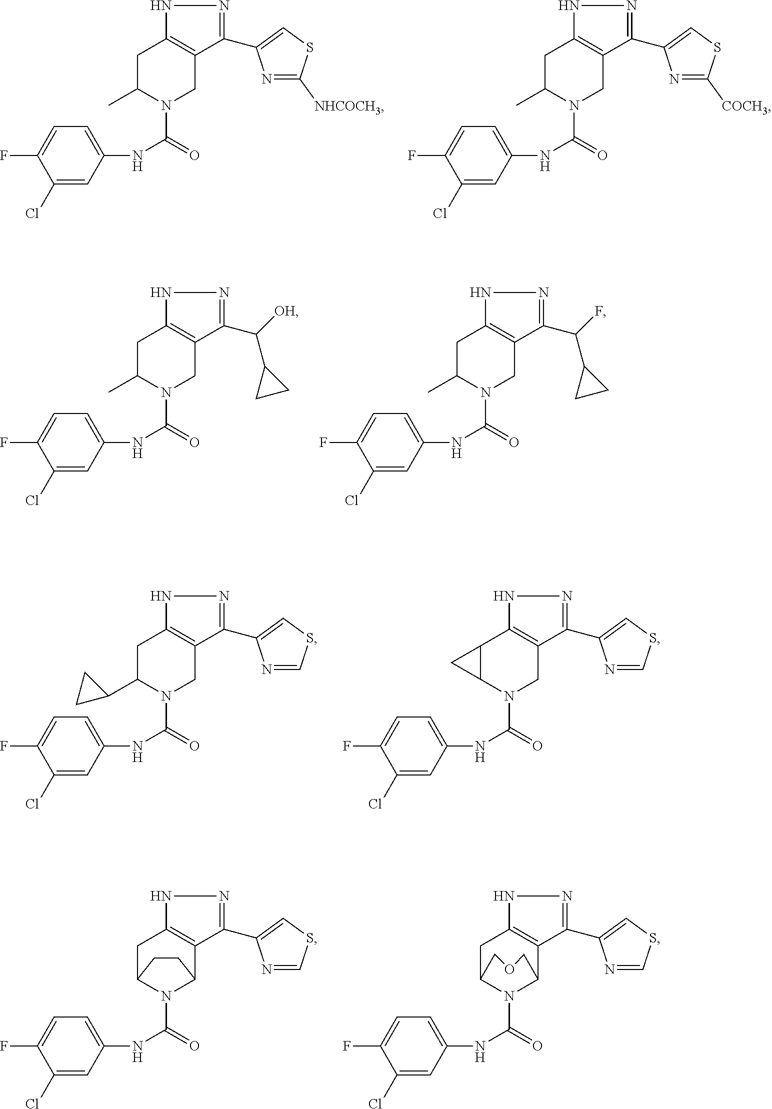 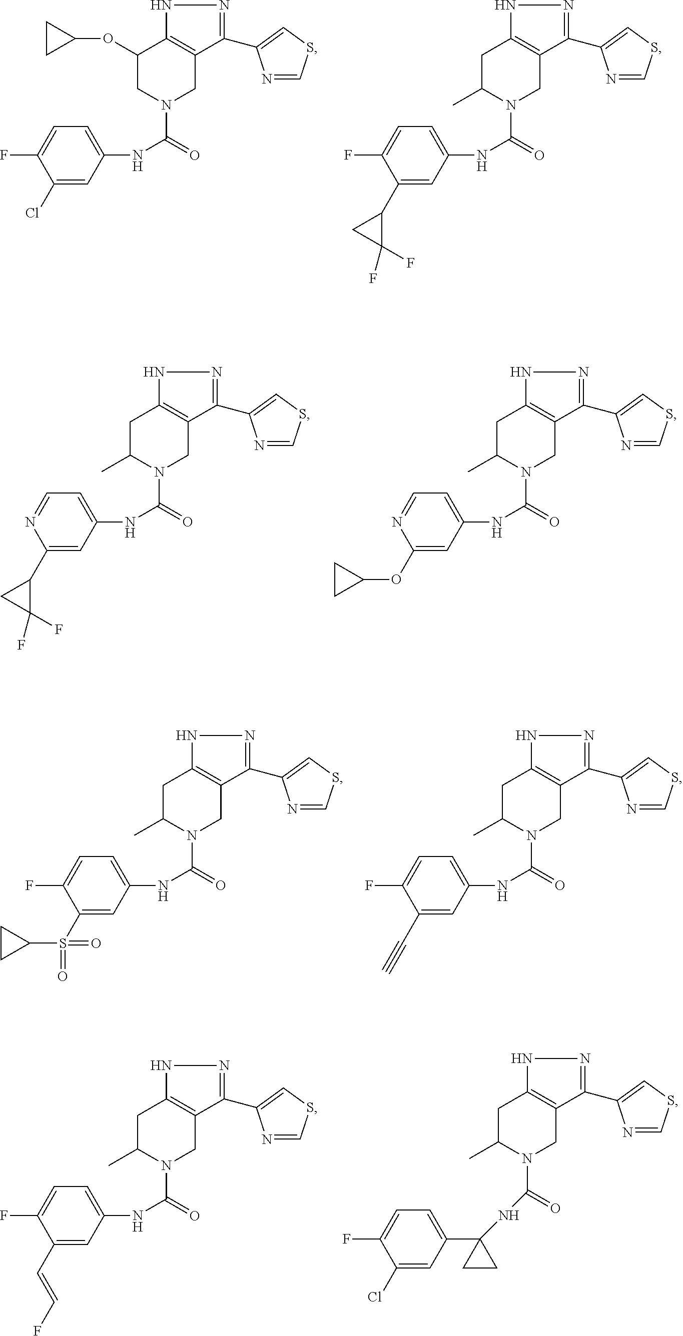 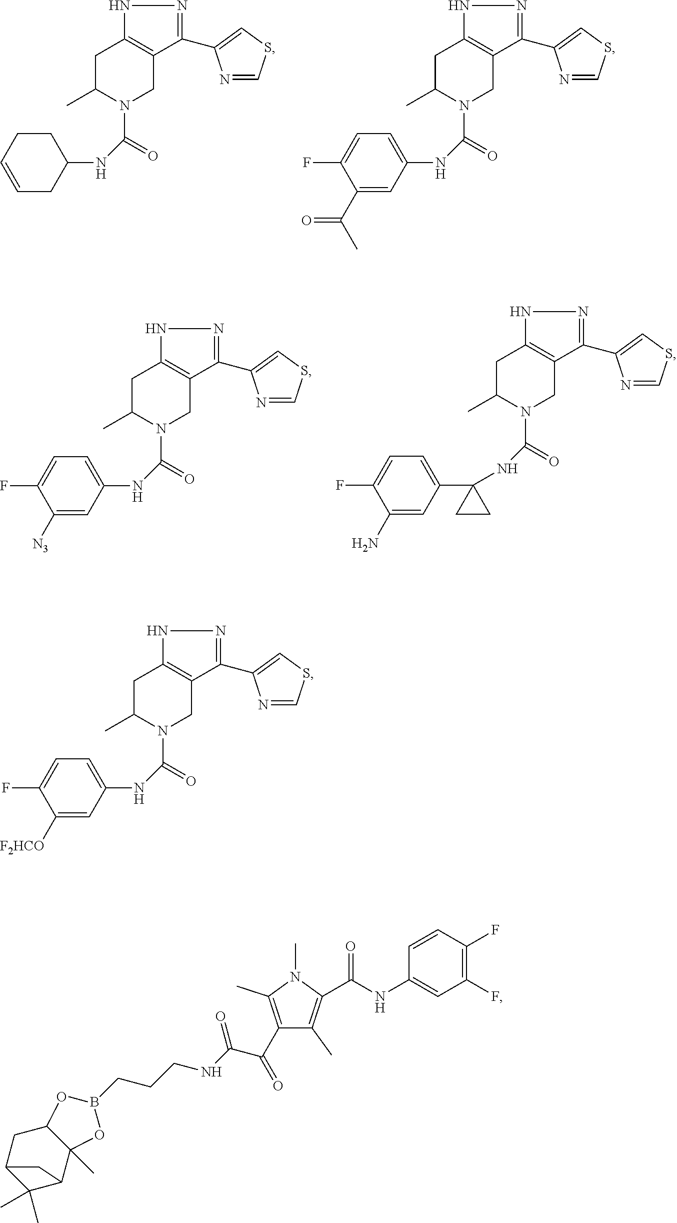 [SMILES: CC1Cc2[nH]nc(-c3cscn3)c2CN1C(=O)NC1CC=CCC1 ; CC(=O)c1cc(NC(=O)N2Cc3c(-c4cscn4)n[nH]c3CC2C)ccc1F ; CC1Cc2[nH]nc(-c3cscn3)c2CN1C(=O)Nc1ccc(F)c(N=[N+]=[N-])c1 ; CC1Cc2[nH]nc(-c3cscn3)c2CN1C(=O)NC1(c2ccc(F)c(N)c2)CC1 ; CC1Cc2[nH]nc(-c3cscn3)c2CN1C(=O)Nc1ccc(F)c(OC(F)F)c1 ; Cc1c(C(=O)C(=O)NCCCB2OC3CC4CC(C4(C)C)C3(C)O2)c(C)n(C)c1C(=O)Nc1ccc(F)c(F)c1] 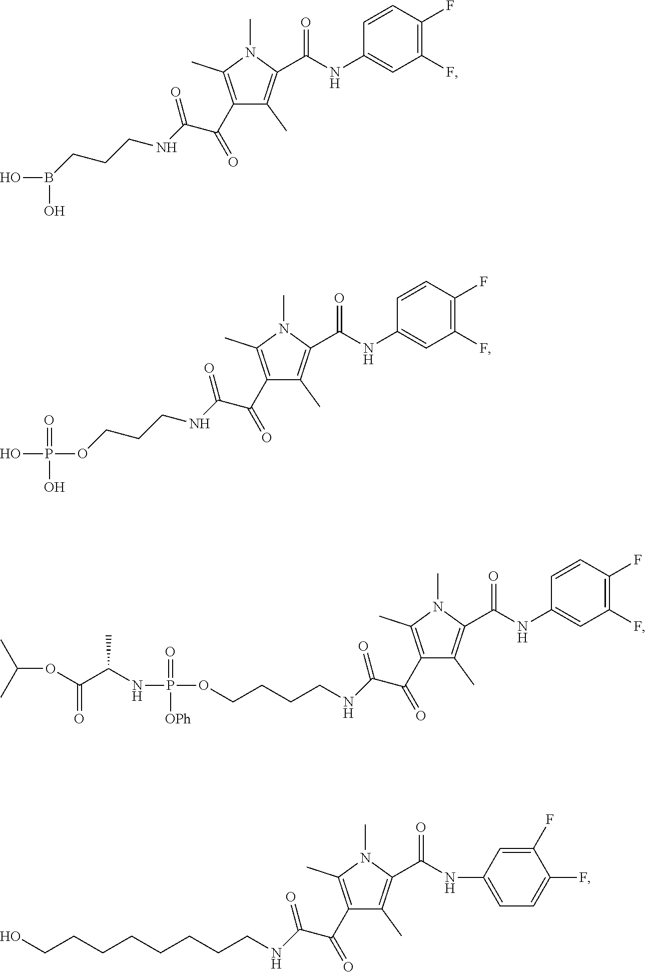 [SMILES: Cc1c(C(=O)C(=O)NCCCB(O)O)c(C)n(C)c1C(=O)Nc1ccc(F)c(F)c1 ; Cc1c(C(=O)C(=O)NCCCOP(=O)(O)O)c(C)n(C)c1C(=O)Nc1ccc(F)c(F)c1 ; Cc1c(C(=O)C(=O)NCCCCOP(=O)(N[C@@H](C)C(=O)OC(C)C)Oc2ccccc2)c(C)n(C)c1C(=O)Nc1ccc(F)c(F)c1 ; Cc1c(C(=O)C(=O)NCCCCCCCCO)c(C)n(C)c1C(=O)Nc1ccc(F)c(F)c1]
and pharmaceutically acceptable salts or prodrugs thereof.

23. A compound selected from the group consisting of: 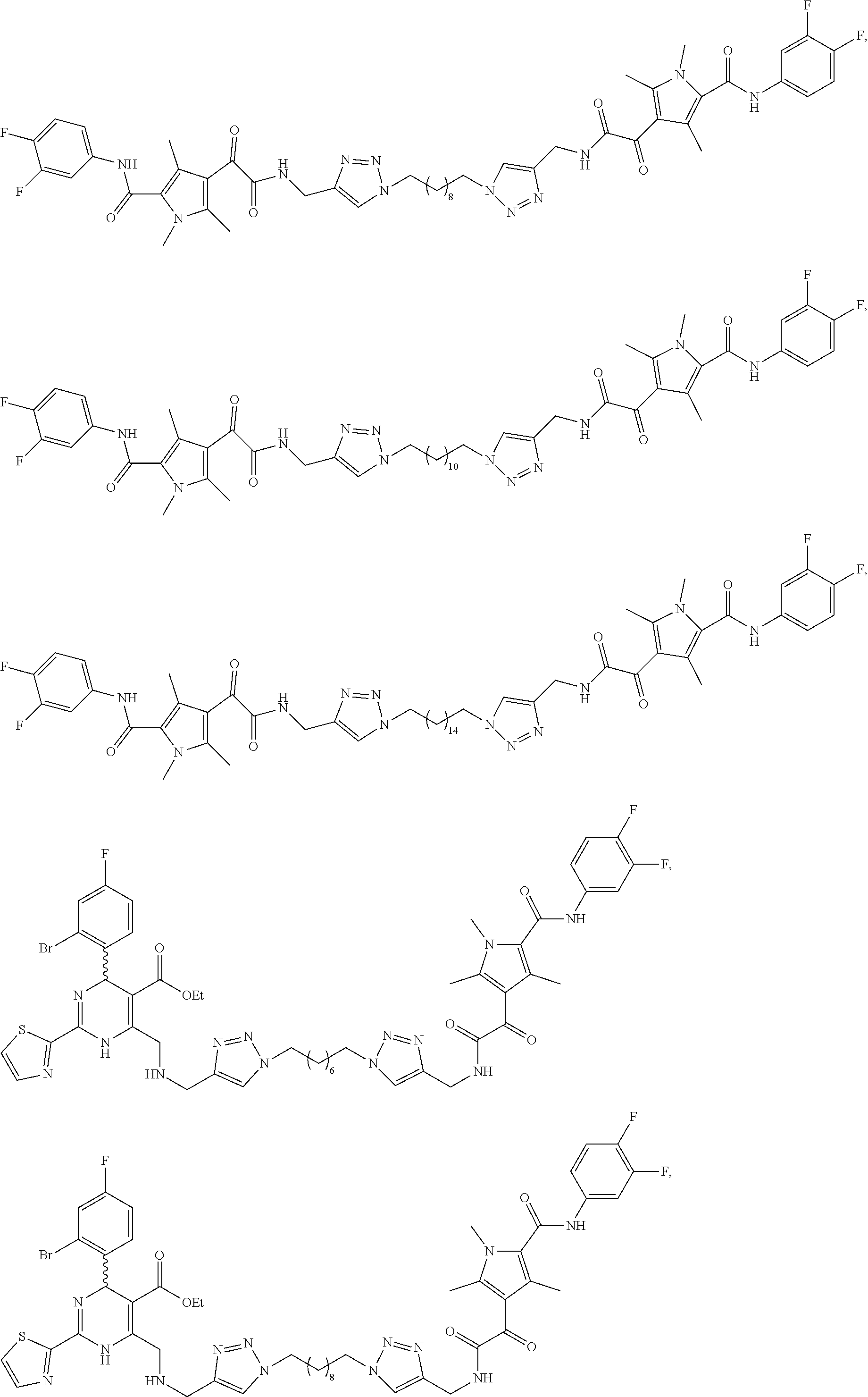 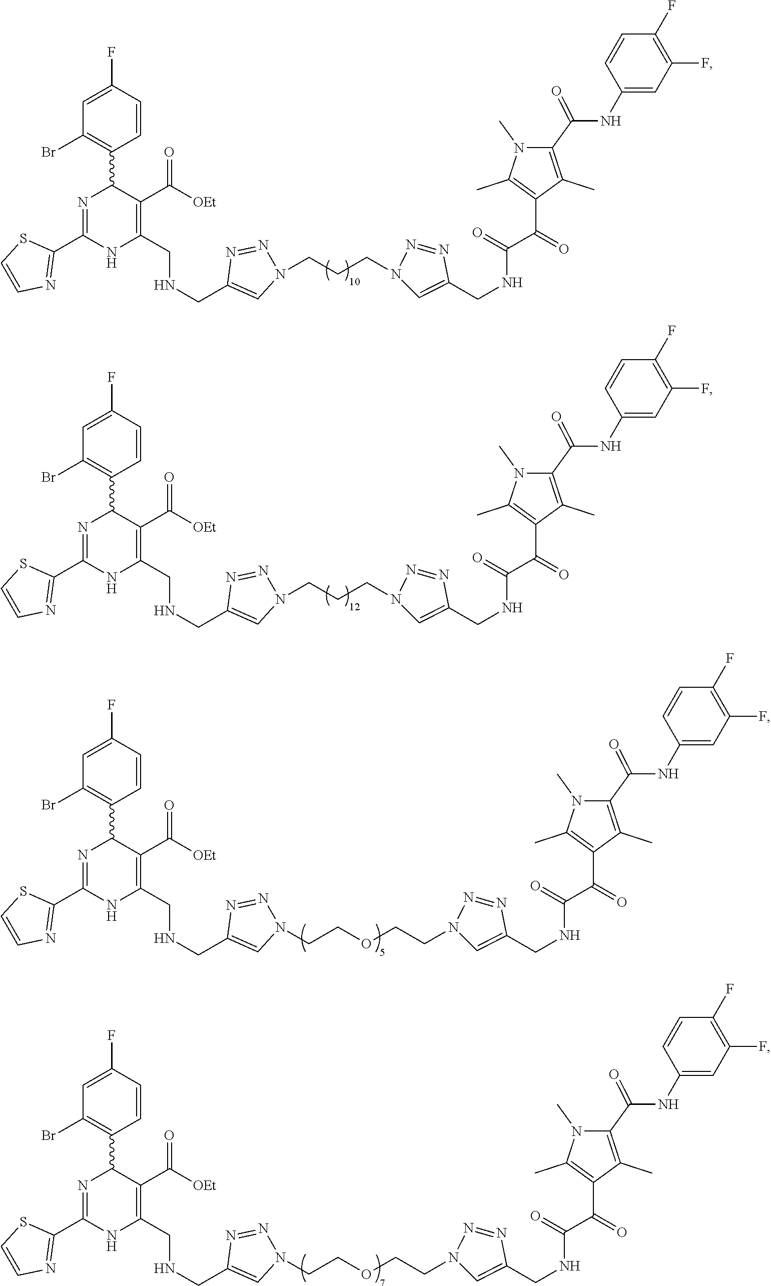 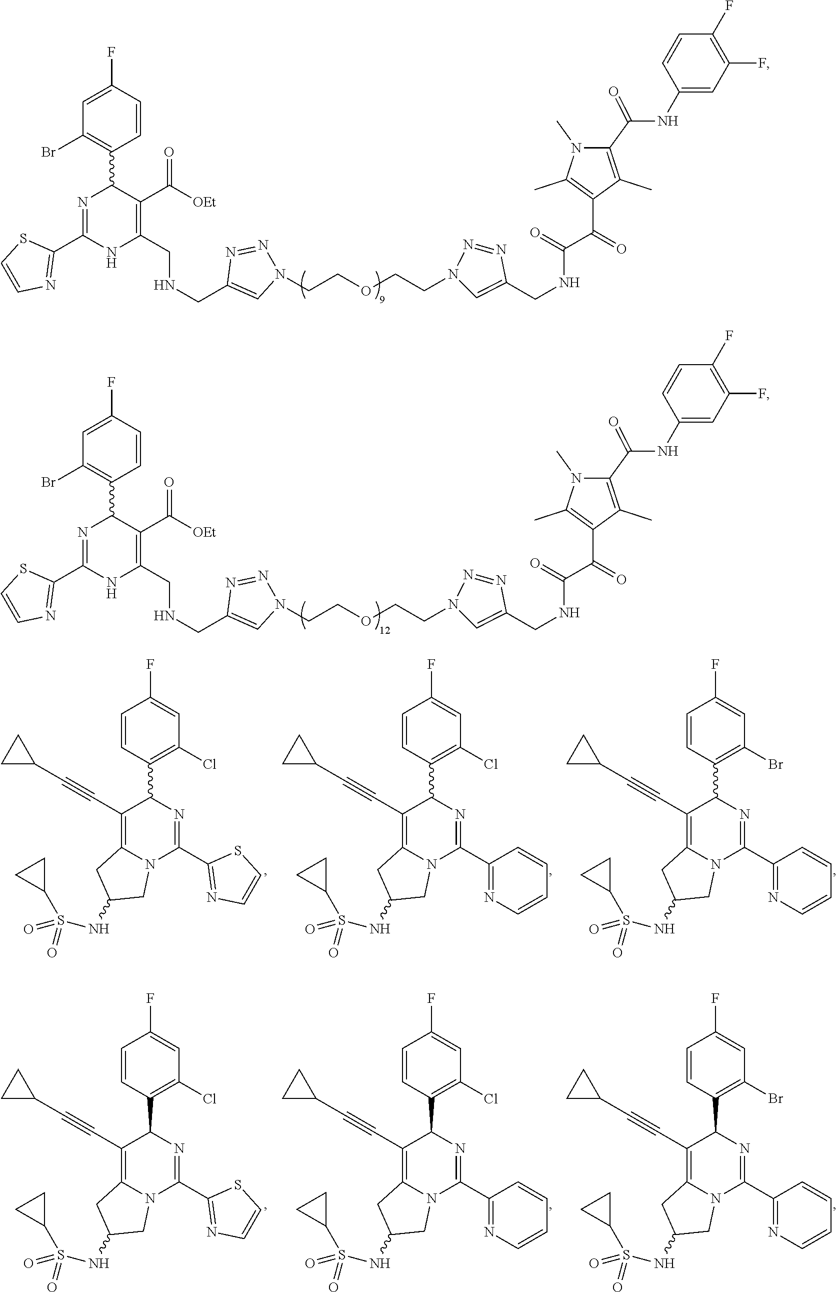 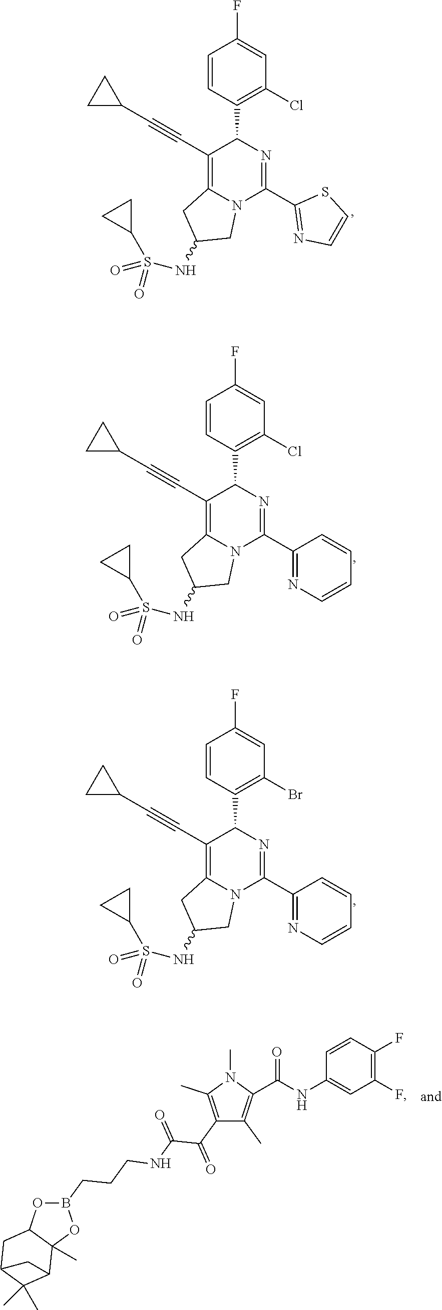 [SMILES: O=S(=O)(NC1CC2=C(C#CC3CC3)[C@H](c3ccc(F)cc3Cl)N=C(c3nccs3)N2C1)C1CC1 ; O=S(=O)(NC1CC2=C(C#CC3CC3)[C@H](c3ccc(F)cc3Cl)N=C(c3ccccn3)N2C1)C1CC1 ; O=S(=O)(NC1CC2=C(C#CC3CC3)[C@H](c3ccc(F)cc3Br)N=C(c3ccccn3)N2C1)C1CC1 ; Cc1c(C(=O)C(=O)NCCCB2OC3CC4CC(C4(C)C)C3(C)O2)c(C)n(C)c1C(=O)Nc1ccc(F)c(F)c1] 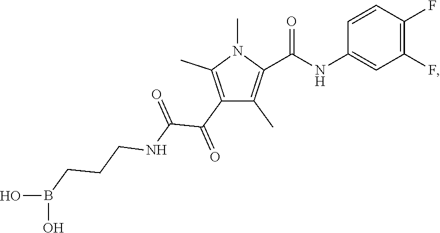 [SMILES: Cc1c(C(=O)C(=O)NCCCB(O)O)c(C)n(C)c1C(=O)Nc1ccc(F)c(F)c1]
and pharmaceutically-acceptable salts and prodrugs thereof.

24. A pharmaceutical composition comprising a compound of any of claims 1 to 23, and a pharmaceutically-acceptable carrier.

25. The composition of claim 24, wherein the composition is a transdermal composition or a nanoparticulate composition.

26. The pharmaceutical composition of claim 24, further comprising a second antiviral agent.

27. The pharmaceutical composition of claim 26, wherein the second antiviral agent is selected from the group consisting of a polymerase inhibitors, viral entry inhibitor, a viral maturation inhibitor, a literature described capsid assembly modulator, an IMPDH inhibitor, a protease inhibitor, an immune-based therapeutic agents, a reverse transcriptase inhibitor, a TLR-agonist, siRNA, shRNA, Talens, Crisper/Cas9, mir (microRNA), an agent of distinct or unknown mechanism, and combinations thereof.

28. Use of a compound of any of claims 1-23 in the preparation of a medicament for treating HBV infection, preventing a HBV infection, or reducing the biological activity of an infection with HBV.

29. A method for treating a host infected with HBV, preventing an HBV infection, curing an HBV infection, or reducing the biological activity of an infection with HBV in a host, comprising administering an effective amount of a compound of any of claims 1-23 to a patient in need of treatment thereof.

30. The method of claim 29, wherein the method further comprising administering another anti-HBV agent in combination or alternation with the compound of any of claims 1-23.

31. A compound of the following formula: 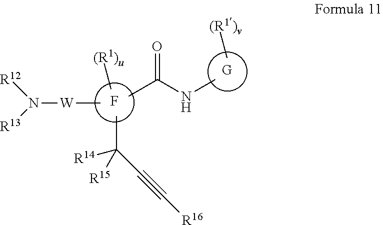
or a pharmaceutically acceptable salt or prodrug thereof, wherein
when R¹ and R^{1′} are attached to a carbon they are, independently, hydrogen, halogen, CF₃, hydroxy, SF₅, N(R′)S(O)₂R′, S(O)₂R′, S(O)₂N(R′)₂, C₁₋₆ alkoxy, cyano, C₂₋₁₀ alkynyl, C₃₋₆ alkoxyalkyl, alkoxycarbonyl, alkoxycarbonylalkyl, C₁₋₆ alkyl, C₁₋₆ haloalkyl, arylalkoxycarbonyl, carboxy, C₁₋₆ haloalkyl, heterocyclylalkyl, or C₁₋₆ hydroxyalkyl;
when R¹ and R^{1′} are attached to a nitrogen they are, independently, hydrogen, C₁₋₆ alkoxy, C₃₋₆ alkoxyalkyl, alkoxycarbonyl, carbonylalkyl, carbonyl aryl, C₁₋₆ alkyl, C₂₋₁₀ alkynyl, C₂₋₆ alkenyl, heterocyclylalkyl, C₁₋₆ hydroxy alkyl, or S(O)₂R′;
each R′ is independently H, C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, or if two R′ reside on the same nitrogen atom they can come together to form a C₃₋₆ alkyl ring optionally containing a N, O, or S; wherein the R′ groups can be substituted with one or more substituents as defined above, for example, C₁₋₆ hydroxyalkyl, aminoalkyl, and alkoxyalkyl;
u and v are, independently, 0, 1, 2, 3, 4 or 5;
G is phenyl, a six-membered heteroaromatic ring containing one, two, or three nitrogen atoms, a five-membered heteroaromatic ring containing one, two, or three heteroatoms, which are, independently, N, O, or S, a C₄₋₁₄ bicyclic ring; alkylheteroaryl, cubane or alkylaryl;
F is selected from the group consisting of a five-membered heteroaromatic ring containing one, two, or three heteroatoms, which are, independently, N, O, or S, a six or seven-membered ring or a six or seven-membered bridged or spiro-fused ring containing zero, one, or two heteroatoms which are, independently, N, O, or S, a five-membered ring containing zero, one, or two heteroatoms which are, independently, N, O, or S; and a four-membered ring containing zero, one, or two heteroatoms which are, independently, N, O, or S,
W is 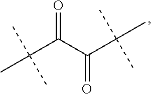
R¹² is H, C₁₋₆ alkyl, C₁₋₆ haloalkyl, C₂₋₆ alkenyl, or C₂₋₆ alkynyl,
R¹³ is C₁₋₆ alkyl, C₂₋₆ alkenyl, C₂₋₆ alkynyl, aryl, heteroaryl, alkylaryl, arylalkyl, a cubane, a C₄₋₁₄ bicyclic ring; a six-membered bridged or spiro-fused ring containing zero, one, or two heteroatoms which are, independently, N, O, S, or a 3-7 membered saturated or partially unsaturated ring containing one or more heteroatoms each independently selected from the group of N, O or S,
R¹³ is optionally substituted with one or more substituents each independently selected from the group consisting of 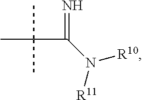
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 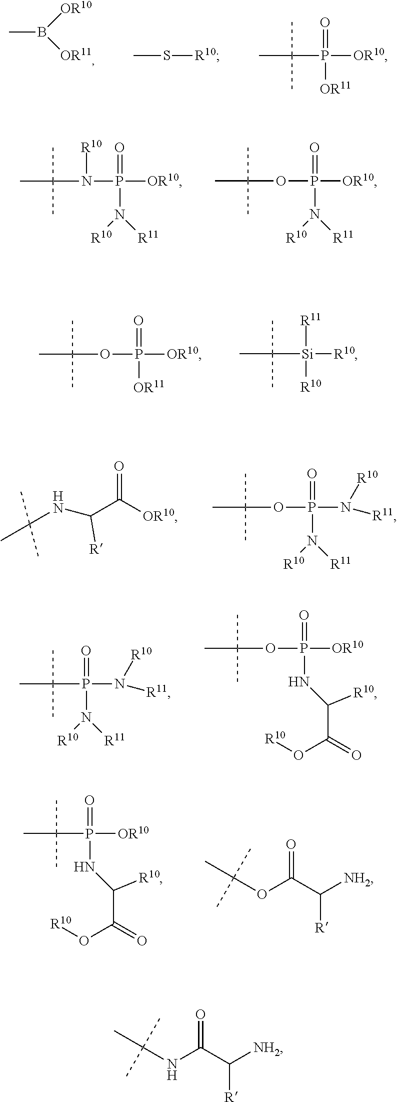
and —O—S(O)₂—OH,
wherein, when G is substituted aryl, or F is substituted five membered ring heteroaryl, the substituents are selected from the group consisting of 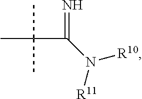
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 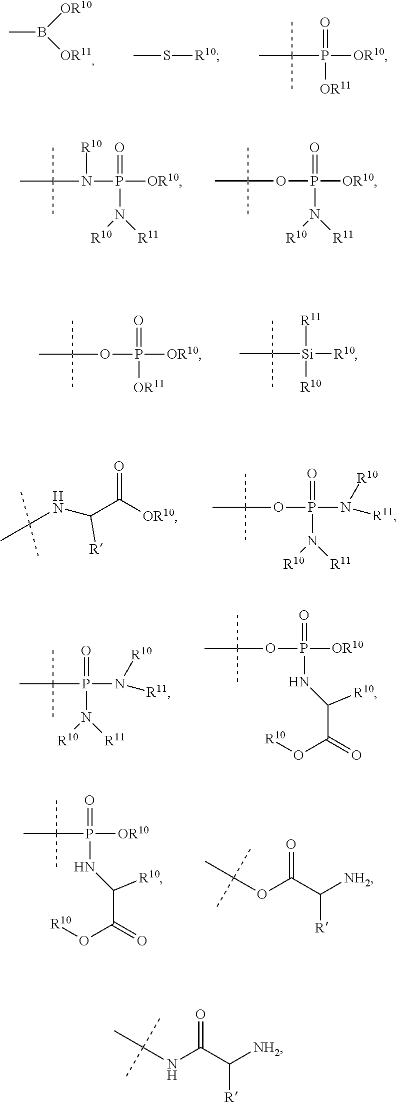
and —O—S(O)₂—OH,
or R¹² and R¹³ together with the nitrogen to which they are attached form a saturated or unsaturated 4 to 7 membered ring optionally substituted with one or more substituents each independently selected from the group consisting of 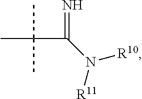
—NH—C(O)—N(R¹⁰)₂, —N(R¹⁰)₂, —OR¹¹, —C(O)OR¹⁰, —C(O)—N(R¹⁰)₂, —C(O)—NHOH, oxo, 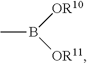
—S—R¹⁰; 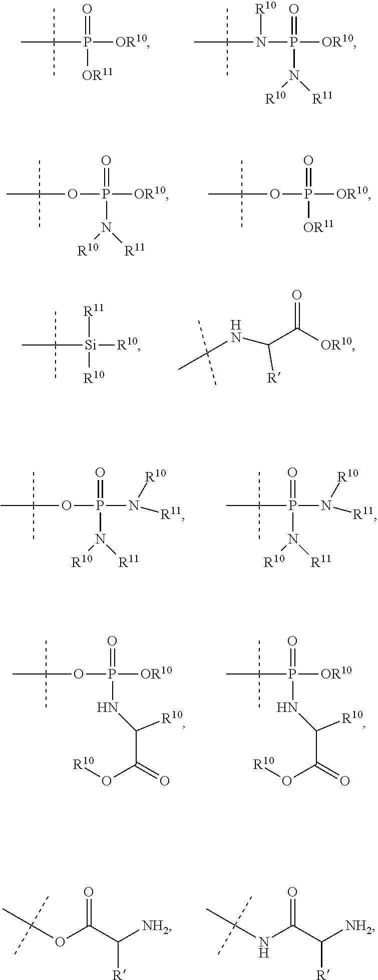
and —O—S(O)₂—OH,
R¹⁰ and R¹¹ are, independently, H, C₁₋₆ alkyl, C₁₋₆ haloalkyl, C₂₋₆ alkenyl, C₂₋₆ alkynyl, aryl, heteroaryl, alkylaryl, arylalkyl, a six-membered ring or a six-membered bridged or spiro-fused ring containing zero, one, or two heteroatoms which are, independently, N, O, or S, a seven-membered bridged or spiro-fused ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero or one heteroatom which are, independently, N, O, or S,
alternatively, R¹⁰ and R¹¹ can come together to form a saturated or partially unsaturated 5 to 8 membered ring or a six-membered bridged or spiro-fused ring containing zero, one, or two heteroatoms which are, independently, N, O, or S, a seven-membered bridged or spiro-fused a saturated or partially unsaturated ring containing zero, one, or two heteroatoms which are, independently, N, O, or S, a five-membered ring containing zero, one, or two heteroatoms which are, independently, N, O, or S; a four-membered ring containing zero, one, or two heteroatoms which are, independently, N, O, or S; or a three membered ring containing zero heteroatoms, a saturated or partially unsaturated 6 to 12 membered hetero-bicyclic ring; or a saturated or partially unsaturated 6 to 12 membered hetero-tricyclic ring; and
R¹⁰ and R¹¹ groups can optionally be substituted with one or more substituents, which substituents are, independently, halo, C₁₋₆ alkyl, C₃₋₆ alkyl, C₁₋₆ haloalkyl, C₂₋₆ alkenyl, C₂₋₆ alkynyl, C₁₋₆ hydroxy alkyl, hydroxyl, carboxyl, acyl, aryl, acyloxy, amino, amido, carboxyl derivatives, alkylamino, dialkylamino, arylamino, alkoxy, alkoxyalkyl, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, oxime, hydrazine, carbamate, phosphonic acid, phosphoramidate or phosphonate.
R¹⁴ and R¹⁵ are independently, H, C₁₋₆ alkyl, C₁₋₆ haloalkyl, C₂₋₆ alkenyl, C₂₋₆ alkynyl,
R¹⁴ and R¹⁵ can come together to form an optionally substituted 3 to 5 membered ring, and
R¹⁶ is H or C₁₋₆ alkyl.

32. The compound of claim 31, wherein R′ is restricted to those occurring in natural amino acids

33. The compound of claim 31, wherein G is phenyl.

34. The compound of claim 31, wherein F is pyrrolyl.

35. The compound of claim 34, wherein the 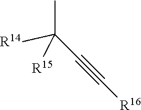
substituent is attached to the ring nitrogen.

36. The compound of claim 31, wherein R¹ is selected from the group consisting of F, CN and OH.

37. The compound of claim 31, wherein R¹ is methyl.

38. A compound selected from the group consisting of: 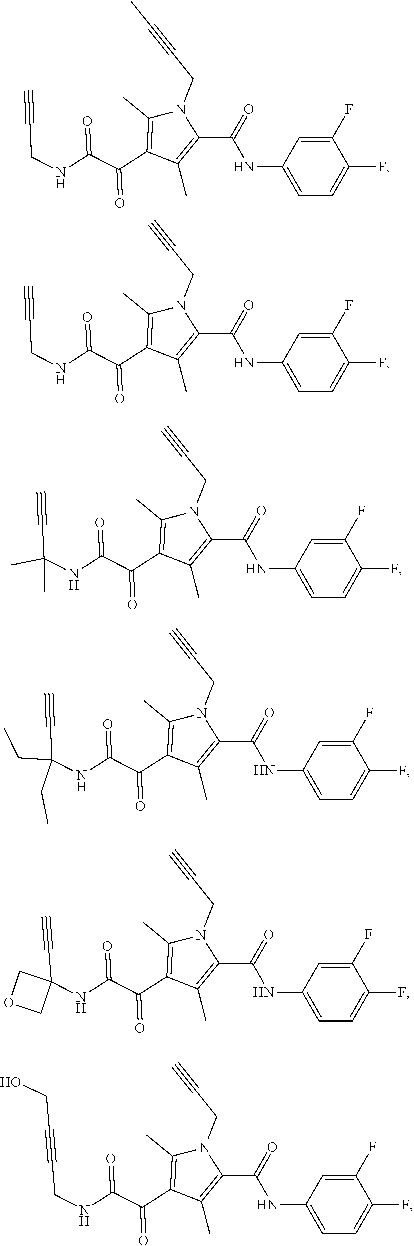 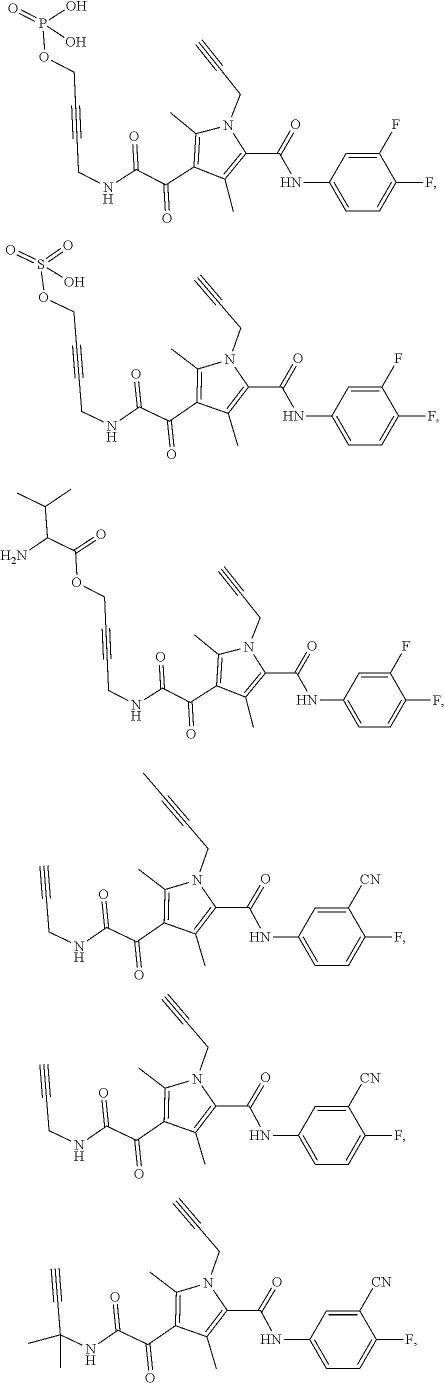 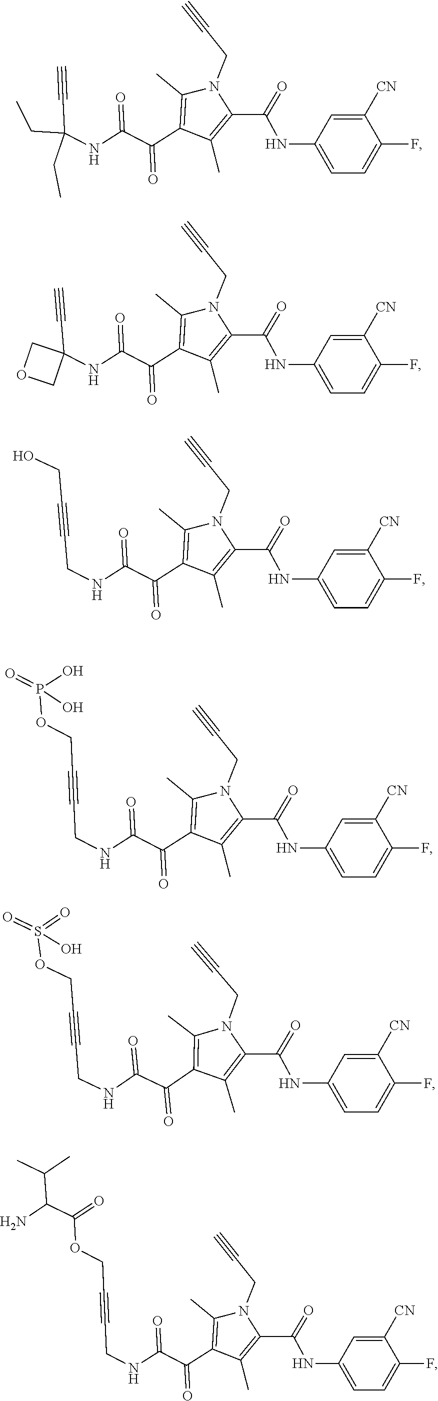 [SMILES: C#CCn1c(C)c(C(=O)C(=O)NC(C#C)(CC)CC)c(C)c1C(=O)Nc1ccc(F)c(C#N)c1 ; C#CCn1c(C)c(C(=O)C(=O)NC2(C#C)COC2)c(C)c1C(=O)Nc1ccc(F)c(C#N)c1 ; C#CCn1c(C)c(C(=O)C(=O)NCC#CCO)c(C)c1C(=O)Nc1ccc(F)c(C#N)c1 ; C#CCn1c(C)c(C(=O)C(=O)NCC#CCOP(=O)(O)O)c(C)c1C(=O)Nc1ccc(F)c(C#N)c1 ; C#CCn1c(C)c(C(=O)C(=O)NCC#CCOS(=O)(=O)O)c(C)c1C(=O)Nc1ccc(F)c(C#N)c1 ; C#CCn1c(C)c(C(=O)C(=O)NCC#CCOC(=O)C(N)C(C)C)c(C)c1C(=O)Nc1ccc(F)c(C#N)c1]
and pharmaceutically acceptable salts and prodrugs thereof.

39. A pharmaceutical composition comprising a compound of any of claims 31 to 38, and a pharmaceutically-acceptable carrier.

40. The composition of claim 39, wherein the composition is a transdermal composition or a nanoparticulate composition.

41. The pharmaceutical composition of claim 40, further comprising a second antiviral agent.

41. The pharmaceutical composition of claim 41, wherein the second antiviral agent is selected from the group consisting of a polymerase inhibitors, viral entry inhibitor, a viral maturation inhibitor, a literature described capsid assembly modulator, an IMPDH inhibitor, a protease inhibitor, an immune-based therapeutic agents, a reverse transcriptase inhibitor, a TLR-agonist, siRNA, shRNA, Talens, Crisper/Cas9, mir (microRNA), an agent of distinct or unknown mechanism, and combinations thereof.

42. Use of a compound of any of claims 31-38 in the preparation of a medicament for treating HBV infection, preventing an HBV infection, or reducing the biological activity of an infection with HBV.

43. A method for treating a host infected with HBV, preventing an HBV infection, curing an HBV infection, or reducing the biological activity of an infection with HBV in a host, comprising administering an effective amount of a compound of any of claims 31-38 to a patient in need of treatment thereof.

44. The method of claim 43, wherein the method further comprising administering another anti-HBV agent in combination or alternation with the compound of any of claims 31-32.

45. The compound of claim 1, wherein LI is a C₂₋₁₆ alkyl, optionally with one or more triazine rings at one or more positions along the alkyl chain.

46. The compound of claim 1, wherein LI is a (CH₂—CH₂—O)ₙ linker, where n is an integer from 2 to 16.

47. A compound having one of the following formulas: 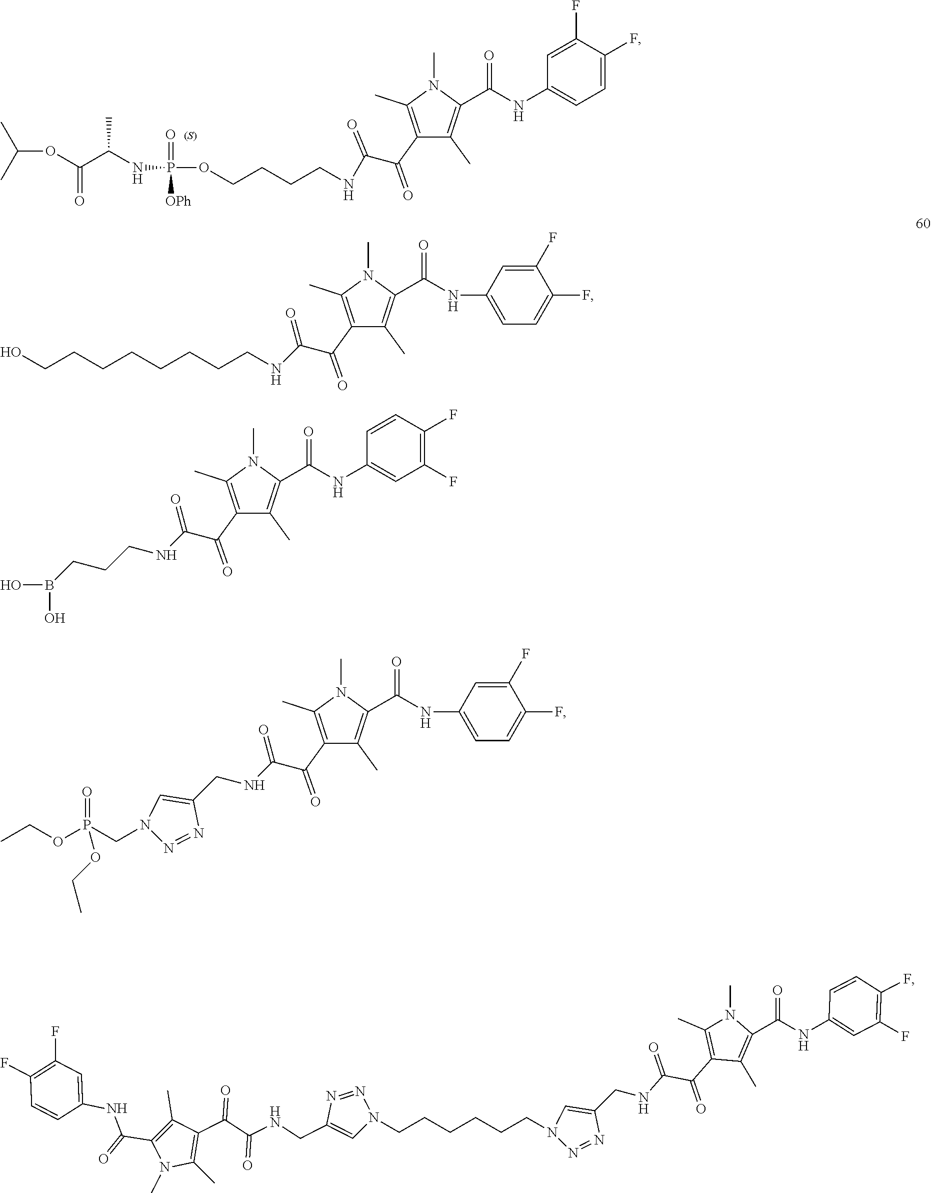 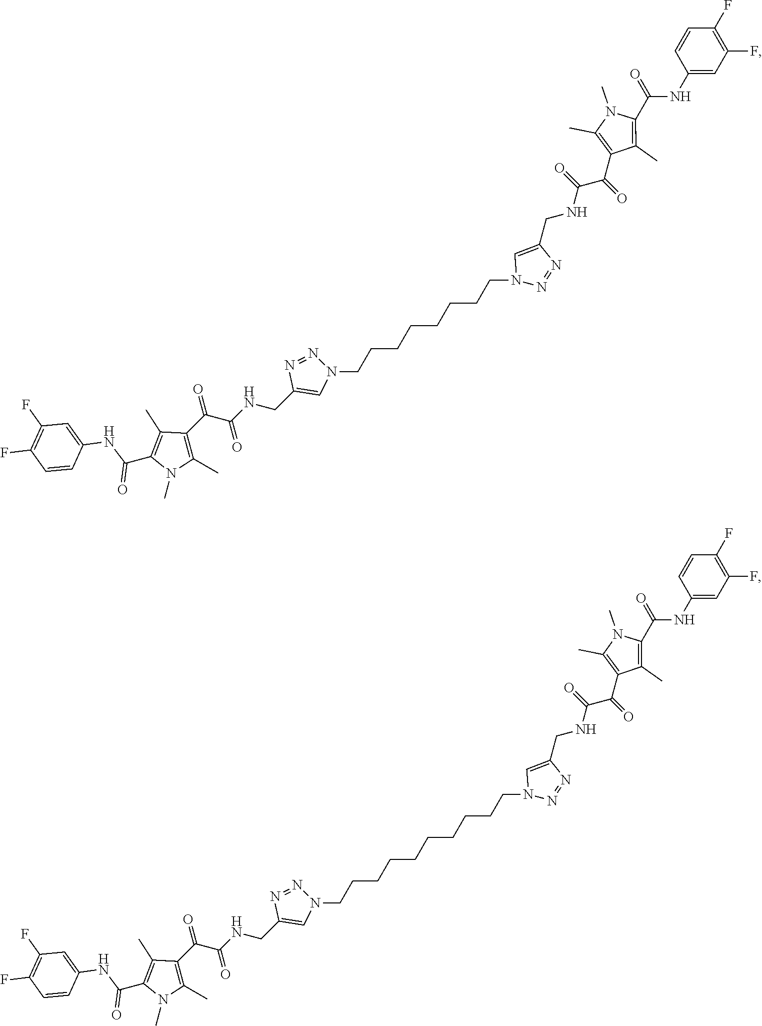 [SMILES: Cc1c(C(=O)C(=O)NCc2cn(CCCCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1 ; Cc1c(C(=O)C(=O)NCc2cn(CCCCCCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1] 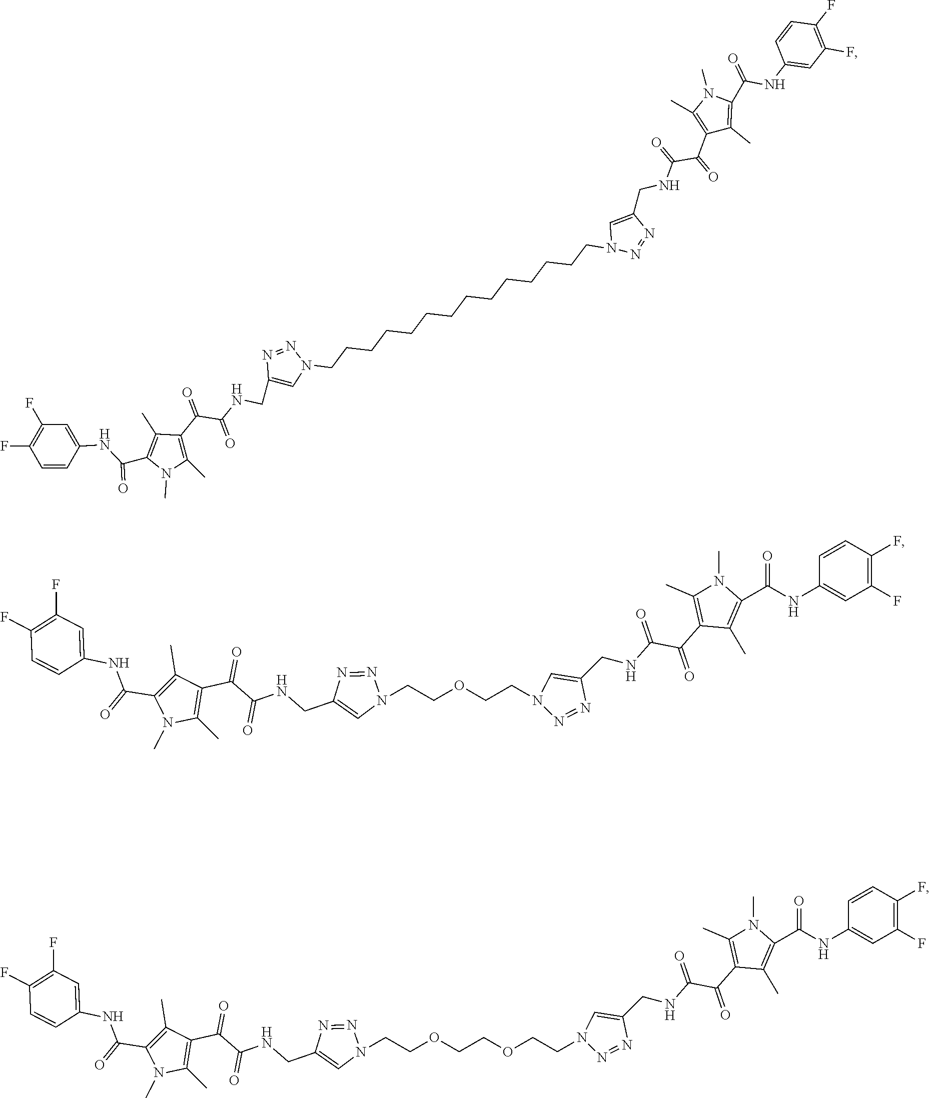 [SMILES: Cc1c(C(=O)C(=O)NCc2cn(CCCCCCCCCCCCCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1 ; Cc1c(C(=O)C(=O)NCc2cn(CCOCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1 ; Cc1c(C(=O)C(=O)NCc2cn(CCOCCOCCn3cc(CNC(=O)C(=O)c4c(C)c(C(=O)Nc5ccc(F)c(F)c5)n(C)c4C)nn3)nn2)c(C)n(C)c1C(=O)Nc1ccc(F)c(F)c1] 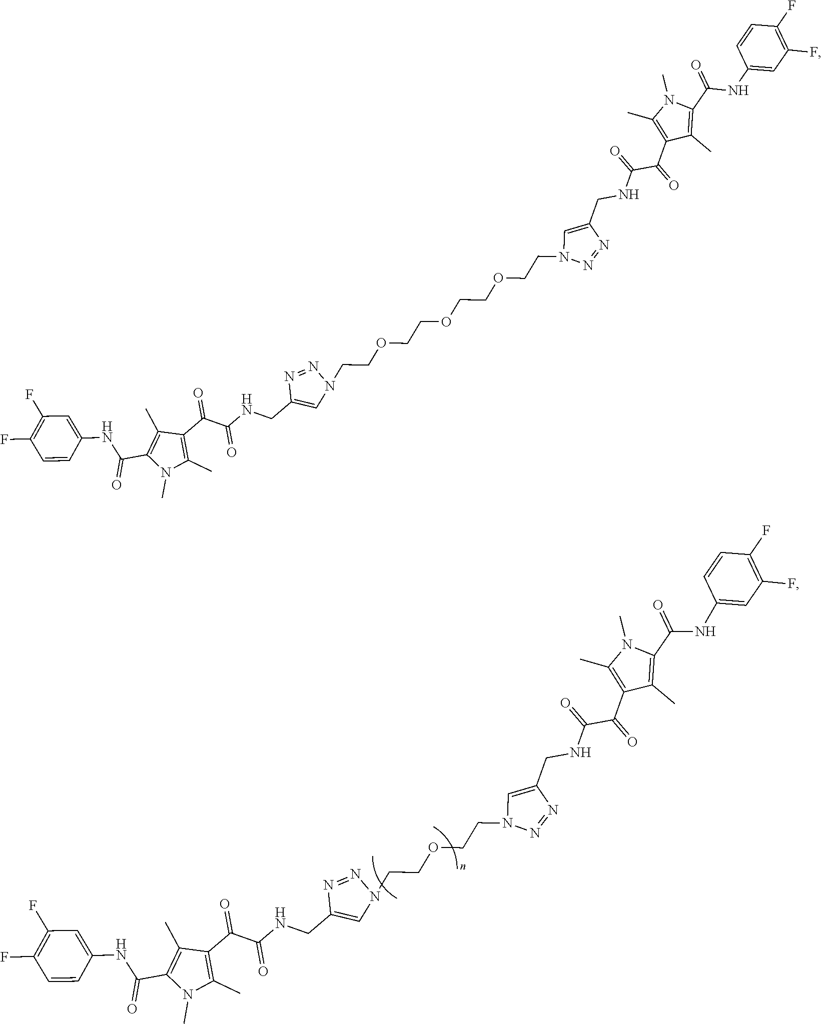 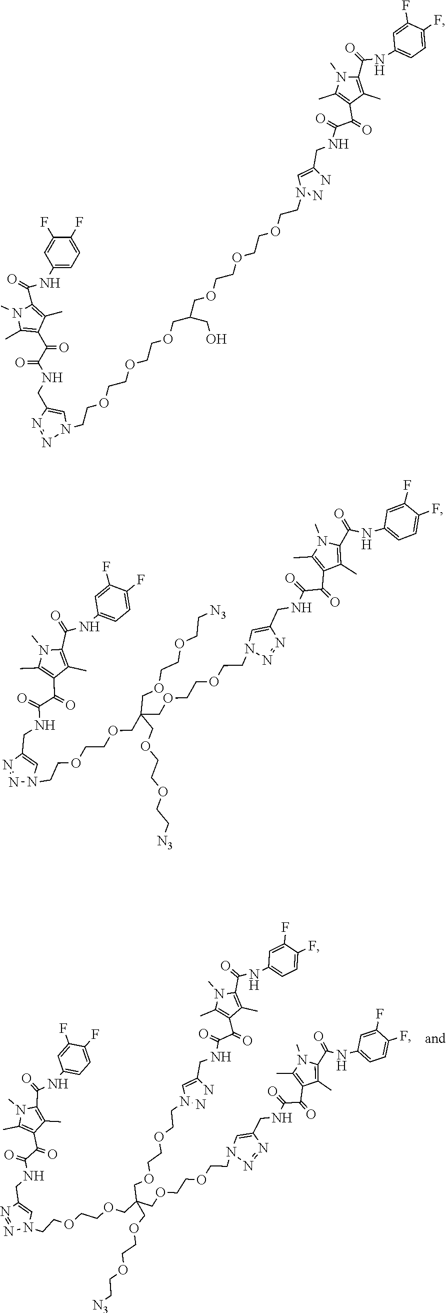 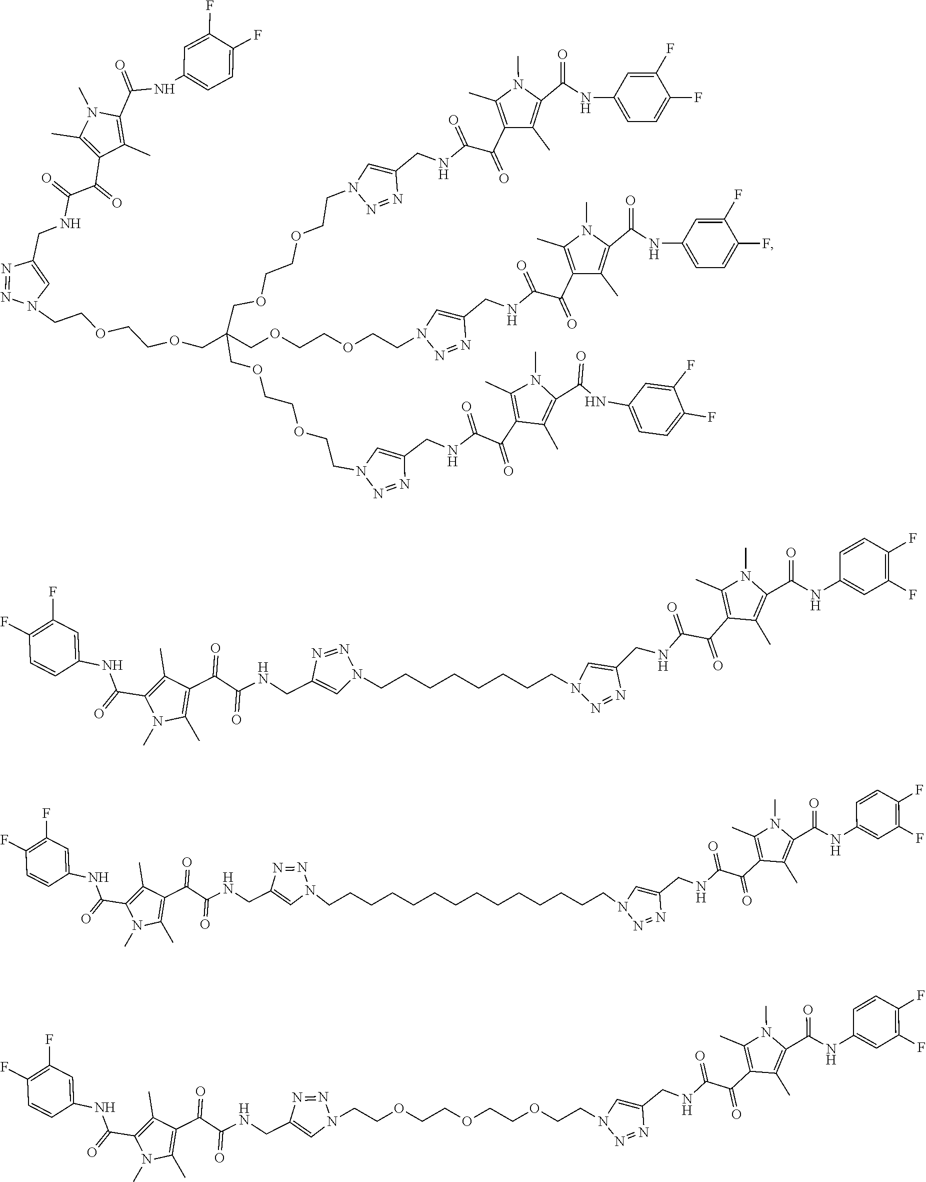
or an enantiomer, diasteroisomer, tautomer, pharmaceutically acceptable salt or prodrug thereof.

42. A compound of the following formula: 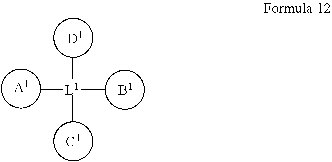
wherein:
A¹, B¹, C¹ and D¹ are, independently, an HBV capsid assembly effector,
each A¹, B¹, C¹ and D¹ may be the same or different;
L¹ is a chemical linker selected from the group consisting of —C—((C₂₋₆ alkyl-O)ₚ)₄—, —C—(C₁₋₁₆ alkyl-)₄, —C(NR′)₃—)₄, C(C₁₋₆ alkyl-W¹—)₄; —C((CH₂—CH₂—O)ₜ—C₁₋₆ alkyl-W¹—)₄; —C((CH₂—CH₂—O)ₛ)₄—; C(C₁₋₃₀ alkyl-W¹—)₄; C—((C₃₋₆ alkyl-O)ₛ—C₁₋₆ alkyl-X—(C₃₋₆ alkyl-O)ₛ—C₁₋₆ alkyl-)₄; C((CH₂—CH₂—O)ₜ—C₁₋₆ alkyl-X—(CH₂−CH₂—O)ₜ—C₁₋₆ alkyl-W¹—)₄, or C(C₁₋₃₀ alkyl-X—C₁₋₃₀ alkyl-W¹—)₄;
p, s and m are integers from 1 to 15;
t is an integer from 3 to 15;
X is Si(R⁷)(R⁸)—, —Si(R⁷)(R⁸)—O—, —O—Si(R⁷)(R⁸)—, —NR′—; —N(R′)C(O)—; —C(O)N(R′)—; —N(R′)SO₂—; —SO₂N(R′)—; —O—; —C(O)—; —OC(O)—; —C(O)O—; —S—; —S(O)—; —SO₂—; —C(═S)—; or —C(═NR′)—;
wherein the optional substituents for the heteroaliphatic, aliphatic, aryl, and heteroaryl moieties are selected from the group consisting of halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, phosphonyl, phosphinyl, phosphoryl, phosphine, thioester, thioether, oxime, hydrazine, carbamate, silyl ether, boronic ester, boronic acid, phosphonic acid, phosphonamidate, and phosphonate,
W¹ is, independently for each occurrence, aryl, heteroaryl, CHR′, NR′, N, O or S;
R′ is H, C₁₋₆ alkyl, optionally unsaturated C₃₋₈ cycloalkyl, C₁₋₆ haloalkyl, C₂₋₈ alkoxyalkyl, C₂₋₆ alkenyl, C₂₋₆ alkynyl, aryl, heteroaryl, alkylaryl, or arylalkyl, R⁷ and R⁸ are, independently, —OH, optionally unsaturated —O—C₁₋₆ alkyl, C₁₋₆ alkyl, C₂-6 alkenyl, C₂₋₆ alkynyl, optionally unsaturated C₃₋₈ cycloalkyl, aryl, heteroaryl, alkylaryl, or arylalkyl, and
alternatively, R⁷ and R⁸, together with the silicon to which they are attached, form a 4-7 membered ring, optionally containing one or more heteroatoms, where each is, independently, O, S or N, wherein the 4-7 membered ring is optionally substituted with one or more substituents selected from the group consisting of C₁-C₃ alkyl, halogen, cyano, oxo and OH;
or an enantiomer, diasteroisomer, tautomer, pharmaceutically acceptable salt or prodrug thereof.
